(19) United States
(12) Patent Application Publication
(10) Pub. No.: US 2026/0224708 A1
(43) Pub. Date: Aug. 6, 2026

(54) CONJUGATE COMPOUNDS AND COMPOSITIONS

(71) Applicant: REZUBIO PHARMACEUTICALS CO., LTD, Zhuhai, Guangdong (CN)

(72) Inventors: Yusheng XIONG, Zhuhai, Guangdong (CN); Hongping GUAN, Zhuhai, Guangdong (CN)

(21) Appl. No.: 19/154,188

(22) PCT Filed: Feb. 1, 2024
(86) PCT No.: PCT/CN2024/075112

§ 371 Date: Aug. 6, 2025

Related U.S. Application Data
(60) Provisional application No. 63/527,807, filed on Jul. 19, 2023.

(30) Foreign Application Priority Data
Feb. 7, 2023 (WO) PCT/CN2023/074733

(51) Int. Cl. A61K 47/54; A61K 45/06; A61P 3/10

(52) U.S. Cl. CPC A61K 47/545; A61K 45/06; A61P 3/10

(57) ABSTRACT

Provided herein are novel compounds of Formula I, pharmaceutical compositions, and methods of using related to membrane bound protein, such as GPR40. The compounds herein are typically GPR40 agonists, which can be used for treating a variety of disorders, conditions or diseases such as Type 2 diabetes.

This application claims the benefit of priority to International Application No. PCT/CN2023/074733, filed Feb. 7, 2023, and U.S. Provisional Application No. 63/527,807, filed Jul. 19, 2023, the content of each of which is herein incorporated by reference in its entirety.

In various embodiments, the present disclosure generally relates to novel compounds, pharmaceutical compositions, and methods of using the same, such as for treating Type 2 diabetes mellitus.

### BACKGROUND

Type 2 diabetes mellitus is a form of diabetes that is characterized by high blood sugar, insulin resistance, and relative lack of insulin. There are several available treatments for Type 2 diabetes, each of which has its own limitations and potential risks. Pharmacologic treatments for diabetes have largely focused on: (1) hepatic glucose production (biguanides, such as phenformin and metformin), (2) insulin resistance (PPAR agonists, such as rosiglitazone, troglitazone, engliazone, balaglitazone, netoglitazone, T-131, LY-300512, LY-818 and pioglitazone), (3) insulin secretion (sulfonylureas, such as tolbutamide, glipizide and glimipiride); (4) incretin hormone mimetics (GLP-1/GIP derivatives and analogs, such as exenatide, liraglutide, dulaglutide, semaglutide, lixisenatide, albiglutide, taspoglutide, and tirzepatide); (5) inhibitors of incretin hormone degradation (DPP-4 inhibitors, such as sitagliptin, alogliptin, vildagliptin, linagliptin, denagliptin and saxagliptin); and (6) SGLT2 inhibitors (canagliflozin, dapagliflozin, empagliflozin, and ertugliflozin).

G-protein-coupled receptor 40 (GPR40) is a cell-surface GPCR that is highly expressed in human (and rodent) islets as well as in insulin-secreting cell lines. The human G-protein-coupled receptor hGPR40 is primarily localized in pancreatic β cells and intestinal enteroendocrine cells. GPR40 is also reported to be expressed in the brain (hippocampus and hypothalamus), hepatocytes, and macrophages. Medium- to long-chain fatty acids (FFAs) are endogenous ligands of GPR40. Upon binding to GPR40, FFAs trigger a signaling cascade that results in increased levels of [Ca²⁺] in β-cells and subsequent stimulation of insulin secretion. In the gut, FFAs also stimulate secretion of incretins, including glucagon-like peptide-1 (GLP-1) and glucose-dependent insulinotropic polypeptide (GIP), cholecystokinin (CCK), and peptide YY (PYY). The recent recognition of the function of GPR40 in modulating insulin secretion has provided insights into regulation of carbohydrate and lipid metabolism in vertebrates, and further provided targets for the development of therapeutic agents for metabolic disorders such as obesity, diabetes, cardiovascular disease and dyslipidemia.

Agonists of G-protein-coupled receptor 40 (GPR40) have been shown to be useful in treating type 2 diabetes mellitus, obesity, hypertension, dyslipidemia, cancer, and metabolic syndrome, as well as cardiovascular diseases, such as myocardial infarction and stroke. New GPR40 agonists that have pharmacokinetic and pharmacodynamic properties suitable for use as human pharmaceuticals are needed. Similarly, new ligands for other membrane bound proteins such as other GPCRs that have pharmacokinetic and pharmacodynamic properties suitable for use as human pharmaceuticals are also needed.

### BRIEF SUMMARY

Provided herein are compounds, pharmaceutical compositions, and methods of using related to membrane bound proteins, such as GPR40. The compounds herein are typically GPR40 agonists, which can be used for treating a disorder, condition or disease such as Type 1 or 2 diabetes, obesity, hyperglycemia, glucose intolerance, insulin resistance, hyperinsulinemia, hypercholesterolemia, hypertension, hyperlipoproteinemia, hyperlipidemia, myocardial infarction, stroke, hypertriglyceridemia, dyslipidemia, metabolic syndrome, syndrome X, cardiovascular disease, atherosclerosis, kidney disease, diabetic kidney disease, ketoacidosis, thrombotic disorders, nephropathy, diabetic neuropathy, diabetic retinopathy, sexual dysfunction, dermatopathy, dyspepsia, hypoglycemia, cancer, edema, nonalcoholic steatohepatitis (NASH), lipodystrophy, Prader Willi syndrome, inflammatory bowel diseases including Crohn's disease and ulcerative colitis, irritable bowel syndrome, short bowel syndrome, lymphocytic colitis, rare microscopic colitis, and/or neurodegenerative diseases including but not limited to Alzheimer's disease, Parkinson's disease, amyotrophic lateral sclerosis.

Some embodiments of the present disclosure are directed to compounds of Formula I, or pharmaceutically acceptable salts or esters thereof:

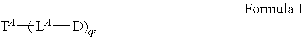

wherein the variables are defined herein. In some embodiments, D can be D-1 (e.g., D-1-A, D-1-B, D-1-A-1, D-1-A-2, D-1-A-3, D-1-A-4, D-1-A-5, D-1-A-6, D-1-A-7, D-1-A-8, D-1-A-9, or D-1-A-10, etc.), as defined herein. In some embodiments, D can be D-2-A (e.g., D-2-A-1, D-2-A-2, or D-2-A-3), as defined herein. In some embodiments, D can be D-2-B (e.g., D-2-B-1, D-2-B-2, or D-2-B-3), as defined herein. In some embodiments, D can be D-3-A (e.g., D-3-A-1, D-3-A-2, or D-3-A-3), as defined herein. In some embodiments, D can be D-3-B (e.g., D-3-B-1, D-3-B-2, or D-3-B-3), as defined herein. In some embodiments, the compound of Formula I can have a subformula according to Formula I-1, I-1-A, I-1-B, I-1-C, I-1-D, I-1-E, I-1-F, I-1-G, I-1-G-1, I-1-G-2, I-1-G-3, I-1-G-4, I-1-G-5, I-1-G-6, I-1-G-7, I-1-G-8, I-1-G-9, I-1-H, I-1-I, I-1-A-1, I-1-B-1, I-1-C-1, I-1-D-1, I-1-E-1, I-1-F-1, I-1-H-1, or I-1-I-1, as defined herein.

In some embodiments, the present disclosure also provides a compound selected from Table 1 herein, or a pharmaceutically acceptable salt or ester thereof. In some embodiments, the present disclosure also provides a compound selected from Examples 1-36 herein, or a pharmaceutically acceptable salt or ester thereof.

In some embodiments, the present disclosure provides a pharmaceutical composition comprising one or more compounds of the present disclosure and optionally a pharmaceutically acceptable excipient. In some embodiments, the pharmaceutical composition comprises a compound of Formula I (e.g., Formula I-1, I-1-A, I-1-B, I-1-C, I-1-D, I-1-E, I-1-F, I-1-G, I-1-G-1, I-1-G-2, I-1-G-3, I-1-G-4, I-1-G-5, I-1-G-6, I-1-G-7, I-1-G-8, I-1-G-9, I-1-H, I-1-I, I-1-A-1, I-1-B-1, I-1-C-1, I-1-D-1, I-1-E-1, I-1-F-1, I-1-H-1, or I-1-I-1), or any of the compounds listed in Table 1 herein, any of the compound according to Examples 1-36 herein, or a pharmaceutically acceptable salt or ester thereof, and a pharmaceutically acceptable excipient. The pharmaceutical composition can be typically formulated for oral administration. In some embodiments, the pharmaceutical composition is administered to a subject in need to deliver an effective amount of GPR40 agonist in the gastrointestinal tract with minimal or no absorption of GPR40 agonist in systemic circulation.

In some embodiments, the present disclosure provides a method of treating or preventing a disorder, condition or disease that may be responsive to the activation of the GPR40 in a subject in need thereof. In some embodiments, the method comprises administering to the subject an effective amount of one or more compounds of the present disclosure or the pharmaceutical composition herein. In some embodiments, the method comprises administering to the subject an effective amount of a compound of Formula I (e.g., Formula I-1, I-1-A, I-1-B, I-1-C, I-1-D, I-1-E, I-1-F, I-1-G, I-1-G-1, I-1-G-2, I-1-G-3, I-1-G-4, I-1-G-5, I-1-G-6, I-1-G-7, I-1-G-8, I-1-G-9, I-1-H, I-1-I, I-1-A-1, I-1-B-1, I-1-C-1, I-1-D-1, I-1-E-1, I-1-F-1, I-1-H-1, or I-1-I-1), or any of the compounds listed in Table 1 herein, any of the compound according to Examples 1-36 herein, or a pharmaceutically acceptable salt or ester thereof, or a pharmaceutical composition comprising the same. In some embodiments, the administering is an oral administration.

In some embodiments, the present disclosure provides a method of treating type 2 diabetes mellitus in a subject in need thereof. In some embodiments, the method comprises administering to the subject a therapeutically effective amount of one or more compounds of the present disclosure or the pharmaceutical composition herein. In some embodiments, the method comprises administering to the subject a therapeutically effective amount of a compound of Formula I (e.g., Formula I-1, I-1-A, I-1-B, I-1-C, I-1-D, I-1-E, I-1-F, I-1-G, I-1-G-1, I-1-G-2, I-1-G-3, I-1-G-4, I-1-G-5, I-1-G-6, I-1-G-7, I-1-G-8, I-1-G-9, I-1-H, I-1-I, I-1-A-1, I-1-B-1, I-1-C-1, I-1-D-1, I-1-E-1, I-1-F-1, I-1-H-1, or I-1-I-1), or any of the compounds listed in Table 1 herein, any of the compound according to Examples 1-36 herein, or a pharmaceutically acceptable salt or ester thereof, or a pharmaceutical composition comprising the same. In some embodiments, the administering is an oral administration.

In some embodiments, the method herein further comprises administering to the subject an additional therapeutic agent. In some embodiments, the additional therapeutic agent can be PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; α-glucosidase inhibitors; agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARα agonists, (v) cholesterol absorption inhibitors, (vi) acyl CoA:cholesterol acyltransferase (ACAT) inhibitors, (vii) CETP inhibitors, (viii) PCSK9 inhibitor or antibodies; (ix) apolipoproteins inhibitors; (x) phenolic anti-oxidants; PPARα/γ dual agonists; PPARδ agonists; PPAR α/δ partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists (peptide and small-molecule); GLP-1/GIP receptor dual agonists; GLP-1/glucagon receptor dual agonists; GLP-1/GIP/insulin receptor triple agonists; GLP-1/GIP/glucagon receptor triple agonists; GIP receptor antibody; GLP-1 analog/GIP receptor antibody; PYY analog; amylin analogs; GPR119 agonist; TGR5 agonist; SSTR2 and/or SSTR5 antagonist or inverse agonist; THRβ agonists; HSD-1 inhibitors; HSD-17 inhibitors and degraders; PNPLA3 inhibitors and degraders; SGLT-2 inhibitors; SGLT-1/SGLT-2 inhibitors; enteric alpha-glucosidase inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin; (xi) anti-amyloid beta antibody; (xii) anti-inflammatory agents including but not limited to PDE4 inhibitors, JAK inhibitors, TYK2 inhibitors, SIP receptor modulators, NLRP3 inhibitors, BTK inhibitors, IRAK1 inhibitors, IRAK4 inhibitors, glucocorticoids, anti-TNFα antibodies, anti-IL-12/IL-23 antibodies, (xiii) anti-integrin antibodies or small-molecule inhibitors of integrins including α4β7, α4, β7, MAdCAM-1, αvβ6 and αvβ1.

It is to be understood that both the foregoing summary and the following detailed description are exemplary and explanatory only, and are not restrictive of the invention herein.

### BRIEF DESCRIPTION OF THE DRAWINGS

FIG. 1 presents a graph showing the changes of blood glucose concentrations over time following treatments of mice with exemplary compounds, Example 17, 18, and 19, or control.

FIG. 2 presents a bar graph showing the relative AUC of blood glucose concentrations over 0-120 minutes following treatments with exemplary compounds, Examples 17, 18, and 19, in comparison with that of control.

### DETAILED DESCRIPTION

In various embodiments, the present disclosure provides compounds that are useful for modulating a membrane bound protein, such as a GPCR, in particular GPR40. The compounds herein typically have no or reduced systemic exposure and therefore are expected to have reduced side effects due to such systemic exposure. In some embodiments, the present disclosure also provides pharmaceutical compositions comprising the compound(s) and methods of using the same, such as in treating type 2 diabetes. As shown in the examples section herein, exemplary compounds were shown to be active in the oral glucose tolerance test with minimal systemic exposure. The oral bioavailability (F %) of the tested compounds (see Biological Example 2) are all below 1%, most of them below 0.5%.

### Compounds

International Application No. PCT/CN2021/109496, filed Jul. 30, 2021, the content of which is incorporated herein by reference in its entirety, describes various conjugates of a GPR40 agonist covalently linked to a carrier. As described in the '496 application and without wishing to be bound by theories, it is believed that when administered, conjugates of GPR40 agonist can have advantages such as modulating GPR40 without side effects or with reduced side effects due to reduced systemic exposure.

International Application No. PCT/CN2023/071833, filed Jan. 12, 2023, now published as WO2023/134712, the content of which is incorporated herein by reference in its entirety, describes that certain GPR40 agonists, if covalently linked to a polar group through a hydrophobic linker with sufficient chain length, the resulted compounds can be highly potent GPR40 agonists, with an EC50 value less than 50 nM, less than 10 nM, or below 1 nM, when tested according to the methods described therein.

In a broad aspect, compounds described herein can be viewed as having one or more ligands of a membrane bound protein, such as GPR40 agonist(s), covalently linked to a hydrophilic group through a linker. Typically, compounds described herein have one GPR40 agonist(s) covalently linked to a hydrophilic group through a linker: (GPR40 agonist)-Linker-Hydrophilic group. In some embodiments, the linker and hydrophilic group together may be viewed as a residue of a surfactant, such as an anionic surfactant, a cationic surfactant, a zwitterionic surfactant, or a nonionic surfactant. For example, in some embodiments, the linker and hydrophilic group can be such that upon binding of the compound herein with a membrane bound protein, such as GPR40, the hydrophobic linker is bound within a cell membrane whereas the hydrophilic group is not.

In some more specific embodiments, the compounds may be typically viewed as connecting the residue of GPR40 agonist of

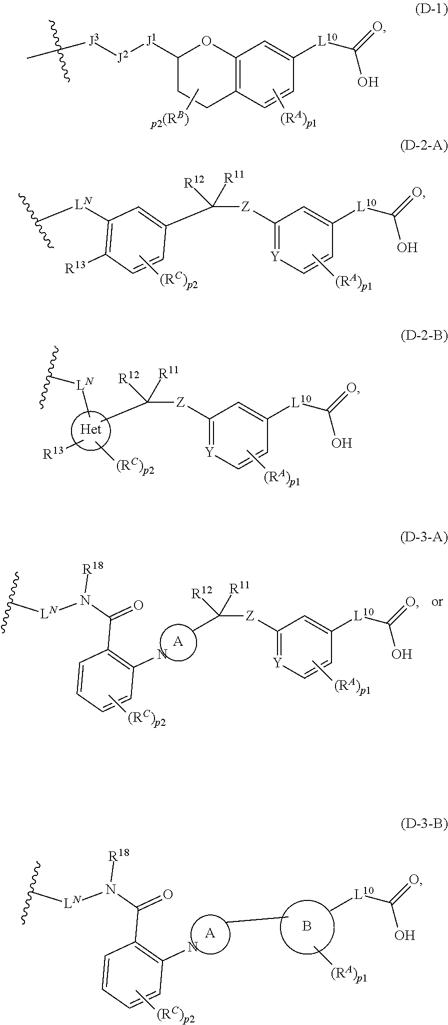

with a hydrophilic group T^{A} through a linker L^{A}, wherein the variables are described and preferred herein. To be clear, the dissection of the compounds as residue of GPR40 agonist, linker, and hydrophilic group is merely for convenience of discussions herein, not to limit the compounds herein in any way. For example, for the same compound, there may be different ways to attribute certain structural fragments to be part of the residue of the GPR40 agonist, L^{A}, or T^{A}. For the purposes herein, in such situations, if under one of the ways of attribution, all of the residue of the GPR40 agonist, L^{A}, and T^{A} of the compound are within a respective definition of a genus of compounds herein, then the compound can be said to be within the scope of that genus. Typically, the variables in D-1, D-2-A, D-2-B, D-3-A, or D-3-B are such that at least one of the corresponding compounds according to Formula GPR-1, GPR-2, GPR-2B, GPR-3, or GPR-3B is a GPR40 agonist, preferably, having an EC50 of less than 100 nM as measured according to Biological Example 1 herein:

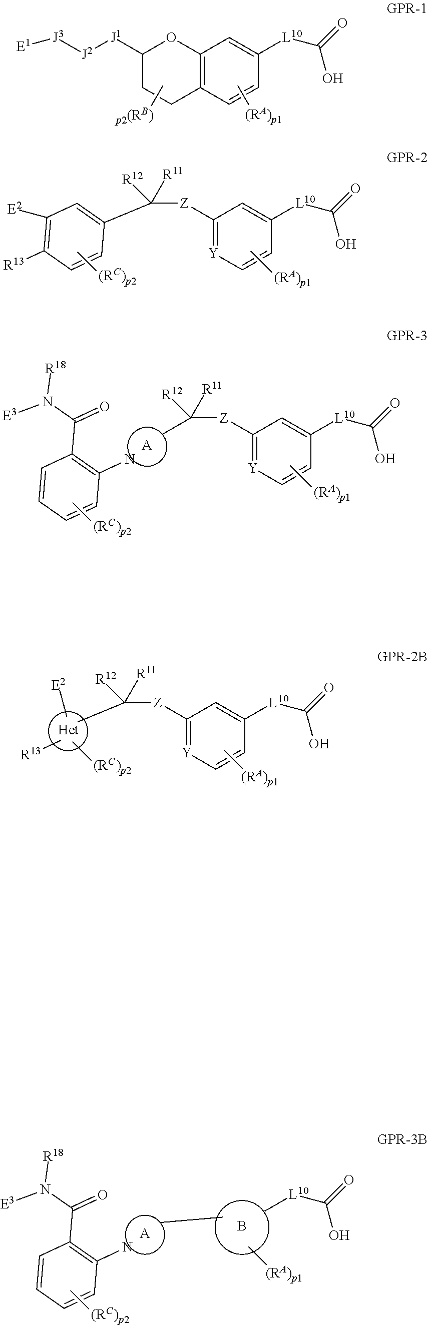

wherein E² is E^{2A} or L^{N}-E^{2A}, wherein E¹ or E^{2A} is hydrogen, C₁₋₄alkyl, N3,

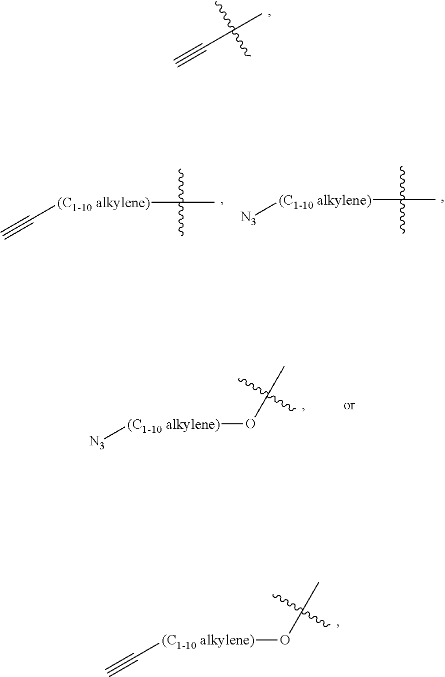

wherein E³ is E^{3A} or L^{N}-E^{3A}, wherein E^{3A} is hydrogen, C₁₋₄ alkyl, N3,

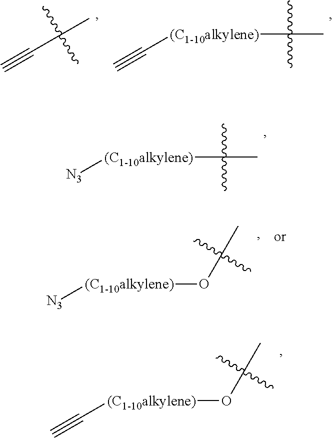

and L^{N} is defined herein (such as null or a C₁₋₆ alkylene). In preferred embodiments, the compounds herein with the residue D-1, D-2-A, D-2-B, D-3-A, or D-3-B covalently linked to the hydrophilic group T^{A} have a similar EC50 (e.g., within 3-fold) or lower EC50 value compared to at least one (preferably all) of the corresponding compounds of Formula GPR-1, GPR-2, GPR-2B, GPR-3, or GPR-3B.

### Formula I

In some embodiments, the present disclosure provides a compound of Formula I, or a pharmaceutically acceptable salt or ester thereof:

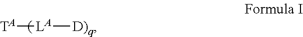

wherein:
D is a residue of a ligand of a membrane bound protein, such as a GPCR, preferably, D is a residue of a GPR40 agonist;
q is an integer of 1-10, preferably, 1 or 2;
L^{A} is a hydrophobic linker; and
T^{A} is a group characterized as having one or more (e.g., 1, 2, or 3) hydrophilic polar groups, preferably, T^{A} is a group characterized as having one or more (e.g., 1, 2, or 3) charged groups, such as having one or more quaternary amine, one or more carboxylic acid, one or more phosphoric acid, and/or one or more sulfonic acid,
   wherein the compound is charge balanced as necessary. The term “polar group(s)” as used herein refer to a functional group that contains at least one heteroatom selected from N, O, P, and S. In some embodiments, a “polar group” can be a charged group, which refers to a functional group that contains at least one charge, such as at least one positive charge, at least one negative charge, or in the case of a zwitterion, both a positive and negative charges, at pH of 7. As used herein, a “hydrophilic” organic group refers to a functional group in which (i) at least one, preferably, at least 2 or at least 3, neutral hydrophile group (e.g., O, OH, etc.) per 5 carbons, and/or (ii) at least one, preferably, at least 2, at least 3, or at least 4, electrically charged hydrophile group (e.g., charged amine groups such as quaternary amine groups, chargeable amine groups, COOH groups, SO3H groups, etc.) per 7 carbons. In some embodiments, a “hydrophilic” organic group is characterized as having a ratio of (total number of nitrogen and oxygen atoms)/(total number of carbon atoms) of 4:1-1:4, such as 3:1, 2:1, 1:1, 1:2, 1:3, or 1:4, or any ranges or values between the recited values. As used herein, a “hydrophobic” molecule general refers to those molecules having a c Log P of at least 3.

In Formula I, the integer q is typically 1, and the compound can have a structure according to Formula I-1:

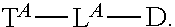

### Hydrophilic Group T^{A}

As shown herein and partly in PCT/CN2023/071833, when D represents a residue of a GPCR ligand, the inventors found that several factors are important for the compound of Formula I to be a potent GPCR ligand, such as a GPR40 agonist. For example, one factor that can determine whether the compound of Formula I (e.g., I-1) can be a potent GPCR ligand, such as GPR40 agonist is the hydrophilicity or polarity of T^{A}, but the exact chemical structure of T^{A} is not as important.

In some preferred embodiments, T^{A} can contain a charged group, including positively charged, such as containing a quaternary nitrogen atom, negatively charged, such as CO₂⁻, SO₃⁻, or containing a zwitterion structure. When T^{A} contains a charged group, it should be understood that a counterion, preferably, a pharmaceutically acceptable anion or cation, if necessary, exists to balance the charges so that the compound of Formula I is overall neutral. Pharmaceutically acceptable anions are known in the art, which are typically derived from a pharmaceutically acceptable acid, e.g., Cl⁻, etc. Pharmaceutically acceptable cations are also known in the art, such as alkali cations such as Na⁺, etc.

In some preferred embodiments, T^{A} is a group characterized as having one or more (e.g., 1, 2, or 3) quaternary amine, one or more (e.g., 1, 2, or 3) carboxylic acid, one or more (e.g., 1, 2, or 3) phosphoric acid, and/or one or more (e.g., 1, 2, or 3) sulfonic acid. For example, in some embodiments, T^{A} includes one or more, such as 1, 2, or 3, quaternary amine groups. In some embodiments, T^{A} includes one or more, such as 1, 2, or 3, carboxylic acid groups. And in some embodiments, T^{A} includes one or more zwitterion.

In some preferred embodiments, T^{A} can be characterized as having certain hydrophilicity as indicated by a c Log P value of a corresponding T^{A} containing molecule. For example, in some embodiments, T^{A} is a hydrophilic group having a terminal atom(s) selected from N, O, S, P, or C, which is covalently bonded with a first end atom of L^{A}, wherein (1) when the terminal atom(s) is N of a basic primary or secondary amine group, then the corresponding compound T^{A}-(C(O)—CH₃)_{q} has a c Log P of less than 0, preferably, less than −1, wherein the —C(O)—CH₃ is bonded with the terminal N atom(s); (2) when the terminal atom(s) is N of a basic tertiary amine group, then the corresponding compound [T^{A}-CH₃]⁺ has a c Log P of less than 0, preferably, less than −1, wherein the —CH₃ is bonded with the terminal N atom(s); (3) when the terminal atom(s) is C of a C(O) group, then the corresponding compound T^{A}-(OH)_{q} has a c Log P of less than 1, wherein the —OH is bonded with the terminal C atom(s); (4) when the terminal atom(s) is S of a SO₂ group, then the corresponding compound T^{A}-(OH)_{q} has a c Log P of less than 1, wherein the —OH is bonded with the terminal S atom(s); or (5) when (1)-(4) do not apply, then the corresponding compound T^{A}-H_{q} has a c Log P of less than 1.

The term “end atom(s)”, “terminal atom(s)”, and the alike as used herein in connection with a structure, such as L^{A} or T^{A} herein for Formula I, should be understood as the attaching point (atom) of the structure with the remainder of the molecule, thus by this definition, these end/terminal atoms are non-hydrogen atoms. For example, an alkylene chain of —(CH₂)₁₀— should be understood as having two end carbon atoms.

In some preferred embodiments, T^{A} is a hydrophilic group having a terminal N atom, which is covalently bonded with the first end atom of L^{A}, wherein the terminal N atom is that of a basic primary or secondary amine group, and the corresponding compound T^{A}-(C(O)—CH₃)_{q} has a c Log P of less than 0, preferably, less than −1 (e.g., less than −2, less than −3, less than −3.5, less than −4, or even lower), wherein the —C(O)—CH₃ is bonded with the terminal N atom. In some embodiments, q is 1, and the corresponding compound T^{A}-C(O)—CH₃ has a c Log P of less than 0, preferably, less than −1 (e.g., less than −2, less than −3, less than −3.5, less than −4, or even lower), wherein the —C(O)—CH₃ is bonded with the terminal N atom.

In some preferred embodiments, q is 1, and T^{A} is a hydrophilic group having a terminal N atom, which is covalently bonded with the first end atom of L^{A}, wherein the terminal N atom is that of a basic tertiary amine group, and the corresponding compound [T^{A}-CH₃]⁺ has a c Log P of less than 0, preferably, less than −1 (e.g., less than −2, less than −3, less than −3.5, less than −4, or even lower), wherein the —CH₃ is bonded with the terminal N atom.

In some embodiments, the terminal atom(s) is C of a C(O) group, and T^{A} is characterized in that the corresponding compound T^{A}-(OH)_{q} has a c Log P of less than 0 (e.g., less than −1, less than −2, less than −3, less than −3.5, less than −4, or even lower), wherein the —OH is bonded with the terminal C atom. In some embodiments, q is 1, and the corresponding compound T^{A}-OH has a c Log P of less than 0, preferably, less than −1 (e.g., less than −2, less than −3, less than −3.5, less than −4, or even lower), wherein the —OH is bonded with the terminal C atom.

In some embodiments, the terminal atom(s) is S in a SO₂ group, and T^{A} is characterized in that the corresponding compound T^{A}-(OH)_{q} has a c Log P of less than 0 (e.g., less than −1, less than −2, less than −3, less than −3.5, less than −4, or even lower), wherein the —OH is bonded with the terminal S atom. In some embodiments, q is 1, and the corresponding compound T^{A}-OH has a c Log P of less than 0, preferably, less than −1 (e.g., less than −2, less than −3, less than −3.5, less than −4, or even lower), wherein the —OH is bonded with the terminal S atom.

In some embodiments, the terminal atom(s) is not N of a basic amine group, C of a C(O) group, or S in a SO₂ group, and T^{A} is characterized in that the corresponding compound T^{A}-H_{q} has a c Log P of less than 0 (e.g., less than −1, less than −2, less than −3, less than −3.5, less than −4, or even lower). In some embodiments, q is 1, and the corresponding compound T^{A}-H has a c Log P of less than 0, preferably, less than −1 (e.g., less than −2, less than −3, less than −3.5, less than −4, or even lower).

In some preferred embodiments, q is 1, and T^{A} in Formula I-1 has a formula according to M-1 or M-2:

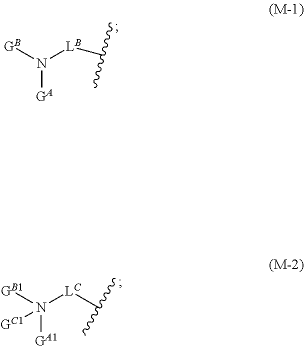

wherein:
each of L^{B} and L^{C} at each occurrence is independently null or represents a divalent group; wherein, in M-1:
   (i) One of G^{A} and G^{B} is hydrogen or is selected from C₁₋₄ alkyl, C₂₋₄ alkenyl, C₂₋₄ alkynyl, or a 3-14 membered ring, each of which is optionally substituted, and the other of G^{A} and G^{B} is a moiety having the structure of M-2, M-3, or M-4 as defined herein; or
   (ii) G^{A} and G^{B}, together with the nitrogen atom they are both attached to, are joined to form an optionally substituted 4-14 membered ring; or
   (iii) each of G^{A} and G^{B} independently represents a moiety having the structure of M-2, M-3, or M-4 as defined herein;
wherein, in M-2
   (i) G^{A1}, G^{B1}, and G^{C1} each independently represents C₁₋₄ alkyl, C₂₋₄ alkenyl, C₂₋₄ alkynyl, a 3-14 membered ring, or a structure according to M-3 or M-4; wherein each of the C₁₋₄alkyl, C₂₋₄ alkenyl, C₂₋₄alkynyl, and 3-14 membered ring is optionally substituted;
   (ii) G^{A1} and G^{B1}, together with the nitrogen atom they are both attached to, are joined to form an optionally substituted 4-14 membered ring; and G^{C1} is as defined in (i); or
   (iii) G^{A1}, G^{B1}, and G^{C1} together with the nitrogen atom they are all attached to, are joined to form an optionally substituted 5-14 membered ring;
wherein M-3 has a structure of

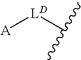

and M-4 has a structure of

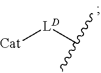

wherein:
   L^{D} is null or represents a divalent group;
   A represents a moiety having an anionic group or a conjugated acid thereof, preferably, the anionic group is selected from COO⁻, SO₃⁻, HPO₃⁻ or PO₃²⁻; and
   Cat represents a moiety having a cationic group that is positively charged regardless of pH or positively chargeable at pH of 7, preferably, the cationic group is a quaternary amine. The “3-14 membered ring”, or “4-14 membered ring”, or “5-14 membered ring” herein (1) can be a monocyclic ring, typically when the number of ring atoms is 3-8, wherein the monocyclic ring (i) can be saturated, partially unsaturated, or aromatic; and (ii) contain 0-4 heteroatoms each independently selected from N, S, O, and P; or (2) can contain a fused, spiro, and/or bridged structure having two or more constituent rings, typically when the total number of ring atoms is 5-14, wherein each constituent ring independently (i) can be saturated, partially unsaturated, or aromatic; and (ii) contain 0-4 heteroatoms each independently selected from N, S, O, and P. It should be also understood that the ring heteroatoms N, S, and P may exist in different oxidation states, for example, S can exist as S, SO, or SO₂, etc., and the nitrogen ring atom may be optionally oxidized or quaternized. Unless otherwise specified, ring structures herein having a different designation of number of ring members should be understood similarly.

In some embodiments, T^{A} has a formula according to M-1. In some embodiments, one of G^{A} and G^{B} is hydrogen or C₁₋₄ alkyl, and the other of G^{A} and G^{B} has a structure according to M-2. In some embodiments, each of G^{A} and G^{B} is independently a structure according to M-2. In some embodiments, one of G^{A} and G^{B} is hydrogen or C₁₋₄ alkyl, and the other of G^{A} and G^{B} has a structure according to M-3. In some embodiments, one of G^{A} and G^{B} is hydrogen or C₁₋₄ alkyl, and the other of G^{A} and G^{B} has a structure according to M-4. In some embodiments, each of G^{A} and G^{B} is independently a structure according to M-3. In some embodiments, each of G^{A} and G^{B} is independently a structure according to M-4. In some embodiments, G^{A} and G^{B}, together with the nitrogen atom they are both attached to, are joined to form an optionally substituted 4-14 membered ring, such as a monocyclic 4-8 membered ring or a 5-14 membered ring structure having two or more constituent rings as defined and exemplified herein. For example, in some embodiments, the compound of Formula I can have a structure according to Formula I-1-A, I-1-B, I-1-C, I-1-D, I-1-E, or I-1-F, counterbalanced as necessary:

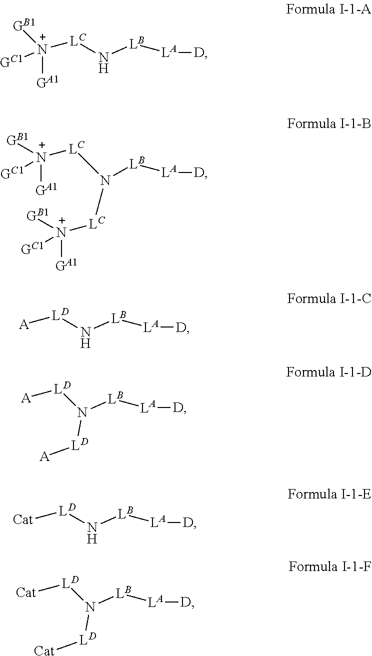

wherein the variables are defined herein. To be clear, in Formula I-1-B, I-1-D, and I-1-F, variables with the same identifier, such as two “A” in Formula I-1-D or two “Cat” in Formula I-1-F, can be the same or different.

In some embodiments, T^{A} has a formula according to M-2. In some embodiments, G^{A1} and G^{B1}, together with the nitrogen atom they are both attached to, are joined to form an optionally substituted 3-14 membered ring; and G^{C1} is C₁₋₄ alkyl. In some embodiments, G^{A1}, G^{B1}, and G^{C1} together with the nitrogen atom they are all attached to, are joined to form an optionally substituted 5-14 membered ring, typically a ring structure having two or more constituent rings as defined and exemplified herein, such as a fused or bridged bicyclic ring structure. In some embodiments, G^{A1}, G^{B1}, and G^{C1} each independently represents C₁₋₄ alkyl or a structure according to M-3 or M-4. In some embodiments, the compound of Formula I can have a structure according to Formula I-1-G, I-1-G-1, I-1-G-2, I-1-G-3, I-1-G-4, I-1-G-5, I-1-G-6, or I-1-G-7, counterbalanced as necessary:

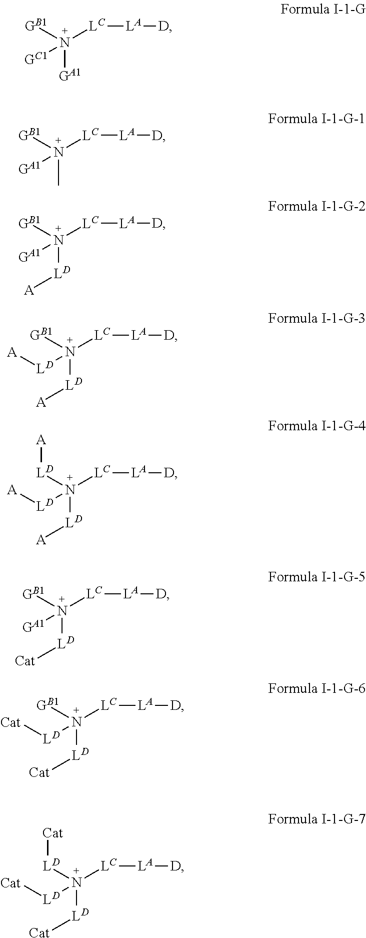

wherein the variables are defined herein.

In some embodiments, T^{A} can also have a formula according to M-3 or M-4, as defined herein, in which L^{D} is attached to L^{A}. For example, in some embodiments, the compound of Formula I can have a structure according to Formula I-1-H or I-1-I:

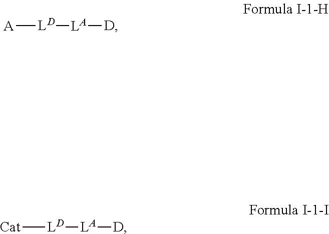

wherein the variables are defined herein.

The divalent linkers L^{B}, L^{C}, and L^{D} in M-1, M-2, M-3, or M-4 herein, as applicable, e.g., in any of the applicable subformulae of Formula I-1, are not particularly limited, which can be null, acyclic or cyclic divalent structure, which can optionally contain heteroatoms (e.g., 1-4 heteroatoms).

In some embodiments, L^{B} can have a structure according to L^{B1} or L^{B1}-L^{B2} wherein L^{B1} is attached to NG^{A}G^{B}, and wherein L^{B1} is null, optionally substituted alkylene, optionally substituted C₁₋₆ heteroalkylene, optionally substituted 3-14 membered ring, or optionally substituted ring/chain structure, and L^{B2} is null, C(O), NH, —SO₂—, or a moiety selected from:

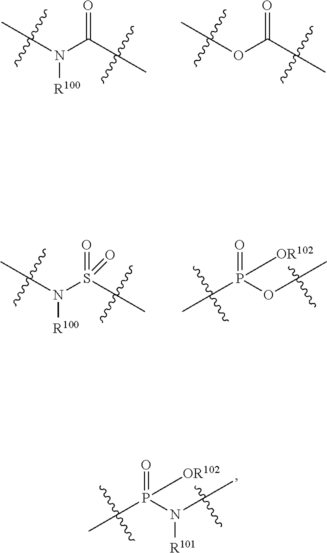

wherein R¹⁰⁰, R¹⁰¹ and R¹⁰² at each occurrence is independently hydrogen, optionally substituted alkyl, or optionally substituted cycloalkyl, and either of the attaching points may be connected to L^{B1}. In some embodiments, L^{B} is L^{B1}. In some embodiments, L^{B} is L^{B1}-NH—. In some embodiments, L^{B} is L^{B1}-C(O)—. For example, in some embodiments, L^{B} or L^{B1} is null. In some embodiments, L^{B} or L^{B1} is an optionally substituted alkylene, such as an optionally substituted C₁₋₆ alkylene. In some embodiments, L^{B} or L^{B1} is an optionally substituted heteroalkylene, for example, an optionally substituted C₁₋₆ heteroalkylene having one or two heteroatoms independently selected from N, O, P, and S, wherein the S and P are optionally oxidized, for example, L^{B} or L^{B1} can be —CH₂—CH₂—O—, —CH₂—CH₂—NH—, —CH₂—CH₂—N(CH₃)—, —CH₂—CH₂—O—CH₂—CH₂—NH—, —CH₂—CH₂—CH₂—NH—, —CH₂—CH₂—S—CH₂—CH₂—NH—, —CH₂—CH₂—SO₂—NH—CH₂—CH₂—NH—, etc. In some embodiments, one or more CH₂ of the heteroalkylene may be optionally substituted with an oxo group, for example, an optionally substituted —CH₂—CH₂—CH₂—NH— includes the structure of —CH₂—CH₂—C(O)—NH—, etc. In some embodiments, L^{B} or L^{B1} is an optionally substituted divalent ring structure which is attached to the remainder of the molecule through two attaching points on the divalent ring structure, e.g., through one ring atom or two ring atoms. In some embodiments, L^{B} or L^{B1} is an optionally substituted divalent ring-chain structure which is attached to the remainder of the molecule through one chain atom and one ring atom. When the compound of Formula I contains two or more L^{B} as variables, each of the L^{B} is independently selected.

In some embodiments, L^{C} in M-2, or any of the subformulae of Formula I having L^{C} as a variable, can have a structure according to L^{C1} or L^{C1}-L^{C2}, wherein L^{C1} is attached to the quaternary nitrogen [NG^{A1}G^{B1}G^{C1}]⁺, wherein L^{C1} is null, optionally substituted alkylene, optionally substituted C₁₋₆ heteroalkylene, optionally substituted 3-14 membered ring, or optionally substituted ring/chain structure, and L^{C2} is null, C(O), NH, —SO₂—, or a moiety selected from:

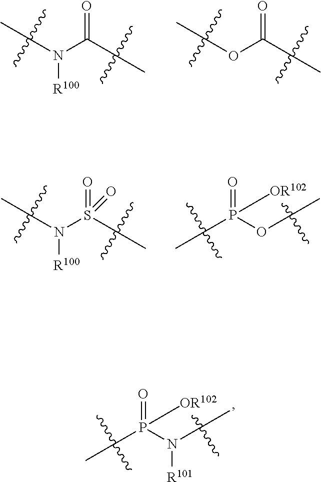

wherein R¹⁰⁰, R¹⁰¹ and R¹⁰² at each occurrence is independently hydrogen, optionally substituted alkyl, or optionally substituted cycloalkyl, and either of the attaching points may be connected to L^{C1}. In some embodiments, L^{C} is L^{C1}. In some embodiments, L^{C} is L^{C1}-NH—. In some embodiments, L^{C} is L^{C1}-C(O)—. In some embodiments, L^{C} or L^{C1} is null. In some embodiments, L^{C} or L^{C1} is an optionally substituted alkylene, such as an optionally substituted C₁₋₆ alkylene. In some embodiments, L^{C} or L^{C1} is an optionally substituted heteroalkylene, for example, an optionally substituted C₁₋₆ heteroalkylene having one or two heteroatoms independently selected from N, O, P, and S, wherein the S or P is optionally oxidized, for example, L^{C} or L^{C1} can be —CH₂—CH₂—O—, —CH₂—CH₂—NH—, —CH₂—CH₂—N(CH₃)—, —CH₂—CH₂—O—CH₂—CH₂—NH—, —CH₂—CH₂—CH₂—NH—, —CH₂—CH₂—S—CH₂—CH₂—NH—, —CH₂—CH₂—SO₂—NH—CH₂—CH₂—NH—, etc. In some embodiments, one or more CH₂ of the heteroalkylene may be optionally substituted with an oxo group. In some embodiments, L^{C} or L^{C1} is an optionally substituted divalent ring structure which is attached to the remainder of the molecule through two attaching points on the divalent ring structure, e.g., through one ring atom or two ring atoms. In some embodiments, L^{C} or L^{C1} is an optionally substituted divalent ring-chain structure which is attached to the remainder of the molecule through one chain atom and one ring atom. When the compound of Formula I contains two or more L^{C} as variables, each of the L^{C} is independently selected.

In some embodiments, L^{D} in M-3 or M-4, or any of the subformulae of Formula I having L^{D} as a variable, can have a structure according to L^{D1} or L^{D1}-L^{D2}, wherein L^{D1} is attached to A or Cat, wherein L^{D1} is null, optionally substituted alkylene, optionally substituted C₁₋₆ heteroalkylene, optionally substituted 3-14 membered ring, or optionally substituted ring/chain structure, and L^{D2} is null, C(O), NH, —SO₂—, or a moiety selected from:

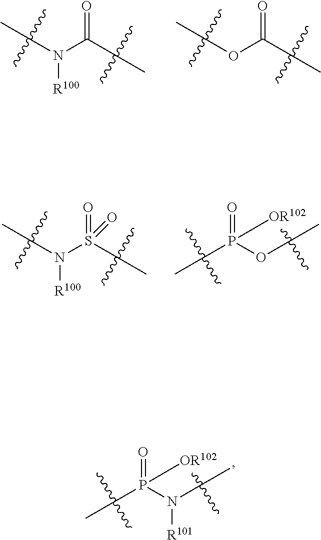

wherein R¹⁰⁰, R¹⁰¹ and R¹⁰² at each occurrence is independently hydrogen, optionally substituted alkyl, or optionally substituted cycloalkyl, and either of the attaching points may be connected to L^{D1}. In some embodiments, L^{D} is L^{D1}. In some embodiments, L^{D} is L^{D1}-NH—. In some embodiments, L^{D} is L^{D1}-C(O)—. In some embodiments, L^{D} or L^{D1} is null. In some embodiments, L^{D} or L^{D1} is an optionally substituted alkylene, such as an optionally substituted C₁₋₆ alkylene. In some embodiments, L^{D} or L^{D1} is an optionally substituted heteroalkylene, for example, an optionally substituted C₁₋₆ heteroalkylene having one or two heteroatoms independently selected from N, O, P, and S, wherein the P or S is optionally oxidized. In some embodiments, one or more CH₂ of the heteroalkylene may be optionally substituted with an oxo group. In some embodiments, L^{D} or L^{D1} is an optionally substituted divalent ring structure which is attached to the remainder of the molecule through two attaching points on the divalent ring structure, e.g., one ring atom or two ring atoms. In some embodiments, L^{D} or L^{D1} is an optionally substituted divalent ring-chain structure which is attached to the remainder of the molecule through one chain atom and one ring atom. When the compound of Formula I contains two or more L^{D} as variables, each of the L^{D} is independently selected.

In some embodiments, in M-2 or any of the subformulae of Formula I having G^{A1}, G^{B1}, and G^{C1} as variables, G^{A1}, G^{B1}, and G^{C1} together with the nitrogen atom they are all attached to, can be joined to form an optionally substituted 5-14 membered ring having 0-4 ring heteroatoms in addition to the nitrogen atom, wherein the additional ring heteroatoms, if present, are independently selected from N, S, and O, wherein the nitrogen can be optionally oxidized or quaternized, the sulfur can be optionally oxidized. Typically, the 5-14 membered ring is a ring structure having two or more constituent rings, for example, the 5-14 membered ring can have a fused ring structure, bridged ring structure, and/or a spiro ring structure, wherein each constituent ring independently is typically a saturated or partially unsaturated heterocyclic ring having 4-8 ring members. For example, in some embodiments, the 5-14 membered ring can be a bridged bicyclic ring, wherein each path between two bridgehead atoms is independently 1, 2, 3, or 4 ring atoms, for example, a 1,1,1-bridged

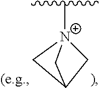 [SMILES: *[N+]12CC(C1)C2]

2,2,2-bridged

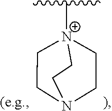 [SMILES: *[N+]12CCN(CC1)CC2]

3,2,1-bridged, 3,3,3-bridged ring, etc. In some embodiments, the 5-14 membered ring can be a fused bicyclic ring. For example, in some preferred embodiments, G^{A1}, G^{B1}, and G^{C1} together with the nitrogen atom they are all attached to, are joined to form a structure of

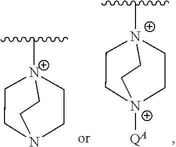

wherein Q^{A} is an optionally substituted C₁₋₄ alkyl, such as methyl. In some preferred embodiments, G^{A1}, G^{B1}, and G^{C1} together with the nitrogen atom they are all attached to, are joined to form

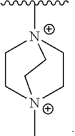 [SMILES: *[N+]12CC[N+](C)(CC1)CC2]

When the compound of Formula I contains two or more sets of G^{A1}, G^{B1}, and G^{C1} as variables, each set of variables is independently selected.

In some embodiments, in M-3 or any of the subformulae of Formula I having A as a variable, A at each occurrence independently represents a group containing an anion of COO⁻, SO₃⁻, HPO₃⁻ or PO₃²⁻, or a conjugated acid thereof. In some embodiments, when more than two A appear in a formula as variables, each A can be the same or different, for example, in some embodiments, when two or more A appear in a formula herein, all A are the same, for example, all A are COO⁻ or COOH.

In some embodiments, in M-4 or any of the subformulae of Formula I having Cat as a variable, Cat at each occurrence is independently a structure containing an amine, preferably, containing a quaternary amine. For example, in some preferred embodiments, Cat at each occurrence is a quaternary amine having a structure of

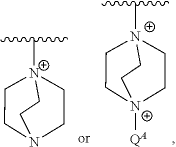

wherein Q^{A} is an optionally substituted C₁₋₄ alkyl, such as methyl. In some embodiments, Cat can be

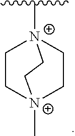 [SMILES: *[N+]12CC[N+](C)(CC1)CC2]

In some embodiments, Cat can be NH2. In some embodiments, Cat can be NH—(CH₂)₂₋₅—NH₂, such as NH—(CH₂)₂—NH₂. In some embodiments, Cat can be

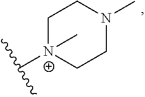

[N(CH₃)₃]⁺, or [N(CH₂CH₃)₃]⁺. In some embodiments, when more than two Cat appear in a formula as a variable, each Cat can be the same or different, preferably, all Cat are the same.

In some preferred embodiments, T^{A} in Formula I-1 represents

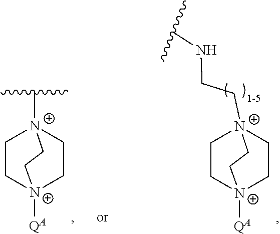

wherein Q^{A} is an optionally substituted C₁₋₄alkyl, such as methyl.

In some preferred embodiments, T^{A} in Formula I-1 represents

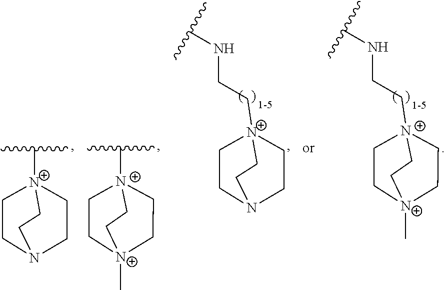

In some preferred embodiments, T^{A} in Formula I-1 represents

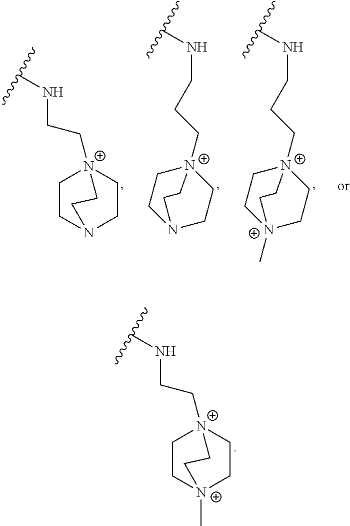 [SMILES: *NCC[N+]12CCN(CC1)CC2 ; *NCCC[N+]12CCN(CC1)CC2 ; *NCCC[N+]12CC[N+](C)(CC1)CC2 ; *NCC[N+]12CC[N+](C)(CC1)CC2]

In some preferred embodiments, T^{A} in Formula I-1 represents

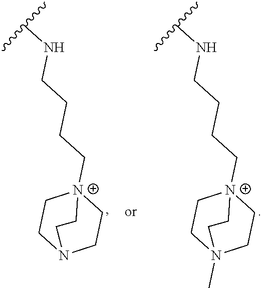

In some embodiments, T^{A} in Formula I-1 represents

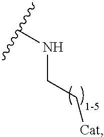

wherein Cat is defined herein, for example, in some embodiments, Cat is

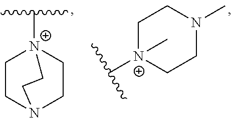

[N(CH₃)₃]⁺, or [N(CH₂CH₃)₃]⁺.

In some embodiments, T^{A} in Formula I-1 represents

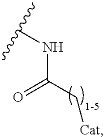

wherein Cat is defined herein, for example, in some embodiments, Cat is

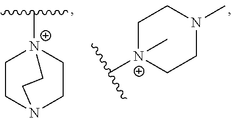

[N(CH₃)₃]⁺, or [N(CH₂CH₃)₃]⁺.

In some preferred embodiments, T^{A} in Formula I-1 represents

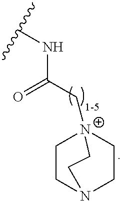

In some preferred embodiments, T^{A} in Formula I-1 represents

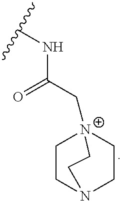 [SMILES: *NC(=O)C[N+]12CCN(CC1)CC2]

In some preferred embodiments, T^{A} in Formula I-1 represents

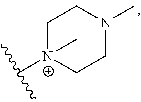

[N(CH₃)₃]⁺, or [N(CH₂CH₃)₃]⁺.

In some preferred embodiments, T^{A} in Formula I-1 represents one of the following structures:

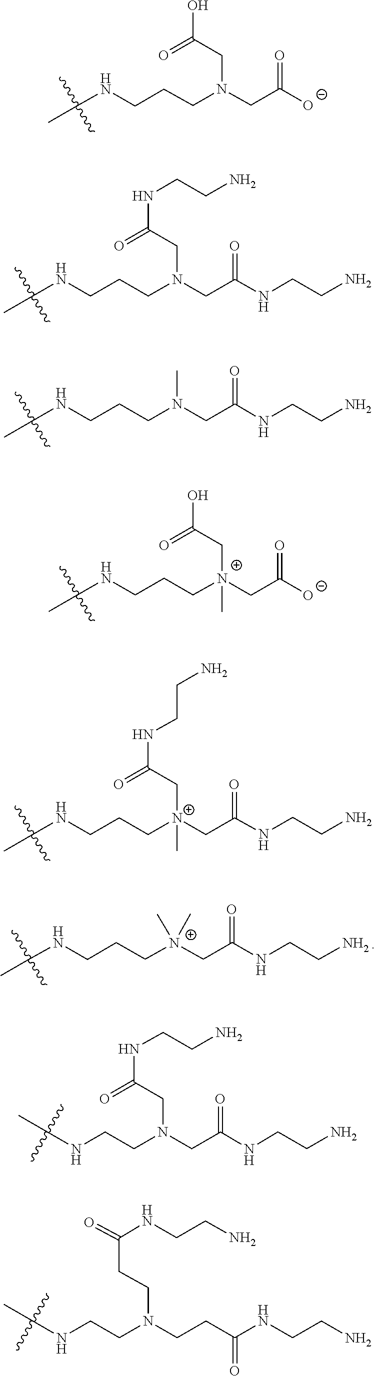

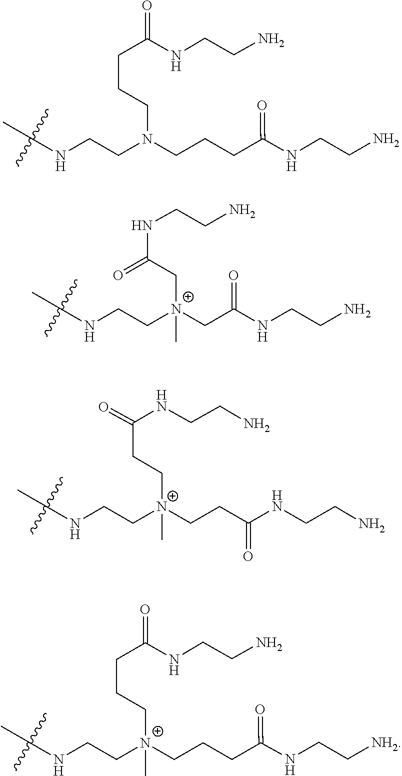 [SMILES: *NCCN(CCCC(=O)NCCN)CCCC(=O)NCCN ; *NCC[N+](C)(CC(=O)NCCN)CC(=O)NCCN ; *NCC[N+](C)(CCC(=O)NCCN)CCC(=O)NCCN ; *NCC[N+](C)(CCCC(=O)NCCN)CCCC(=O)NCCN]

In some preferred embodiments, T^{A} in Formula I-1 represents

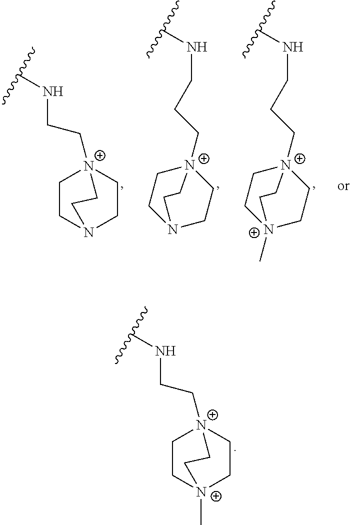 [SMILES: *NCC[N+]12CCN(CC1)CC2 ; *NCCC[N+]12CCN(CC1)CC2 ; *NCCC[N+]12CC[N+](C)(CC1)CC2 ; *NCC[N+]12CC[N+](C)(CC1)CC2]

In some preferred embodiments, T^{A} in Formula I-1 represents

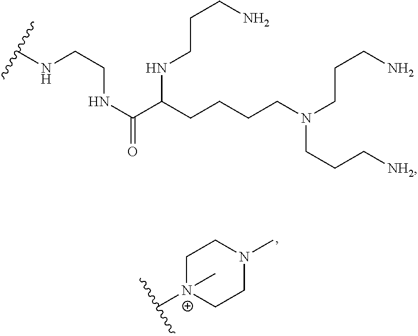

[N(CH₃)₃]⁺, [N(CH₂CH₃)₃]⁺,

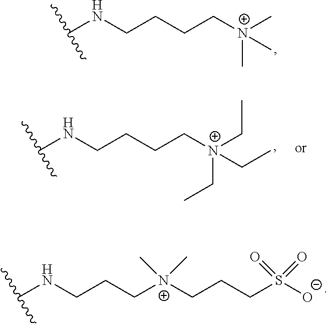

### Linker L^{A}

When D represents a residue of a GPCR ligand, another factor that can determine whether the compound of Formula I can be a potent GPCR ligand, such as a GPR40 agonist is the length of the linker (e.g., L^{A}) but not the exact chemical structure of the linker. As shown in PCT/CN2023/071833, when the length of the linker is below certain threshold, the EC50 value as GPR40 agonists can increase significantly. Accordingly, the present inventors envision that the maximum length between the two end atoms of linker L^{A} in Formula I should be at least that between the two end carbon atoms of an alkylene chain —(CH₂)₁₀—. In other words, the longest chain length of L^{A} should be equal to or greater than the longest chain length of the alkylene chain —(CH₂)₁₀—. To further explain, the linker L^{A} can be viewed as having the following structure with two end atoms:

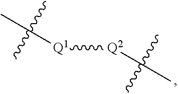

wherein Q¹ represents a non-hydrogen atom of the first end of L^{A} that is bonded with an terminal atom in T^{A}, and Q² represents a non-hydrogen atom of the second end of L^{A} that is bonded with an attaching point in D, and Q¹ and Q² are connected through a chain or ring/chain structure. Thus, the maximum distance between the two end atoms of linker L^{A} should be understood as the maximum distance between the two connecting points Q¹ and Q² in L^{A}, which under the definition above, should be equal to or greater than the maximum length between the two end carbons of the alkylene chain —(CH₂)₁₀—. In other words, the maximum distance between the two connecting points Q¹ and Q² in L^{A} should be equal to or greater than that when L^{A} is —(CH₂)₁₀—. Unless otherwise specified or obvious contrary from context, the alkylene chain such as —(CH₂)₁₀— and the alike herein is not in a cyclic structure. The maximum length between the two end carbons of the —(CH₂)₁₀— can be estimated by computer modeling, measuring the distance between the two end carbons when the alkylene chain is fully stretched in one direction. Similarly, the maximum length between Q¹ and Q² in L^{A} can be estimated by computer modeling, measuring the distance between Q¹ and Q² when the chain(s) in L^{A} is fully stretched in one direction. Using this method, it would be apparent that a linear alkylene chain having more than 10 carbons in the chain will have a maximum length longer than that of —(CH₂)₁₀—. Similarly, a linear saturated chain structure having more than 10 non-hydrogen atoms in the chain will also have a maximum length longer than that of —(CH₂)₁₀—. In some embodiments, L^{A} can be characterized in that the maximum length between the two end atoms of L^{A} is at least that between the two end carbon atoms of —(CH₂)₁₂—, preferably, at least that of —(CH₂)₁₄—, more preferably, at least that of —(CH₂)₁₆—. In some embodiments, L^{A} can be characterized in that the maximum length between the two end atoms of L^{A} is between (i) the maximum length between the two end carbon atoms of —(CH₂)₁₂— and (ii) the maximum length between the two end carbon atoms of —(CH₂)₅₀—.

Another factor that can determine whether the compound of Formula I can be a potent GPCR ligand, such as a GPR40 agonist is the hydrophobicity of the linker (e.g., L^{A}) but not the exact chemical structure of the linker. In general, L^{A} should be a hydrophobic moiety. In some embodiments, both end atoms of L^{A} are C of a C(O) or S of a SO2 group, in such embodiments, the hydrophobicity of L^{A} can be typically characterized in that the corresponding compound HO-L^{A}-OH has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc., wherein each of the —OH is bonded with the end C(O) or SO2 group. In some embodiments, only one end atom of L^{A} is C of a C(O) or S of a SO2 group, in such embodiments, the hydrophobicity of L^{A} can be typically characterized in that the corresponding compound H-L^{A}-OH has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc., wherein the —OH is bonded with the end C(O) or SO2 group. In some embodiments, neither of the end atoms of L^{A} is C of a C(O) or S of a SO2 group, in such embodiments, in such embodiments, the hydrophobicity of L^{A} can be characterized in that the corresponding compound H-L^{A}-H has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc.

In some embodiments, in Formula I (e.g., Formula I-1), L^{A} can be represented by a formula of (X)ₘ, wherein X at each occurrence is independently CR₂, C(═O), —C(R)═C(R)—,

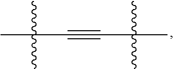 [SMILES: *C#C*]

SiR₂, O, S, SO, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc. To be clear, the formula (X)ₘ should be understood as a linear structure having each X connected to another two X groups except the two end X groups (the two X groups that are directly connected to T^{A} or D), i.e., —X—X—X— . . . —X—, with the total number of X being m. In cases wherein X is a ring structure, preferably 3-10 membered ring structure, it should be understood that the ring structure is attached to two adjacent X units through one or two ring atoms, for example, X can have a structure such as

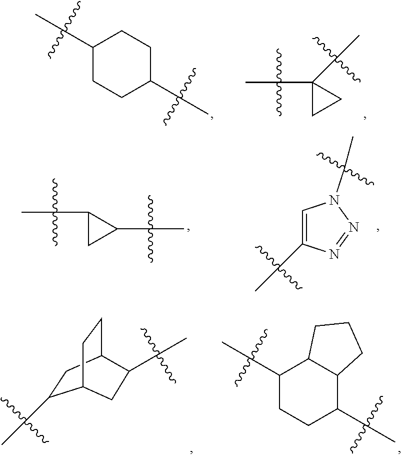 [SMILES: *C1CCC(*)CC1 ; *C1(*)CC1 ; *C1CC1* ; *c1cn(*)nn1 ; *C1CC2CCC1CC2* ; *C1CCC(*)C2CCCC12]

etc. The “ring structure” or “3-10 membered ring structure” and the alike as used herein is not limited to any particular ring system and can include a carbocyclic ring, a heterocyclic ring, an aromatic ring, a heteroaryl ring, or a combination thereof, which can be substituted or unsubstituted. For example, the “3-10 membered ring structure” can be monocyclic, bicyclic, or tricyclic, which can include a fused, spiro, or bridged ring system. For clarity, two ring systems connected through a single bond should be viewed as separate ring system and can each account for one X unit herein. For example, for a structure like

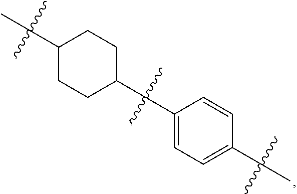

it may be viewed as two X units connected, with one X being a cyclohexylene and the other X being a phenylene. Typically, in L^{A}, 0, 1, or 2 instances of X representing a ring structure, preferably 3-10 membered ring structure (such as a C₃₋₆ cycloalkyl such as cyclopropyl, a 5 or 6 membered heteroaryl, such as a triazole ring). In some embodiments, two consecutive X can be —C(O)O— or —C(O)NR—. In some embodiments, one instance of X can be a cyclopropane, cyclobutane or bicyclobutane[1.1] ring. In some embodiments, one instance of X can be a 5 or 6-membered heteroaryl, such as a triazole ring. In some embodiments, one instance of X can be a cyclopentane, cyclohexane or cycloheptane ring. In some embodiments, X at each occurrence is independently CR₂, —C(R)═C(R)—,

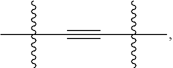 [SMILES: *C#C*]

or a 3-10 membered ring, wherein R at each occurrence is independently hydrogen or C₁₋₄ alkyl; more preferably, X at each occurrence is independently CR₂, wherein R at each occurrence is independently hydrogen or C₁₋₄ alkyl. Typically, one of the end X group connects to T^{A} through a carbon atom. The total number of non-hydrogen atoms of L^{A} can be typically between 10-100, such as 12-30, 14-50, 16-50, 18-100, etc.

For example, in some embodiments, L^{A} is (X)ₘ₋₁—C(O)—, wherein the C(O) end is bonded with T^{A}, wherein X at each occurrence is independently CR₂, C(═O), —C(R)═C(R)—,

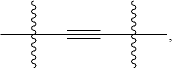 [SMILES: *C#C*]

SiR₂, O, S, SO₂, NR, [NR₂]⁺ or a ring structure, preferably 3-10 membered ring structure, provided that the end X group (i.e., the X group in L^{A} that is furthest away from the C(O) end) is not C(O) or SO₂, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc., and the hydrophobicity of —(X)ₘ₋₁—C(O)— is characterized in that the corresponding compound H—(X)ₘ₋₁—COOH should have a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc. The “m−1” should be understood as the integer m minus 1, not to be misunderstood as a different designated variable. In some embodiments, X at each occurrence is independently CR₂, —C(R)═C(R)—,

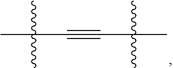 [SMILES: *C#C*]

or a 3-10 membered ring, wherein R at each occurrence is independently hydrogen or C₁₋₄ alkyl; more preferably, X at each occurrence is independently CR₂, wherein R at each occurrence is independently hydrogen or C₁₋₄ alkyl.

In some embodiments, L^{A} is (X)ₘ, wherein X at each occurrence is independently CR₂, C(═O), —C(R)═C(R)—,

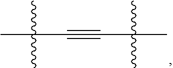 [SMILES: *C#C*]

SiR₂, O, S, SO₂, NR, [NR₂]⁺ or a ring structure, preferably 3-10 membered ring structure, provided that neither of the end X groups is C(O) or SO₂, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc., then the corresponding compound H—(X)ₘ—H should have a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc. In some embodiments, X at each occurrence is independently CR₂, —C(R)═C(R)—,

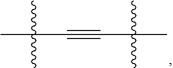 [SMILES: *C#C*]

or a 3-10 membered ring, wherein R at each occurrence is independently hydrogen or C₁₋₄ alkyl; more preferably, X at each occurrence is independently CR₂, wherein R at each occurrence is independently hydrogen or C₁₋₄ alkyl.

As would be apparent to those skilled in the art, the term c Log P (or C Log P) refers to calculated Log P. For the purpose of this application, the c Log P value can be obtained using PerkinElmer's ChemDraw Professional software, version 20.0.0.41 or equivalent software using the same calculation method. The following shows exemplary c Log P values of a few compounds using the ChemDraw Professional software above:

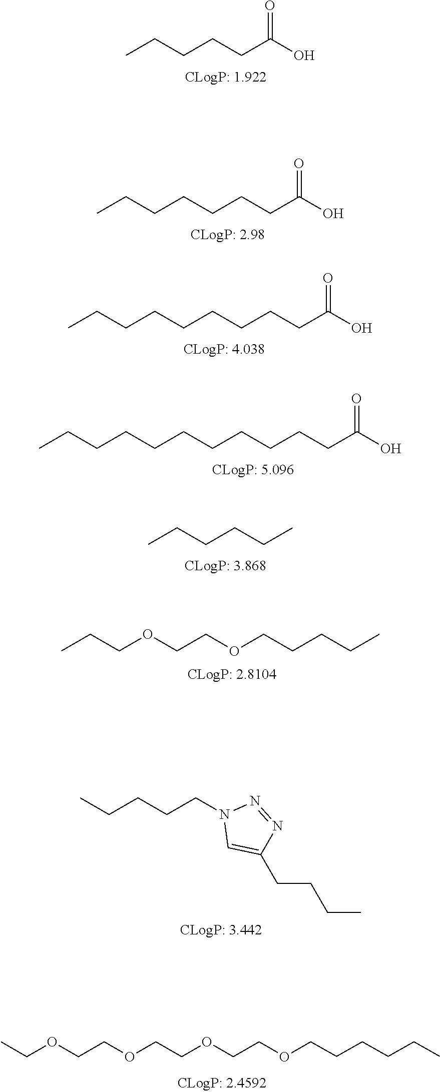

Thus, a compound having a c Log P of at least 3 should be about the same or more hydrophobic than octanoic acid. A compound having a c Log P of at least 4 should be about the same or more hydrophobic than decanoic acid. A compound having a c Log P of at least 5 should be about the same or more hydrophobic than lauric acid.

In some embodiments, L^{A} is —X₁₂₋₃₀— (e.g., —X₁₄—, —X₁₆—, —X₁₈—, —X₂₀—, —X₂₄—, —X₁₄₋₃₀—, —X₁₆₋₃₀—, —X₁₈₋₃₀—, etc.), wherein X at each occurrence is independently CR₂, C(═O), —C(R)═C(R)—,

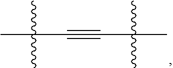 [SMILES: *C#C*]

SiR₂, O, S, SO₂, NR, [NR₂]⁺, or a ring structure, preferably 3-10 membered ring structure, provided that neither of the end X groups is C(O) or SO₂; wherein R at each occurrence is independently hydrogen, halogen, optionally substituted C₁₋₄ alkyl, or optionally substituted C₁₋₄ alkoxy, typically hydrogen or C₁₋₄ alkyl, and wherein the hydrophobicity of L^{A} can be characterized in that the corresponding compound H-L^{A}-H has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc. In some embodiments, the total number of non-hydrogen atoms of L^{A} is between 12-100, such as 12, 14, 16, 18, 20, 24, 30, 40, 50, 100, or any ranges between the recited values, such as 12-30, 14-50, 16-50, 18-100, etc. In some embodiments, two consecutive X can represent —C(O)O— or —C(O)NR—. In some embodiments, one or more (e.g., 1 or 2) instances of X can be a ring structure selected from cyclopropane, cyclobutane, bicyclobutane[1.1], cyclopentane, cyclohexane or cycloheptane. In some embodiments, one or more (e.g., 1) instances of X can be a ring structure selected from phenyl or 5 or 6-membered heteroaryl, such as triazole. In some embodiments, R is hydrogen. In some embodiments, X at each occurrence is independently CR₂, —C(R)═C(R)—,

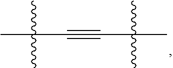 [SMILES: *C#C*]

or a 3-10 membered ring, wherein R at each occurrence is independently hydrogen or C₁₋₄ alkyl; more preferably, X at each occurrence is independently CR₂, wherein R at each occurrence is independently hydrogen or C₁₋₄ alkyl.

In some embodiments, L^{A} is —X₁₂₋₃₀—C(O)— (e.g., —X₁₄—C(O)—, —X₁₆—C(O)—, —X₁₈—C(O)—, —X₂₀—C(O)—, —X₂₄—C(O)—, —X₁₄₋₃₀—C(O)—, —X₁₆₋₃₀—C(O)—, —X₁₈₋₃₀—C(O)—, etc.), wherein the C(O) is directly bonded with T^{A}, and wherein X at each occurrence is independently CR₂, C(═O), —C(R)═C(R)—,

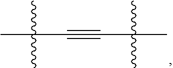 [SMILES: *C#C*]

SiR₂, O, S, SO₂, NR, [NR₂]⁺, or a ring structure, preferably 3-10 membered ring structure, provided that the end X group is not C(O) or SO2; wherein R at each occurrence is independently hydrogen, halogen, optionally substituted C₁₋₄ alkyl, or optionally substituted C₁₋₄ alkoxy, typically hydrogen or C₁₋₄ alkyl, and wherein the hydrophobicity of L^{A} can be characterized in that the corresponding compound H—X₁₂₋₃₀—C(O)—OH has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, two consecutive X can be —C(O)O— or —C(O)NR—. In some embodiments, one or more instances (e.g., 1 or 2) of X can be a ring structure selected from cyclopropane, cyclobutane, bicyclobutane[1.1], cyclopentane, cyclohexane or cycloheptane. In some embodiments, one or more instances (e.g., 1) of X can be a ring structure selected from phenyl or 5 or 6-membered heteroaryl, such as triazole. In some embodiments, R is hydrogen. In some embodiments, X at each occurrence is independently CR₂, —C(R)═C(R)—,

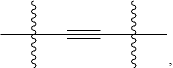 [SMILES: *C#C*]

or a 3-10 membered ring, wherein R at each occurrence is independently hydrogen or C₁₋₄ alkyl; more preferably, X at each occurrence is independently CR₂, wherein R at each occurrence is independently hydrogen or C₁₋₄ alkyl.

In some preferred embodiments, L^{A} is —C₂₋₃₀ alkylene- or —C₁₂₋₃₀ alkylene-C(O)—, wherein the —C₁₂₋₃₀ alkylene- is optionally substituted, wherein the optional substituents can optionally be joined together to form a double bond, triple bond, or a ring structure (typically a 3-10 membered ring structure), wherein the end carbon atoms of the —C₁₂₋₃₀ alkylene- are not substituted with oxo (═O), wherein the longest chain length of L^{A} is at least that of —(CH₂)₁₂—, such as at least that of —(CH₂)₁₄—, at least that of —(CH₂)₁—, at least that of —(CH₂)₁₈—, and wherein the hydrophobicity of L^{A} can be characterized in that the corresponding compound H—C₁₂₋₃₀ alkylene-H or H—C₁₂₋₃₀ alkylene-C(O)—OH has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A} is unsubstituted —C₂₋₃₀ alkylene-, such as a linear or branched C₁₂₋₃₀ alkylene. In some embodiments, L^{A} is unsubstituted —C₁₂₋₃₀ alkylene-C(O)—, wherein the C₁₂₋₃₀ alkylene can be linear or branched. In some preferred embodiments, L^{A} is —C₁₂₋₃₀ alkylene- or —C₁₂₋₃₀ alkylene-C(O)—, wherein the C₁₂₋₃₀ alkylene is a linear and unsubstituted —C₁₂₋₃₀ alkylene-.

In some embodiments, L^{A} is a 12-30 membered heteroalkylene or -(12-30 membered heteroalkylene)-C(O)—, wherein the 12-30 membered heteroalkylene is optionally substituted and contains 1-6 heteroatoms independently selected from O, N, and S, wherein the sulfur atom(s), if present, is optionally oxidized, wherein the optional substituents can optionally be joined together to form a double bond, triple bond, or a ring structure (typically a 3-10 membered ring structure), wherein the end atoms of the 12-30 membered heteroalkylene are not C of a C(O) or S of a SO2 group, wherein the longest chain length of L^{A} is at least that of —(CH₂)₁₂—, such as at least that of —(CH₂)₁₄—, at least that of —(CH₂)₁₆—, at least that of —(CH₂)₁₈—, and wherein the hydrophobicity of L^{A} can be characterized in that the corresponding compound H-(12-30 membered heteroalkylene)-H or H-(12-30 membered heteroalkylene)-C(O)—OH has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A} is unsubstituted 12-30 membered heteroalkylene, such as a linear or branched 12-30 membered heteroalkylene. In some embodiments, L^{A} is unsubstituted -(12-30 membered heteroalkylene)-C(O)—, wherein the 12-30 membered heteroalkylene can be linear or branched. In some preferred embodiments, L^{A} is 12-30 membered heteroalkylene or -(12-30 membered heteroalkylene)-C(O)—, wherein the 12-30 membered heteroalkylene is a linear and unsubstituted. In some embodiments, the 12-30 membered heteroalkylene includes 1, 2, 3, 4, or 5 heteroatoms independently selected from O, S, and N.

In some preferred embodiments, L^{A} can be characterized as having a structure according to

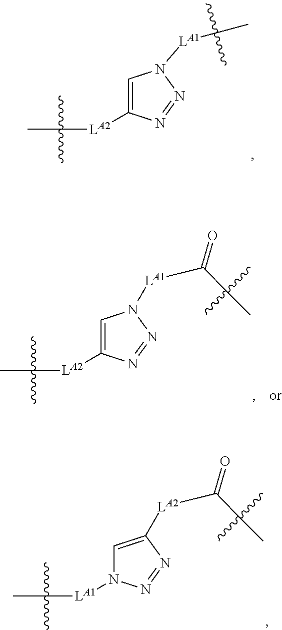

wherein the carbonyl is directly bonded with T^{A}, wherein L^{A1} and L^{A2} are each independently a bond, an optionally substituted —C₁₋₃₀ alkylene-, or an optionally substituted —C₁₋₃₀ heteroalkylene-containing 1-6 heteroatoms independently selected from O, N, and S, wherein the sulfur atom(s), if present, is optionally oxidized, wherein the optional substituents can optionally be joined together to form a double bond, triple bond, or a ring structure, wherein the end atoms of the L^{A1} and L^{A2} that are bonded with T^{A} or D, as applicable, are not C of a C(O) or S of a SO₂ group, wherein the longest chain length of L^{A} is at least that of —(CH₂)₁₂—, such as at least that of —(CH₂)₁₄—, at least that of —(CH₂)₁₆—, at least that of —(CH₂)₁₈—, and wherein the hydrophobicity of L^{A} can be characterized in that the corresponding compound

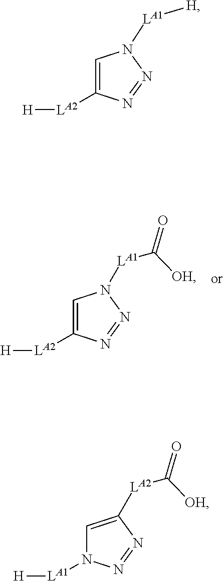

has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a bond. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

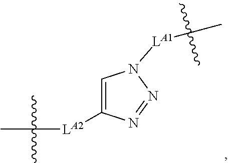

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atoms of the L^{A1} and L^{A2} that are bonded with T^{A} or D, as applicable, are not C of a C(O) or S of a SO₂ group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of (CH₂)₁₂—, such as at least that of (CH₂)₁₄—, at least that of —(CH₂)₁₆—, at least that of —(CH₂)₁₈—, wherein the hydrophobicity of L^{A} can be characterized in that the corresponding compound H-L^{A}-H has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc. In some embodiments, L^{A1} is a bond (i.e., not present, the triazole nitrogen atom is directly bonded with D or T^{A}). In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

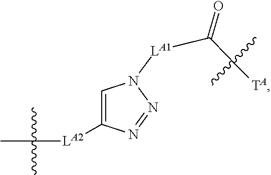

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atom of the L^{A2} that is bonded with D is not C of a C(O) or S of a SO₂ group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of —(CH₂)₁₂—, such as at least that of —(CH₂)₁₄—, at least that of —(CH₂)₁₆—, at least that of —(CH₂)₁₈—, wherein the hydrophobicity of L^{A} can be characterized in that the corresponding compound

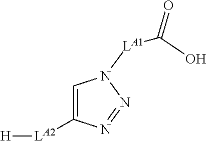

has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc. In some embodiments, L^{A1} is a bond. In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

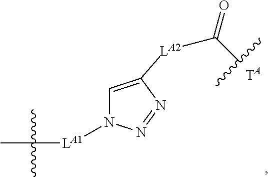

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atom of the LAI that is bonded with D is not C of a C(O) or S of a SO₂ group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of —(CH₂)₁₂—, such as at least that of —(CH₂)₁₄—, at least that of —(CH₂)₁₆—, at least that of —(CH₂)₁₈—, wherein the hydrophobicity of L^{A} can be characterized in that the corresponding compound

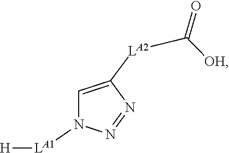

has a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc. In some embodiments, L^{A1} is a bond. In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.
Examples of L^{A}-T^{A}

The covalent bond formed between L^{A} and T^{A} is not particularly limited. For example, in some embodiments, the covalent bond(s) between the terminal atom(s) of T^{A} and the end carbon or nitrogen atom(s) of L^{A} is an amide bond. In some embodiments, the covalent bond(s) between the terminal atom(s) of T^{A} and the end atom(s) of L^{A} is a non-amide carbon-nitrogen bond, an ester bond, a non-ester carbon-oxygen bond, a carbon-carbon bond, or a carbon-sulfur bond.

In some embodiments, the compound of Formula I-1 can have a structure according to any of the following:

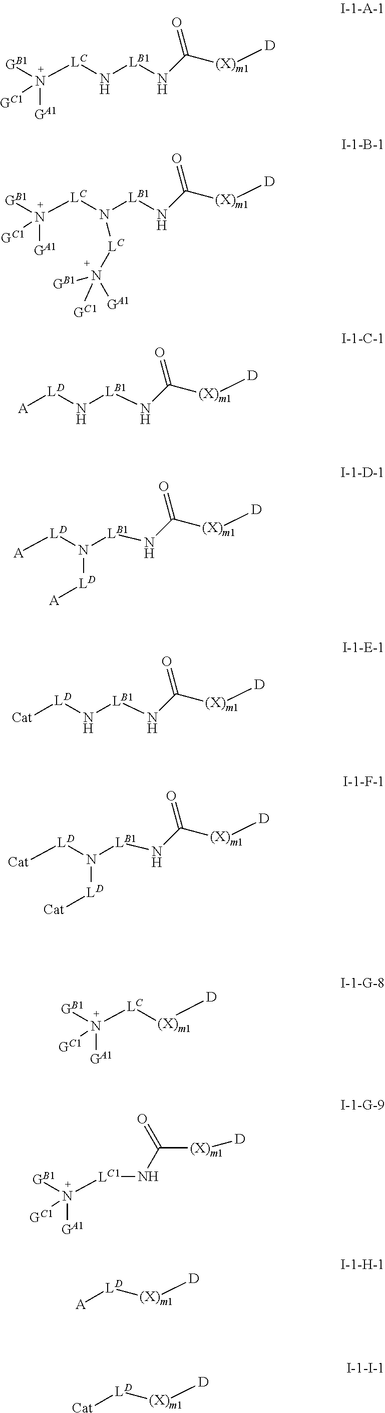

the integer m1 is at least 10, such as at least 12, at least 14, at least 16, at least 18, at least 20, at least 50, such as 12-50, 16-50, etc., and
the other variables are defined and preferred herein.
The charge or charges appearing in the formulae above, if necessary, is counter balanced with a counterion, so that the compound as a whole is neutral.

In some embodiments, L^{B1} is an optionally substituted C₁₋₆ alkylene (e.g., ethylene, n-propylene, n-butylene, etc.) or an optionally substituted C₁₋₆ heteroalkylene having one or two heteroatoms independently selected from N, O, P, and S, wherein the P or S is optionally oxidized, preferably, the atom that is bonded with the amide NH in the above formulae I-1-A-1, I-1-B-1, I-1-C-1, I-1-D-1, I-1-E-1, or I-1-F-1 is not a heteroatom.

In some embodiments, L^{C1} is an optionally substituted C₁₋₆ alkylene (e.g., ethylene, n-propylene, n-butylene, etc.) or an optionally substituted C₁₋₆ heteroalkylene having one or two heteroatoms independently selected from N, O, P, and S, wherein the P or S is optionally oxidized, preferably, the atom that is bonded with the amide NH in the above formula I-1-G-9 is not a heteroatom.

In some preferred embodiments, in Formula I-1-A-1, I-1-B-1, I-1-G-8 or I-1-G-9, G^{A1}, G^{B1}, and G^{C1} together with the nitrogen atom they are all attached to, are joined to form a structure of

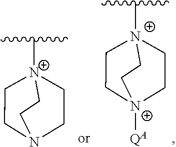

wherein Q^{A} is an optionally substituted C₁₋₄ alkyl, such as methyl. In some preferred embodiments, G^{A1}, G^{B1}, and G^{C1} together with the nitrogen atom they are all attached to, are joined to form

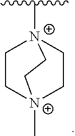 [SMILES: *[N+]12CC[N+](C)(CC1)CC2]

In some preferred embodiments, G^{A1}, G^{B1}, and G^{C1} together with the nitrogen atom they are all attached to, represents

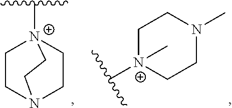

[N(CH₃)₃]⁺, or [N(CH₂CH₃)₃]⁺.

In some embodiments, in Formula I-1-A-1, I-1-B-1, or I-1-G-8, L^{C} is an optionally substituted C₁₋₆ alkylene (e.g., ethylene, n-propylene, n-butylene, etc.). In some embodiments, in Formula I-1-G-8, L^{C} is null.

In some embodiments, X at each occurrence is independently CR₂, —C(R)═C(R)—,

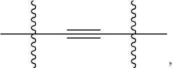 [SMILES: *C#C*]

or a 3-10 membered ring, wherein R at each occurrence is independently hydrogen or C₁₋₄ alkyl; more preferably, X at each occurrence is independently CR₂, wherein R at each occurrence is independently hydrogen or C₁₋₄ alkyl. In some embodiments, at most one X is a 3-10 membered ring. In some embodiments, no X contains a heteroatom. In some embodiments, all X is independently CR₂, wherein R at each occurrence is independently hydrogen or methyl, provided that at most 10 R (e.g., 1, 2, 3, 4, 5, or 6 R) are methyl groups.

In some embodiments, the hydrophobicity of —(X)ₘ₁—C(O)— in Formula I-1-A-1, I-1-B-1, I-1-C-1, I-1-D-1, I-1-E-1, or I-1-F-1, or I-1-G-9 is characterized in that the corresponding compound H—(X)ₘ₁—COOH should have a c Log P of at least 3, such as between 3-15, preferably, at least 4 (e.g., at least 4.5, at least 5, at least 5.5, at least 6, at least 6.5, at least 7, etc.), such as has a c Log P of 3-10, 4-12, 4.5-9, 5-11, 5.5-10.5, 6-14, 6.5-13, etc.

In some embodiments, —(X)ₘ₁— is an optionally substituted linear C₁₀₋₅₀ alkylene, such as an optionally substituted linear C₁₀₋₃₀ alkylene (e.g., linear C₁₀, C₁₂, C₁₄, C₁₆, C₁₈, C₂₀, C₂₂, or C₂₄ alkylene, etc.). In some embodiments, 1-10 (e.g., 1, 2, 3, 4, 5, or 6) CH₂ in the linear alkylene chain may be substituted, for example, each of the 1-10 CH₂ can be independently substituted with one or two methyl groups. In some embodiments, —(X)ₘ₁— can be a group derived from replacing 1-10 (e.g., 1, 2, 3, 4, 5, or 6) CH₂ in the foregoing linear alkylene chain each with a double bond, for example, when one CH₂ in a C10 alkylene is replaced with a double bond, the resulted group is a C11 alkenylene having one double bond.

In some embodiments, D is a residue having the formula of D-1, D-2-A, D-2-B, D-3-A, or D-3-B, or a subformula thereof, as defined herein.

### Residue of a Ligand of a Membrane Bound Protein

In Formula I, D is typically a residue of a ligand of a GPCR, although residues of ligands of other membrane bound proteins are also suitable. In some particular embodiments, D is a residue of a GPR40 agonist.

In some embodiments, D is a residue having the formula of D-1:

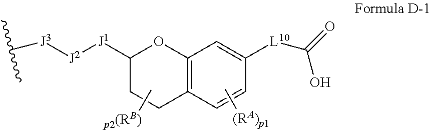

wherein:
L¹⁰ is an alkylene (e.g., a C₁₋₆ alkylene), optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{B} at each occurrence is independently halogen, hydroxyl, amino, substituted amino, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{B} are joined to form an optionally substituted ring structure; p2 is 0, 1, 2, 3, or 4;
J¹ is a bond, an optionally substituted aryl or heteroaryl ring, C₁₋₆alkylene-N(R¹⁰⁰)—, 3-14 membered optionally substituted heterocyclylene containing at least one ring nitrogen atom, or —C₁₋₆alkylene-(3-14 membered optionally substituted heterocyclylene containing at least one ring nitrogen atom)-, wherein R¹⁰⁰ is hydrogen, optionally substituted alkyl, or optionally substituted cycloalkyl;
J² is a bond or an alkylene, optionally substituted with 1-3 substituents independently selected from halogen, optionally substituted C₁₋₆ alkyl, optionally substituted C₂—, alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, or two substituents are joined to form an optionally substituted ring structure; and
J³ is an optionally substituted cycloalkyl, heterocyclyl, aryl or heteroaryl ring.

The variables L¹⁰, J¹, J², J³, R^{A}, R^{B}, p1, and p2 in Formula D-1 are not particularly limited. However, in preferred embodiments, the variables in Formula D-1 are such that at least one corresponding compound according to Formula GPR-1,

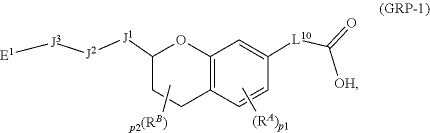

wherein E¹ is hydrogen, C₁₋₄ alkyl, N3,

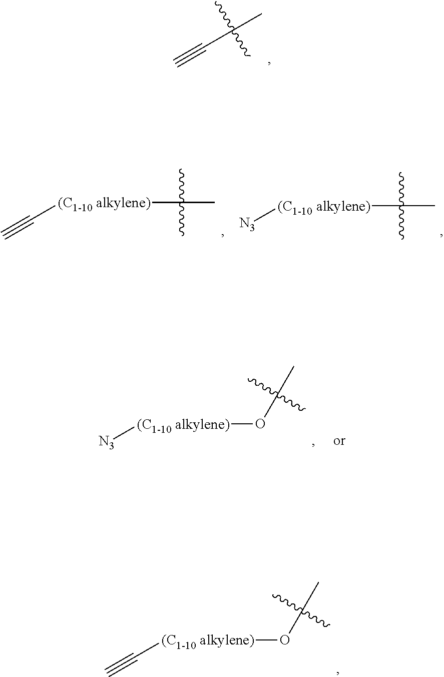

is a GPR40 agonist, preferably, having an EC50 of less than 100 nM as measured according to Biological Example 1 herein.

Typically, p1 in Formula D-1 is 0.

In some embodiments, p1 in Formula D-1 is 1, and R^{A} is F, Cl, CN, C₁₋₄ alkyl optionally substituted with 1-3 fluorine, or C₁₋₄ alkoxy optionally substituted with 1-3 fluorine.

Typically, p2 in Formula D-1 is 0.

In some embodiments, p2 in Formula D-1 is 1 or 2, and R^{B} at each occurrence is independently F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

In some preferred embodiments, L¹⁰ is an optionally substituted ethylene. When substituted, the ethylene is typically substituted with one or two substituents, each independently a C₁₋₄ alkyl or a C₃₋₆ cycloalkyl. For example, in some embodiments, L¹⁰ is

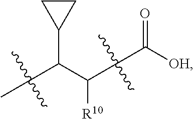

wherein R¹⁰ is hydrogen or C₁₋₄ alkyl. In some preferred embodiments, Formula D-1 can be characterized as having a formula of

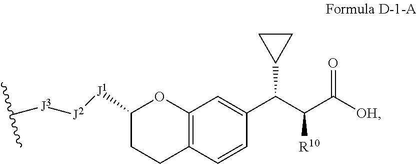

wherein R¹⁰ is hydrogen or C₁₋₄ alkyl (preferably methyl), wherein J¹, J², and J³ are defined herein.

In some embodiments, J¹ in Formula D-1 (e.g., Formula D-1-A) is C₁₋₆alkylene-N(R¹⁰⁰), such as CH₂—N(C₁₋₄ alkyl). Typically, in Formula D-1, J¹ is a 4-12 membered optionally substituted heterocyclic ring having one or two ring nitrogen atoms. In some embodiments, JP in Formula D-1 (e.g., Formula D-1-A) is a 4-12 membered optionally substituted heterocyclic ring having one or two ring nitrogen atoms. For example, in some embodiments, J¹ is a 4-8 (e.g., 4, 5, 6, or 7) membered monocyclic optionally substituted saturated heterocyclic ring having one or two ring heteroatoms independently selected from S, O, and N, provided at least one of the ring heteroatom is nitrogen. In some embodiments, J¹ is selected from the following (J² is included to show direction of connections):

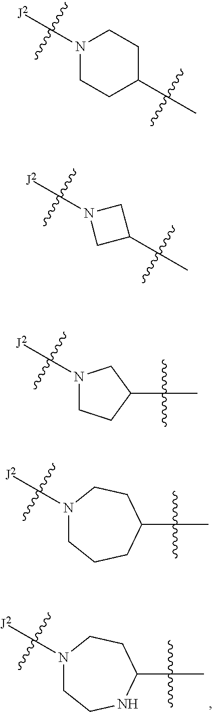

each of which is optionally substituted with 1-2 substituents independently selected from F, OH, NH₂, NH(C₁₋₄ alkyl), N(C₁₋₄ alkyl)(C₁₋₄ alkyl), C₁₋₄ alkyl optionally substituted with 1-3 fluorine, and C₁₋₄ alkoxy optionally substituted with 1-3 fluorine.

In some embodiments, J¹ in Formula D-1 (e.g., Formula D-1-A) can also be a bicyclic or polycyclic 6-12 membered optionally substituted saturated heterocyclic ring having one or two ring heteroatoms independently selected from S, O, and N, provided at least one of the ring heteroatom is nitrogen. For example, in some embodiments, J is selected from the following (J² is included to show direction of connections):

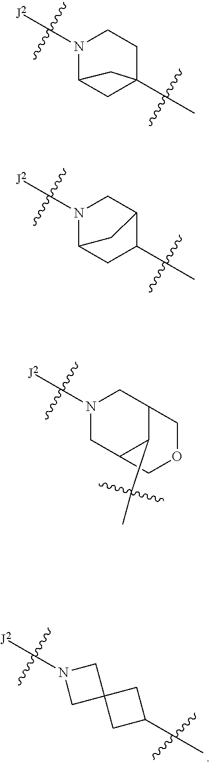

In some embodiments, J² in Formula D-1 (e.g., Formula D-1-A) is a straight chain or branched C₁₋₄ alkylene, optionally substituted with 1-3 fluorine. For example, in some embodiments, J² is CH₂ or —CH(CH₃)—.

J³ in Formula D-1 (e.g., Formula D-1-A) is typically an aryl (e.g., phenyl) or heteroaryl ring (e.g., pyridyl), each of which is unsubstituted or substituted with one or more (e.g., 1, 2, or 3) substituents independently selected from 1) halogen, CN, —CF₃, OH, amino, substituted amino, ester, amide, carbonate, or carbamate; and 2) C₁₋₆ alkyl, C₂₋₆ alkenyl, C₂₋₆ alkynyl, C₁₋₆ heteroalkyl, C₃₋₆ cycloalkyl, C₁₋₆ alkoxy, C₃₋₆ cycloalkoxy, aryl, heteroaryl, 3-8 membered heterocycloalkyl having one or two ring heteroatoms independently selected from N, O, and S, wherein each of which is optionally substituted with one or more (e.g., 1, 2, or 3) substituents independently selected from F, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy.

In some embodiments, J³ in Formula D-1 (e.g., Formula D-1-A) is a phenyl ring, which is substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F. For example, in some embodiments, the phenyl ring can be substituted with one or two substituents independently selected from C₁₋₄ alkyl optionally substituted with fluorine, e.g., CF₃, and C₁₋₆ alkoxy optionally substituted with fluorine, such as methoxy, ethoxy, isopropoxy, or O—CF₃.

In some embodiments, J³ in Formula D-1 (e.g., Formula D-1-A) is a 5-10 membered monocyclic or bicyclic heteroaryl ring, which is substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

For example, in some embodiments, J³ in Formula D-1 (e.g., Formula D-1-A) is selected from:

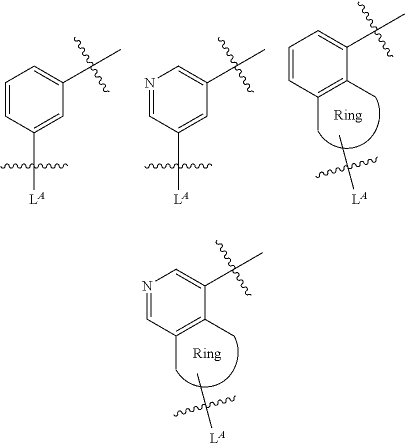

wherein: Ring represents an aromatic or non-aromatic ring structure,
wherein each of the phenyl, pyridyl, or fused ring structure is optionally substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F. For example, in some embodiments, the phenyl, pyridyl, or fused ring structure can be substituted with one or two substituents independently selected from C₁₋₄ alkyl optionally substituted with fluorine, e.g., CF₃, and C₁₋₆ alkoxy optionally substituted with fluorine, such as methoxy, ethoxy, isopropoxy, or O—CF₃.

In some embodiments, J³ in Formula D-1 (e.g., Formula D-1-A) is selected from:

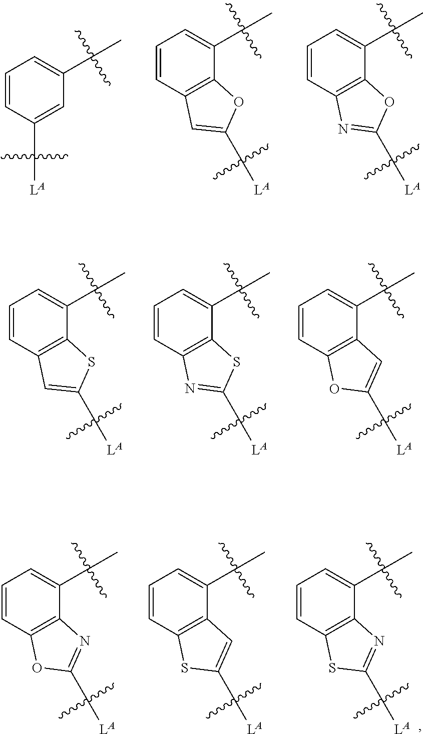

wherein each of which is optionally substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

In some embodiments, J³ in Formula D-1 (e.g., Formula D-1-A) is selected from:

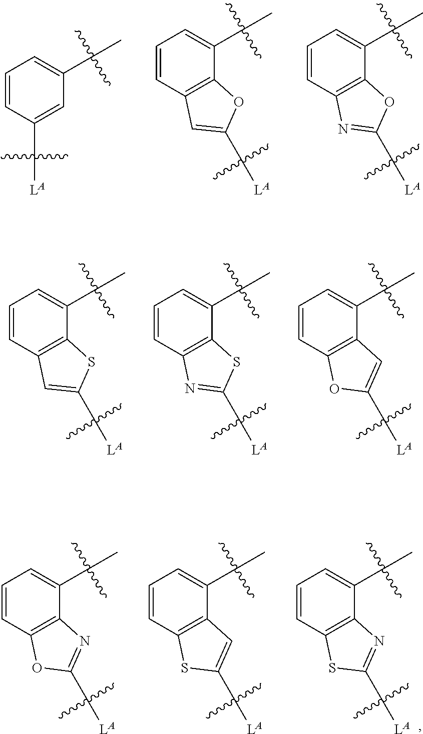

wherein each of which is optionally substituted with 1-3 substituents independently selected from F, Cl, CN, OH, C₁₋₆ alkyl optionally substituted with F (e.g., CF₃), cyclopropyl, cyclobutyl, C₁₋₆ alkoxy optionally substituted with F (e.g., —O—CF₃), or C₃₋₆ cycloalkoxy. For example, in some embodiments, the phenyl, benzofuran, benzothiophene, benzoxazol, or benzothiazol ring can be substituted with one or two substituents independently selected from C₁₋₄ alkyl optionally substituted with fluorine, e.g., CF₃, C₁₋₆ alkoxy optionally substituted with fluorine, such as methoxy, ethoxy, isopropoxy, or O—CF₃. Preferably, the one substituent is ortho to J².

In some embodiments, D is characterized as having a Formula D-1-A-1, D-1-A-2, D-1-A-3, D-1-A-4, or D-1-A-5:

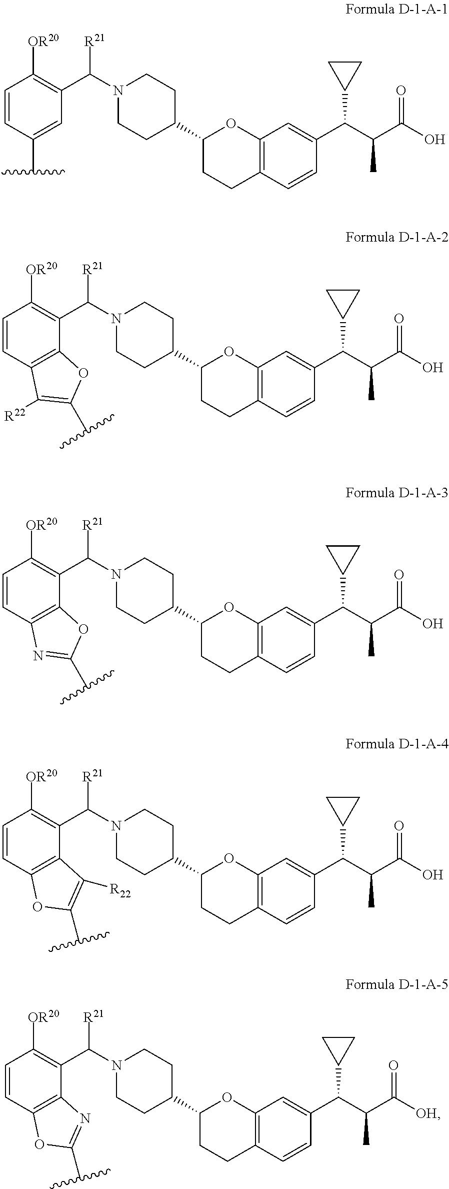

wherein:
R²⁰ is C₁₋₆ alkyl or fluorine substituted C₁₋₆ alkyl, R²¹ is hydrogen or C₁₋₆ alkyl, and R²² is hydrogen, halogen, CN, C₁₋₆ alkyl or fluorine substituted C₁₋₆ alkyl or a C₃₋₆ cycloalkyl, wherein L^{A} and T^{A} include any of those described herein in any combinations. In some embodiments, R²⁰ is methyl, ethyl, n-propyl, isopropyl, or CF₃. In some embodiments, R²¹ is hydrogen, methyl, ethyl, n-propyl, or isopropyl. In some embodiments, R²⁰ is CF₃ and R²¹ is hydrogen or methyl. In some embodiments, R²⁰ is CH₃ and R²¹ is hydrogen or methyl. In some embodiments, R²² is hydrogen. In some embodiments, R²² is methyl. In some embodiments, R²² is cyclopropyl.

In some embodiments, D is characterized as having a Formula D-1-A-6, D-1-A-7, D-1-A-8, D-1-A-9, or D-1-A-10:

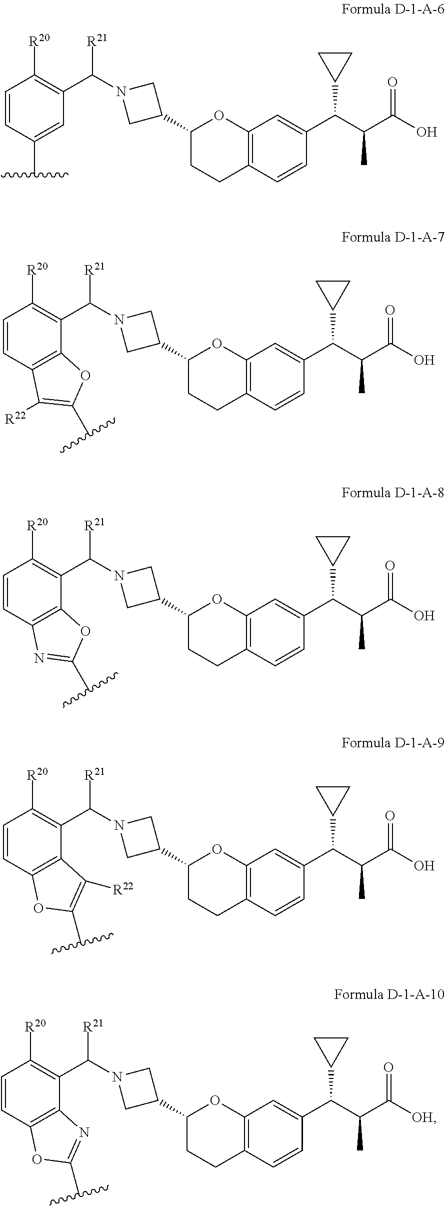

wherein:
R²⁰ is C₁₋₆ alkyl or fluorine substituted C₁₋₆ alkyl, R²¹ is hydrogen or C₁₋₆ alkyl, and R²² is hydrogen, halogen, CN, C₁₋₆ alkyl or fluorine substituted C₁₋₆ alkyl or a C₃₋₆ cycloalkyl, wherein L^{A} and T^{A} include any of those described herein in any combinations. In some embodiments, R²⁰ is methyl, ethyl, n-propyl, isopropyl, or CF₃. In some embodiments, R²¹ is hydrogen, methyl, ethyl, n-propyl, or isopropyl. In some embodiments, R²⁰ is CF₃ and R²¹ is hydrogen or methyl. In some embodiments, R²⁰ is CH₃ and R²¹ is hydrogen or methyl. In some embodiments, R²² is hydrogen. In some embodiments, R²² is methyl. In some embodiments, R²² is cyclopropyl.

In some embodiments, D in Formula I (e.g., I-1) is a residue having the formula of D-2-A or D-2-B:

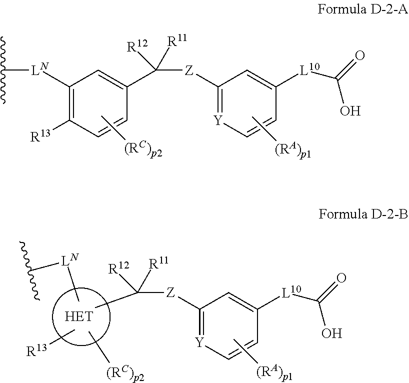

wherein:
Y is CH, CR^{A}, or N;
Z is O, S, NH, or N(C₁₋₄ alkyl);
HET ring stands for an optionally substituted heteroaryl ring (e.g., a 5 or 6-membered heteroaryl, such as a triazole ring);
R¹¹ and R¹² are each independently hydrogen or C₁₋₄ alkyl;
L^{N} is null, an optionally substituted C₁₋₆ alkylene, or an optionally substituted C₁₋₆ heteroalkylene having 1-3 heteroatoms;
L¹⁰ is an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{C} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two RC are joined to form an optionally substituted ring structure; p2 is 0, 1, 2, or 3; and
R¹³ is hydrogen, an optionally substituted phenyl or an optionally substituted heteroaryl.

In some embodiments, D has a structure according to Formula D-2-A.

In some embodiments, D has a structure according to Formula D-2-B.

In some embodiments according to Formula D-2-A or D-2-B, L^{N} is null, i.e., L^{A}(in Formula I) is directly connected to the phenyl ring drawn in Formula D-2-A or the HET ring in D-2-B. To be clear, for the purposes herein, the definition of L^{N} as null should not be interpreted such that in such embodiments, L^{A} cannot contain a fragment that fits into one or more of the definitions of L^{N} described herein. Rather, in some embodiments, when L^{N} is defined as null, the variable L^{A} can have any of the definitions herein described for L^{N}-L^{A} in embodiments where L^{N} is not null.

In some embodiments according to Formula D-2-A or D-2-B, L^{N} is a branched or straight chained C₁₋₆ alkylene, such as

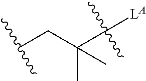

(L^{A} is shown to show direction of connection). In some embodiments according to Formula D-2-A or D-2-B, L^{N} is

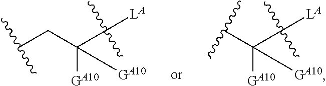

(L^{A} is shown to show direction of connection), wherein G^{A10} at each occurrence is independently hydrogen or an optionally substituted C₁₋₄ alkyl, or two G^{A10} are joined to form a 3-6 membered ring, such as a cyclopropyl or cyclobutyl ring. In some preferred embodiments, G^{A10} at each occurrence is methyl.

In some embodiments according to Formula D-2-A or D-2-B, L^{N} is a branched or straight chained C₁₋₆ heteroalkylene having one or two oxygen atoms, such as

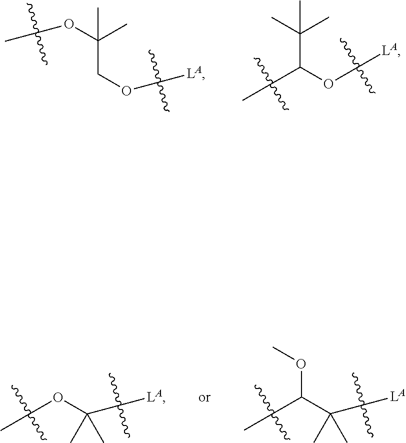

(L^{A} is shown to show direction of connection). In some embodiments according to Formula D-2-A or D-2-B, L^{N} is

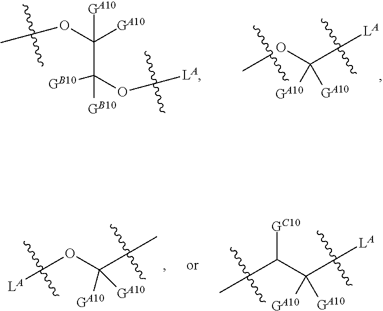

(L^{A} is shown to show direction of connection), wherein G^{A10} at each occurrence is independently hydrogen or an optionally substituted C₁₋₄ alkyl, or two G^{A10} are joined to form a 3-6 membered ring, such as a cyclopropyl or cyclobutyl ring, wherein G^{B10} at each occurrence is independently hydrogen or an optionally substituted C₁₋₄ alkyl, or two G^{B10} or one G^{A10} and one G^{B10} are joined to form a 3-6 membered ring, such as a cyclopropyl or cyclobutyl ring, wherein G^{C10} is hydrogen, an optionally substituted C₁₋₄ alkyl, or an optionally substituted C₁₋₄ heteroalkyl (e.g., C₁₋₄ alkoxy). In some preferred embodiments, G^{A10} at each occurrence is methyl. In some preferred embodiments, G^{B10} at each occurrence is hydrogen. In some preferred embodiments, G^{C10} is hydrogen or C₁₋₄ alkoxy such as methoxy.

In some embodiments, Formula D-2-A can be characterized as having a Formula D-2-A-1:

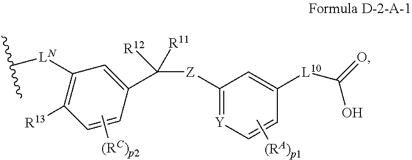

wherein the variables are defined herein.

In some embodiments, Formula D-2-B can be characterized as having a Formula D-2-B-1:

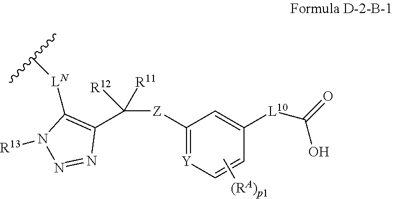

wherein the variables are defined herein.

The variables L¹⁰, R¹¹, R¹², R¹³, R^{A}, R^{C}, Y, Z, HET, p1, and p2 in Formula D-2-A or D-2-B are not particularly limited. However, in preferred embodiments, the variables in Formula D-2-A or D-2-B are such that at least one corresponding compound according to Formula GPR-2,

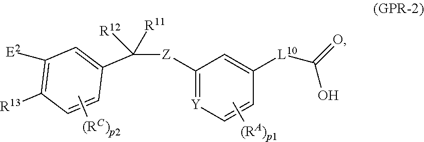

### or Formula GPR-2B,

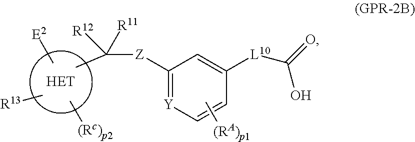

wherein E² is E^{2A} or L^{N}-E^{2A}, wherein E^{2A} is hydrogen, N3,

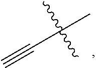 [SMILES: *C#C]

C₁₋₄ alkyl,

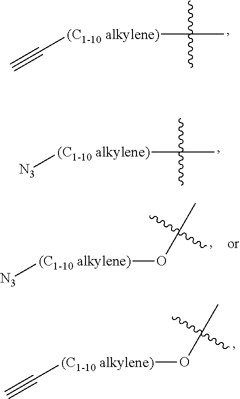

and L^{N} is defined herein (such as null or a C₁₋₆ alkylene), is a GPR40 agonist, preferably, having an EC50 of less than 100 nM as measured according to Biological Example 1 herein.

Typically, p1 in Formula D-2-A or D-2-B is 0.

In some embodiments, p1 in Formula D-2-A or D-2-B is 1.

Typically, R^{A} at each occurrence is independently F, Cl, CN, C₁₋₄ alkyl optionally substituted with 1-3 fluorine, or C₁₋₄ alkoxy optionally substituted with 1-3 fluorine.

Typically, p2 in Formula D-2-A or D-2-B is 0.

In some embodiments, p2 in Formula D-2-A or D-2-B is 1, and R^{C} is F, Cl, CN, C₁₋₄ alkyl optionally substituted with 1-3 fluorine, or C₁₋₄ alkoxy optionally substituted with 1-3 fluorine.

In Formula D-2-A or D-2-B, Y is typically CH.

Preferably, Z in Formula D-2-A or D-2-B is O.

In some embodiments, R¹¹ and R¹² are both hydrogen.

In some preferred embodiments, L¹⁰ is characterized as having a structure of

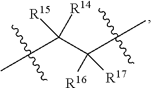

wherein CR¹⁶R¹⁷ is bonded to the COOH group, and wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and
R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S. In some embodiments, R¹⁶ and R¹⁷ are both hydrogen, or one of R¹⁶ and R¹⁷ is hydrogen and the other of R¹⁶ and R¹⁷ is methyl. In some embodiments, one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl. In some embodiments, R¹⁴ and R¹⁵ are joined to form a C₃₋₆ cycloalkyl.

For example, in some embodiments, L¹⁰ is

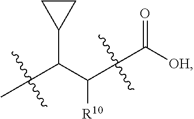

wherein R¹⁰ is hydrogen or C₁₋₄ alkyl, such as methyl.

R¹³ in Formula D-2-A or D-2-B is typically an optionally substituted phenyl or an optionally substituted 5 or 6 membered heteroaryl having 1-4 ring heteroatoms. In some embodiments, R¹³ in Formula D-2-A or D-2-B can also be hydrogen.

In some embodiments, R¹³ is an optionally substituted phenyl. In some embodiments, R¹³ is a phenyl ring, which is unsubstituted. In some embodiments, R¹³ is a phenyl ring, which is substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

In some embodiments, R¹³ is an optionally substituted 6-membered heteroaryl ring. In some embodiments, R¹³ is a 6-membered heteroaryl ring, such as a pyridyl ring,

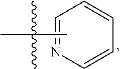

which is optionally substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

In some preferred embodiments, Formula D-2-A or D-2-B can be characterized as having a Formula D-2-A-2 or Formula D-2-B-2:

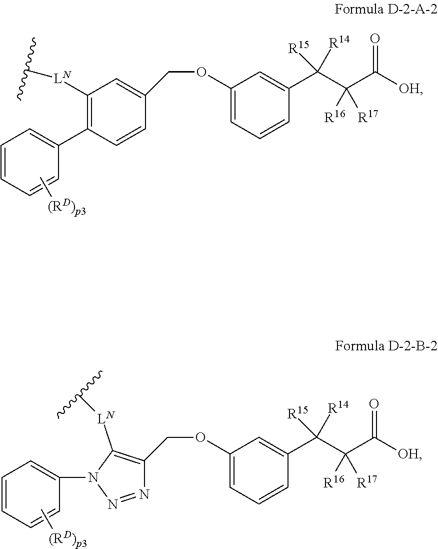

wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S;
R^{D} at each occurrence is independently F, Cl, C₁₋₄ alkyl optionally substituted with 1-3 F, or C₁₋₄ alkoxy optionally substituted with 1-3 F, and
wherein p3 is 0, 1, 2, or 3,
and L^{N} is described and preferred herein. For example, in some embodiments, L^{N} is null. In some embodiments, L^{N} is a branched or straight chained C₁₋₆ alkylene, such as

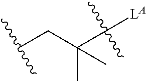

(L^{A} is shown to show direction of connection). In some embodiments, R¹⁶ and R¹⁷ are both hydrogen. In some embodiments, one of R¹⁶ and R¹⁷ is hydrogen and the other of R¹⁶ and R¹⁷ is methyl. In some embodiments, one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl. In some embodiments, R¹⁴ and R¹⁵ are joined to form a C₃₋₆ cycloalkyl.

In some preferred embodiments, D has a structure according to Formula D-2-A-3 or D-2-B-3:

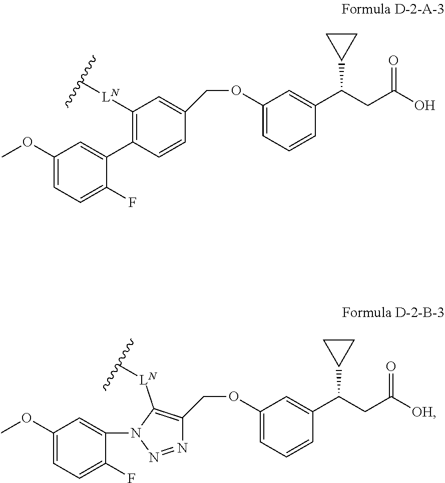

wherein L^{N} is described and preferred herein. For example, in some embodiments, L^{N} is null. In some embodiments, L^{N} is a branched or straight chained C₁₋₆ alkylene, such as

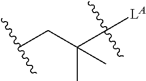

(L^{A} is shown to show direction of connection).

In some embodiments, D in Formula I (e.g., I-1) can have a structure according to Formula D-3-A or D-3-B:

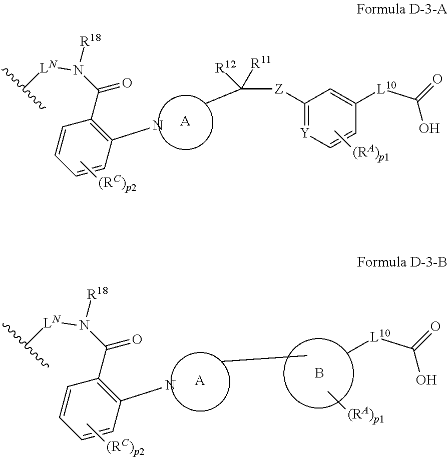

wherein:
Y is CH, CR^{A}, or N;
Z is O, S, NH, or N(C₁₋₄ alkyl);
R¹¹ and R¹² are each independently hydrogen or C₁₋₄ alkyl;
Ring A is an optionally substituted 4-12 membered nitrogen-containing ring;
Ring B is an optionally substituted monocyclic heteroaryl or a bicyclic aryl or heteroaryl ring, such as a benzofuran ring;
L^{N} is null, an optionally substituted C₁₋₆ alkylene, or an optionally substituted C₁₋₆ heteroalkylene having 1-3 heteroatoms;
L¹⁰ is an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{C} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two RC are joined to form an optionally substituted ring structure; p2 is 0, 1, 2, or 3; and
R¹⁸ is an optionally substituted phenyl or an optionally substituted heteroaryl.

In some embodiments, D has a structure according to Formula D-3-A.

In some embodiments, D has a structure according to Formula D-3-B.

In some embodiments, D has a structure according to Formula D-3-A-1 or D-3-B-1:

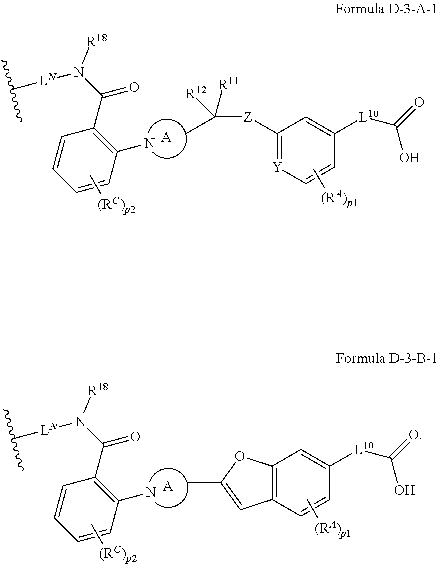

In some embodiments according to Formula D-3-A or D-3-B, L^{N} is null, i.e., L^{A}(in Formula I) is directly connected to the amide nitrogen atom shown in Formula D-3-A or D-3-B. To be clear, for the purposes herein, the definition of L^{N} as null should not be interpreted such that in such embodiments, L^{A} cannot contain a fragment that fits into one or more of the definitions of L^{N} described herein. Rather, in embodiments, when L^{N} is defined as null, the variable L^{A} can have any of the definitions herein described for L^{N}-L^{A} in embodiments where L^{N} is not null.

In some embodiments according to Formula D-3-A or D-3-B, L^{N} is a branched or straight chained C₁₋₆ alkylene, such as

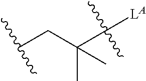

(L^{A} is shown to show direction of connection). In some embodiments according to Formula D-3-A or D-3-B, L^{N} is

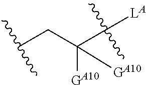

(L^{A} is shown to show direction of connection), wherein G^{A10} at each occurrence is independently hydrogen or an optionally substituted C₁₋₄ alkyl, or two G^{A10} are joined to form a 3-6 membered ring, such as a cyclopropyl or cyclobutyl ring. In some preferred embodiments, G^{A10} at each occurrence is methyl.

The variables L¹⁰, R¹¹, R¹², R¹⁸, R^{A}, R^{C}, ring A, ring B, Y, Z, p1, and p2 in Formula D-3-A or D-3-B are not particularly limited. However, in preferred embodiments, the variables in Formula D-3-A or D-3-B are such that at least one corresponding compound according to Formula GPR-3,

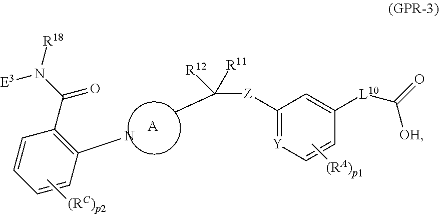

### or Formula GPR-3B,

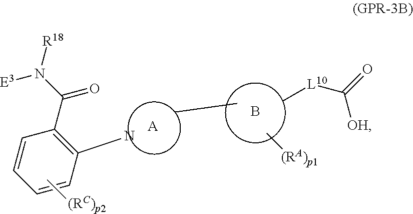

wherein E³ is E^{3A} or L^{N}-E^{3A}, wherein E^{3A} is hydrogen, N3,

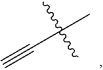 [SMILES: *C#C]

C₁₋₄ alkyl,

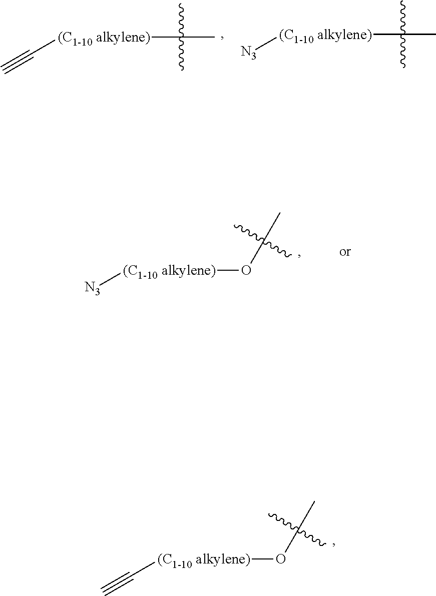

and L^{N} is defined herein (such as null or a C₁₋₆ alkylene), is a GPR40 agonist, preferably, having an EC50 of less than 100 nM as measured according to Biological Example 1 herein.

Typically, p1 in Formula D-3-A or D-3-B is 0.

In some embodiments, p1 in Formula D-3-A or D-3-B is 1.

Typically, R^{A} at each occurrence is independently F, Cl, CN, C₁₋₄ alkyl optionally substituted with 1-3 fluorine, or C₁₋₄ alkoxy optionally substituted with 1-3 fluorine.

Typically, p2 in Formula D-3-A or D-3-B is 0.

In some embodiments, p2 in Formula D-3-A or D-3-B is 1, and R^{C} is F, Cl, CN, C₁₋₄ alkyl optionally substituted with 1-3 fluorine, or C₁₋₄ alkoxy optionally substituted with 1-3 fluorine.

In Formula D-3-A, Y is typically N.

Preferably, Z in Formula D-3-A is O.

In some embodiments, R¹ and R¹² are both hydrogen.

In some preferred embodiments, L¹⁰ is characterized as having a structure of

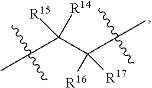

wherein CR¹⁶R¹⁷ is bonded to the COOH group, and wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S. In some embodiments, R¹⁶ and R⁷ are both hydrogen, or one of R¹⁶ and R⁷ is hydrogen and the other of R¹⁶ and R¹⁷ is methyl. In some embodiments, one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl. In some embodiments, R¹⁴ and R¹⁵ are joined to form a C₃₋₆ cycloalkyl.

For example, in some embodiments, L¹⁰ is

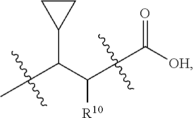

wherein R¹⁰ is hydrogen or C₁₋₄ alkyl, such as methyl.

R¹⁸ in Formula D-3-A or D-3-B is typically an optionally substituted phenyl or an optionally substituted 5 or 6 membered heteroaryl having 1-4 ring heteroatoms.

In some embodiments, R¹⁸ is an optionally substituted 6-membered heteroaryl ring. In some embodiments, R¹⁸ is a 6-membered heteroaryl ring, such as a pyridyl ring,

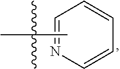

which is optionally substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F. In some embodiments, R¹⁸ is

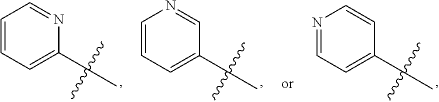 [SMILES: *c1ccccn1 ; *c1cccnc1 ; *c1ccncc1]

each of which is optionally substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

Ring A for Formula D-3-A or D-3-B is a nitrogen containing heterocyclic structure, with at least one nitrogen that is bonded with the phenyl ring shown in Formula D-3-A or D-3-B.

In some embodiments, Ring A for Formula D-3-A or D-3-B is a 4-8 membered optionally substituted monocyclic saturated heterocyclic ring having one or two ring heteroatoms independently selected from S, O, and N, provided at least one of the ring heteroatom is nitrogen. For example, in some embodiments, Ring A is selected from:

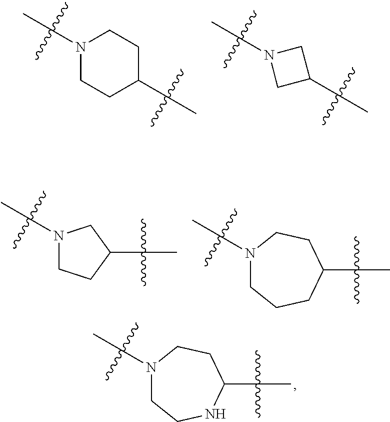 [SMILES: *C1CCN(*)CC1 ; *C1CN(*)C1 ; *C1CCN(*)C1 ; *C1CCCN(*)CC1 ; *C1CCN(*)CCN1]

each of which is optionally substituted with 1-2 substituents independently selected from F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

In some embodiments, Ring A for Formula D-3-A or D-3-B can also be a bicyclic or polycyclic 6-12 membered optionally substituted saturated heterocyclic ring having one or two ring heteroatoms independently selected from S, O, and N, provided at least one of the ring heteroatom is nitrogen.

In some preferred embodiments, D has a structure according to Formula D-3-A-2 or D-3-B-2:

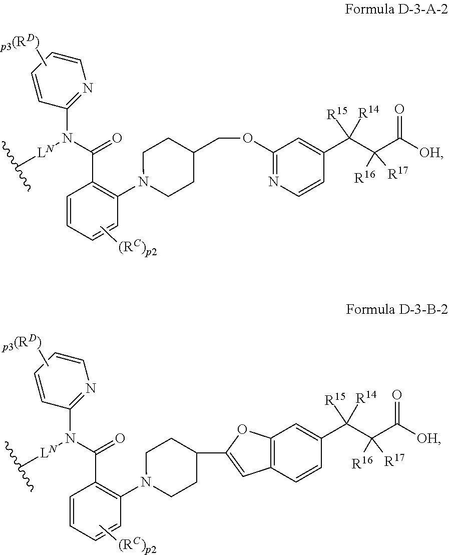

wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S;
R^{D} at each occurrence is independently F, Cl, C₁₋₄ alkyl optionally substituted with 1-3 F, or C₁₋₄ alkoxy optionally substituted with 1-3 F, and
wherein p3 is 0, 1, 2, or 3,
and L^{N} is described and preferred herein. For example, in some embodiments, L^{N} is null. In some embodiments, L^{N} is a branched or straight chained C₁₋₆ alkylene, such as

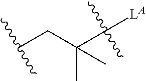

(L^{A} is shown to show direction of connection). In some embodiments, R¹⁶ and R¹⁷ are both hydrogen. In some embodiments, one of R¹⁶ and R¹⁷ is hydrogen and the other of R⁶ and R¹⁷ is methyl. In some embodiments, one of R⁴ and R¹⁵ is hydrogen, and the other of R¹⁴ and R¹⁵ is C₁₋₆ alkyl, C₂—, alkenyl, C₂₋₆ alkynyl, or C₃₋₆ cycloalkyl. In some embodiments, R¹⁴ and R¹⁵ are joined to form a C₃₋₆ cycloalkyl.

In some preferred embodiments, D has a structure according to Formula D-3-A-3 or D-3-B-3:

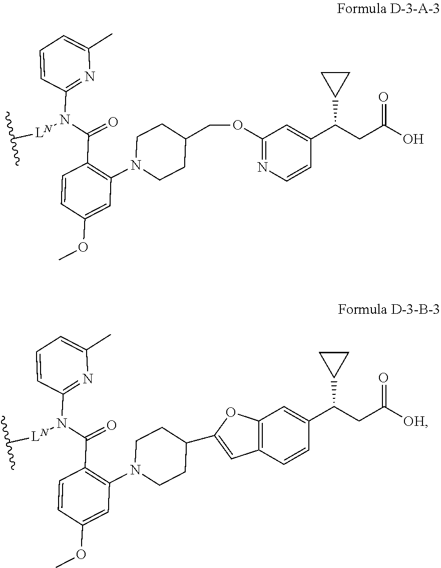

wherein L^{N} is described and preferred herein. For example, in some embodiments, L^{N} is null. In some embodiments, L^{N} is a branched or straight chained C₁₋₆ alkylene, such as

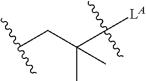

(L^{A} is shown to show direction of connection).

In some embodiments, the present disclosure also provides a compound selected from Table 1 below, or a pharmaceutically acceptable salt or ester thereof, wherein Ct^{⊖} represents a counterion, preferably, a pharmaceutically acceptable anion, such as Cl⁻, etc. It should be clear that under certain conditions, the compound shown in Table 1 may exist as an internal salt, i.e., a zwitterion structure, in which case, [symbol] may not be needed for balancing the charge shown or in cases when [symbol] are shown in the structure, only one of the [symbol] is needed. As used herein, an internal salt derivable from a compound shown in Table 1 (with or without any [symbol] ) is within the definition of the compound shown in Table 1, or a pharmaceutically acceptable salt thereof.

TABLE 1
List of Exemplary Compounds

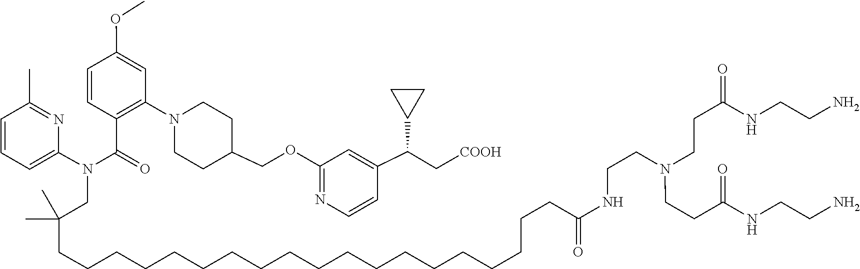 [SMILES: COc1ccc(C(=O)N(CC(C)(C)CCCCCCCCCCCCCCCCCCCC(=O)NCCN(CCC(=O)NCCN)CCC(=O)NCCN)c2cccc(C)n2)c(N2CCC(COc3cc([C@@H](CC(=O)O)C4CC4)ccn3)CC2)c1]

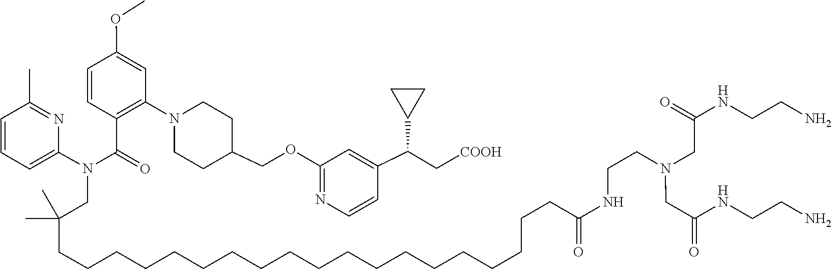 [SMILES: COc1ccc(C(=O)N(CC(C)(C)CCCCCCCCCCCCCCCCCCCC(=O)NCCN(CC(=O)NCCN)CC(=O)NCCN)c2cccc(C)n2)c(N2CCC(COc3cc([C@@H](CC(=O)O)C4CC4)ccn3)CC2)c1]

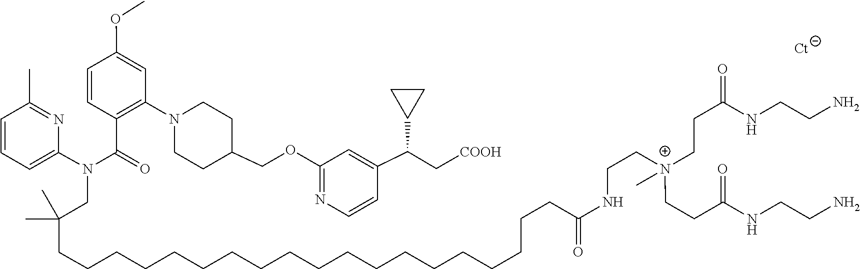 [SMILES: COc1ccc(C(=O)N(CC(C)(C)CCCCCCCCCCCCCCCCCCCC(=O)NCC[N+](C)(CCC(=O)NCCN)CCC(=O)NCCN)c2cccc(C)n2)c(N2CCC(COc3cc([C@@H](CC(=O)O)C4CC4)ccn3)CC2)c1 ; "Ct"]

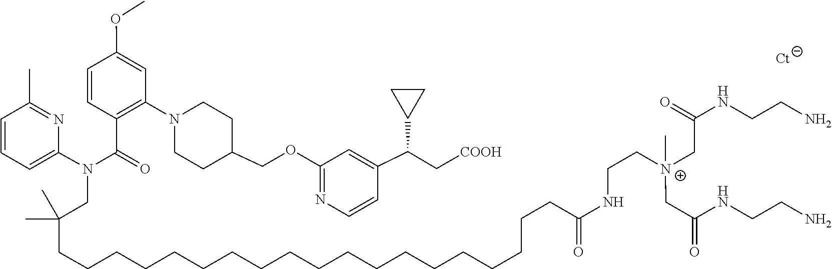

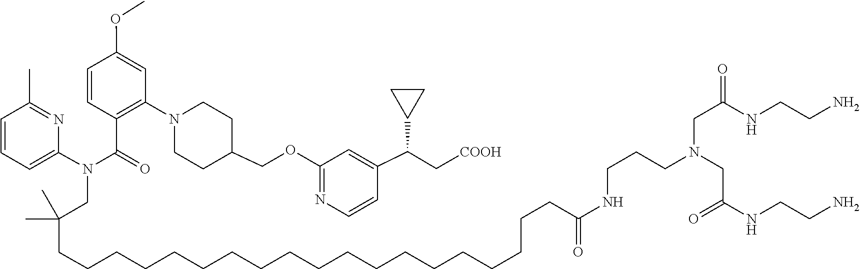 [SMILES: COc1ccc(C(=O)N(CC(C)(C)CCCCCCCCCCCCCCCCCCCC(=O)NCCCN(CC(=O)NCCN)CC(=O)NCCN)c2cccc(C)n2)c(N2CCC(COc3cc([C@@H](CC(=O)O)C4CC4)ccn3)CC2)c1]

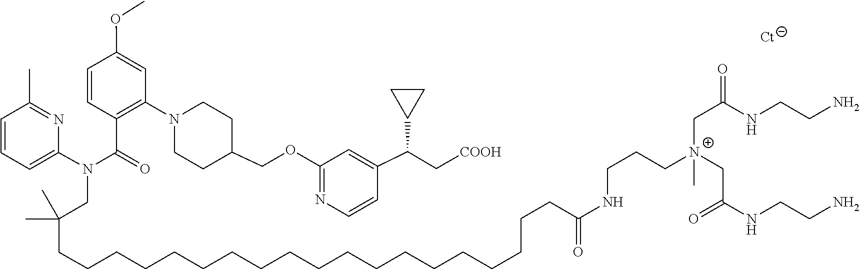 [SMILES: COc1ccc(C(=O)N(CC(C)(C)CCCCCCCCCCCCCCCCCCCC(=O)NCCC[N+](C)(CC(=O)NCCN)CC(=O)NCCN)c2cccc(C)n2)c(N2CCC(COc3cc([C@@H](CC(=O)O)C4CC4)ccn3)CC2)c1]

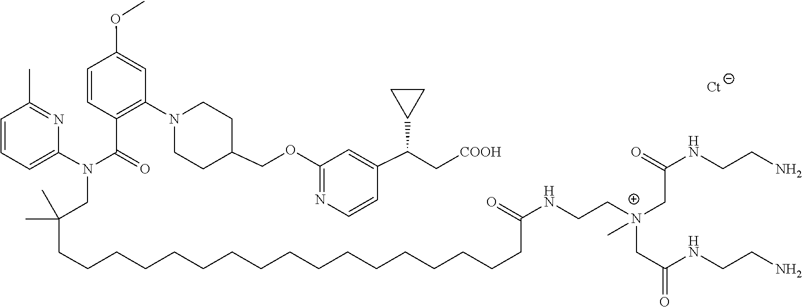 [SMILES: COc1ccc(C(=O)N(CC(C)(C)CCCCCCCCCCCCCCCCCC(=O)NCC[N+](C)(CC(=O)NCCN)CC(=O)NCCN)c2cccc(C)n2)c(N2CCC(COc3cc([C@@H](CC(=O)O)C4CC4)ccn3)CC2)c1]

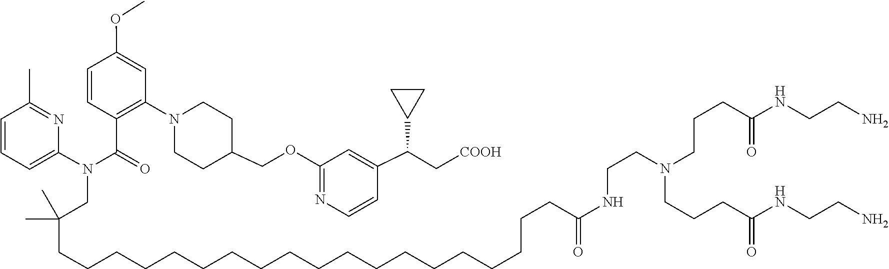 [SMILES: COc1ccc(C(=O)N(CC(C)(C)CCCCCCCCCCCCCCCCCCCC(=O)NCCN(CCCC(=O)NCCN)CCCC(=O)NCCN)c2cccc(C)n2)c(N2CCC(COc3cc([C@@H](CC(=O)O)C4CC4)ccn3)CC2)c1]

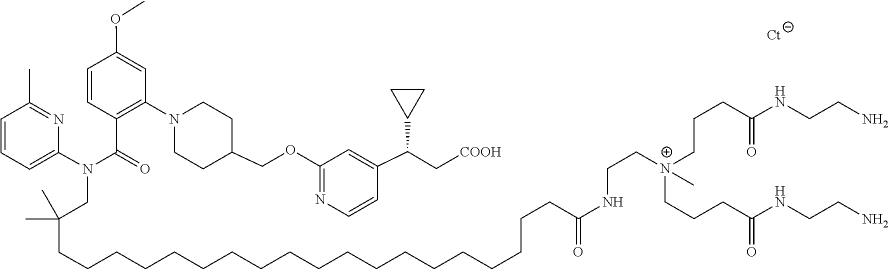 [SMILES: COc1ccc(C(=O)N(CC(C)(C)CCCCCCCCCCCCCCCCCCCC(=O)NCC[N+](C)(CCCC(=O)NCCN)CCCC(=O)NCCN)c2cccc(C)n2)c(N2CCC(COc3cc([C@@H](CC(=O)O)C4CC4)ccn3)CC2)c1]

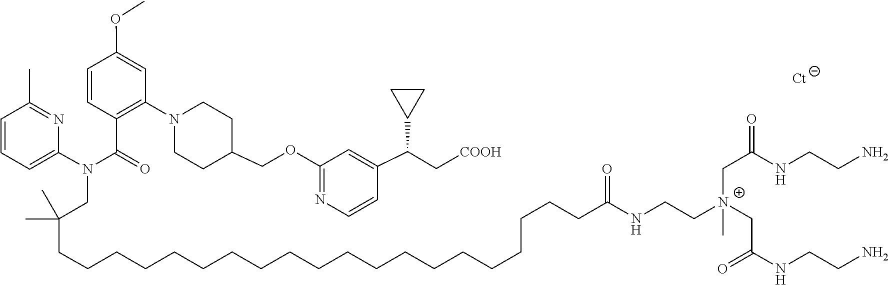 [SMILES: COc1ccc(C(=O)N(CC(C)(C)CCCCCCCCCCCCCCCCCCCCC(=O)NCC[N+](C)(CC(=O)NCCN)CC(=O)NCCN)c2cccc(C)n2)c(N2CCC(COc3cc([C@@H](CC(=O)O)C4CC4)ccn3)CC2)c1]

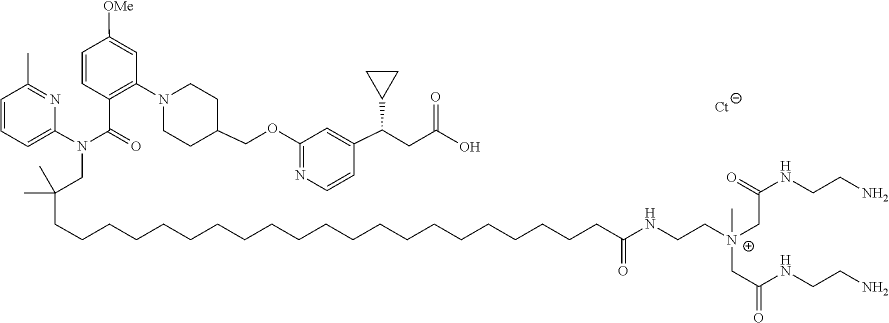 [SMILES: COc1ccc(C(=O)N(CC(C)(C)CCCCCCCCCCCCCCCCCCCCCC(=O)NCC[N+](C)(CC(=O)NCCN)CC(=O)NCCN)c2cccc(C)n2)c(N2CCC(COc3cc([C@@H](CC(=O)O)C4CC4)ccn3)CC2)c1]

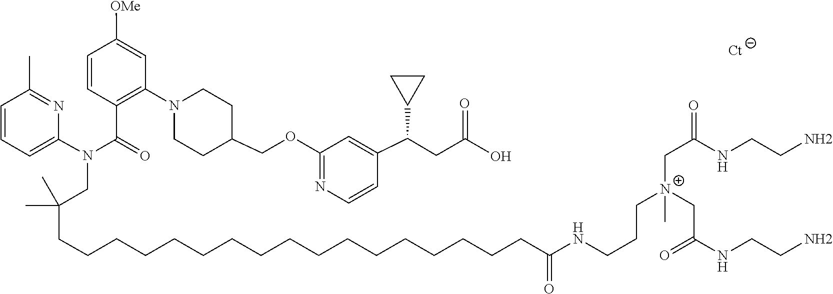 [SMILES: COc1ccc(C(=O)N(CC(C)(C)CCCCCCCCCCCCCCCCCC(=O)NCCC[N+](C)(CC(=O)NCCN)CC(=O)NCCN)c2cccc(C)n2)c(N2CCC(COc3cc([C@@H](CC(=O)O)C4CC4)ccn3)CC2)c1]

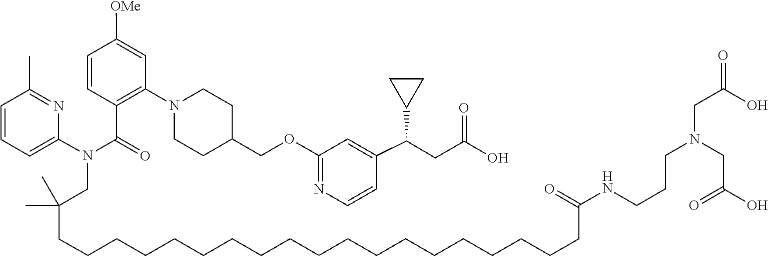 [SMILES: COc1ccc(C(=O)N(CC(C)(C)CCCCCCCCCCCCCCCCCCCC(=O)NCCCN(CC(=O)O)CC(=O)O)c2cccc(C)n2)c(N2CCC(COc3cc([C@@H](CC(=O)O)C4CC4)ccn3)CC2)c1]

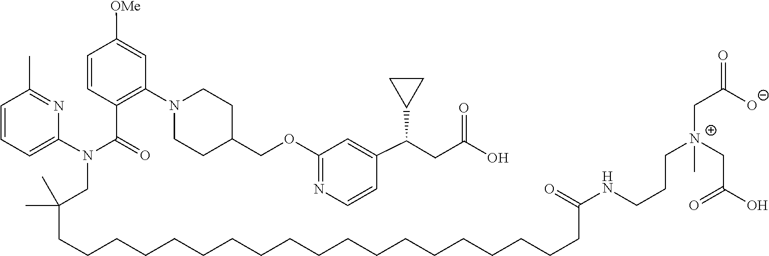 [SMILES: COc1ccc(C(=O)N(CC(C)(C)CCCCCCCCCCCCCCCCCCCC(=O)NCCC[N+](C)(CC(=O)[O-])CC(=O)O)c2cccc(C)n2)c(N2CCC(COc3cc([C@@H](CC(=O)O)C4CC4)ccn3)CC2)c1]

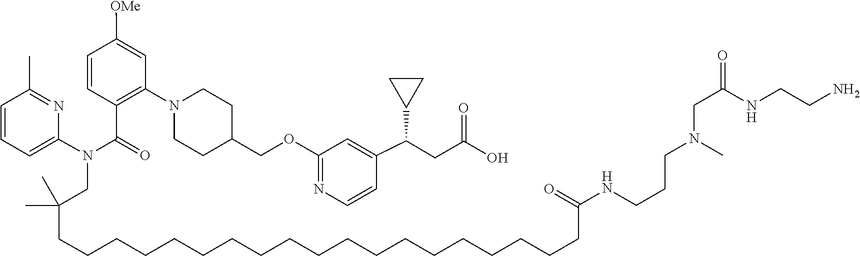 [SMILES: COc1ccc(C(=O)N(CC(C)(C)CCCCCCCCCCCCCCCCCCCC(=O)NCCCN(C)CC(=O)NCCN)c2cccc(C)n2)c(N2CCC(COc3cc([C@@H](CC(=O)O)C4CC4)ccn3)CC2)c1]

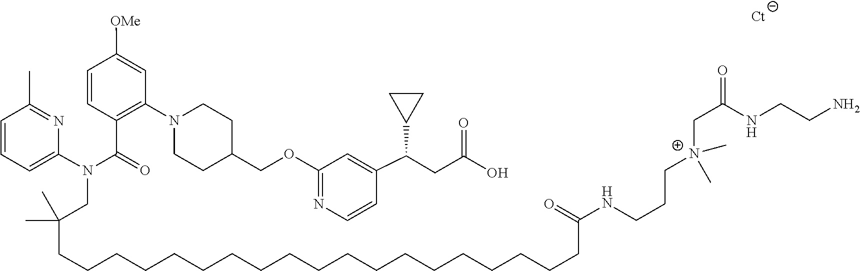 [SMILES: COc1ccc(C(=O)N(CC(C)(C)CCCCCCCCCCCCCCCCCCCC(=O)NCCC[N+](C)(C)CC(=O)NCCN)c2cccc(C)n2)c(N2CCC(COc3cc([C@@H](CC(=O)O)C4CC4)ccn3)CC2)c1]

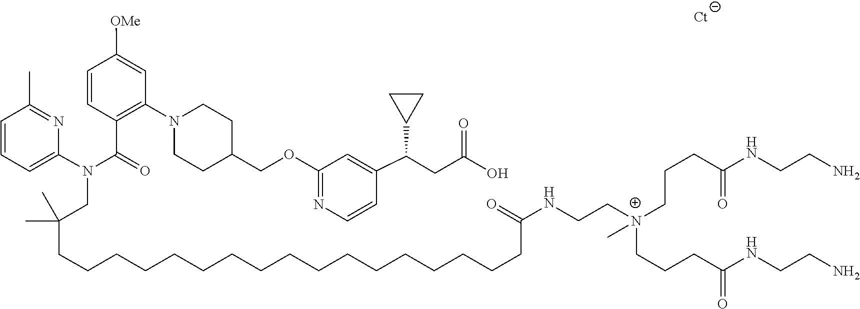 [SMILES: COc1ccc(C(=O)N(CC(C)(C)CCCCCCCCCCCCCCCCCC(=O)NCC[N+](C)(CCCC(=O)NCCN)CCCC(=O)NCCN)c2cccc(C)n2)c(N2CCC(COc3cc([C@@H](CC(=O)O)C4CC4)ccn3)CC2)c1]

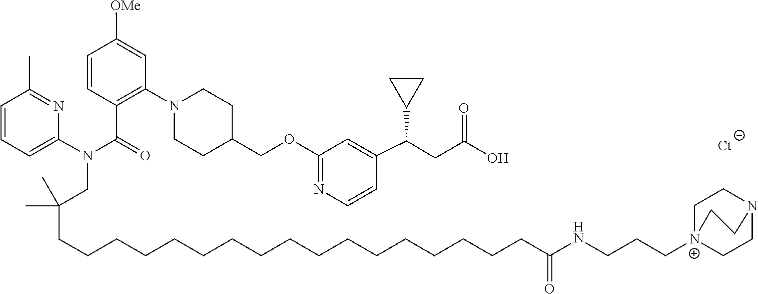 [SMILES: COc1ccc(C(=O)N(CC(C)(C)CCCCCCCCCCCCCCCCCC(=O)NCCC[N+]23CCN(CC2)CC3)c2cccc(C)n2)c(N2CCC(COc3cc([C@@H](CC(=O)O)C4CC4)ccn3)CC2)c1]

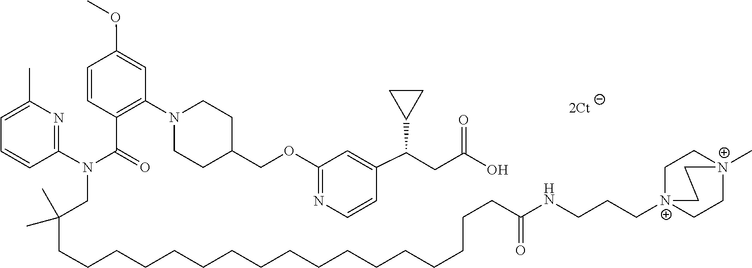 [SMILES: COc1ccc(C(=O)N(CC(C)(C)CCCCCCCCCCCCCCCCCC(=O)NCCC[N+]23CC[N+](C)(CC2)CC3)c2cccc(C)n2)c(N2CCC(COc3cc([C@@H](CC(=O)O)C4CC4)ccn3)CC2)c1]

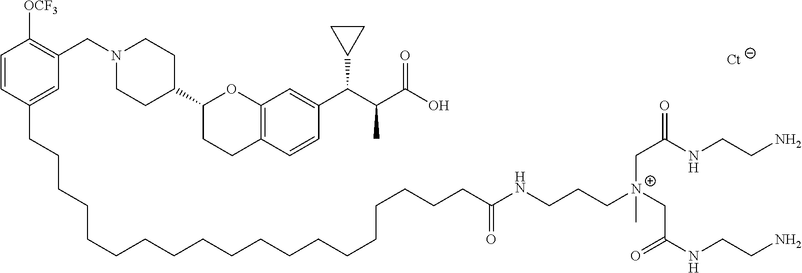 [SMILES: C[C@H](C(=O)O)[C@H](c1ccc2c(c1)O[C@@H](C1CCN(Cc3cc(CCCCCCCCCCCCCCCCCCCC(=O)NCCC[N+](C)(CC(=O)NCCN)CC(=O)NCCN)ccc3OC(F)(F)F)CC1)CC2)C1CC1]

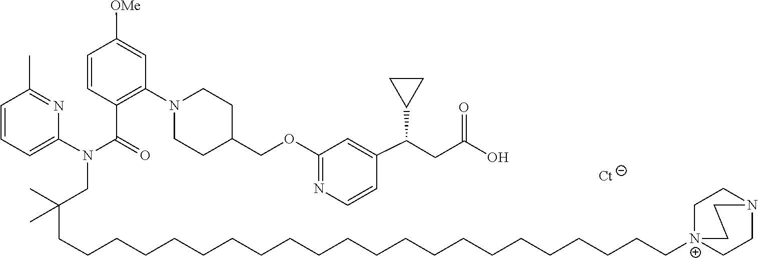 [SMILES: COc1ccc(C(=O)N(CC(C)(C)CCCCCCCCCCCCCCCCCCCCCC[N+]23CCN(CC2)CC3)c2cccc(C)n2)c(N2CCC(COc3cc([C@@H](CC(=O)O)C4CC4)ccn3)CC2)c1]

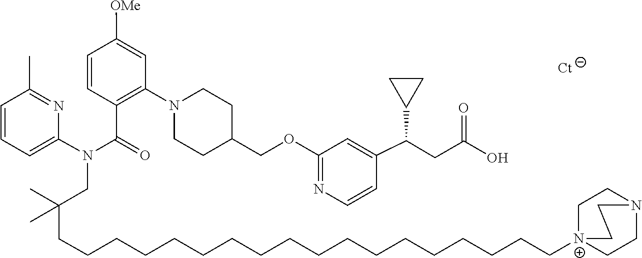 [SMILES: COc1ccc(C(=O)N(CC(C)(C)CCCCCCCCCCCCCCCCCC[N+]23CCN(CC2)CC3)c2cccc(C)n2)c(N2CCC(COc3cc([C@@H](CC(=O)O)C4CC4)ccn3)CC2)c1]

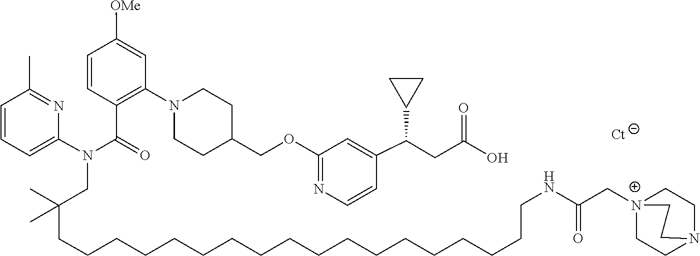 [SMILES: COc1ccc(C(=O)N(CC(C)(C)CCCCCCCCCCCCCCCCCCNC(=O)C[N+]23CCN(CC2)CC3)c2cccc(C)n2)c(N2CCC(COc3cc([C@@H](CC(=O)O)C4CC4)ccn3)CC2)c1]

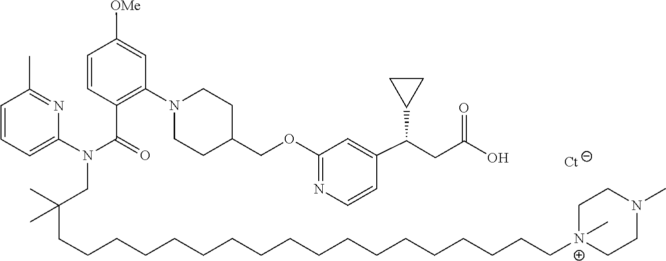

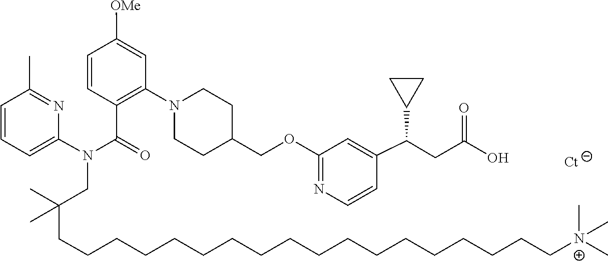 [SMILES: COc1ccc(C(=O)N(CC(C)(C)CCCCCCCCCCCCCCCCCC[N+](C)(C)C)c2cccc(C)n2)c(N2CCC(COc3cc([C@@H](CC(=O)O)C4CC4)ccn3)CC2)c1]

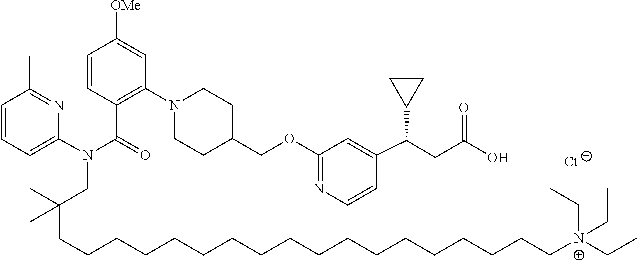 [SMILES: CC[N+](CC)(CC)CCCCCCCCCCCCCCCCCCC(C)(C)CN(C(=O)c1ccc(OC)cc1N1CCC(COc2cc([C@@H](CC(=O)O)C3CC3)ccn2)CC1)c1cccc(C)n1]

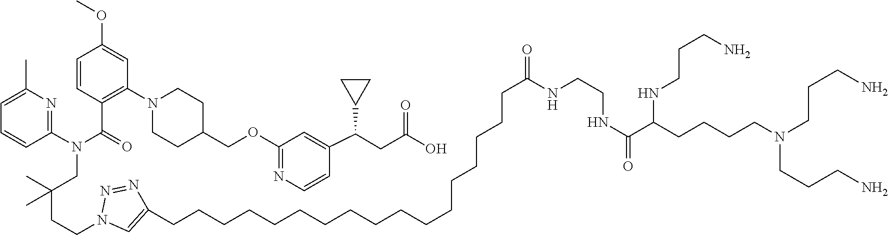 [SMILES: COc1ccc(C(=O)N(CC(C)(C)CCn2cc(CCCCCCCCCCCCCCCCCC(=O)NCCNC(=O)C(CCCCN(CCCN)CCCN)NCCCN)nn2)c2cccc(C)n2)c(N2CCC(COc3cc([C@@H](CC(=O)O)C4CC4)ccn3)CC2)c1]

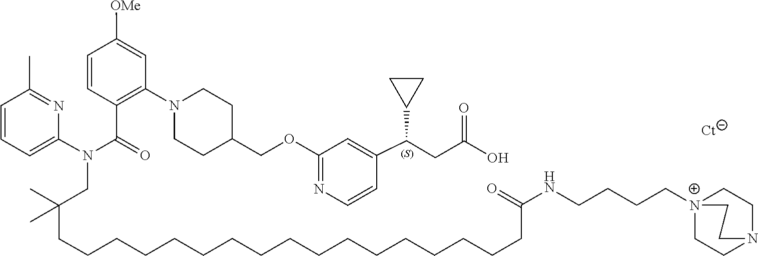

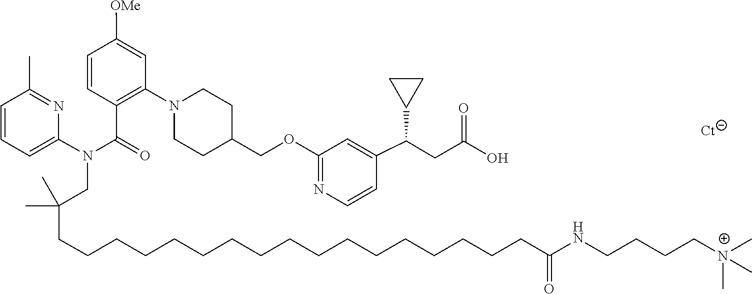 [SMILES: COc1ccc(C(=O)N(CC(C)(C)CCCCCCCCCCCCCCCCCC(=O)NCCCC[N+](C)(C)C)c2cccc(C)n2)c(N2CCC(COc3cc([C@@H](CC(=O)O)C4CC4)ccn3)CC2)c1]

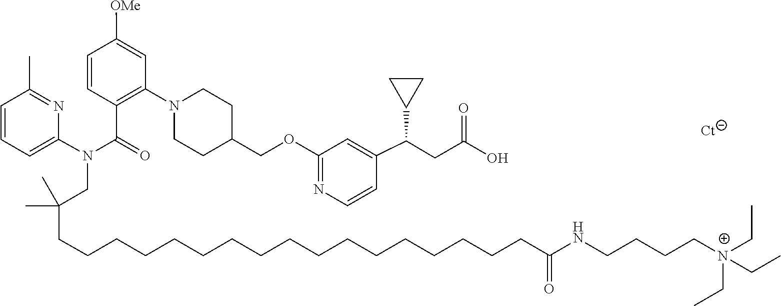 [SMILES: CC[N+](CC)(CC)CCCCNC(=O)CCCCCCCCCCCCCCCCCC(C)(C)CN(C(=O)c1ccc(OC)cc1N1CCC(COc2cc([C@@H](CC(=O)O)C3CC3)ccn2)CC1)c1cccc(C)n1]

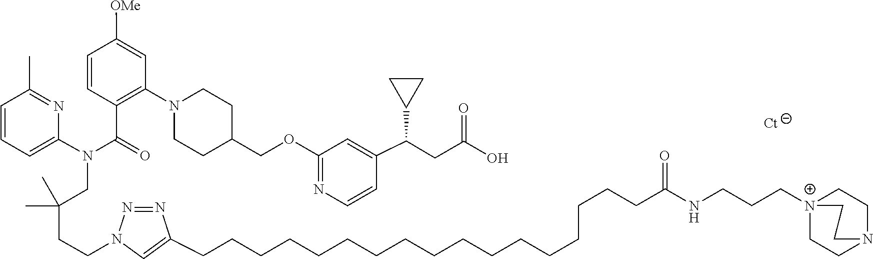 [SMILES: COc1ccc(C(=O)N(CC(C)(C)CCn2cc(CCCCCCCCCCCCCCCCCC(=O)NCCC[N+]34CCN(CC3)CC4)nn2)c2cccc(C)n2)c(N2CCC(COc3cc([C@@H](CC(=O)O)C4CC4)ccn3)CC2)c1]

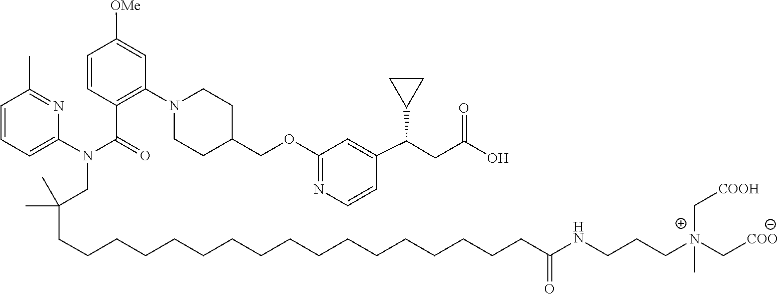

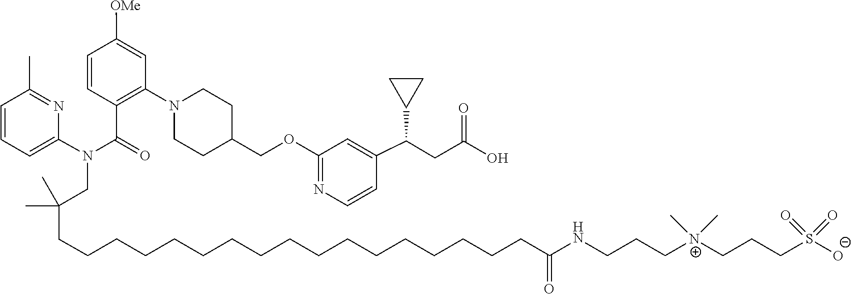 [SMILES: COc1ccc(C(=O)N(CC(C)(C)CCCCCCCCCCCCCCCCCC(=O)NCCC[N+](C)(C)CCCS(=O)(=O)[O-])c2cccc(C)n2)c(N2CCC(COc3cc([C@@H](CC(=O)O)C4CC4)ccn3)CC2)c1]

In some embodiments, the compound of any one of the compounds in Table 1 can be present in a form of a pharmaceutically acceptable salt.

In some embodiments, the compound of any one of the compounds in Table 1 can be present in a form of a pharmaceutically acceptable ester, or a pharmaceutically acceptable salt thereof.

In some embodiments, the present disclosure also provides a compound according to any of Examples 1-36, or a pharmaceutically acceptable salt thereof. A compound according to any of Examples 1-36 should be understood as the same compound as drawn in Examples 1-36 without considering its salt form and/or any counterion(s); the compound may exist in a different salt form and/or containing a different counterion(s).

In some embodiments, to the extent applicable, the genus of compounds in the present disclosure also excludes any of the compounds specifically prepared and disclosed prior to this disclosure, such as any of those compounds described in International Application No. PCT/CN2021/109496 and PCT/CN2023/071833, such as

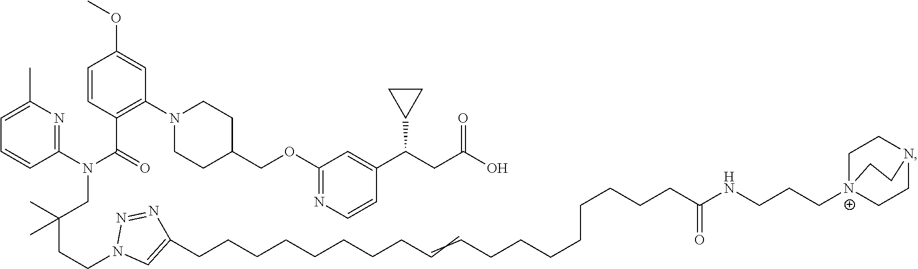 [SMILES: COc1ccc(C(=O)N(CC(C)(C)CCn2cc(CCCCCCCCC=CCCCCCCCCC(=O)NCCC[N+]34CCN(CC3)CC4)nn2)c2cccc(C)n2)c(N2CCC(COc3cc([C@@H](CC(=O)O)C4CC4)ccn3)CC2)c1]

or a pharmaceutically acceptable salt or ester thereof.

In some embodiments, the present disclosure also provides a compound according to any of GPR-1, GPR-2, GPR-2B, GPR-3, or GPR-3B, or a pharmaceutically acceptable salt or ester thereof.

The compounds herein can be prepared by those skilled in the art in view of the present disclosure. Exemplary syntheses are shown in the Examples section, such as those shown in the schemes in the Examples section, which can be adopted by those skilled in the art to synthesize other compounds of the present disclosure.

As exemplified herein, compounds of present disclosure can be typically prepared by a coupling reaction to link the residue of a ligand of a membrane bound protein, such as GPCR, in particular, a GPR40 agonist with a hydrophilic molecule. Suitable coupling reactions are not particularly limited, which will depend on the structural features of the compound.

In some embodiments, an amide coupling can be used to link the residue of a GPR40 agonist with a hydrophilic molecule. For example, a compound of Formula I-1-A-1 can be prepared according to the synthetic sequence shown in Scheme A-1, A-2, or A-3, when D is D-1, D-2, or D-3, which can react an acid of S-1, S-3, or S-4 with an amine of S-2 under amide coupling conditions, which can then be followed by deprotection to provide the compound of Formula I-1-A-1-D2, I-1-A-1-D3, or I-1-A-1-D1, respectively. Although not drawn, it should be understood that the amine S-2, as well as the compound of Formula I-1-A-1-D2, I-1-A-1-D3, or I-1-A-1-D1 are charge balanced as necessary so that the overall molecule is neutral. The Pg¹ in S-1, S-3, or S-4 refers to a carboxylic acid protecting group, such as a tert-butyl group. The variables shown in the schemes are described and preferred herein for such variables respectively.

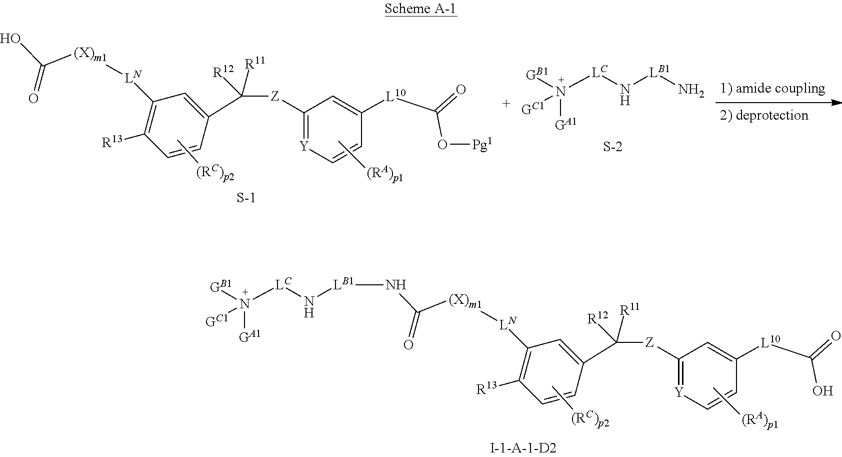

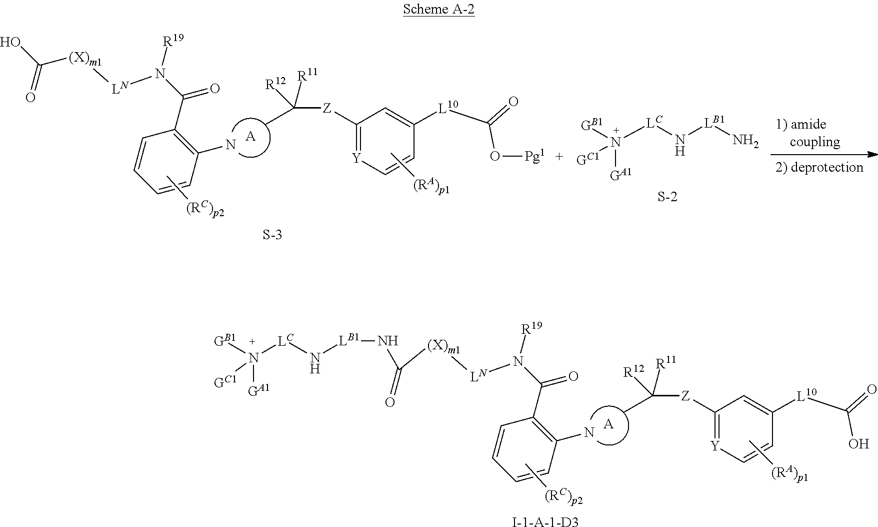

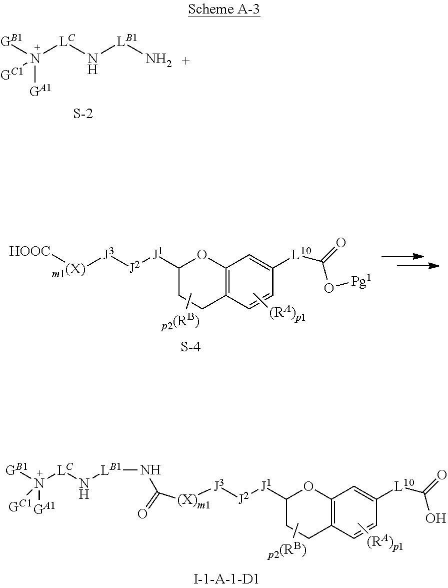

Other compounds of Formula I can be prepared similarly to those shown in Schemes A-1, A-2, and A-3. Exemplified procedures are also shown in the Examples section herein.

In some embodiments, the present disclosure also provides synthetic intermediates and methods according to any of those described herein, such as those shown in Schemes A-1, A-2, and A-3 and those shown in the schemes in the Examples section. To be clear, the synthetic intermediates shown in the Examples section in a different salt form and/or having a different counterion(s) are within the scope of this disclosure. In some embodiments, the present disclosure provides a novel synthetic intermediate shown in the Examples section herein, which as applicable, may exist in any salt form and/or have a different counterion(s) as those shown in the Examples section herein.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof; described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., Formula I-1, I-1-A, I-1-B, I-1-C, I-1-D, I-1-E, I-1-F, I-1-G, I-1-G-1, I-1-G-2, I-1-G-3, I-1-G-4, I-1-G-5, I-1-G-6, I-1-G-7, I-1-G-8, I-1-G-9, I-1-H, I-1-I, I-1-A-1, I-1-B-1, I-1-C-1, I-1-D-1, I-1-E-1, I-1-F-1, I-1-H-1, or I-1-I-1), or any of the compounds listed in Table 1 herein, any of the compound according to Examples 1-36 herein, or a pharmaceutically acceptable salt or ester thereof) and a pharmaceutically acceptable excipient. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., Formula I-1, I-1-A, I-1-B, I-1-C, I-1-D, I-1-E, I-1-F, I-1-G, I-1-G-1, I-1-G-2, I-1-G-3, I-1-G-4, I-1-G-5, I-1-G-6, I-1-G-7, I-1-G-8, I-1-G-9, I-1-H, I-1-I, I-1-A-1, I-1-B-1, I-1-C-1, I-1-D-1, I-1-E-1, I-1-F-1, I-1-H-1, or I-1-I-1), or any of the compounds listed in Table 1 herein, any of the compound according to Examples 1-36 herein, or a pharmaceutically acceptable salt or ester thereof, e.g., in a therapeutically effective amount. In any of the embodiments described herein, the pharmaceutical composition can comprise a therapeutically effective amount of a compound selected from the compounds listed in Table 1 herein, or a pharmaceutically acceptable salt or ester thereof. In any of the embodiments described herein, the pharmaceutical composition can comprise a therapeutically effective amount of a compound according to Examples 1-36 herein, or a pharmaceutically acceptable salt thereof.

In some embodiments, the pharmaceutical composition can be formulated for oral administration. Typically, the pharmaceutical composition is administered to a subject in need to deliver an effective amount of GPR40 agonist in the gastrointestinal tract with minimal or no absorption of GPR40 agonist in systemic circulation. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

Compounds of the present disclosure can be used alone, in combination with each other, or in combination with one or more additional therapeutic agents, e.g., PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; α-glucosidase inhibitors; agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARα agonists, (v) cholesterol absorption inhibitors, (vi) acyl CoA:cholesterol acyltransferase (ACAT) inhibitors, (vii) CETP inhibitors, (viii) PCSK9 inhibitor or antibodies; (ix) apolipoproteins inhibitors; (x) phenolic anti-oxidants; PPARα/γ dual agonists; PPARδ agonists; PPAR α/δ partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists (peptide and small-molecule); GLP-1/GIP receptor dual agonists; GLP-1/glucagon receptor dual agonists; GLP-1/GIP/insulin receptor triple agonists; GLP-1/GIP/glucagon receptor triple agonists; GIP receptor antibody; GLP-1 analog/GIP receptor antibody; PYY analog; amylin analogs; GPR119 agonist; TGR5 agonist; SSTR2 and/or SSTR5 antagonist or inverse agonist; THRβ agonists; HSD-1 inhibitors; HSD-17 inhibitors and degraders; PNPLA3 inhibitors and degraders; SGLT-2 inhibitors; SGLT-1/SGLT-2 inhibitors; enteric alpha-glucosidase inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin; (xi) anti-amyloid beta antibody; (xii) anti-inflammatory agents including but not limited to PDE4 inhibitors, JAK inhibitors, TYK2 inhibitors, S1P receptor modulators, NLRP3 inhibitors, BTK inhibitors, IRAK1 inhibitors, IRAK4 inhibitors, glucocorticoids, anti-TNFα antibodies, anti-IL-12/IL-23 antibodies, (xiii) anti-integrin antibodies or small-molecule inhibitors of integrins including α4β7, α4, β7, MAdCAM-1, αvβ6 and αvβ1. These additional therapeutic agents are known in the art, some of which are exemplified in the background section. Additional example can be found in various patent literatures, for example, as described in U.S. Published Application No. 20190367495, the content of which is herein incorporated by reference.

When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disorder, condition or disease as described herein, such as type 1 or 2 diabetes, obesity, and/or eating disorder, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Method of Treatment/Use

Compounds of the present disclosure have various utilities. For example, compounds of the present disclosure can be used as therapeutic active substances for the treatment and/or prophylaxis of disorders, conditions or diseases that are associated with G-protein-coupled receptor 40 (“GPR40”). Accordingly, some embodiments of the present disclosure are also directed to methods of using one or more compounds of the present disclosure or pharmaceutical compositions herein for treating or preventing a disorder, condition or disease that may be responsive to the agonism of the G-protein-coupled receptor 40 (“GPR40”) in a subject in need thereof, such as for treating type 1 or 2 diabetes mellitus, obesity, and/or eating disorder, in a subject in need thereof.

In some embodiments, the present disclosure provides a method of treating or preventing a disorder, condition or disease that may be responsive to the agonism of the G-protein-coupled receptor 40 (“GPR40”) in a subject in need thereof. In some embodiments, the method comprises administering an effective amount of a compound of the present disclosure (e.g., a compound of Formula I (e.g., Formula 1-1, I-1-A, I-1-B, I-1-C, I-1-D, I-1-E, I-1-F, I-1-G, I-1-G-1, I-1-G-2, I-1-G-3, I-1-G-4, I-1-G-5, I-1-G-6, I-1-G-7, I-1-G-8, I-1-G-9, I-1-H, I-1-I, I-1-A-1, I-1-B-1. I-1-C-1, I-1-D-1, I-1-E-1, I-1-F-1, I-1-H-1, or I-1-I-1), or any of the compounds listed in Table 1 herein, any of the compound according to Examples 1-36 herein, or a pharmaceutically acceptable salt or ester thereof) or an effective amount of a pharmaceutical composition described herein. In some embodiments, the disorder, condition or disease that may be responsive to agonism of GPR40 is Type 1 or 2 diabetes, obesity, hyperglycemia, glucose intolerance, insulin resistance, hyperinsulinemia, hypercholesterolemia, hypertension, hyperlipoproteinemia, hyperlipidemia, myocardial infarction, stroke, hypertriglyceridemia, dyslipidemia, metabolic syndrome, syndrome X, cardiovascular disease, atherosclerosis, kidney disease, diabetic kidney disease, ketoacidosis, thrombotic disorders, nephropathy, diabetic neuropathy, diabetic retinopathy, sexual dysfunction, dermatopathy, dyspepsia, hypoglycemia, cancer, edema, nonalcoholic steatohepatitis (NASH), lipodystrophy, Prader Willi syndrome, inflammatory bowel diseases including Crohn's disease and ulcerative colitis, irritable bowel syndrome, short bowel syndrome, lymphocytic colitis, rare microscopic colitis, and/or neurodegenerative diseases including but not limited to Alzheimer's disease, Parkinson's disease, amyotrophic lateral sclerosis.

In some embodiments, the present disclosure also provides a method of treating type 2 diabetes mellitus in a subject in need thereof. In some embodiments, the method comprises administering an effective amount of a compound of the present disclosure (e.g., a compound of Formula I (e.g., Formula I-1, I-1-A, I-1-B, I-1-C, I-1-D, I-1-E, I-1-F, I-1-G, I-1-G-1, I-1-G-2, I-1-G-3, I-1-G-4, I-1-G-5, I-1-G-6, I-1-G-7, I-1-G-8, I-1-G-9, I-1-H, I-1-I, I-1-A-1, I-1-B-1, I-1-C-1, I-1-D-1, I-1-E-1, I-1-F-1, I-1-H-1, or I-1-I-1), or any of the compounds listed in Table 1 herein, any of the compound according to Examples 1-36 herein, or a pharmaceutically acceptable salt or ester thereof) or an effective amount of a pharmaceutical composition described herein.

The administering in the methods herein is not limited. In some embodiments, the administering is orally.

As discussed herein, compounds of the present disclosure can be used as a monotherapy or in a combination therapy. In some embodiments according to the methods described herein, compounds of the present disclosure can be administered as the only active ingredient(s).

In some embodiments according to the methods described herein, compounds of the present disclosure can also be co-administered with an additional therapeutic agent, either concurrently or sequentially in any order, to the subject in need thereof. In some embodiments, the additional therapeutic agent can be PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; α-glucosidase inhibitors; agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARα agonists, (v) cholesterol absorption inhibitors, (vi) acyl CoA:cholesterol acyltransferase (ACAT) inhibitors, (vii) CETP inhibitors, (viii) PCSK9 inhibitor or antibodies; (ix) apolipoproteins inhibitors; (x) phenolic anti-oxidants; PPARα/γ dual agonists; PPARδ agonists; PPAR α/δ partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists (peptide and small-molecule); GLP-1/GIP receptor dual agonists; GLP-1/glucagon receptor dual agonists; GLP-1/GIP/insulin receptor triple agonists; GLP-1/GIP/glucagon receptor triple agonists; GIP receptor antibody; GLP-1 analog/GIP receptor antibody; PYY analog; amylin analogs; GPR119 agonist; TGR5 agonist; SSTR2 and/or SSTR5 antagonist or inverse agonist; THRβ agonists; HSD-1 inhibitors; HSD-17 inhibitors and degraders; PNPLA3 inhibitors and degraders; SGLT-2 inhibitors; SGLT-1/SGLT-2 inhibitors; enteric alpha-glucosidase inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin; (xi) anti-amyloid beta antibody; (xii) anti-inflammatory agents including but not limited to PDE4 inhibitors, JAK inhibitors, TYK2 inhibitors, SIP receptor modulators, NLRP3 inhibitors, BTK inhibitors, IRAK1 inhibitors, IRAK4 inhibitors, glucocorticoids, anti-TNFα antibodies, anti-IL-12/IL-23 antibodies, (xiii) anti-integrin antibodies or small-molecule inhibitors including α4β7, α4, β7, MAdCAM-1, αvβ6 and αvβ1.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

When a variable or structure herein defined as containing a charged group, such as those containing a quaternary nitrogen atom, it should be understood that the compound containing such variable or structure is overall neutral; in other words, any charge associated with the variable or structure is balanced with a counterion as necessary to maintain the compound's overall electronic neutrality, whether or not the counterion is explicitly drawn or described. Further, when the charge of a variable or structure is balanced through an internal salt (or zwitterion structure) such that the variable or structure is overall neutral, it should be understood that a counterion is not necessary to maintain electronic neutrality; in such cases, even if a counterion is explicitly drawn or described, such counterion should be understood as non-existent.

Suitable counterions are not particularly limited, however, preferably, the counterion herein is a pharmaceutically acceptable counterion, such as a pharmaceutically acceptable anion, which may be monovalent (e.g., including one formal negative charge) or multivalent (e.g., including more than one formal negative charge), such as divalent or trivalent. Non-limiting exemplary suitable counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, tartrate, citrate, fumarate, maleate, malate, malonate, gluconate, succinate, glutarate, adipate, pimelate, suberate, azelate, sebacate, Salicylate, phthalates, aspartate, glutamate, and the like), BF₄⁻, PF₄⁻, PF₆⁻, AsF₆⁻, SbF₆⁻, B[3,5-(CF₃)₂C₆H₃]₄]⁻, BPh₄⁻, Al(OC(CF₃)₃)₄, carborane anions (e.g., CB₁₁H₁₂⁻ or (HCB₁₁Me₅Br₆)⁻), CO₃²⁻, HPO₄²⁻, PO₄³⁻, B₄O₇²⁻, SO₄²⁻, S₂O₃²⁻, etc.

It is also meant to be understood that a specific embodiment of a variable moiety herein can be the same or different as another specific embodiment having the same identifier.

Suitable groups for in compounds of Formula I or a subformula thereof, as applicable, are independently selected. The described embodiments of the present disclosure can be combined. Such combination is contemplated and within the scope of the present disclosure. For example, it is contemplated that the definition(s) of any one or more of q, T^{A}, L^{A}, and D of Formula I can be combined with the definition of any one or more of the other(s) of q, T^{A}, L^{A}, and D as applicable, and the resulted compounds from the combination are within the scope of the present disclosure. Combinations of other variables for other Formulae should be understood similarly. To be clear, it should be understood that with respect to any formula herein, unless specified or contrary from context, the definition and preferred definition of a variable appearing in a formula can be any of those respective definition and preferred definition shown herein for the variable in connection with a parent formula (or any of the sub-formulae of the parent formula) or any other formula that is indicated as applicable. For example, unless specified or contrary from context, a variable appearing in Formula I-1-A can have a definition as defined for the variable in connection with Formula I or any of its other sub-formulae (e.g., I-1, I-1-B, etc.). As a further example, unless specified or contrary from context, the definition and preferred definition of L^{A} and/or T^{A} in connection with any formula herein is generally applicable to all other formulae herein. Preferred definition of L^{A} and/or T^{A} in connection with any formula herein also includes those shown in the specific compounds prepared herein, such as in Examples 1-36.

The symbol, [symbol] , displayed perpendicular to (or otherwise crossing) a bond, indicates the point at which the displayed moiety is attached to the remainder of the molecule. It should be noted that in some chemical drawings herein, the immediately connected group or groups are shown beyond the symbol, [symbol] , to indicate direction of attachment, as would be understood by those skilled in the art.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC area, or both, or with a non-detectable amount of the other stereoisomer(s). In some preferred embodiments, the compound herein can exist predominantly as the as-drawn stereoisomer, with an enantiomeric excess (“ee”) of at least 70%, for example, with an ee of at least 80%, at least 85%, at least 90%, at least 95%, at least 98%, or at least 99%, or the other enantiomer is non-detectable. The presence and/or amounts of stereoisomers can be determined by those skilled in the art in view of the present disclosure, including through the use of chiral HPLC or other methods.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” refers to any of the compounds described herein according to Formula I (e.g., Formula I-1, I-1-A, I-1-B, I-1—C, I-1-D, I-1-E, I-1-F, I-1-G, I-1-G-1, I-1-G-2, I-1-G-3, I-1-G-4, I-1-G-5, I-1-G-6, I-1-G-7, I-1-G-8, I-1-G-9, I-1-H, I-1-I, I-1-A-1 (e.g., I-1-A-1-D1, I-1-A-1-D2, I-1-A-1-D3), I-1-B-1, I-1-C-1, I-1-D-1, I-1-E-1, I-1-F-1, I-1-H-1, or I-1-I-1), or any of the compounds listed in Table 1 herein, any of the compound according to Examples 1-36 herein, isotopically labeled compound(s) thereof (such as a deuterated analog wherein at least one of the hydrogen atoms is substituted with a deuterium atom with an abundance above its natural abundance), possible regioisomers, possible stereoisomers thereof (including diastereoisomers, enantiomers, and racemic mixtures), tautomers thereof, conformational isomers thereof, pharmaceutically acceptable esters thereof, a zwitterion structure thereof, and/or possible pharmaceutically acceptable salts thereof (e.g., acid addition salt such as HCl salt or base addition salt such as Na salt). Hydrates and solvates of the compounds of the present disclosure are considered compositions of the present disclosure, wherein the compound(s) is in association with water or solvent, respectively. To be clear, as used herein, a compound according to Examples 1-36 herein or a pharmaceutically acceptable salt thereof should be understood as encompassing any compound, or pharmaceutically acceptable salt thereof, having the structure of any of Examples 1-36 as shown in the Examples section herein, except that the counterion and/or salt form may be different. For example, a compound according to Example 1 or a pharmaceutically acceptable salt thereof should be understood as encompassing a base form of Example 1, a pharmaceutically acceptable salt thereof, which is not limited to the HCl addition salt, or any combinations thereof. Similarly, a compound according to Example 3 or a pharmaceutically acceptable salt thereof should be understood as encompassing a base form of Example 3 with a counterion for the quaternary nitrogen being Cl⁻ or any other pharmaceutically acceptable counterion or an internal counterion, a pharmaceutically acceptable salt thereof, which is not limited to the salt form of HCl addition salt and a counterion of Cl⁻ for the quaternary nitrogen, or any combinations thereof.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl which can include one to twelve carbon atoms (i.e., C₁₋₁₂ alkyl) or the number of carbon atoms designated. In one embodiment, the alkyl group is a straight chain C₁₋₁₀ alkyl group (alternatively referred to as linear C₁₋₁₀ alkyl). In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group includes methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl.

As used herein, the term “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ haloalkyl group. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of which has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. For example, C₁₋₄ heteroalkyl include but not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, and —CH₂—CH₂—S(O)₂—CH₃, C₂ heteroalkyl such as —O—CH₂—CH₃ and C₁ heteroalkyl such as O—CH₃, etc. To be clear, when the heteroalkyl is referred to as xx-membered, the number of carbon and heteroatoms forming the heteroalkyl should be counted together, but not the potential oxidation, for example, sulfur oxide or N-oxide is counted as one member. For example, —CH₂—CH₂—N(CH₃)—CH₃ or —CH₂—[N(CH₃)₃]⁺ may be considered a five-membered heteroalkyl. Additionally, as an example, a four-membered heteroalkyl includes —CH₂—CH₂—O—CH₃, —CH₂—CH₂—NH—CH₃, —CH₂—S—CH₂—CH₃, —CH₂—CH₂—S(O)—CH₃, and —CH₂—CH₂—S(O)₂—CH₃, a three-membered heteroalkyl includes —O—CH₂—CH₃, and a two-membered heteroalkyl includes O—CH₃. Similarly, for the purposes herein, when counting the number of heteroatoms in a heteroalkyl group, the oxygen atom from potential oxidation is not counted, thus, —CH₂—S—CH₂—CH₃, —CH₂—CH₂—S(O)—CH₃, and —CH₂—CH₂—S(O)₂—CH₃ should all be considered as a 4-membered, C₃ heteroalkyl having one heteroatom, S, in which the S is optionally oxidized. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Carbocyclyl” or “carbocyclic” as used by itself or as part of another group refers to a radical of a non-aromatic cyclic hydrocarbon group having at least 3 carbon atoms, e.g., from 3 to 10 ring carbon atoms (“C₃₋₁₀ carbocyclyl”), and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl. As used herein, the term “carbocyclylene” as used by itself or as part of another group refers to a divalent radical derived from the carbocyclyl group defined herein.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In preferred embodiments, the cycloalkyl is a monocyclic ring. As used herein, the term “cycloalkylene” as used by itself or as part of another group refers to a divalent radical derived from a cycloalkyl group, for example,

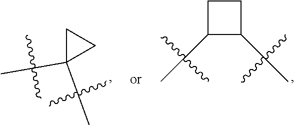 [SMILES: *C1(*)CC1 ; *C1CCC1*]

etc.

“Heterocyclyl” or “heterocyclic” as used by itself or as part of another group refers to a radical of a 3-membered or greater, such as 3- to 14-membered, non-aromatic ring system having ring carbon atoms and at least one ring heteroatom, such as 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is on the heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. As used herein, the term “heterocyclylene” as used by itself or as part of another group refers to a divalent radical derived from the heterocyclyl group defined herein. For example, a piperidinylene group includes two attaching points from the piperidine ring:

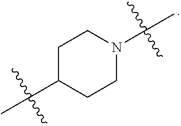 [SMILES: *C1CCN(*)CC1]

The heterocyclyl or heterocylylene can be optionally linked to the rest of the molecule through a carbon or nitrogen atom.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. As used herein, the term “arylene” as used by itself or as part of another group refers to a divalent radical derived from the aryl group defined herein. For example, a phenylene group includes two attaching points from the benzene ring, for example, 1,3-phenylene, 1,4-phenylene:

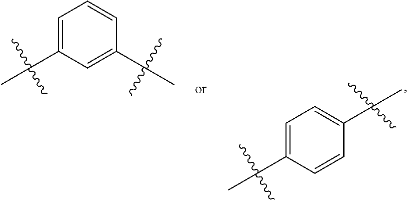 [SMILES: *c1cccc(*)c1 ; *c1ccc(*)cc1]

etc.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-14 membered monocyclic, bicyclic, or tricyclic 4n+2 aromatic ring system (e.g., having 6 or 10 pi electrons shared in a cyclic array) having ring carbon atoms and at least one, preferably, 1-4, ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl). As used herein, the term “heteroarylene” as used by itself or as part of another group refers to a divalent radical derived from the heteroaryl group defined herein. For example, a pyridinylene group includes two attaching points from the pyridine ring, for example, 2,4-pyridinylene, 2,5-pyridinylene:

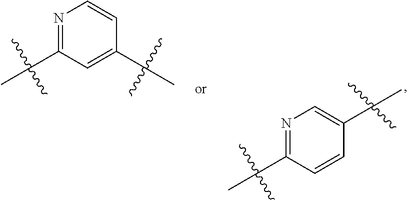 [SMILES: *c1ccnc(*)c1 ; *c1ccc(*)nc1]

etc.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable. Two of the optional substituents can join to form an optionally substituted cycloalkyl, heterocyclyl, aryl, or heteroaryl ring. Substitution can occur on any available carbon, oxygen, or nitrogen atom, and can form a spirocycle. Typically, substitution herein does not result in an O—O, O—N, S—S, S—N(except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —NHC(O)H, —NHC(O) (C₁-C₈)alkyl groups, —NHC(O) (C₃-C₈)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, —N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

Exemplary carbon atom substituents include, but are not limited to, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trimethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkylene, alkenyl, alkynyl, carbocyclic, carbocyclylene, cycloalkyl, cycloalkylene, alkoxy, cycloalkoxy, heterocyclyl, or heterocyclylene herein can each be independently unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl, arylene, heteroaryl or heteroarylene group herein can each be independently unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

“Halo” or “halogen” refers to fluorine (fluoro, —F), chlorine (chloro, —Cl), bromine (bromo, —Br), or iodine (iodo, —I).

The term “leaving group” is given its ordinary meaning in the art of synthetic organic chemistry and refers to an atom or a group capable of being displaced by a nucleophile. See, for example, Smith, March Advanced Organic Chemistry 6^{th} ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates.

The term “pharmaceutically acceptable salt”, “pharmaceutically acceptable anion” or “pharmaceutically acceptable cation” refers to those salts, anions or cations, which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts, anions, or cations are well known in the art.

The term “pharmaceutically acceptable ester” refers to those esters which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable esters are well known in the art, for example, a C₁₋₄ alkyl ester, such as ethyl ester.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of the preferred embodiments can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. Exemplary embodiments of steps for performing the synthesis of products described herein are described in greater detail infra. The examples are illustrative only and do not limit the claimed invention in any way. Further, in the structures shown in the Examples section herein, a salt form and/or a counterion may be shown (the stoichiometry may or may not be shown, and the charge of the counterion may or may not be shown) to be associated with a particular structure. However, it should be understood that the Examples and/or Intermediates herein are not limited to any of the particular salt forms and/or counterions as drawn, for example, the Examples and/or Intermediates herein may exist in the form of an internal salt and/or an external salt with a pharmaceutically acceptable counterion.

The abbreviations used in the Examples section should be understood as having their ordinary meanings in the art unless specifically indicated otherwise or obviously contrary from context. The following shows a list of some of the abbreviations used in the Examples section and their ordinary meanings in the art:
AIBN azobisisobutyronitrile
ACN acetonitrile
Bn benzyl
DBU 1,8-Diazabicyclo[5.4.0]undec-7-ene
DCM dichloromethane
DEAD Diethyl azodicarboxylate
DHP 3,4-dihydropyran
DIBAL-H Diisobutylaluminium hydride
DMF dimethylformamide
DMP Dess-Martin periodinane
DMSO Dimethyl sulfoxide
DPPA Diphenylphosphoryl azide
Dppf 1,1′-Bis(diphenylphosphino)ferrocene
EA or EtOAc ethyl acetate
EDCI N-(3-Dimethylaminopropyl)-N′-ethylcarbodiimide
HMDS Hexamethyldisilazane
IPA isopropyl alcohol
LAH Lithium Aluminium hydride
LDA Lithium diisopropylamide
MTBE Methyl tertiary-butyl ether
NMP N-methylpyrrolidinone
NBS N-Bromosuccinimide
NIS N-Iodosuccinimide
O/N overnight
PCC pyridinium chlorochromate
PE petroleum ether
PPTS Pyridinium p-toluenesulfonate
Rt retention time (e.g., when describing HPLC peaks)
RT room temperature (describing reaction conditions)
TBAF tetra-n-butylammonium fluoride
TBS tert-butyldimethylsilyl (or TBDMS)
TBDPS tert-butyldiphenylsilyl
TEA triethyl amine
TFA trifluoroacetic acid
THF tetrahydrofuran
THP tetrahydropyran
TMS Trimethylsilyl
TPP triphenyl phosphine
TLC thin-layer chromatography
TsOH p-Toluenesulfonic acid (or PTSA)
Z benzyloxycarbonyl (benzyl chloroformate (Z—Cl))

### Synthesis of Intermediates

### Intermediate 1

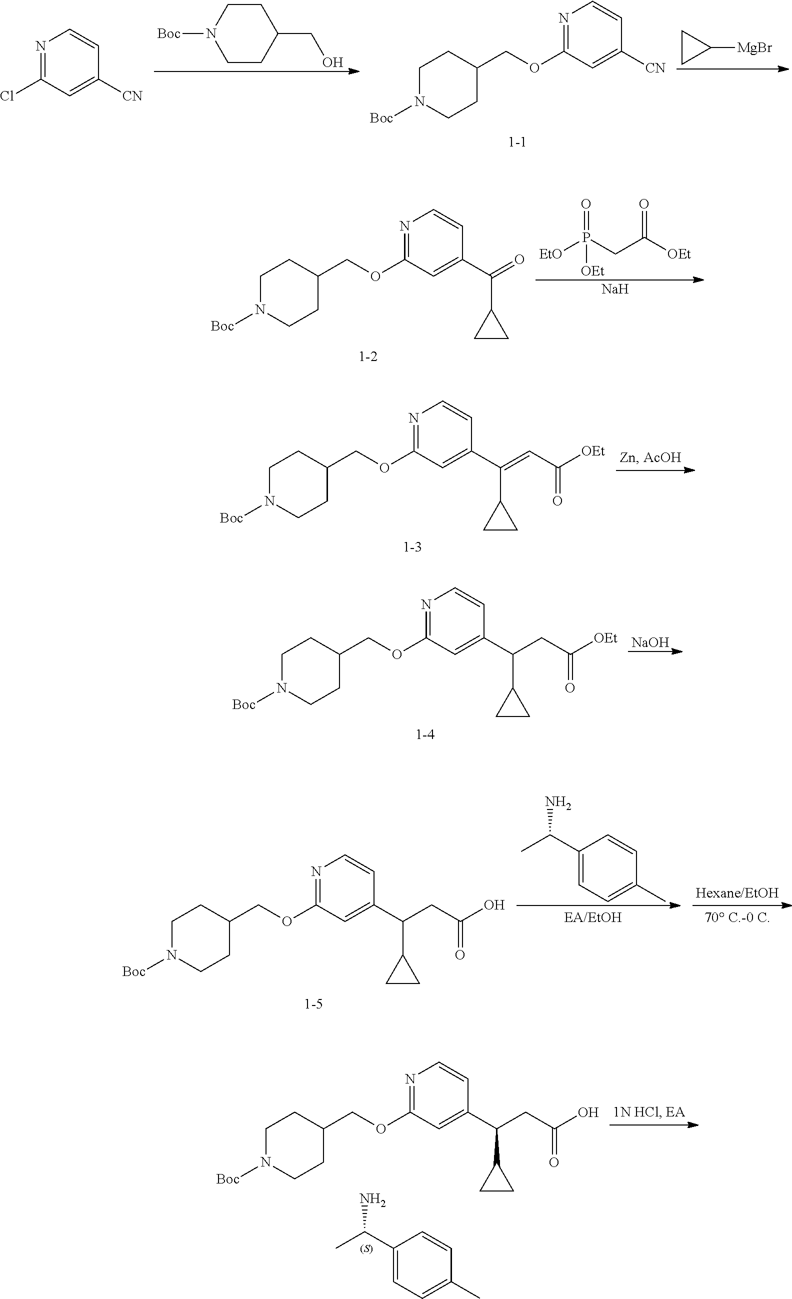

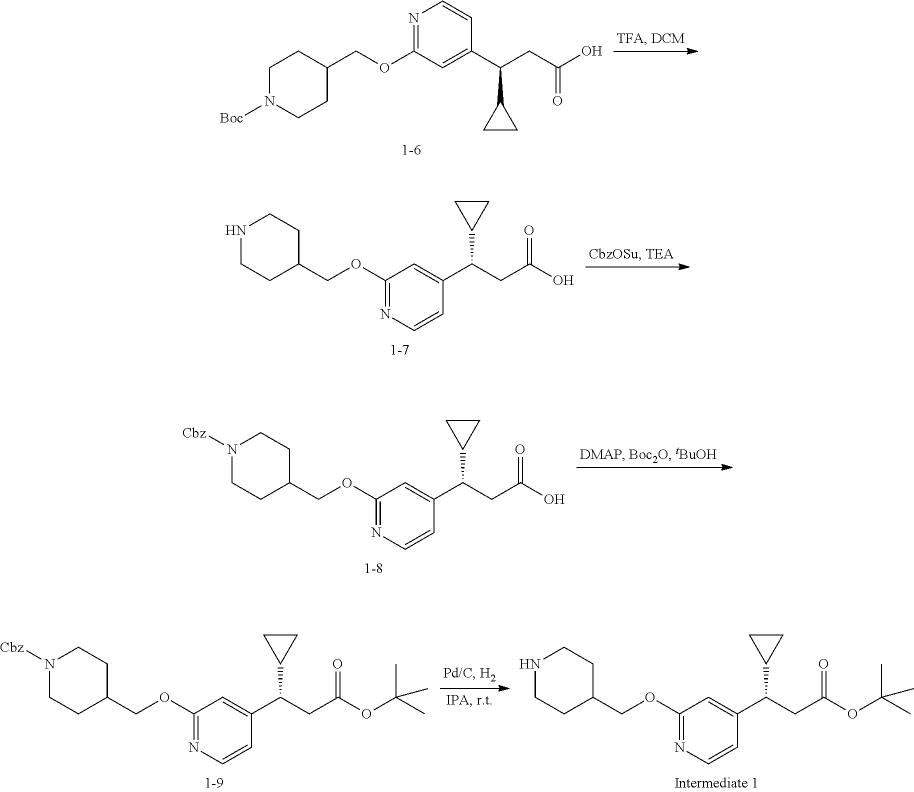 [SMILES: CC(C)(C)OC(=O)N1CCC(COc2cc([C@@H](CC(=O)O)C3CC3)ccn2)CC1 ; O=C(O)C[C@H](c1ccnc(OCC2CCNCC2)c1)C1CC1 ; O=C(O)C[C@H](c1ccnc(OCC2CCN(C(=O)OCc3ccccc3)CC2)c1)C1CC1 ; CC(C)(C)OC(=O)C[C@H](c1ccnc(OCC2CCN(C(=O)OCc3ccccc3)CC2)c1)C1CC1 ; CC(C)(C)OC(=O)C[C@H](c1ccnc(OCC2CCNCC2)c1)C1CC1]

Step 1. To a solution of compound (1) 2-chloroisonicotinonitrile (10 g, 0.072 mol) in DMF (200 mL) was added NaH (4.32 g, 0.108 mol) at 0° C., followed by a solution of (2) tert-butyl 4-(hydroxyethyl) piperidine-1-carboxylate (15.5 g, 0.072 mol) in DMF (100 mL). The mixture was stirred at 0° C. for 2 hrs. The mixture was diluted with EA and water, and was extracted with EA. The organic layer was separated, washed with brine, dried over Na₂SO₄, and concentrated in vacuo to give a white powder. The powder was collected by filtration, washed with PE, and dried to give (3) tert-butyl 4-(((4-cyanopyridin-2-yl)oxy) methyl)piperidine-1-carboxylate, 1-1, (11 g, 48% yield) as a white powder. ¹H NMR (300 MHz, CDCl3): δ 8.28 (d, J=5.1 Hz, 1H), 7.06 (d, J=5.1 Hz, 1H), 6.98 (s, 1H), 4.18-4.10 (m, 4H), 2.74 (t, J=12.1 Hz, 2H), 2.04-1.89 (m, 1H), 1.79 (d, J=12.6 Hz, 2H), 1.46 (s, 9H), 1.27-1.23 (m, 2H).

Step 2. To a solution of 1-1 (16.7 g, 0.053 mol) in THF (100 mL) was added dropwise to cyclopropylmagnesium bromide (1 M in THF, 100, 0.106 mol) at room temperature. The mixture was stirred at the same temperature for 2 hrs. The resulting mixture was slowly quenched with 1 N aq HCl below 10° C. and extracted with EA at this temperature. The organic layer was washed with water and brine, dried over Na₂SO₄, and concentrated in vacuo to give tert-butyl 4-(((4-(cyclopropanecarbonyl)pyridin-2-yl)oxy) methyl)piperidine-1-carboxylate, 1-2, (16.7 g, crude) as a brown oil which was used for the next reaction without further purifications. MS (ESI) m/z 361.2 [M+]+.

Step 3. To a suspension of NaH (3.4 g, 0.087 mol) in THF (50 mL) was added ethyl 2-(diethoxyphosphoryl)acetate (20 g, 0.087 mol) at 0° C. and stirred for 30 min. To the mixture was added a solution of 1-2 (15.6 g, 0.043 mol) in THF (50 mL) and stirred at 80° C. for 3 h. The reaction mixture was poured into sat. NH₄Cl aq (50 mL), and the mixture was extracted with EA. The organic layer was separated, washed with brine, dried over Na₂SO₄, and concentrated in vacuo to give tert-butyl (E/Z)-4-(((4-(1-cyclopropyl-3-ethoxy-3-oxoprop-1-en-1-yl)pyridin-2-yl)oxy)methyl)piperidine-1-carboxylate, 1-3, (17.5 g, crude) as a brown oil which was used for the next reaction without further purifications. MS (ESI) m/z 431.3 [M+]⁺.

Step 4. Zinc powder (15.8 g, 0.24 mol) was added portion wise to a solution of 1-3 (17.5 g, 0.041 mol) in HOAc (80 mL) at room temperature. The mixture was stirred at room temperature for 2 hrs. The reaction mixture was filtered through a celite pad, and the filtrate was concentrated in vacuo. The residue was basified with NaHCO₃ and extracted with EtOAc. The organic layer was separated, washed with sat. NaHCO₃ aq and brine, dried over Na₂SO₄, and concentrated in vacuo. The residue was purified by column chromatography (PE/EA=5/1) to give tert-butyl4-(((4-(1-cyclopropyl-3-ethoxy-3-oxopropyl)pyridine-2-yl)oxy)methyl)-piperidine-1-carboxylate, 1-4, (13.2 g, 75% yield) as a pale-yellow oil. MS (ESI) m/z 433.3 [M+]⁺. ¹H NMR (300 MHz, CDCl₃): δ 8.08-8.07 (m, 1H), 6.80-6.78 (m, 1H), 6.64 (s, 1H), 4.17-4.07 (m, 6H), 2.83-2.73 (m, 4H), 2.35-2.32 (m, 1H), 1.87-1.82 (m, 2H), 1.50 (s, 9H), 1.32-1.19 (m, 5H), 1.03-1.00 (m, 1H), 0.65-0.62 (m, 1H), 0.52-0.48 (m, 1H), 0.35-0.30 (m, 1H), 0.22-0.19 (m, 1H).

Step 5. To a mixture of 1-4 (13.2 g, 0.031 mol) in MeOH/H₂O (50 mL/30 mL) was added NaOH (4.89 g, 0.122 mol) at room temperature. After stirring at ambient temperature for 2 hrs, the mixture was concentrated in vacuo to remove MeOH. The residue was diluted with H₂O and washed with EA. The aqueous phase was acidified with 1 N HCl until pH reached 4-5. The solid was collected by filtration, washed with PE, and dried in vacuo to give 3-(2-((1-(tert-butoxycarbonyl)piperidin-4-yl)methoxy)pyridin-4-yl)-3-cyclo propylpropanoic acid, 1-5, (9.9 g, 79% yield) as a white powder. MS (ESI) m/z 405.3 [M+]⁺.

Step 6. To a solution of 1-5 (9.9 g, 0.025 mol) in EtOH (100 mL) was added (S)-1-(p-tolyl)ethanamine (3.375 g, 0.025 mol) in EA (30 mL). After stirring at ambient temperature for 12 hrs, the mixture was filtered and the filter cake was washed with EtOH/EA (v:v=1/2, 30 mL×3). The solid was dissolved in EtOH/Hexane (v:v=1/2, 350 mL) at 70° C., and upon cooling to room temperature the solid was collected by filtration. The solid was suspended in ethyl acetate (50 mL), and a solution of 1N HCl (99 mL) was added dropwise at 0° C. and stirred for 30 mins. The mixture was extracted with EA and EA/THF (v:v=1/1). The combined organic phase was dried and concentrated in vacuo to give (S)-3-(2-((1-(tert-butoxycarbonyl)piperidin-4-yl)methoxy)pyridin-4-yl)-3-cyclopropyl propanoic acid, 1-6, (4 g, 40% yield) as a white solid. Chiral HPLC analysis: e.e. 96.5%. Method Info: Column: Chiralpak IA, Mobile phase: Hex:IPA:TFA=90:10:0.2, peak 1, Rt=7.493; peak 2, Rt=9.199.

Step 7. To a mixture of 1-6 (11 g, 27.2 mmol) in DCM (100 mL) at 0° C. was added TFA (25 mL) dropwise. After addition the resulting mixture was stirred for 1.5 hrs at r. t. Solvent was removed and the residue was co-evaporated with toluene twice to give crude (S)-3-cyclopropyl-3-(2-(piperidin-4-ylmethoxy)pyridin-4-yl)propanoic acid, 1-7, (TFA salt, 20 g crude) as a pale-yellow gum. MS (ESI) m/z=305.2 [M+H]+.

Step 8. To a mixture of 1-7 (TFA salt, 20 g crude, 0.0272 mmol) and TEA (13.9 g, 0.136 mol) in THF (150 mL) at 0° C. was added CbzOSu (8.13 g, 0.0326 mol) in portions. After addition the resulting suspension was stirred for 12 hrs at r. t. The reaction mixture became clear. Solvent was removed and the residue was diluted with water (100 mL), extracted with EA (60 mL×3), dried and concentrated. The residue was purified by flash chromatography (20% to 35% of EA in PE) to give (S)-3-(2-((1-((benzyloxy)carbonyl) piperidin-4-yl)methoxy)-pyridin-4-yl)-3-cyclopropylpropanoic acid, 1-8, (10.2 g, yield: 85%) as a colorless gum. MS (ESI) m/z=439.2 [M+H]+. ¹H NMR (400 MHz, DMSO-d6) δ 12.01 (s, 1H), 8.02-8.01 (d, 1H), 7.39-7.29 (m, 5H), 6.91-6.89 (d, 1H), 6.69 (s, 1H), 5.07 (s, 2H), 4.09-4.00 (m, 2H), 2.90-2.76 (m, 2H), 2.68-2.65 (m, 2H), 2.25-2.21 (m, 1H), 1.98-1.92 (m, 1H), 1.78-1.72 (m, 2H), 1.24-1.12 (m, 2H), 1.04-0.94 (m, 1H), 0.54-0.47 (m, 1H), 0.35-0.23 (m, 2H), 0.18-0.13 (m, 1H).

Step 9. To a mixture of 1-8 (10.2 g, 0.0233 mol) in tBuOH (110 mL) was added Boc2O (10.1 g, 0.0466 mol) and DMAP (850 mg, 6.98 mmol). After addition the resulting mixture was stirred for 2 hrs at 35° C. LCMS indicated the completion of reaction. Solvent was removed and the residue was purified by flash chromatography (10% of EA in PE) to give benzyl (S)-4-(((4-(3-(tert-butoxy)-1-cyclopropyl-3-oxopropyl)pyridin-2-yl)oxy)methyl) piperidine-1-carboxylate, 1-9, (7.5 g, yield: 65%) as a colorless gum. MS (ESI) m/z=439.1 [M+H]⁺.

Step 10. A flask charged with 1-9 (7.5 g, 15.15 mmol) and Pd/C (wet, ~1.4 g) in isopropyl alcohol (120 mL) was degassed and filled with hydrogen using a balloon. The resulting mixture was then hydrogenated for 1.5 hrs at r. t. LCMS indicated the completion of reaction. The mixture was filtered over celite, and the filter cake was washed with EA/MeOH. The filtrate was combined and concentrated to give tert-butyl (S)-3-cyclopropyl-3-(2-(piperidin-4-ylmethoxy)pyridin-4-yl)propanoate, Intermediate 1, (5.5 g, 100% yield) as a colorless gum. MS (ESI) m/z=361.2 [M+H]+. 1H NMR (400 MHz, DMSO-d6) δ 8.02-8.01 (d, 1H), 6.89-6.87 (dd, 1H), 6.67 (s, 1H), 4.06-4.04 (d, 2H), 2.95-2.92 (m, 2H), 2.69-2.62 (m, 2H), 2.45-2.41 (m, 2H), 2.25-2.15 (m, 1H), 1.83-1.77 (m, 1H), 1.66-1.63 (m, 2H), 1.26 (s, 9H), 1.17-1.07 (m, 2H), 1.02-0.96 (m, 1H), 0.54-0.48 (m, 1H), 0.37-0.22 (m, 2H), 0.17-0.13 (m, 1H).

### Intermediate 2

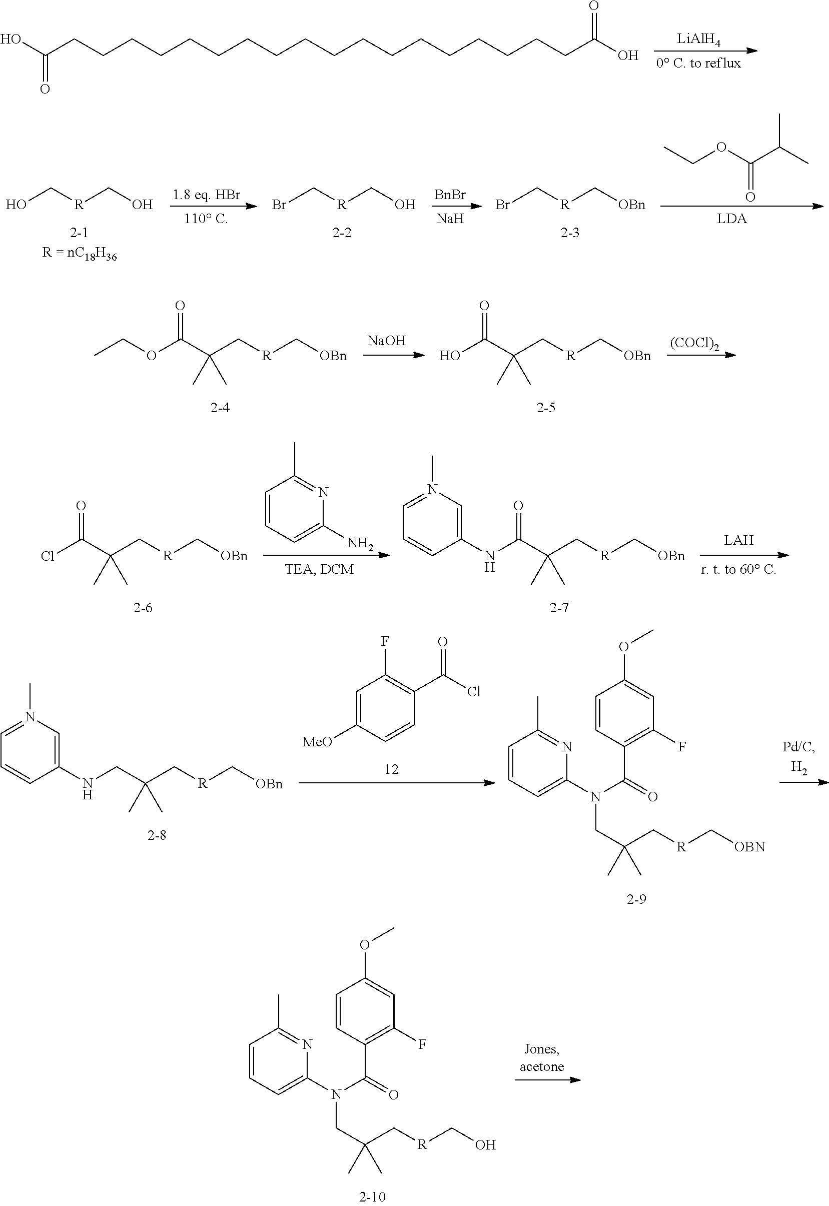

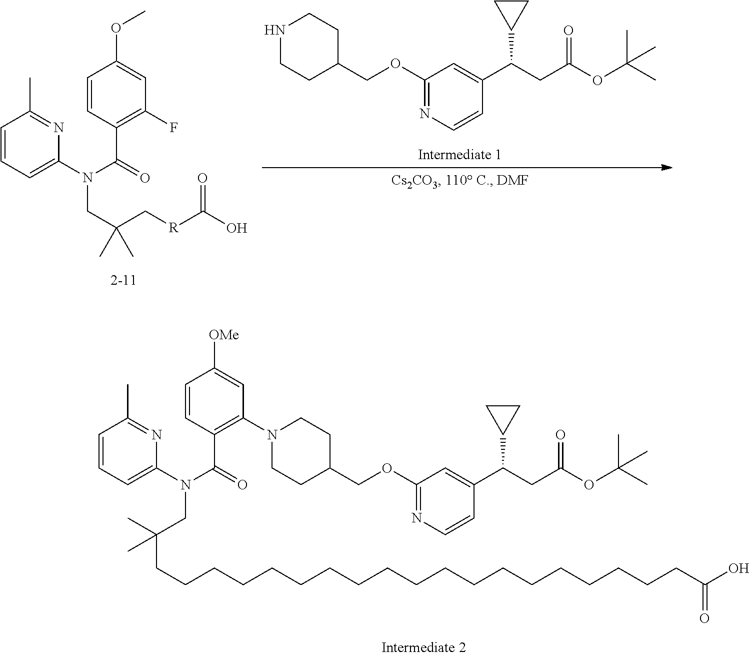

Step 1. To a solution of icosanedioic acid (6.9 g, 20.2 mmol) in THF (300 mL) was added LiAlH₄ (3.07 g, 80.8 mmol) in portions at 0° C. The mixture was stirred at 80° C. overnight under N2. Water (3.1 mL) was added to the reaction at 0° C. followed by NaOH (15% solution, 3.1 mL) and more water (9.3 mL). The resulting mixture was diluted with THF, dried with Na₂SO₄, filtered, and the filtrate was concentrated to give icosane-1, 20-diol, 2-1, (10.2 g, 81% yield) as a white solid. 1H NMR (400 MHz, CDCl3) δ 3.64 (t, 4H), 1.57 (br, 4H), 1.43-1.25 (m, 32H).

Step 2. To a solution of 2-1 (8.38 g, 26.7 mmol) in toluene (60 mL) was added HBr (9.01 g, 53.4 mmol). The reaction mixture was stirred at 110° C. overnight under N2. The reaction mixture was concentrated under reduced pressure to remove excess reagent and solvent. The crude product was purified by column chromatograph (PE/EA=4/1) to give 20-bromoicosan-1-ol, 2-2, (4.4 g, 44% yield) as a white solid. ¹H NMR (400 MHz, CDCl3) δ 3.64 (t, 2H), 3.41 (t, 2H), 1.85 (m, 2H), 1.44-1.40 (m, 2H), 1.40-1.26 (m, 32H).

Step 3. To a solution of 2-2 (4.4 g, 11.75 mmol) in THF (20 mL) was added NaH (338.8 mg, 23.49 mmol) at 0° C. The reaction mixture was stirred at 0° C. for 1 hr and followed by dropwise addition of BnBr (4.01 g, 23.4 mmol). The reaction mixture was further stirred at room temperature overnight. The reaction mixture was quenched with saturated aqueous NH₄Cl (1 mL) at 0° C. The resulting solution was extracted with EA (20 mL×3), dried over Na₂SO₄ and filtered. The filtrate was concentrated, and the crude product was purified by silica gel column (PE/EA=10/1) to give (((20-bromoicosyl)oxy)methyl)-benzene, 2-3, (3.6 g, 66% yield) as an orange-yellow solid. 1H NMR (400 MHz, CDCl3) δ 7.35-7.26 (m, 4H), 7.30-7.25 (m, 1H), 4.50 (s, 2H), 3.46 (t, 2H), 3.41 (t, 2H), 1.85 (m, 2H), 1.61 (m, 2H), 1.41-1.26 (m, 32H).

Step 4. To a solution of ethyl isobutyrate (2.02 g, 17.47 mmol) in THF (50 mL) was added LDA (8.7 mL, 17.47 mmol) at −78° C. under N2 dropwise. The reaction mixture was stirred at −78° C. for 1 hr. To the mixture was added 2-3 (3.6 g, 13.3 mmol) at −78° C. dropwise. The reaction mixture was warmed up to room temperature and stirred overnight. The reaction mixture was quenched by saturated aqueous NH4Cl (10 mL) at 0° C. The resulting solution was extracted with EA (50 mL×3), dried over Na2SO4 and filtered. The filtrate was concentrated, and the crude product was chromatographed on silica gel (PE/EA=10/1) to give ethyl 22-(benzyloxy)-2,2-dimethyldocosanoate, 2-4, (4.3 g, 97% yield) as a pale-yellow solid. 1H NMR (400 MHz, CDCl3) δ 7.34-7.26 (m, 4H), 7.30-7.25 (m, 1H), 4.50 (s, 2H), 4.11 (q, 2H), 3.46 (t, 2H), 1.63-1.57 (m, 2H), 1.51-1.47 (m, 2H), 1.37-1.22 (m, 37H), 1.15 (s, 6H).

Step 5. To a solution of 2-4 (4.3 g, 7.56 mmol) in THF/MeOH/H2O (20 mL/20 mL/10 mL) was added NaOH (7.84 g, 196 mmol). The reaction mixture was stirred at 60° C. overnight. The reaction mixture was concentrated under reduced pressure to remove MeOH and THF. The residue was diluted with solvent and poured into water. The aqueous phase was acidified with aqueous HCl and extracted with EA (50 mL×4). The combined organic layers were dried over Na₂SO₄ and concentrated. The crude product was chromatographed on silica gel (PE/EA=1/1) to give 22-(benzyloxy)-2,2-dimethyldocosanoic acid, 2-5, (2.55 g, 71% yield) as a white solid. 1H NMR (400 MHz, CDCl3) δ 7.34-7.29 (m, 4H), 7.30-7.25 (m, 1H), 4.50 (s, 2H), 3.46 (t, 2H), 1.61-1.49 (m, 4H), 1.31-1.25 (m, 34H), 1.19 (s, 6H).

Step 6. To a solution of 2-5 (2.55 g, 5.4 mmol) in dry DCM (15 mL) was added (COCl)₂ (1.02 g, 8.1 mmol) and DMF (0.5 mL) at 0° C. under N2 atmosphere. The reaction mixture was stirred at room temperature for 2 hrs. The reaction mixture was concentrated to give 22-(benzyloxy)-2,2-dimethyldocosanoyl chloride, 2-6, as a yellow oil.

Step 7. To a solution of 6-methylpyridin-2-amine (2.5 g, 23.2 mmol) and TEA (3.55 g, 34.8 mmol) in THF (25 mL) was added a solution of 2-6 (5.72 g, 11.6 mmol) in THF (25 mL) dropwise. The reaction mixture was stirred at room temperature overnight under N2. The reaction mixture was extracted with EA (50 mL×4). The combined organic layers were dried with Na₂SO₄ and concentrated. The crude product was chromatographed on silica gel (PE/EA=10/1) to give 22-(benzyloxy)-2,2-dimethyl-N-(6-methylpyridin-2-yl) docosanamide, 2-7, (6.3 g, 97% yield) as an orange solid. 1H NMR (400 MHz, CDCl3) δ 8.06 (d, J=11.2 Hz, 1H), 7.90 (brs, 1H), 7.58 (t, J=10.4 Hz, 1H), 7.35-7.26 (m, 5H), 6.88 (d, J=10.0 Hz, 1H), 4.50 (s, 2H), 3.46 (t, J=8.8 Hz, 2H), 2.45 (s, 3H), 1.63-1.57 (m, 6H), 1.28 (s, 6H), 1.28-1.24 (m, 32H).

Step 8. To a solution of 2-7 (1.23 mg, 2.2 mmol) in THF (10 mL) was added LiAlH₄ (836 mg, 22 mmol) in portions at room temperature. The mixture was stirred at 60° C. overnight under N2. The reaction was complete detected by TLC. The reaction mixture was added H₂O (0.8 mL) at 0° C. Then 15% of NaOH aqueous solution (0.8 mL) and water (2.4 mL) were added. The resulting mixture was diluted with THF, dried with Na₂SO₄, filtered, and the filtrate was concentrated to give N-(22-(benzyloxy)-2,2-dimethyldocosyl)-6-methyl pyridin-2-amine, 2-8, (1.2 g, crude) as a pale-green oil. ¹H NMR (400 MHz, CDCl3) δ 7.36-7.26 (m, 6H), 6.41 (d, J=9.6 Hz, 1H), 6.21 (d, J=11.2 Hz, 1H), 4.50 (s, 2H), 3.46 (t, J=8.8 Hz, 2H), 2.98 (d, J=8.0 Hz, 2H), 2.36 (s, 3H), 1.69-1.57 (m, 6H), 1.43-1.25 (m, 32H), 0.94 (s, 6H).

Step 9. To a solution of 2-8 (1.2 g, 2.1 mmol) and TEA (636.3 mg, 6.3 mmol) in THF (10 mL) was added a solution of 2-fluoro-4-methoxybenzoyl chloride in THF (10 mL). The reaction mixture was stirred at 40° C. for 4 hrs. The reaction mixture was quenched with H₂O (40 mL) and was extracted by EA (50 mL×2). The combined organic layers were dried with Na₂SO₄ and concentrated. The crude product was chromatographed on silica gel (PE/EA=10/1) to give N-(22-(benzyloxy)-2,2-dimethyldocosyl)-2-fluoro-4-methoxy-N-(6-methyl pyridin-2-yl)benzamide, 2-9, (1.2 g, 70% yield) as a white solid. ¹H NMR (400 MHz, CDCl₃) δ 7.35-7.31 (m, 4H), 7.29-7.25 (m, 1H), 7.25-7.23 (m, 1H), 7.11 (t, 1H), 6.85-6.83 (d, 1H), 6.60-6.58 (d, 1H), 6.54-6.51 (dd, 1H), 6.40-6.36 (dd, 1H), 4.50 (s, 2H), 4.13 (s, 2H), 3.73 (s, 3H), 3.48-3.46 (t, 2H), 2.47 (s, 3H), 1.62-1.57 (m, 2H), 1.41-0.97 (m, 36H), 0.83 (s, 6H).

Step 10. To a solution of 2-9 (1.2 g, 1.7 mmol) in MeOH (12 mL) was added Pd/C (120 mg) and Pd(OH)₂ (120 mg). The reaction mixture was stirred at room temperature overnight under H₂. The mixture was filtered and the filtrate was concentrated to give 2-fluoro-N-(22-hydroxy-2,2-dimethyldocosyl)-4-methoxy-N-(6-methylpyridin-2-yl)benzamide, 2-10, (930 mg, 89% yield) as a yellow oil. 1H NMR (400 MHz, DMSO-d6) δ: 7.46 (t, J=7.6 Hz, 1H), 7.07 (t, J=8.0 Hz, 1H), 6.97 (d, J=7.2 Hz, 1H), 6.74 (d, J=7.6 Hz, 1H), 6.67-6.62 (m, 2H), 4.30 (br, 1H), 3.98 (s, 2H), 3.70 (s, 3H), 3.36 (t, J=6.4 Hz, 2H), 2.35 (s, 3H), 1.39 (t, J=6.8 Hz, 2H), 1.29-0.87 (m, 36H), 0.77 (m, 6H). MS (ESI) m/z 613.5 [M+1]+.

Step 11. To a solution of 2-10 (930 mg, 1.51 mmol) in acetone (10 mL) was added Jones reagent (3.7 mL, 7.55 mmol) at 0° C. dropwise. The reaction mixture was stirred at room temperature for 1 hr. The mixture was quenched by H2O (30 mL) and extracted by EA (50 mL×3). The combined organic layers were dried, concentrated to give crude product. The crude product was purified by silica gel column (PE/EA=3/1) to give 22-(2-fluoro-4-methoxy-N-(6-methylpyridin-2-yl)benzamido)-21,21-dimethyldocosanoic acid, 2-11, (750 mg, 78% yield) as a yellow oil. 1H NMR (300 MHz, DMSO-d6) δ:11.94 (s, 1H), 7.49 (t, J=7.8 Hz, 1H), 7.10 (t, J=8.1 Hz, 1H), 6.98-6.96 (d, J=7.8 Hz, 1H), 6.74-6.72 (d, J=7.8 Hz, 1H), 6.70-6.66 (m, 2H), 3.98 (s, 2H), 3.70 (s, 3H), 2.34 (s, 3H), 2.18 (t, J=7.2 Hz, 1H), 1.50-1.45 (m, 2H), 1.26-0.99 (m, 34H), 0.77 (m, 6H). MS (ESI) m/z 627.3 [M+1]+.

Step 12. To a solution of 2-11 (550 mg, 0.88 mmol) and Intermediate 1 (622 mg, 1.75 mmol) in DMF (1 mL) was added Cs₂CO₃ (860 mg, 2.64 mmol) and TBA (65 mg, 0.076 mmol). The reaction mixture was stirred at 110° C. for 3 days. The reaction mixture was acidified to pH=5, and extracted by EA (30 mL×3). The combined organic layers were dried, concentrated to give crude product. The crude product was purified by silica gel column (PE/EA/HOAc=10/1/0.001) to give (S)-22-(2-(4-(((4-(3-(tert-butoxy)-1-cyclopropyl-3-oxopropyl)pyridin-2-yl)oxy)methyl)piperidin-1-yl)-4-methoxy-N-(6-methylpyridin-2-yl)benzamido)-21,21-dimethyldocosanoic acid, Intermediate 2, (210 mg, 24% yield) as a yellow oil. 1H NMR (300 MHz, DMSO-d6): 8.05-8.03 (d, 1H), 7.28-7.24 (t, 1H), 7.10-7.08 (d, 1H), 6.91-6.90 (d, 1H), 6.85-6.83 (m, 1H), 6.69 (s, 1H), 6.52-6.49 (dd, 1H), 6.43 (br, 1H), 6.22 (s, 1H), 4.20-4.10 (m, 2H), 4.10-3.98 (m, 2H), 3.68 (s, 3H), 2.65-2.61 (m, 2H), 2.36 (s, 3H), 2.22-2.15 (m, 3H), 1.72 (br, 1H), 1.68-1.61 (m, 2H), 1.50-1.82 (m, 2H), 1.29-1.05 (m, 32H), 1.02-0.85 (m, 5H), 0.80-0.68 (m, 6H), 0.57-0.48 (m, 1H), 0.36-0.23 (m, 2H), 0.18-0.12 (m, 1H). MS (ESI) m/z 484.2 [M/2+1]+.

The following intermediates were prepared in the same way as Intermediate 2.

### Intermediate 3

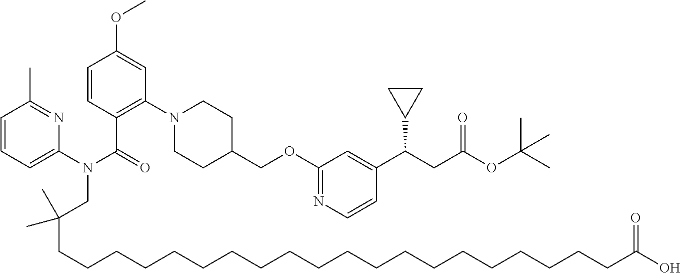 [SMILES: COc1ccc(C(=O)N(CC(C)(C)CCCCCCCCCCCCCCCCCCCCC(=O)O)c2cccc(C)n2)c(N2CCC(COc3cc([C@@H](CC(=O)OC(C)(C)C)C4CC4)ccn3)CC2)c1]

MS (ESI) m/z 317.2 [M+]⁺. ¹H NMR (400 MHz, DMSO-d₆): 8.04-8.03 (d, 1H), 7.27-7.25 (t, 1H), 7.10-7.08 (d, 1H), 6.91-6.89 (d, 1H), 6.85-6.83 (m, 1H), 6.69 (s, 1H), 6.52-6.49 (dd, 1H), 6.42 (br, 1H), 6.22 (s, 1H), 4.17-4.12 (m, 2H), 4.05-3.96 (m, 2H), 3.68 (s, 3H), 2.65-2.61 (m, 2H), 2.36 (s, 3H), 2.22-2.15 (m, 3H), 1.72 (br, 1H), 1.68-1.61 (m, 2H), 1.50-1.82 (m, 2H), 1.29-1.05 (m, 34H), 1.02-0.88 (m, 5H), 0.80-0.68 (m, 6H), 0.57-0.48 (m, 1H), 0.36-0.23 (m, 2H), 0.18-0.12 (m, 1H).

### Intermediate 4

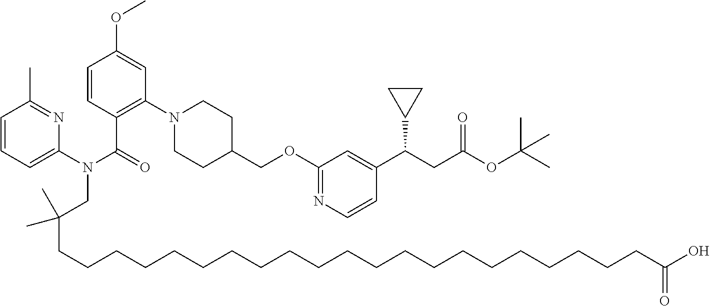 [SMILES: COc1ccc(C(=O)N(CC(C)(C)CCCCCCCCCCCCCCCCCCCCCC(=O)O)c2cccc(C)n2)c(N2CCC(COc3cc([C@@H](CC(=O)OC(C)(C)C)C4CC4)ccn3)CC2)c1]

MS (ESI) m/z 317.2 [M+]⁺. ¹H NMR (400 MHz, DMSO-d₆): 8.05-8.03 (d, 1H), 7.26-7.23 (t, 1H), 7.10-7.08 (d, 1H), 6.91-6.89 (d, 1H), 6.85-6.83 (m, 1H), 6.69 (s, 1H), 6.52-6.49 (dd, 1H), 6.42 (br, 1H), 6.22 (s, 1H), 4.17-4.12 (m, 2H), 4.05-3.96 (m, 2H), 3.68 (s, 3H), 2.65-2.61 (m, 2H), 2.36 (s, 3H), 2.22-2.15 (m, 3H), 1.72 (br, 1H), 1.68-1.61 (m, 2H), 1.50-1.82 (m, 2H), 1.29-1.05 (m, 36H), 1.02-0.88 (m, 5H), 0.80-0.68 (m, 6H), 0.57-0.48 (m, 1H), 0.36-0.23 (m, 2H), 0.18-0.12 (m, 1H).

### Intermediate 5

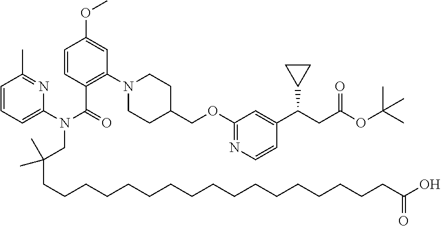 [SMILES: COc1ccc(C(=O)N(CC(C)(C)CCCCCCCCCCCCCCCCCC(=O)O)c2cccc(C)n2)c(N2CCC(COc3cc([C@@H](CC(=O)OC(C)(C)C)C4CC4)ccn3)CC2)c1]

MS (ESI) m/z 317.2 [M+]⁺. ¹H NMR (400 MHz, DMSO-d₆): 8.05-8.03 (d, 1H), 7.28-7.25 (t, 1H), 7.10-7.08 (d, 1H), 6.91-6.90 (d, 1H), 6.85-6.84 (m, 1H), 6.69 (s, 1H), 6.52-6.49 (dd, 1H), 6.42 (br, 1H), 6.22 (s, 1H), 4.17-4.12 (m, 2H), 4.05-3.96 (m, 2H), 3.68 (s, 3H), 2.65-2.61 (m, 2H), 2.36 (s, 3H), 2.22-2.15 (m, 3H), 1.72 (br, 1H), 1.68-1.61 (m, 2H), 1.50-1.46 (m, 2H), 1.29-1.15 (m, 28H), 1.02-0.88 (m, 5H), 0.76-0.68 (m, 6H), 0.57-0.48 (m, 1H), 0.36-0.23 (m, 2H), 0.18-0.12 (m, 1H).

### Intermediate 6

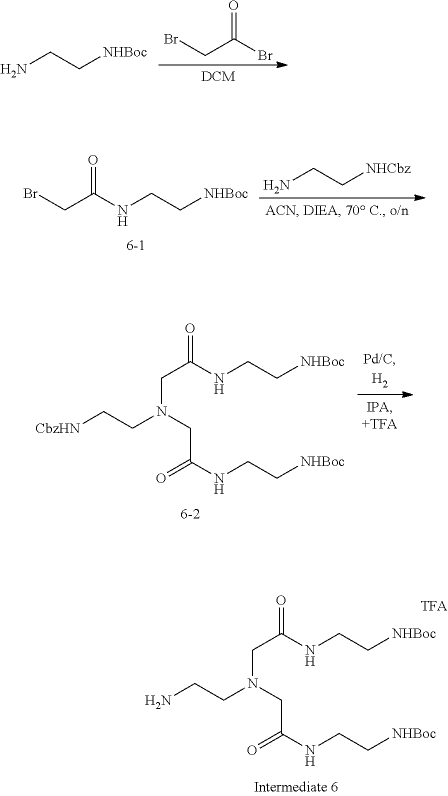 [SMILES: CC(C)(C)OC(=O)NCCN ; O=C(Br)CBr ; CC(C)(C)OC(=O)NCCNC(=O)CBr ; NCCNC(=O)OCc1ccccc1 ; CC(C)(C)OC(=O)NCCNC(=O)CN(CCNC(=O)OCc1ccccc1)CC(=O)NCCNC(=O)OC(C)(C)C ; CC(C)(C)OC(=O)NCCNC(=O)CN(CCN)CC(=O)NCCNC(=O)OC(C)(C)C]

Step 1. To a mixture of tert-butyl (2-aminoethyl)carbamate (20 g, 0.125 mol) and TEA (18.95 g, 0.187 mol) in DCM (200 mL) at 0° C. was added dropwise 2-bromoacetyl bromide (30 g, 0.15 mol) in DCM (30 mL). The resulting mixture was stirred for 12 hrs at room temperature, followed by addition of water (100 mL) and extraction with DCM. The combined organic phase was dried and concentrated. The residue was purified by silica gel column chromatography (PE/EA=5:1 to 2:1) to give tert-butyl (2-(2-bromoacetamido)-ethyl) carbamate, 6-1, (9.5 g, yield: 27%) as pale yellow solid. MS (ESI) m/z 303.1 [M+Na]. ¹H NMR (400 MHz, DMSO-d6): 8.26 (s, 1H), 6.80 (s, 1H), 3.83 (s, 2H), 3.12-3.07 (m, 2H), 3.00-2.96 (m, 2H), 1.38 (s, 9H).

Step 2. A mixture of 6-1 (9.1 g, 32.5 mmol), benzyl (2-aminoethyl)carbamate (2.4 g, 12.3 mmol) and DIEA (6.68 g, 51.3 mmol) in MeCN (100 mL) was heated at 70° C. for 16 hrs. The product benzyl (11-(2-((2-((tert-butoxycarbonyl)amino)ethyl)amino)-2-oxoethyl)-2,2-dimethyl-4,9-dioxo-3-oxa-5,8,11-triazatridecan-13-yl)carbamate, 6-2, was collected by filtration as white solid (5.6 g, yield: 76%). MS (ESI) m/z 595.4 [M+H]. ¹H NMR (400 MHz, DMSO-d6): 8.03 (s, 2H), 7.35-7.30 (m, 5H), 7.21 (s, 1H), 6.80 (s, 2H), 5.01 (s, 2H), 3.18 (s, 4H), 3.11-3.08 (m, 4H), 3.01-2.98 (m, 4H), 2.55-2.53 (m, 2H), 1.37 (s, 18H).

Step 3. A flask containing 6-2 (520 mg, 0.85 mmol) and Pd/C (wet, ~100 mg) in isopropyl alcohol (25 mL) was degassed and filled with hydrogen using a balloon. The resulting mixture was then hydrogenated for 1.5 hrs at r. t. The mixture was filtered over celite, washed with EA and IPA. The filtrate was combined and concentrated to give crude di-tert-butyl (((2,2′-((2-aminoethyl)azanediyl)bis(acetyl))bis(azanediyl))bis(ethane-2,1-diyl))dicarbamate, Intermediate 6, (490 mg, crude) as a pale-yellow gum which was directly used in the next step.

### Intermediate 7

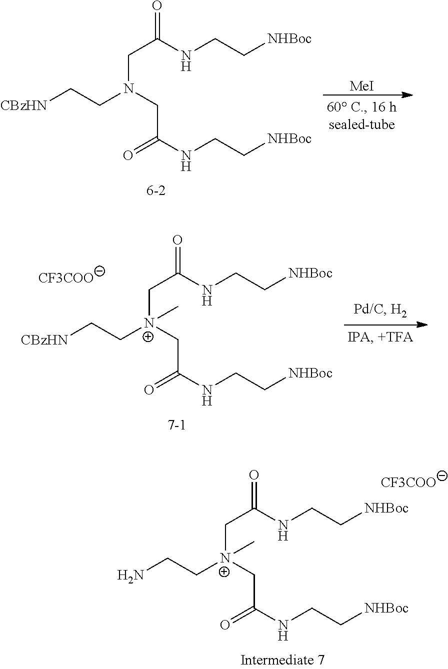

Step 1. A pressure tube charged with 6-2 (500 mg) and Mel (2 mL, 32 mmol) in ACN (7 mL) was sealed and heated at 60° C. for 20 hrs. The reaction mixture was concentrated and the residue was purified by prep-HPLC to give N-(2-(((benzyloxy)carbonyl) amino)ethyl)-2-((2-((tert-butoxycarbonyl)amino)ethyl)amino)-N-(2-((2-((tert-butoxy carbonyl)amino) ethyl)amino)-2-oxoethyl)-N-methyl-2-oxoethan-1-aminium 2,2,2-trifluoroacetate, 7-1, (490 mg, 81%) as a white solid after freeze drying. MS (ESI) m/z 609.3 [M+]. ¹H NMR (400 MHz, DMSO-d6): 8.64 (t, 2H), 7.58 (s, tH), 7.39-7.32 (m, 5H), 6.84 (mt, 2H), 5.05 (s, 2H), 4.29 (s, 4H), 3.79-3.75 (m, 2H), 3.51-3.46 (m, 2H), 3.34 (s, 3H), 3.13-3.10 (m, 4H), 3.03-3.00 (m, 4H), 1.37 (s, 18H).

Step 2. A flask charged with 7-1 (520 mg, 0.85 mmol) and Pd/C (wet, ~100 mg) in isopropyl alcohol (25 mL) was degassed and filled with hydrogen using a balloon. The resulting mixture was then hydrogenated for 1.5 hrs at r. t. The mixture was filtered over celite, washed with EA and IPA. The filtrate was combined and concentrated to give crude N-(2-aminoethyl)-2-((2-((tert-butoxycarbonyl)amino)ethyl)amino)-N-(2-((2-((tert-butoxycarbonyl)amino)ethyl) amino)-2-oxoethyl)-N-methyl-2-oxoethan-1-aminium 2,2,2-trifluoroacetate, Intermediate 7, (490 mg, crude) as a pale-yellow gum which was directly used in the next step. Note: TFA (200 mg, 1.75 mmol) was added to stabilize the product before hydrogenation.

### Intermediate 8

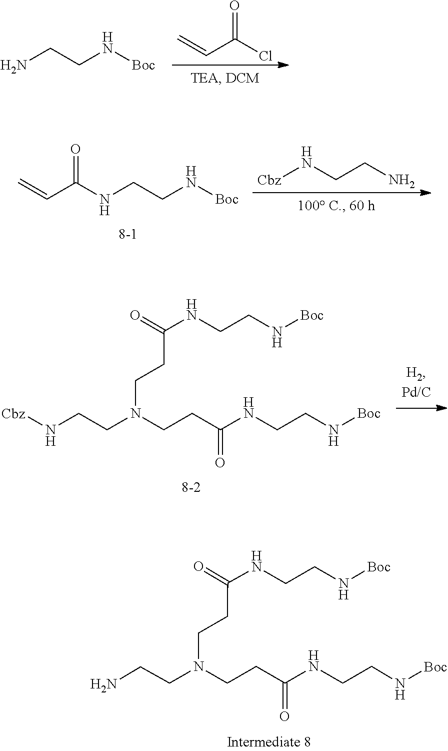

Step 1. To a mixture of (2-amino-ethyl)-carbamic acid tert-butyl ester (8 g, 0.005 mol) and TEA (15.3 g, 0.15 mol) in DCM (120 mL) at 0° C. was added acryloyl chloride (6.787 g, 0.075 mol, caution: tears!) dropwise during 5 min. After addition, the resulting mixture was stirred for 16 hr, allowing the temperature slowly warm to r. t. The reaction mixture was quenched with aq. NaHCO₃ and separated, extracted with DCM (50 mL×2). The combined organic phase was dried and concentrated. The residue was purified by flash chromatography (60% EA in PE) to give (2-acryloylamino-ethyl)-carbamic acid tert-butyl ester 8-1 (5.5 g, yield: 51%) as pale-yellow solid. ¹H NMR (400 MHz, CDCl₃): δ=6.46 (br, 1H), 6.28-6.24 (d, J=16.8 Hz, 1H), 6.13-6.07 (dd, J=16.8 Hz, 10.4 Hz, 1H), 5.65-5.62 (d, J=10.4 Hz, 1H), 4.98 (br, 1H), 3.46-3.42 (m, 2H), 3.33-3.29 (m, 2H), 1.43 (s, 9H).

Step 2. A pressure tube charged with (2-amino-ethyl)-carbamic acid benzyl ester (500 mg, 2.577 mmol) and 8-1 (2.76 g, 12.886 mmol) in sat. aq. HBO₃ (5 mL) was sealed and heated at 100° C. for 2 days. The reaction mixture was diluted with water (10 mL), extracted with DCM (30 mL×4) and concentrated. The residue was purified by flash chromatography (10% MeOH in DCM, @214 nm) to give (2-{bis-[2-(2-tert-butoxycarbonyl amino-ethylcarbamoyl)-ethyl]-amino}-ethyl)-carbamic acid benzyl ester, 8-2 (550 mg, yield: 35%) as white solid. MS (ESI) m/z 623.1 [M+H]⁺.

Step 3. A flask charged with 8-2 (550 mg, 0.884 mmol) and Pd/C (~270 mg, 50% w.t.) in MeOH (50 mL) was degassed and filled with hydrogen using a balloon. The resulting mixture was then hydrogenated at 25° C. for 16 hrs. The reaction was filter over celite and concentrated. The residue was purified by flash chromatography (28% MeOH in DCM, 0.5% NH₃·H₂O) to give [2-(3-{(2-amino-ethyl)-[2-(2-tert-butoxycarbonylamino-ethylcarbamoyl)-ethyl]-amino}-propionylamino)-ethyl]-carbamic acid tert-butyl ester, Intermediate 8 (330 mg, yield: 76) as pale-yellow solid. MS (ESI) m/z 489.5 [M+H]⁺. ¹H NMR (400 MHz, DMSO-d₆): δ=7.99 (br, NH, 2H), 6.86 (br, NH, 2H), 3.11-3.07 (m, 4H), 3.03-2.99 (m, 4H), 2.66-2.62 (m, 6H), 2.45-2.40 (m, 2H), 2.24-2.20 (m, 4H), 1.42 (s, 18H).

### Intermediate 9

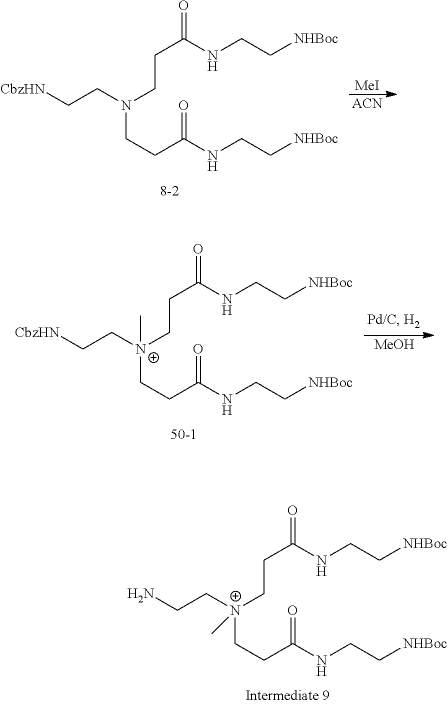

Step 1. A pressure tube charged with a suspension of benzyl (12-(3-((2-((tert-butoxycarbonyl)amino)ethyl)amino)-3-oxopropyl)-2,2-dimethyl-4,9-dioxo-3-oxa-5,8,12-triazatetradecan-14-yl)carbamate, 8-2, (500 mg, 0.80 mmol) and Mel (2 mL, 32 mmol) in ACN (5 mL) was sealed and heated at 60° C. for 20 hrs. The reaction mixture was concentrated and the residue was purified by prep-HPLC to give N-(2-(((benzyloxy) carbonyl)amino)ethyl)-3-((2-((tert-butoxycarbonyl)amino)ethyl)amino)-N-(3-((2-((tert-butoxycarbonyl)amino) ethyl)amino)-3-oxopropyl)-N-methyl-3-oxopropan-1-aminium 2,2,2-trifluoroacetate, 9-1, (450 mg, 97% yield) as a white solid. ¹H NMR (400 MHz, DMSO-d6): 8.15 (t, 1H), 7.60-7.55 (t, 1H), 7.39-7.31 (m, 5H), 6.83-6.80 (t, 2H), 5.06 (s, 2H), 3.58-3.54 (m, 4H), 3.44-3.42 (m, 2H), 3.35-3.33 (m, 2H), 3.10-3.07 (m, 4H), 3.01 (s, 3H), 3.01-2.98 (m, 4H), 2.63-2.60 (m, 4H), 1.37 (s, 18H). MS (ESI) m/z 637.4 [M+].

Step 2. A flask charged with 9-1 (450 mg, 0.71 mmol) and Pd/C (wet, ~100 mg) in MeOH (25 mL) was degassed and filled with hydrogen using a balloon. The resulting mixture was then hydrogenated for 2 hrs at room temperature. The mixture was filtered over celite, washed with EA and MeOH. The filtrate was combined and concentrated to give crude N-(2-aminoethyl)-3-((2-((tert-butoxycarbonyl)amino)ethyl)amino)-N-(3-((2-((tert-butoxycarbonyl)amino)ethyl) amino)-3-oxopropyl)-N-methyl-3-oxopropan-1-aminium 2,2,2-trifluoroacetate, Intermediate 9, (300 mg, 84% yield) as a yellow oil. ¹H NMR (400 MHz, DMSO-d6): 8.18 (t, 2H), 6.82 (t, 2H), 3.58-3.54 (m, 4H), 3.36-3.30 (m, 2H), 3.16-3.10 (m, 2H), 3.09-3.06 (m, 4H), 3.03 (s, 3H), 3.01-2.97 (m, 4H), 2.64-2.61 (m, 4H), 1.37 (m, 18H). MS (ESI) m/z 503.4 [M+].

### Intermediate 10

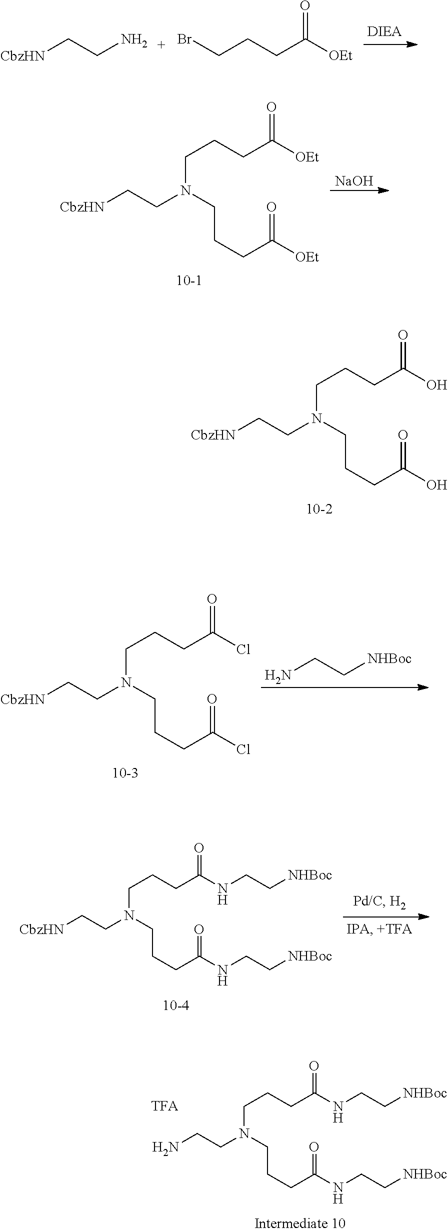

Step 1. A mixture of (2-amino-ethyl)-carbamic acid benzyl ester (10 g, 0.043 mol), 4-bromo-butyric acid ethyl ester (42 g, 0.217 mmol) and DIEA (33 g, 0.258 mol) in MeCN (150 mL) was heated at 70° C. for 16 hrs. Solvent was removed and the residue was purified by silica gel column chromatography (PE/EA=2:1) to give 4-[(2-benzyloxycarbonyl amino-ethyl)-(3-ethoxycarbonyl-propyl)-amino]-butyric acid ethyl ester, 10-1, (14.5 g, yield: 79%) as a yellow oil. MS (ESI) m/z 423.2 [M+H]⁺.

Step 2. To a mixture of 10-1 (14.5 g, 0.034 mol) in MeOH (50 mL) and H₂O (10 mL) was added NaOH (6.9 g, 0.171 mol). The resulting mixture was stirred for 24 hrs at r. t. The mixture was diluted with HCl (1 M) until pH reached 2-3 and concentrated. The residue was treated with MeOH (40 mL) and filtered. The filtrate was concentrated in vacuum to give a residue which was purified by chromatograph to give 4-[(2-benzyloxycarbonylamino-ethyl)-(3-carboxy-propyl)-amino]-butyric acid, 10-2, (9 g, yield: 72%) as a white solid. MS (ESI) m/z 367.2 [M+H]⁺. ¹H NMR (400 MHz, DMSO-d6): 7.38-7.29 (m, 5H), 7.14 (t, J=5.6 Hz, 1H), 5.00 (s, 2H), 3.06-3.01 (m, 2H), 2.45-2.37 (m, 6H), 2.16 (t, J=7.2 Hz, 4H), 1.61-1.54 (m, 4H).

Step 3. To a suspension of 10-2 (1 g, 2.729 mmol) in DCM (10 mL) was added (COCl)₂ (0.9 mL. 10.917 mmol) and DMF (2 drops) at 0° C. After addition the resulting mixture was stirred for 16 hrs at r. t. Solvent was remove to give crude {2-[bis-(3-chlorocarbonyl-propyl)-amino]-ethyl}-carbamic acid benzyl ester, 10-3, (1.2 g, crude) as a yellow oil.

Step 4. To a mixture of (2-amino-ethyl)-carbamic acid tert-butyl ester (1.1 g, 2.727 mmol) and TEA (1.1 g, 10.91 mmol) in DCM at 0° C. was added 10-3 (1.2 g, crude, 10.91 mmol). After addition the resulting mixture was stirred for 12 hrs at r. t. Solvent was removed and the residue was purified by silica gel column chromatography (DCM/MeOH=15:1) and slurred with MeCN to give (2-{bis-[3-(2-tert-butoxycarbonylamino-ethyl carbamoyl)-propyl]-amino}-ethyl)-carbamic acid benzyl ester, 10-4, (500 mg, yield: 28%) as a white solid. MS (ESI) m/z 651.5 [M+H]⁺. ¹H NMR (400 MHz, CDCl3): 7.36-7.26 (m, 5H), 6.71 (br s, 2H), 5.53 (br s, 1H), 5.24 (br s, 2H), 5.10 (s, 2H), 3.32-3.30 (m, 4H), 3.22-3.20 (m, 6H), 2.49-2.46 (m, 2H), 2.37 (t, J=6.4 Hz, 4H), 2.18 (t, J=7.2 Hz, 4H), 1.74-1.67 (m, 4H), 1.42 (s, 18H).

Step 5. A flask charged with 10-4 (200 mg, 0.31 mmol) and Pd/C (wet, ~50 mg) in isopropyl alcohol (10 mL) was degassed and filled with hydrogen using a balloon. The resulting mixture was then hydrogenated for 2 hrs at 30° C. The mixture was filtered over celite, washed with EA and IPA. The filtrate was combined and concentrated to give crude [2-(4-{(2-amino-ethyl)-[3-(2-tert-butoxycarbonylamino-ethylcarbamoyl)-propyl]-amino}-butyrylamino)-ethyl]-carbamic acid tert-butyl ester, Intermediate 10, (150 mg, yield: 94%) as a white gum. MS (ESI) m/z 517.3 [M+H]⁺.

### Intermediate 11

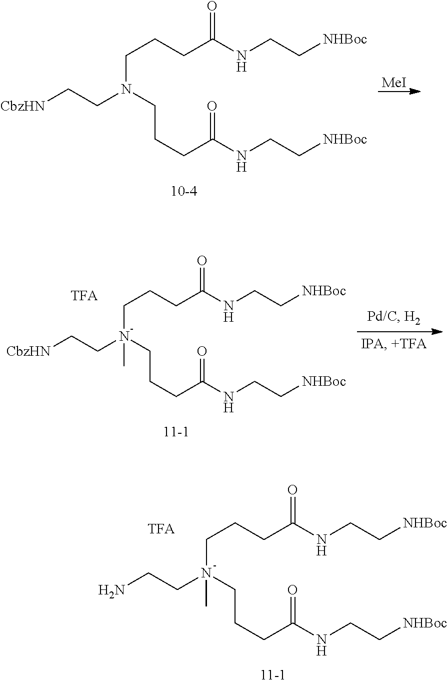

Step 1. A pressure tube charged with compound 10-4 (200 mg, 0.31 mmol) and Mel (1 mL, 16 mmol) in ACN (3 mL) was sealed and heated to 70° C. for 20 hrs. The reaction mixture was concentrated and the residue was purified by prep-HPLC to give N-(2-(((benzyloxy)carbonyl)amino)ethyl)-4-((2-((tert-butoxycarbonyl)amino)ethyl)amino)-N-(4-((2-((tert-butoxycarbonyl)amino)ethyl)amino)-4-oxobutyl)-N-methyl-4-oxobutan-1-aminium, 11-1, (140 mg, yield: 68% yield) as a white gum after freeze drying. MS (ESI) m/z 665.4 [M+]⁺. ¹H NMR (400 MHz, DMSO-d6): 7.96 (t, J=5.6 Hz, 2H), 7.56 (s, 1H), 7.39-7.30 (m, 5H), 6.80 (t, J=5.2 Hz, 2H), 5.05 (s, 2H), 3.45-3.42 (m, 2H), 3.32 (br, 2H), 3.28-3.24 (m, 4H), 3.08-3.04 (m, 4H), 3.20 (s, 3H), 3.00-2.96 (m, 4H), 2.14 (t, J=6.4 Hz, 4H), 1.90-1.86 (m, 4H), 1.37 (s, 18H).

Step 2. A flask charged with 11-1 (140 mg, 0.21 mmol) and Pd/C (wet, ~30 mg) in isopropyl alcohol (10 mL) was degassed and filled with hydrogen using a balloon. The mixture was then hydrogenated for 2 hrs at 30° C. The mixture was filtered over celite, washed with EA and IPA. The filtrate was combined and concentrated to give crude N-(2-aminoethyl)-4-((2-((tert-butoxycarbonyl)amino)ethyl)amino)-N-(4-((2-((tert-butoxycarbonyl) amino)ethyl) amino)-4-oxobutyl)-N-methyl-4-oxobutan-1-aminium, Intermediate 11, (90 mg, yield: 81%) as a white gum. MS (EST) m/z 531.3 [M+]⁺.

### Intermediate 12

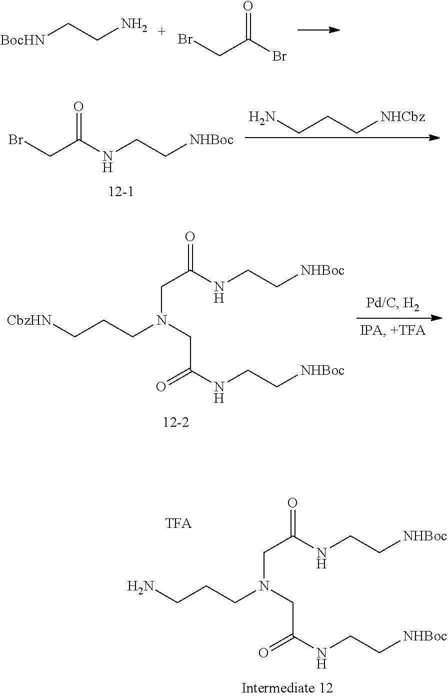 [SMILES: CC(C)(C)OC(=O)NCCN ; O=C(Br)CBr ; CC(C)(C)OC(=O)NCCNC(=O)CBr ; NCCCNC(=O)OCc1ccccc1 ; CC(C)(C)OC(=O)NCCNC(=O)CN(CCCNC(=O)OCc1ccccc1)CC(=O)NCCNC(=O)OC(C)(C)C ; CC(C)(C)OC(=O)NCCNC(=O)CN(CCCN)CC(=O)NCCNC(=O)OC(C)(C)C]

Step 1. To a mixture of tert-butyl (2-aminoethyl)carbamate (20 g, 0.125 mol) and TEA (18.95 g, 0.187 mol) in DCM (200 mL) at 0° C. was added dropwise 2-bromoacetyl bromide (30 g, 0.15 mol) in DCM (30 mL). The mixture was stirred for 12 hrs at r. t., quenched with water (100 mL) and extracted with DCM. The combined organic phase was dried and concentrated. The residue was purified by silica gel column chromatography (PE/EA=5:1 to 2:1) to give tert-butyl (2-(2-bromoacetamido)ethyl)carbamate, 12-1, (9.5 g, yield: 27%) as pale yellow solid. MS (ESI) m/z 181.1 [M+H-100]. ¹H NMR (400 MHz, DMSO-d6): 8.26 (s, 1H), 6.80 (s, 1H), 3.83 (s, 2H), 3.12-3.07 (m, 2H), 3.00-2.96 (m, 2H), 1.37 (s, 9H).

Step 2. A mixture of 12-1 (5 g, 17.79 mmol), (3-amino-propyl)-carbamic acid benzyl ester (1.5 g, 6.15 mmol) and DIEA (4.0 g, 30.7 mmol) in MeCN (100 mL) was heated at 70° C. for 16 hrs. The reaction was cooled to r. t. and the product (3-{bis-[(2-tert-butoxy carbonylamino-ethylcarbamoyl)-methyl]-amino}-propyl)-carbamic acid benzyl ester, 12-2, was collected by filtration as pale-yellow solid (2.3 g, yield: 61%). MS (ESI) m/z 609.3 [M+H]⁺. ¹H NMR (400 MHz, DMSO-d₆): 8.06 (t, J=5.6 Hz, 2H), 7.37-7.28 (m, 5H), 7.23-7.20 (m, 1H), 6.79 (t, J=5.2 Hz, 2H), 4.99 (s, 2H), 3.12-3.09 (m, 4H), 3.03-2.98 (m, 10H), 2.49-2.43 (m, 2H), 1.55-1.51 (m, 2H), 1.36 (s, 18H).

Step 3. A flask charged with 12-2 (500 mg, 0.821 mmol) and Pd/C (wet, ~100 mg) in isopropyl alcohol (30 mL) was degassed and filled with hydrogen using a balloon. The mixture was then hydrogenated for 2 hrs at 25° C. The mixture was filtered over celite, washed with EA and IPA. The filtrate was combined and concentrated to give crude [2-(4-{(2-amino-ethyl)-[3-(2-tert-butoxycarbonylamino-ethylcarbamoyl)-propyl]-amino}-butyrylamino)-ethyl]-carbamic acid tert-butyl ester, Intermediate 12, (390 mg, yield: ~100%) as a white gum. MS (ESI) m/z 475.3 [M+H]⁺. ¹H NMR (400 MHz, DMSO-d₆): 8.13 (t, NH, 2H), 6.84 (t, NH, 2H), 3.17-3.10 (m, 4H), 3.03 (s, 4H), 3.00-2.97 (m, 4H), 2.58-2.55 (m, 2H), 2.50-2.45 (m, 2H), 1.49-1.45 (m, 2H), 1.30 (s, 18H).

### Intermediate 13

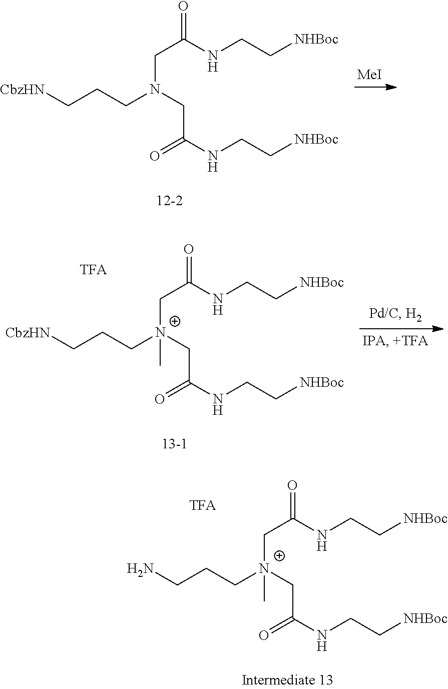 [SMILES: "MeI" ; CC(C)(C)OC(=O)NCCNC(=O)CN(CCCNC(=O)OCc1ccccc1)CC(=O)NCCNC(=O)OC(C)(C)C ; CC(C)(C)OC(=O)NCCNC(=O)C[N+](C)(CCCNC(=O)OCc1ccccc1)CC(=O)NCCNC(=O)OC(C)(C)C ; CC(C)(C)OC(=O)NCCNC(=O)C[N+](C)(CCCN)CC(=O)NCCNC(=O)OC(C)(C)C]

Step 1. A pressure tube charged with 12-2 (600 mg, 0.985 mmol) and Mel (2 mL, 32 mmol) in ACN (7 mL) was sealed and heated to 60° C. for 20 hrs. The reaction mixture was concentrated and the residue was purified by prep-HPLC to give 3-(((benzyloxy) carbonyl)amino)-N,N-bis(2-((2-((tert-butoxycarbonyl)amino)ethyl)amino)-2-oxoethyl)-N-methylpropan-1-aminium, 13-1, (450 mg, yield: 73% yield) as a white gum after freeze drying. MS (ESI) m/z 623.3 [M+]⁺. ¹H NMR (400 MHz, DMSO-d6): 8.65 (t, J=5.6 Hz, 2H), 7.41-7.31 (m, 5H), 6.84 (t, J=5.6 Hz, 2H), 5.02 (s, 2H), 4.23 (s, 4H), 3.68-3.63 (m, 2H), 3.29 (s, 3H), 3.13-2.91 (m, 10H), 1.89-1.85 (m, 2H), 1.37 (s, 18H).

Step 2. A flask charged with 13-1 (250 mg, 0.21 mmol) and Pd/C (wet, ~50 mg) in isopropyl alcohol (20 mL) was degassed and filled with hydrogen using a balloon. The mixture was then hydrogenated for 2 hrs at 30° C. The mixture was filtered over celite, washed with EA and IPA. The filtrate was combined and concentrated to give crude 3-amino-N,N-bis(2-((2-((tert-butoxycarbonyl)amino)ethyl)amino)-2-oxoethyl)-N-methylpropan-1-aminium, Intermediate 13, (190 mg, yield: 97%) as a white gum. MS (ESI) m/z 489.3 [M+]⁺. ¹H NMR (400 MHz, DMSO-d6): 8.76 (t, J=5.6 Hz, 2H), 8.00 (brs, 2H), 6.87-6.82 (m, 2H), 4.30-4.22 (m, 4H), 3.77-3.73 (m, 2H), 3.31 (s, 3H), 3.15-3.12 (m, 4H), 3.05-3.00 (m, 4H), 2.90-2.86 (m, 2H), 2.07-1.99 (m, 2H), 1.37 (s, 18H).

### Intermediate 14

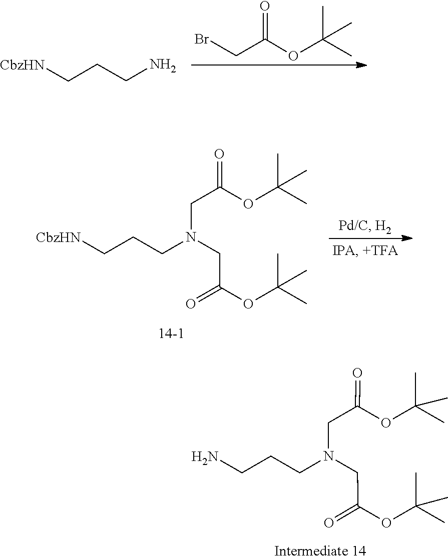

Step 1. To a mixture of (3-amino-propyl)-carbamic acid benzyl ester (HCl salt, 500 mg, 2.409 mmol) and DIEA (1.05 g, 8 mmol) in MeCN (40 mL) at r. t. was added dropwise bromo-acetic acid tert-butyl ester (1.0 g, 5.12 mmol). After addition the resulting mixture was heated at 75° C. for 16 hrs. Solvent was removed and the residue was diluted with EA (20 mL), washed with water and brine and concentrated. The residue was purified by silica gel column chromatography (PE/EA=8:1 to 4:1) to give [(3-benzyloxycarbonyl amino-propyl)-tert-butoxycarbonylmethyl-amino]-acetic acid tert-butyl ester, 14-1, (700 mg, yield: 78.4) as a brown gum. MS (ESI) m/z 437.2 [M+H]⁺. ¹H NMR (400 MHz, DMSO-d₆) 7.35-7.29 (m, 5H), 7.17 (t, J=5.6 Hz, 1H), 4.99 (s, 2H), 3.33 (s, 4H), 3.05-3.01 (m, 2H), 2.59 (t, J=7.2 Hz, 2H), 1.53-1.49 (m, 2H), 1.39 (s, 18H).

Step 2. A flask charged with 14-1 (790 mg, 0.821 mmol) and Pd/C (wet, ~200 mg) in isopropyl alcohol (50 mL) was degassed and filled with hydrogen using a balloon. The mixture was then hydrogenated for 2 hrs at 25° C. The mixture was filtered over celite, washed with EA and IPA. The filtrate was combined and concentrated to give crude [(3-amino-propyl)-tert-butoxycarbonylmethyl-amino]-acetic acid tert-butyl ester, Intermediate 14 (540 mg, yield: 98%) as a pale-yellow gum. MS (ESI) m/z 303.2 [M+H]⁺. ¹H NMR (400 MHz, DMSO-d₆) 3.33 (s, 4H), 2.61 (t, J=6.8 Hz, 2H), 2.54-2.50 (m, 2H), 1.46-1.42 (m, 2H), 1.40 (s, 18H).

### Intermediate 15

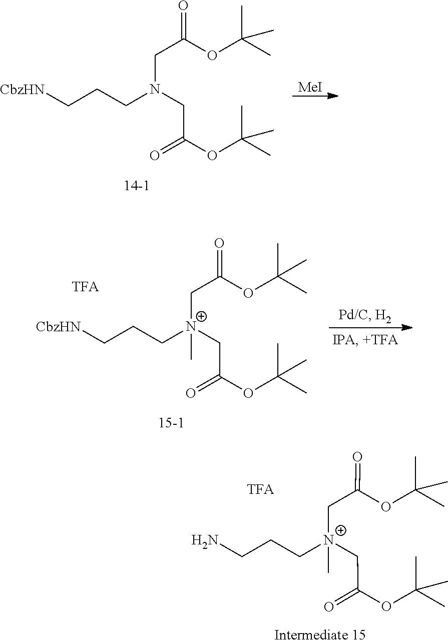 [SMILES: "MeI" ; CC(C)(C)OC(=O)CN(CCCNC(=O)OCc1ccccc1)CC(=O)OC(C)(C)C ; CC(C)(C)OC(=O)C[N+](C)(CCCNC(=O)OCc1ccccc1)CC(=O)OC(C)(C)C ; CC(C)(C)OC(=O)C[N+](C)(CCCN)CC(=O)OC(C)(C)C]

Step 1. A pressure tube charged with 14-1 (260 mg, 0.595 mmol) and Mel (1 mL, 16 mmol) in ACN (2 mL) was sealed and heated at 60° C. for 20 hrs. The reaction mixture was concentrated and the residue was purified by prep-HPLC to give 3-(((benzyloxy) carbonyl)amino)-N,N-bis(2-(tert-butoxy)-2-oxoethyl)-N-methylpropan-1-aminium, 15-1, (130 mg, yield: 48% yield) as a yellow gum after freeze drying. MS (ESI) m/z 451.3 [M+]⁺. ¹H NMR (400 MHz, DMSO-d₆): 7.41-7.31 (m, 6H), 5.01 (s, 2H), 4.46 (s, 4H), 3.62-3.58 (m, 2H), 3.29 (s, 3H), 3.09-3.05 (m, 2H), 1.87-1.83 (m, 2H), 1.46 (s, 18H).

Step 2. A flask charged with 15-1 (180 mg, 0.399 mmol) and Pd/C (wet, ~50 mg) in isopropyl alcohol (20 mL) was degassed and filled with hydrogen using a balloon. The resulting mixture was then hydrogenated for 2 hrs at 30° C. LCMS indicated the completion of reaction. The mixture was filtered over celite, washed with EA and IPA. The filtrate was combined and concentrated to give crude 3-amino-N,N-bis(2-(tert-butoxy)-2-oxoethyl)-N-methylpropan-1-aminium, Intermediate 15, (120 mg, yield: 95%) as a white gum. MS (ESI) m/z 317.2 [M+]⁺. ¹H NMR (400 MHz, DMSO-d6): 7.82 (br, 2H), 4.50 (s, 4H), 3.69-3.64 (m, 2H), 3.29 (s, 3H), 2.85 (t, J=6.8 Hz, 2H), 2.01-1.97 (m, 2H), 1.47 (s, 18H).

### Intermediate 16

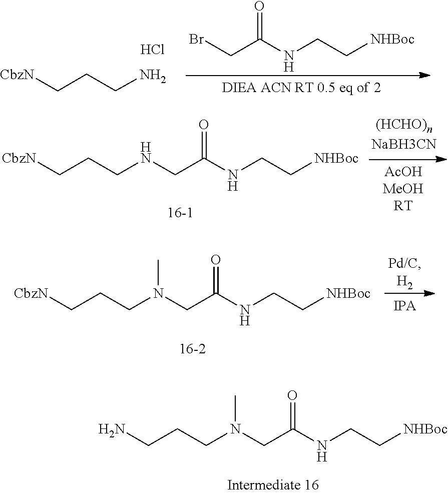

Step 1. To a mixture of benzyl (3-aminopropyl)carbamate) (hydrogen chloride, 1, 2.77 g, 11.3 mmol, 2.0 eq) in 20 mL ACN was added DIEA (2.93 g, 22.64 mmol, 4.0 eq) and tert-butyl (2-(2-bromoacetamido)ethyl)carbamate (1.59 g, 5.66 mmol, 1.0 eq) at RT. The reaction mixture was stirred at 40° C. for 15 hrs. The reaction mixture was concentrated and the residue was diluted with water, extracted with EA. The combined organic layer was dried and concentrated. The residue was purified by silica column chromatography (DCM/MeOH=20/1) to give benzyl (2,2-dimethyl-4,9-dioxo-3-oxa-5,8,11-triazatetradecan-14-yl)carbamate, 16-1, (3, 1.28 g, yield=38.3%) as yellow solid. MS (ESI) m/z 409.3[M+1]+. ¹H NMR (400 MHz, DMSO-d6): δ=7.80 (t, J=5.6 Hz, 1H), 7.38-7.29 (m, 5H), 7.22 (t, J=5.6 Hz, 1H), 6.81 (t, J=4.8 Hz, 1H), 5.00 (s, 2H), 3.14-3.10 (m, 2H), 3.07-3.02 (m, 4H), 3.00-2.96 (m, 2H), 2.45 (t, J=6.8 Hz, 2H), 1.57-1.50 (m, 2H), 1.37 (s, 9H).

Step 2. To a solution of 16-1 (3, 1.28 g, 3.13 mmol, 1.0 eq) was added 1.2 g of HOAc and HCHO (40% aq, 0.188 g, 6.27 mmol, 2.0 eq). The reaction mixture was stirred at RT for 30 mins. NaBH₃CN (0.394 g, 6.27 mmol, 2.0 eq) was added to the mixture in batches. The reaction mixture was stirred at RT overnight. The reaction was quenched with NaHCO₃aq and extracted with DCM. The combined organic layer was dried and concentrated. The residue was purified by silica column chromatography (DCM/MeOH=20/1) to give benzyl (2,2,11-trimethyl-4,9-dioxo-3-oxa-5,8,11-triazatetradecan-14-yl)carbamate, 16-2, (4, 348 mg, yield=26.3%) as white solid. MS (ESI) m/z 423.3[M+1]⁺. ¹H NMR (400 MHz, DMSO-d6): δ=7.71 (t, J=4.4 Hz, 1H), 7.36-7.30 (m, 5H), 7.23 (t, J=5.2 Hz, 1H), 6.80 (t, J=4.4 Hz, 1H), 5.01 (s, 2H), 3.14-3.10 (m, 2H), 3.06-2.97 (m, 2H), 2.85 (s, 2H), 2.34 (t, J=7.2 Hz, 2H), 2.14 (s, 3H), 1.57-1.52 (m, 2H), 1.36 (s, 9H).

Step 3. To a mixture of 16-2 (4, 112 mg, 0.265 mmol, 1.0 eq) in IPA (2 mL) was added Pd/C (wet, ~30 mg.) The reaction mixture was degassed and filled with hydrogen using a balloon. The resulting mixture was then hydrogenated at r. t. for 2 hrs. The mixture was filtered over celite and the filtrate was concentrated to give tert-butyl (2-(2-((3-aminopropyl)(methyl)amino) acetamido)ethyl) carbamate, Intermediate 16, (5, Tail 32, 50 mg, yield: 65.7%) as colorless oil. MS (ESI) m/z 282.3[M+1]⁺. ¹H NMR (400 MHz, DMSO-d6): δ=7.85 (m, 1H), 6.83 (t, 1H), 3.15-3.10 (m, 2H), 3.02-2.97 (m, 2H), 2.86 (s, 2H), 2.56 (t, J=6.8 Hz, 2H), 2.36 (t, J=7.2 Hz, 2H), 2.16 (s, 3H), 1.52-1.45 (m, 2H), 1.37 (s, 9H).

### Intermediate 17

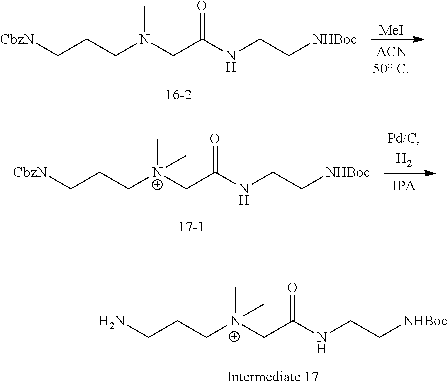

Step 1. A pressure tube charged with 16-2 (236 mg, 0.559 mmol, 1.0 eq) and Mel (1.5 mL) in ACN (5 mL) was sealed. The reaction mixture was then heated at 50° C. for 18 hrs. The reaction was monitored by LCMS. Solvent was removed and the residue was purified by prep-HPLC (5%-95% TFA, C8) to give 3-(((benzyloxy)carbonyl)amino)-N-(2-((2-((tert-butoxycarbonyl) amino)ethyl)amino)-2-oxoethyl)-N,N-dimethylpropan-1-aminium, 17-1, (TFA salt, 327 mg, crude) as colorless oil. MS (ESI) m/z 437.3[M⁺]. ¹H NMR (400 MHz, DMSO-d₆): δ=8.59 (t, J=4.8 Hz, 1H), 7.40-7.32 (m, 5H), 6.86 (s, 1H), 5.02 (s, 2H), 5.02 (brs, 1H), 3.98 (s, 2H), 3.48-3.44 (m, 2H), 3.16 (s, 6H), 3.16-3.14 (m, 2H), 3.08-3.02 (m, 4H), 1.87-1.83 (m, 2H), 1.38 (s, 9H).

Step 2. To a mixture of 17-1 (327 mg, 0.559 mmol, 1.0 eq) in IPA (5 mL) was added Pd/C (50 mg). The reaction mixture was degassed and filled with hydrogen using a balloon. The resulting mixture was then hydrogenated at r. t. for 3 hrs. LCMS indicated the completion of reaction. The mixture was filtered over celite and concentrated to give 3-amino-N-(2-((2-((tert-butoxycarbonyl)amino)ethyl)amino)-2-oxoethyl)-N,N-dimethylpropan-1-aminium, Intermediate 17, (Tail 33, TFA salt, 180 mg, yield: 77% over 2 steps) as colorless oil. MS (ESI) m/z 303.2 [M⁺]. ¹H NMR (400 MHz, DMSO-d₆): δ=8.67 (t, NH, 1H), 7.48 (brs, 3H), 6.88 (t, NH, 1H), 4.34-4.33 (d, 1H), 4.01 (s, 2H), 3.56-3.51 (m, 2H), 3.18 (s, 6H), 3.18-3.13 (m, 2H), 3.06-3.01 (m, 2H), 2.86-2.82 (t, 2H), 1.98-1.94 (m, 2H), 1.38 (s, 9H).

### Intermediate 18

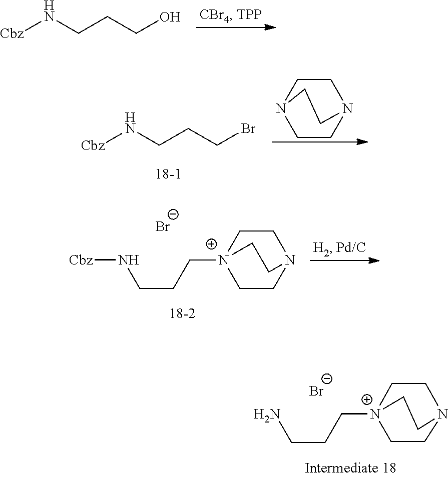

Step 1. To a solution of benzyl (3-hydroxypropyl) carbamate (10 g, 48 mmol) in DCM (120 mL) was added CBr₄ (31.8 g, 96 mmol) and triphenylphosphine (25.2 g, 96 mmol) under N2 atmosphere. The resulting mixture was stirred for 2 hours at room temperature. Solvent was removed and the residue was purified by column chromatography (PE ot PE:EA=10:1) to give the benzyl (3-bromopropyl) carbamate, 18-1 (12.2 g, yield: 93.7%) as a yellow oil. ¹H NMR (400 MHz, DMSO-d6): 7.38-7.28 (m, 5H), 5.01 (s, 2H), 3.51 (t, J=6.8 Hz, 2H), 3.14-3.09 (m, 2H), 1.98-1.91 (m, 2H).

Step 2. A mixture of compound 18-1 (0.7 g, 2.6 mmol) and 1,4-diazabicyclo[2.2.2]octane (0.58 g, 5.2 mmol) in ACN (20 mL) was stirred for 2 hours at room temperature. Solvent was removed to give a thick residue which was washed with EA (5 mL×5) to give 1-(3-(((benzyloxy)carbonyl)amino)propyl)-1,4-diazabicyclo[2.2.2]octan-1-ium bromide, 18-2, (1.1 g, mixed with DABCO, yield: 110%) as a yellow gum. MS (ESI) m/z 304.2 [M]+. ¹H NMR (400 MHz, DMSO-d6): 7.37-7.32 (m, 5H), 5.03 (s, 2H), 3.28-3.18 (m, 8H), 3.09-3.06 (m, 2H), 3.03-2.99 (m, 6H), 1.85-1.81 (m, 2H).

Step 3. A flask charged with 18-2 and Pd/C (wet, about 50 mg) in IPA (10 mL) was degassed and filled with hydrogen using a balloon. The resulting mixture was then hydrogenated at r. t for 40 hrs. LC-MS indicated the completion of the reaction. The reaction mixture was then filtered over celite and concentrated to give crude 1-(3-aminopropyl)-1,4-diazabicyclo [2.2.2]octan-1-ium bromide, Intermediate 18, (contaminated with some DABCO, yield: 80.4%) as a yellow gum. MS (ESI) m/z 170.2 [M]+. ¹H NMR (400 MHz, DMSO-d6): 3.28-3.22 (m, 10H), 3.04-3.00 (m, 6H), 1.77-1.70 (m, 2H).

### Intermediate 19

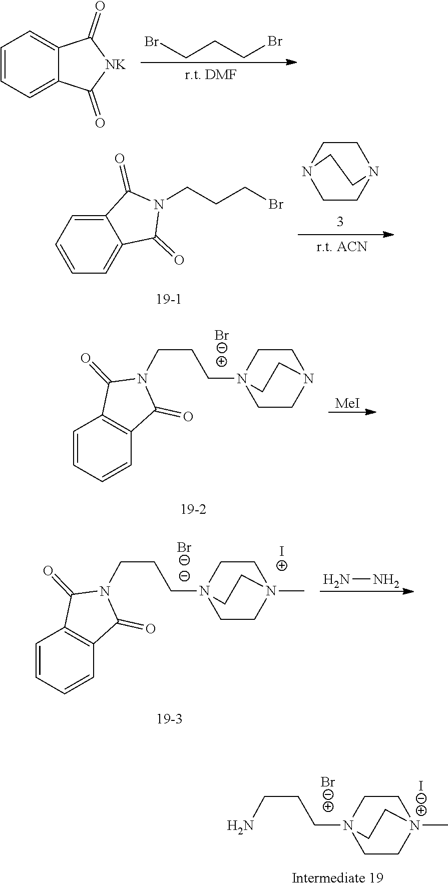

Step 1. To a mixture of 1,3-dibromopropane (2.73 g, 13.5 mmol) in DMF (10 mL) was added potassium 1,3-dioxoisoindolin-2-ide (0.5 g, 2.7 mmol). The resulting mixture was then stirred at room temperature for 16 hours. DMF was removed in vacuum and the residue was purified by column chromatography (PE to PE:EA=10:1) to give crude 2-(3-bromo propyl)isoindoline-1,3-dione 2, 19-1, (610 mg, not pure, mixed with dimer, yield: 85%) as white gum. MS (ESI) m/z 268.0 [M+H]+

Step 2. A mixture of crude 19-1 (610 mg, 2.2 mmol) and 1,4-diazabicyclo[2.2.2]octane (0.5 g, 4.4 mmol) in ACN (20 mL) was stirred for 3 hours at room temperature. Solvent was removed and the residue was triturated and washed with EA (10 mL) 3 times to give 1-(3-(((benzyloxy)carbonyl)amino)propyl)-1,4-diazabicyclo[2.2.2]octan-1-ium bromide, 19-2, (510 mg, yield 60% over 2 steps) as a white solid. MS (ESI) m/z 300.2 [M+]+. 1H NMR (400 MHz, DMSO-d6): 7.92-7.83 (m, 4H), 3.67 (t, J=6.0 Hz, 2H), 3.31-3.22 (m, 8H), 3.01-2.97 (m, 6H), 2.08-1.99 (m, 2H).

Step 3. A pressure tube charged with 19-2 (100 mg, 0.3 mmol) and Mel (0.1 mL, excess) in ACN (0.8 mL)/H2O (0.2 mL) was sealed and heated at 60° C. for 20 hrs. Solvent was removed and the residue was treated with MeCN twice to give the 1-(3-(1,3-dioxo isoindolin-2-yl)propyl)-4-methyl-1,414-diazabicyclo[2.2.2]octan-1-ium salt, 19-3, (70 mg, Yield: 44.8%). as a brown solid. MS (ESI) m/z 314.2 [M−]+. 1H NMR (400 MHz, DMSO-d6): 7.92-7.86 (m, 4H), 3.83-3.78 (m, 12H), 3.69 (t, J=6.4 Hz, 2H), 3.59-3.55 (m, 2H), 3.25 (s, 3H), 2.09-2.06 (m, 2H).

Step 4. A mixture of 19-3 (1.0 g, 1.92 mmol) and hydrazinium hydroxide (85%, 500 mg, 7.66 mmol) in EtOH (6 mL) and water (1 mL) was heated at reflux (85° C.) for 20 hrs. The mixture was cooled and filtered. The filtrate was concentrated and the residue was dissolved in water (4 mL), washed with EA 3 timed. The water phase was lyophilized to give 1-(3-aminopropyl)-4-methyl-1,4-diazabicyclo[2.2.2]octane-1,4-diium salt, Intermediate 19, (crude, 600 mg, mixed with hydrazine hydrate and some phthalhydrazide) as yellow solid. ¹H NMR (400 MHz, DMSO-d6): 3.99-3.89 (br, 12H), 3.62-3.58 (m, 2H), 3.31 (s, 3H), 2.65 (t, J=6.4 Hz, 2H), 1.83-1.75 (m, 2H).

### Intermediate 20

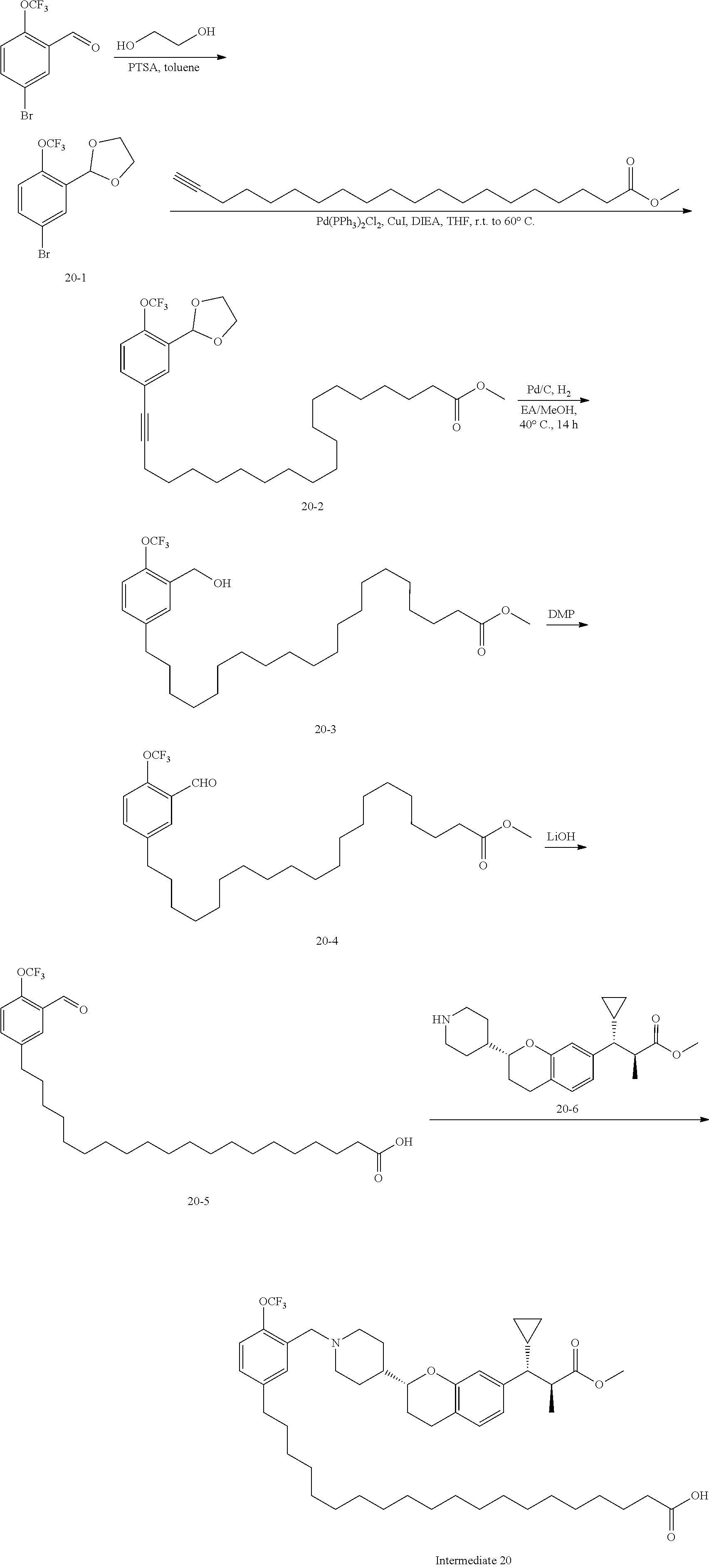

Step 1. A mixture of 5-bromo-2-(trifluoromethoxy)benzaldehyde (3.0 g, 11.15 mmol), ethane-1,2-diol (1.3 g, 20.96 mmol) and PTSA·H₂O (420 mg, 2.19 mmol) in toluene (80 mL) was heated at 100° C. for 10 hrs under N2 atmosphere. Solvent was removed and the residue was purified by flash chromatography (PE to 5% EA in PE) to give 2-(5-bromo-2-(trifluoromethoxy) phenyl)-1,3-dioxolane, 20-1, (3.1 g, yield: 88.7%) as colorless oil. ¹H NMR (400 MHz, CDCl₃) δ 7.76 (d, J=2.4 Hz, 1H), 7.52-7.49 (m, 1H), 7.15-7.13 (m, 1H), 6.03 (s, 1H), 4.16-4.13 (m, 2H), 4.07-4.03 (m, 2H).

Step 2. A flask charged with Pd(PPh₃)₂Cl₂ (219 mg, 0.312 mmol) and CuI (59 mg, 0.312 mmol) was degassed and filled with N2. Then a solution of 20-1 (1.0 g, 3.12 mmol) and methyl icos-19-ynoate (1.77 g, 5.75 mmol) in TEA (5 ml) and THF (50 mL) was added via syringe. After addition, the resulting mixture was heated at 60° C. for 6 hrs. The brown reaction mixture was then concentrated. The residue was purified by flash chromatography (PE to 6% of EA in PE) to give methyl 20-(3-(1,3-dioxolan-2-yl)-4-(trifluoromethoxy)phenyl)icos-19-ynoate, 20-2, (1.2 g, mixed with 3, purity ~20%, yield: 13.9%) as yellow solid. ¹H NMR (400 MHz, CDCl3) δ 7.44-7.42 (m, 2H), 7.15-7.16 (m, 1H), 6.04 (s, 1H), 4.16-4.14 (m, 2H), 4.06-4.03 (m, 2H), 3.66 (s, 3H), 2.60 (t, J=7.6 Hz, 2H), 2.30 (t, J=7.6 Hz, 2H), 1.65-1.56 (m, 4H), 1.30-1.25 (m, 26H).

Step 3. A flask charged with 20-2 (1.2 g, purity ~20%, 0.433 mmol) and Pd/C (wet, ~300 mg) in EA (10 mL) and MeOH (10 mL) was degassed and filled with hydrogen using a balloon. The resulting mixture was then hydrogenated at 40° C. for 14 hrs. Filtered over celite and concentrated. The residue was purified by flash chromatography (PE:EA=40:1 to 5:1) to give methyl 20-(3-(hydroxymethyl)-4-(trifluoromethoxy)phenyl)icosanoate, 20-3, (160 mg, yield: 71.7%) as yellow solid. 1H NMR (400 MHz, CDCl3) δ 7.33 (s, 1H), 7.12-7.11 (m, 2H), 4.75 (s, 2H), 3.66 (s, 3H), 2.60 (t, J=7.6 Hz, 2H), 2.29 (t, J=7.6 Hz, 2H), 1.61-1.56 (m, 4H), 1.30-1.25 (m, 30H).

Step 4. To a mixture of methyl 20-3 (160 mg, 0.31 mmol) in DCM (10 mL) at r. t. was added Dess-Martin periodinane (263 mg, 0.62 mmol) in portions. The resulting mixture was stirred for 10 h at r. t. The reaction mixture was filtered and washed with DCM (5 mL×5), the organic phase was combined and concentrated. The residue was purified by prep-TLC (PE:EA=10:1) to give methyl 20-(3-formyl-4-(trifluoromethoxy)phenyl)icosanoate, 20-4, (130 mg, yield: 81.7%) as pale-yellow solid. ¹H NMR (400 MHz, CDCl₃) δ 10.35 (s, 1H), 7.75 (d, J=2.0 Hz, 1H), 7.46-7.43 (m, 1H), 7.26-7.24 (m, 1H), 3.66 (s, 3H), 2.65 (t, J=7.6 Hz, 2H), 2.29 (t, J=7.6 Hz, 2H), 1.63-1.59 (m, 4H), 1.33-1.24 (m, 30H).

Step 5. A mixture of 20-4 (130 mg, 0.253 mmol) and LiOH·H₂O (32 mg, 0.758 mmol) in THF (5 mL), MeOH (1 mL) and water (1 mL) was heated at 40° C. for 12 hrs. The mixture was acidified with 1M HCl until pH reached 3 and concentrated. The residue was purified by prep-TLC (DCM) to give 20-(3-formyl-4-(trifluoromethoxy)phenyl)icosanoic acid, 20-5, (80 mg, yield: 63.5%) as white solid. MS (ESI) m/z 501.3[M+H]⁺.

Step 6. To a mixture of 20-5 (70 mg, 0.14 mmol) and (2S,3R)-3-cyclopropyl-2-methyl-3-((R)-2-(piperidin-4-yl)chroman-7-yl)propanoate (20-6, see Intermediate 1 of WO2023134712) (40 mg, 0.12 mmol) in MeOH (3 mL) and DCM (3 mL) was added ZnCl₂ (38 mg, 0.28 mmol) and NaBH₃CN (26 mg, 0.42 mmol). The resulting mixture was heated at 40° C. for 20 h. Solvent was removed and the residue was purified by prep-TLC (DCM:MeOH=10:1) to give 20-(3-((4-((R)-7-((1R,2S)-1-cyclopropyl-3-methoxy-2-methyl-3-oxopropyl)chroman-2-yl)piperidin-1-yl)methyl)-4-(trifluoromethoxy)phenyl) icosanoic acid, Intermediate 20, (25 mg, yield: 26.9%) as yellow gum. MS (ESI) m/z 422.0 [M/2+H]⁺.

### Intermediate 21

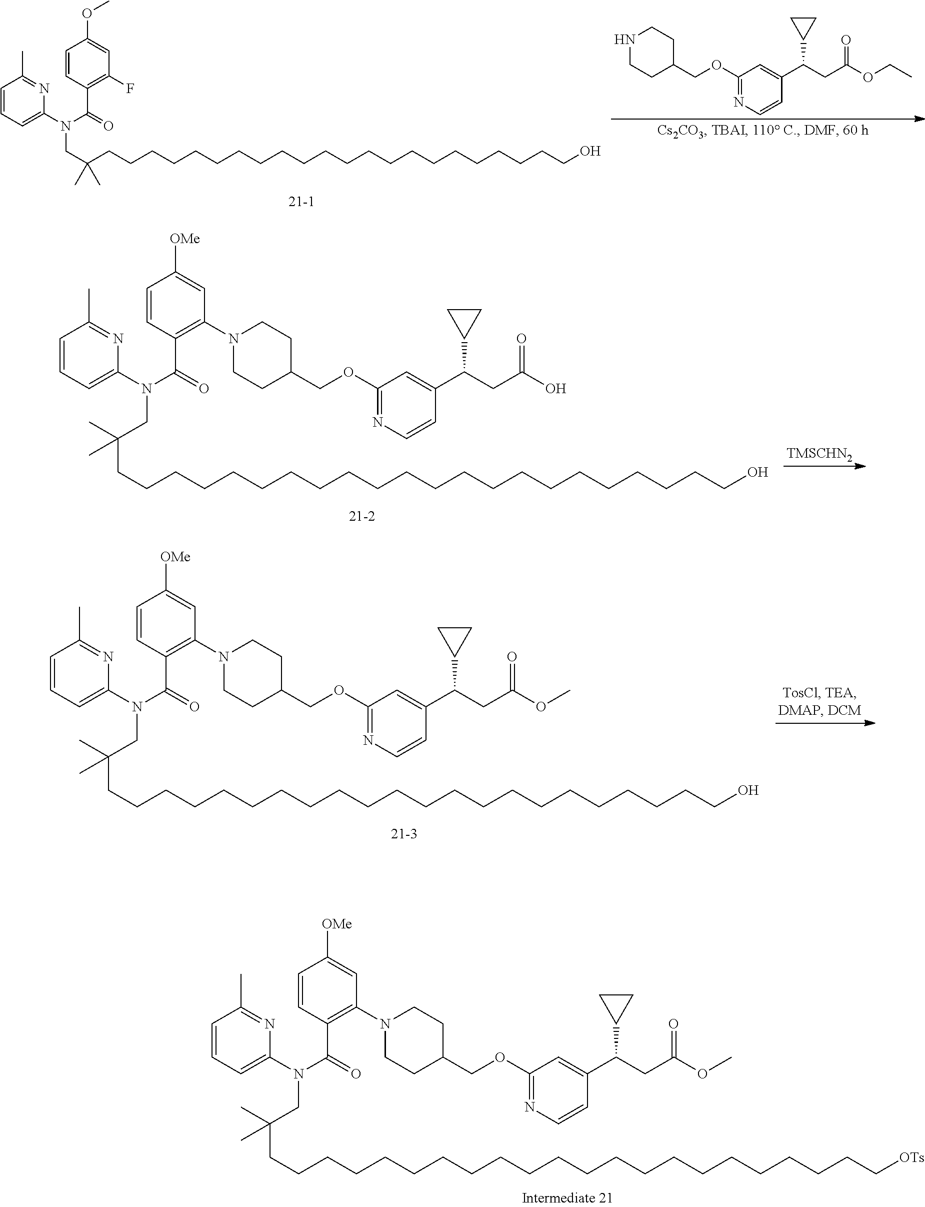 [SMILES: COc1ccc(C(=O)N(CC(C)(C)CCCCCCCCCCCCCCCCCCCCCCO)c2cccc(C)n2)c(F)c1 ; CCOC(=O)C[C@H](c1ccnc(OCC2CCNCC2)c1)C1CC1 ; COc1ccc(C(=O)N(CC(C)(C)CCCCCCCCCCCCCCCCCCCCCCO)c2cccc(C)n2)c(N2CCC(COc3cc([C@@H](CC(=O)O)C4CC4)ccn3)CC2)c1 ; COC(=O)C[C@H](c1ccnc(OCC2CCN(c3cc(OC)ccc3C(=O)N(CC(C)(C)CCCCCCCCCCCCCCCCCCCCCCO)c3cccc(C)n3)CC2)c1)C1CC1 ; COC(=O)C[C@H](c1ccnc(OCC2CCN(c3cc(OC)ccc3C(=O)N(CC(C)(C)CCCCCCCCCCCCCCCCCCCCCCOS(=O)(=O)c3ccc(C)cc3)c3cccc(C)n3)CC2)c1)C1CC1]

Step 1. Intermediate 21-1, prepared in a similar way as for Intermediate 2-10 (500 mg, 0.78 mmol), ethyl (S)-3-cyclopropyl-3-(2-(piperidin-4-ylmethoxy)pyridin-4-yl)propanoate (390 mg, 1.17 mmol), Cs₂CO₃ (635 mg, 1.95 mmol) and TBAI (29 mg, 0.078 mmol) in DMF (1.2 mL) was sealed and heated at 110° C. for 48 h under N₂. The mixture was cooled and diluted with water (15 mL), acidified with 1M HCl until the pH reached 3 and then extracted with EA (20 ml×3). The combined organic phase was dried and concentrated. The residue was purified by pre-TLC (DCM/MeOH=10:1) to give (S)-3-cyclopropyl-3-(2-((1-(2-((24-hydroxy-2,2-dimethyltetracosyl)(6-methylpyridin-2-yl)carbamoyl)-5-methoxyphenyl)piperidin-4-yl)methoxy)pyridin-4-yl)propanoic acid, 21-2, as a brown gum. MS (ESI) m/z 463.5 [M/2+H]+. ¹H NMR (400 MHz, DMSO-d6): 11.95 (br, 1H), 8.04-8.02 (m, 1H), 7.28-7.24 (m, 1H), 6.91-6.82 (m, 2H), 6.70 (s, 1H), 6.51-6.49 (m, 1H), 6.33 (br, 1H), 6.23 (s, 1H), 4.21-3.95 (m, 4H), 3.69 (s, 3H), 3.38-3.34 (m, 2H), 2.74-2.32 (m, 7H), 2.36 (s, 3H), 2.27-2.19 (m, 1H), 1.80-1.35 (m, 5H), 1.29-0.85 (m, 42H), 0.80-0.68 (m, 6H), 0.53-0.48 (m, 1H), 0.37-0.24 (m, 2H), 0.18-0.14 (m, 1H).

Step 2. To a mixture of 21-2 (3, 140 mg, 0.151 mmol) in MeOH (2 mL)/DCM (8 mL) at 0° C. was added TMSCHN₂ (2.0 M in hexane, 0.22 mL) dropwise. After addition, the resulting mixture was stirred for 2 h at r. t. Solvent was removed to give a crude methyl (S)-3-cyclopropyl-3-(2-((1-(2-((24-hydroxy-2,2-dimethyltetracosyl)(6-methylpyridin-2-yl)carbamoyl)-5-methoxyphenyl)piperidin-4-yl)methoxy)pyridin-4-yl)propanoate, 21-3, as a yellow solid. MS (ESI) m/z 470.5 [M/2+H]+.

Step 3. To a solution of 21-3 (150 mg, 0.16 mmol) in DCM (3 mL) was added 4-methylbenzenesulfonyl chloride (46 mg, 0.23 mmol), TEA (49 mg, 0.48 mmol) and DMAP (1.9 mg, 0.016 mmol). The reaction mixture was then stirred at 30° C. for 14 hours. The reaction mixture was quenched with water and extracted with DCM (5 mL×3). The combined organic layers were washed with water (5 mL×2) and brine (5 mL), dried over Na₂SO₄. The organic layer was concentrated to give crude produce. The crude produce was purified by prep-TLC (DCM/MeOH=20/1) to give methyl (S)-3-cyclopropyl-3-(2-((1-(2-((2,2-dimethyl-24-(tosyloxy)tetracosyl)(6-methylpyridin-2-yl)carbamoyl)-5-methoxyphenyl)piperidin-4-yl)methoxy)pyridin-4-yl)propanoate, Intermediate 21, as a yellow gum. MS (ESI) m/z 547.5 [M/2+H]+.

### Intermediate 22

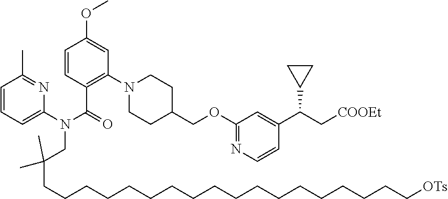 [SMILES: CCOC(=O)C[C@H](c1ccnc(OCC2CCN(c3cc(OC)ccc3C(=O)N(CC(C)(C)CCCCCCCCCCCCCCCCCCOS(=O)(=O)c3ccc(C)cc3)c3cccc(C)n3)CC2)c1)C1CC1]

Intermediate 22 was prepared in a similar way as for Intermediate 21. MS (ESI) m/z 526.4 [(M+2H]/2].

### Intermediate 23

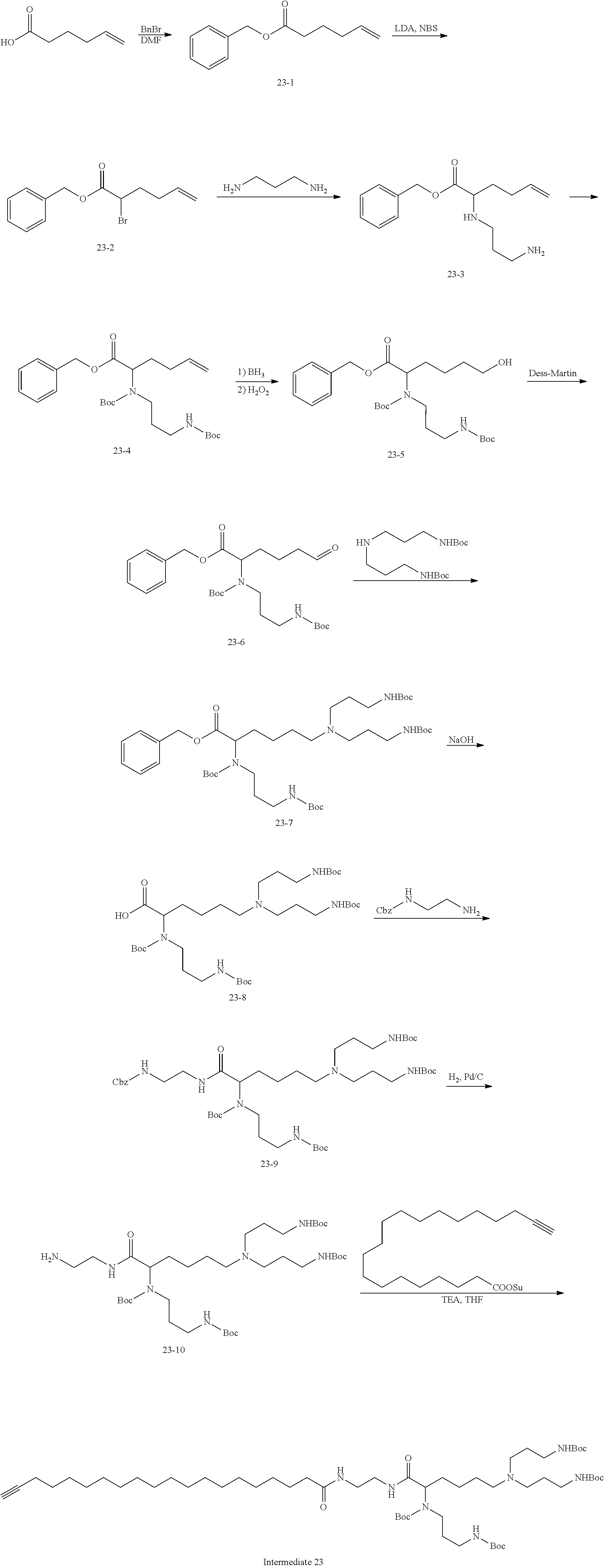

Step 1. To a solution of hex-5-enoic acid (2.03 g, 17.8 mmol) in DMF (30 mL) at 0° C. was added K₂CO₃ (7.37 g, 53.4 mmol) and BnBr (3.66 g, 21.4 mmol). The reaction mixture was stirred at 40° C. overnight under N2. The reaction mixture was quenched with water and extracted with EA (20 mL×4). The combined organic layers were dried over Na₂SO₄ and concentrated. The crude product was chromatographed on silica gel (Petroleum ether/EtOAc 1:0→20:1→10:1) to give benzyl hex-5-enoate, 23-1, as a colorless liquid. MS (ESI) m/z 205.2 [M+H]+. ¹H NMR (300 MHz, CDCl3) δ 7.40-7.26 (m, 5H), 5.84-5.70 (m, 1H), 5.12 (s, 2H), 5.04-4.96 (m, 2H), 2.37 (t, J=7.5 Hz, 2H), 2.13-2.05 (m, 2H), 1.75 (qt, J=7.4 Hz, 2H).

Step 2. To a solution of 23-1 (3.0 g, 14.7 mmol) and TMSCl (2.39 g, 22.1 mmol) in THE (30 mL) was added dropwise LDA (2 M, 8.8 mL, 17.6 mmol) at −78° C. under N2 atmosphere. The reaction mixture was stirred at −78° C. for 4 h. Then to the mixture was added dropwise NBS (3.13 g, 17.6 mmol) in THF (40 mL). The reaction mixture was warmed up to room temperature and stirred overnight. The reaction mixture was quenched by the addition of saturated aqueous NaHCO3 at 0° C. The resulting solution was extracted with EA (20 mL×3), dried over Na2SO4 and filtered. The filtrate was concentrated, and the crude product was chromatographed on silica gel (Petroleum ether/EtOAc 20:1→10:1→4:1) to give benzyl 2-bromohex-5-enoate, 23-2, as an orange liquid. ¹H NMR (300 MHz, CDCl3) δ 7.37-7.26 (m, 5H), 5.78-5.66 (m, 1H), 5.20 (s, 2H), 5.12-5.00 (m, 2H), 4.27 (t, J=6.6 Hz, 1H), 2.24-2.08 (m, 4H).

Step 3. To a solution of 23-2 (4.06 g, 14.3 mmol) in DCM (40 mL) at room temperature was added propane-1,3-diamine (3.17 g, 42.9 mmol). The reaction mixture was stirred at 30° C. overnight under N2. The reaction mixture was diluted with solvent and extracted with DCM (20 mL×3). The combined organic layers were dried over Na₂SO₄ and concentrated. The crude product was chromatographed on silica gel (Petroleum ether/EtOAc 10:1→MeOH/DCM 10:1→4:1) to give benzyl 2-((3-aminopropyl)amino)hex-5-enoate, 23-3, as an orange liquid. MS (ESI) m/z 277.2 [M+H]+. ¹H NMR (300 MHz, CDCl3) δ 7.40-7.26 (m, 5H), 5.82-5.68 (m, 1H), 5.17 (d, J=3.9 Hz, 2H), 5.02-4.95 (m, 2H), 3.29 (t, J=6.8 Hz, 1H), 2.86 (t, J=6.2 Hz, 2H), 2.71-2.63 (m, 1H), 2.61-2.55 (m, 1H), 2.09 (q, J=7.3 Hz, 2H), 1.82-1.64 (m, 4H).

Step 4. To a solution of 23-3 (2.42 g, 8.8 mmol), TEA (2.67 g, 26.4 mmol) in DCM (20 mL) at 0° C. was added (Boc)₂O (11.51 g, 52.8 mmol). The reaction mixture was stirred at 30° C. overnight under N2. The reaction mixture was concentrated under reduced pressure to remove DCM and TEA. The crude product was chromatographed on silica gel (Petroleum ether/EtOAc 0→5→10→30%) to give benzyl 2-((tert-butoxycarbonyl)(3-((tert-butoxycarbonyl)amino) propyl)amino)hex-5-enoate, 23-4, as an orange liquid. MS (ESI) m/z 499.3 [M+Na]+. ¹H NMR (300 MHz, CDCl3) δ 7.34-7.26 (m, 5H), 5.82-5.71 (m, 1H), 5.29-5.09 (m, 2H), 5.06-5.00 (m, 2H), 3.87-3.10 (m, 2H), 3.10-2.96 (m, 3H), 2.13-2.07 (m, 3H), 1.67-1.61 (m, 3H), 1.43 (s, 18H).

Step 5. To a mixture of 23-4 (1.15 g, 2.6 mmol) in THF (15 mL) cooled to 0° C. was added dropwise BH₃ (2.0 M in THF, 5.3 mL, 10.6 mmol) under N2. The reaction mixture was stirred at 30° C. for 6 hours. The reaction mixture was added NaOAc (3.3 mL) and H₂O₂ (3.3 mL) at 0° C. The reaction mixture was stirred at 30° C. overnight under N2. The reaction mixture was diluted with EA (30 mL) and washed with Na2S2O3 (15 mL×2) and brine (15 mL×2). The combined organic layers were dried over Na₂SO₄ and concentrated. The crude product was chromatographed on silica gel (Petroleum ether/EtOAc 10→20→100%) to give benzyl 2-((tert-butoxycarbonyl)(3-((tert-butoxycarbonyl)amino)propyl)amino)-6-hydroxy hexanoate, 23-5, as a colorless gum. MS (ESI) m/z 495.3 [M+H]+.

Step 6. To a mixture of 23-5 (292 mg, 0.6 mmol) in dry DCM (10 mL) was added DMP (382 mg, 0.9 mmol). The reaction mixture was stirred at 30° C. overnight under N2. The reaction mixture was washed with NaHCO₃ (10 mL×2), Na₂S₂O₃ (10 mL×2) and brine (10 mL×2). The combined organic layers were dried over Na₂SO₄ and concentrated. The crude product was chromatographed on silica gel (Petroleum ether/EtOAc 10→20→50→100) to give benzyl 2-((tert-butoxycarbonyl)(3-((tert-butoxycarbonyl)amino)propyl)amino)-6-oxohexanoate, 23-6, as a pale-yellow gum. MS (ESI) m/z 515.3 [M+Na]+. ¹H NMR (400 MHz, CDCl3) δ 9.76 (s, 1H), 7.33-7.26 (m, 5H), 5.15-5.09 (m, 2H), 4.11-4.09 (m, 1H), 2.63-2.47 (m, 8H), 2.29-2.20 (m, 4H), 1.43 (s, 18H).

Step 7. To a solution of 23-6 (443 mg, 0.9 mmol), di-tert-butyl (azanediylbis (propane-3,1-diyl))dicarbamate (398 mg, 1.2 mmol) in MeOH (10 mL) was added NaBH₃CN (76 mg, 1.2 mmol) and AcOH (1 drop). The reaction mixture was stirred at 30° C. for 12 h under N2. The reaction mixture was concentrated under reduced pressure to remove MeOH. The crude product was chromatographed on silica gel (Petroleum ether/EtOAc 10→20→100%) to give benzyl N2-(tert-butoxycarbonyl)-N2,N6,N6-tris(3-((tert-butoxycarbonyl) amino)propyl) lysinate, 23-7, as a pale-yellow solid. MS (ESI) m/z 808.6 [M+H]+. ¹H NMR (300 MHz, CDCl3) δ 7.34-7.26 (m, 5H), 5.30 (s, 2H), 4.11-4.09 (m, 1H), 3.80-4.72 (m, 4H), 3.20-2.98 (m, 10H), 1.80-1.32 (m, 12H), 1.43 (s, 36H).

Step 8. To a solution of 23-7 (471 mg, 0.6 mmol) in MeOH (5 mL), THF (5 mL), H₂O (5 mL) at room temperature was added NaOH (72 mg, 1.8 mmol). The reaction mixture was stirred at r. t. for 10 h. The reaction mixture was concentrated under reduced pressure to remove MeOH and THF. The residue was acidified with 1 M HCl until pH reached 3 to 4. Then the mixture was extracted with DCM (15 mL×4). The combined organic layers were dried over Na₂SO₄ and concentrated. The residue (in MeOH) was purified by preparative HPLC to give N2-(tert-butoxycarbonyl)-N2,N6,N6-tris(3-((tert-butoxy carbonyl)amino) propyl)lysine, 23-8, as a pale-yellow gum. MS (ESI) m/z 716.5 [M−H]−.

Step 9. To a solution of 23-8 (227 mg, 0.316 mmol), DIEA (194 mg, 1.48 mmol), HATU (148 mg, 0.39 mmol) in DMF (7.5 mL) at room temperature was added benzyl (2-aminoethyl)carbamate (126 mg, 0.65 mmol). The reaction mixture was stirred at r. t. for 12 h. The reaction mixture was diluted with water (10 mL) and extracted with EA (10 mL×4). The combined organic layers were dried over Na₂SO₄ and concentrated. The residue (in MeOH) was purified by preparative HPLC to give tert-butyl(3-((tert-butoxycarbonyl) amino)propyl) (14-(3-((tert-butoxycarbonyl)amino)propyl)-21,21-dimethyl-3,8,19-trioxo-1-phenyl-2,20-dioxa-4,7,14,18-tetraazadocosan-9-yl)carbamate, 23-9, as a pale-yellow gum. MS (ESI) m/z 894.6 [M+H]+.

Step 10. A mixture of 23-9 (70 mg, 0.08 mmol) and Pd/C (wet, ~500 mg) in MeOH (10 mL) was hydrogenated using a balloon at 30° C. for 14 h. The mixture was filtered through a Celite pad and concentrated to give crude tert-butyl (18-amino-9-(3-((tert-butoxy carbonyl)amino) propyl)-2,2-dimethyl-4,15-dioxo-3-oxa-5,9,16-triazaoctadecan-14-yl)(3-((tert-butoxy carbonyl)amino)propyl)carbamatee, 23-10, as pale-yellow gum. MS (ESI) m/z 760.6 [M+H]+.

Step 11. A mixture of crude 23-10 (65 mg, 0.1 mmol), TEA (15 mg, 0.15 mmol) and 2,5-dioxopyrrolidin-1-yl icos-19-ynoate (45 mg, 0.11 mmol) in THF (5 mL) was heated at 40° C. for 16 h. Solvent was removed and the residue (in MeOH) was purified by preparative HPLC to give tert-butyl (3-((tert-butoxycarbonyl)amino)propyl)(9-(3-((tert-butoxycarbonyl)amino)propyl)-2,2-dimethyl-4,15,20-trioxo-3-oxa-5,9,16,19-tetraaza nonatriacont-38-yn-14-yl)carbamate Intermediate 23, as a yellow gum. MS (ESI) m/z 1051.9 [M+H]+. ¹H NMR (400 MHz, d6-DMSO): δ=7.55 (br, 2H), 6.73 (s, 2H), 3.43-3.02 (m, 12H), 2.92-2.86 (m, 6H), 2.71 (s, 1H), 2.37-2.31 (m, 5H), 2.14-2.12 (m, 2H), 2.02 (t, J=7.0 Hz, 2H), 1.61-1.41 (m, 12H), 1.36 (s, 36H), 1.25-1.08 (m, 24H).

### Intermediate 24

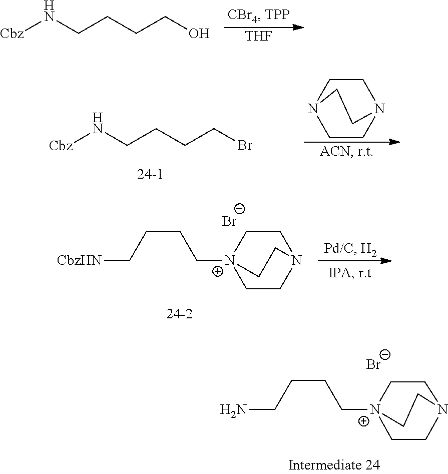

Step 1. To a mixture of CBr₄ (6 g, 18 mmol) and triphenylphosphine (4.72 g, 18 mmol) in 30 mL THF was added benzyl (4-hydroxybutyl) carbamate (2 g, 9.0 mmol) at room temperature. The resulting mixture was stirred at room temperature for 16 hrs. Solvent was concentrated and the residue was purified by silica gel column chromatography (DCM/MeOH 20/1 to 10/1) to afford benzyl (4-bromobutyl) carbamate, 24-1, as a yellow oil. ¹H-NMR (400 MHz, DMSO-d6): 7.38-7.28 (m, 6H), 5.05-5.03 (m, 2H), 3.53 (t, J=7.6, 2H), 3.02 (q, J=6.4, 2H), 1.83-1.75 (m, 2H), 1.55-1.48 (m, 2H).

Step 2. To a stirred solution of 24-1 (400 mg, 1.4 mmol) in ACN (8 mL) was added 1,4-diazabicyclo [2.2.2]octane (172.5 mg, 1.54 mmol) at room temperature. The mixture was stirred at room temperature for 16 hrs. Solvent was concentrated and the residue was washed with EA (10 mL). EA was decanted and the insoluble gum was dried over vacuum to give crude 1-(4-(((benzyloxy)carbonyl) amino) butan-1-ylium-1-yl)-1,4-diaza bicyclo[2.2.2]octan-1-ium bromide, 24-2, as a yellow gum. MS (ESI) m/z 318.2 [M+]+.

Step 3. A flask charged with 24-2 (620 mg, crude, 1.4 mmol) and Pd/C (wet, about 300 mg) in IPA (10 mL) was degassed and filled with hydrogen using a balloon. The resulting mixture was then hydrogenated at r. t for 40 hrs. The reaction mixture was then filtered over celite and concentrated to give crude 1-(4-aminobutan-1-ylium-1-yl)-1,4-diazabicyclo[2.2.2]octan-1-ium bromide, Intermediate 24, as a yellow gum. MS (ESI) m/z 185.2 [M+H]+.

### Intermediate 25

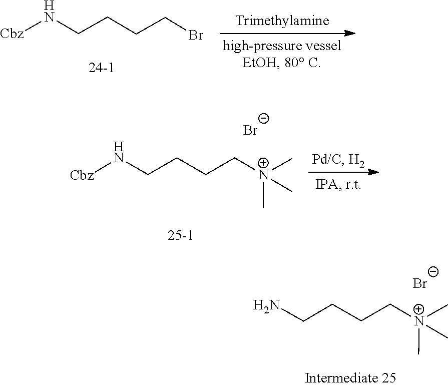

Step 1. A pressure tube charged with a mixture of benzyl (4-bromobutyl) carbamate, 24-1, (400 mg, 1.398 mmol) and trimethylamine (2.0 M in EtOH, 10 mL, 20 mmol) was sealed and heated for 16 hrs at 80° C. The mixture was cooled to r. t. and concentrated. The residue was triturated with EA (8 mL×3). The insoluble sticky substance was collected and dried over vacuum to give the crude 4-(((benzyloxy)carbonyl)amino)-N,N,N-trimethylbutan-1-aminium bromide, 25-1, as a yellow gum. MS (ESI) m/z 265.2 [M+].

Step 2. A flask charged with 25-1 (490 mg, 1.398 mmol) and Pd/C (wet, ~200 mg) in isopropyl alcohol (30 mL) was degassed and filled with hydrogen using a balloon. The resulting mixture was then hydrogenated for 16 hrs at 30° C. The mixture was filtered over celite, washed with EA and IPA. The filtrate was combined and concentrated to give crude 4-amino-N,N,N-trimethylbutan-1-aminium bromide, Intermediate 25, as a pale-yellow oil. MS (ESI) m/z 131.2 [M+]+.

### Intermediate 26

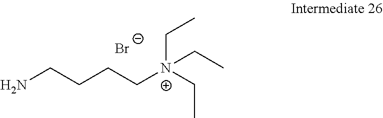

Intermediate 26 was prepared in a similar way as for Intermediate 25. MS: m/z 173.2 [M+].

The syntheses of the following examples, if not specified, were all carried out by coupling of the corresponding acid and amine intermediates with HATU, in a similar way as described for Example 11.

### Example 1

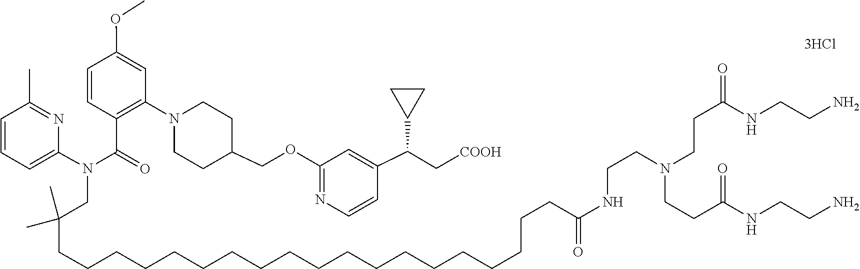 [SMILES: COc1ccc(C(=O)N(CC(C)(C)CCCCCCCCCCCCCCCCCCCC(=O)NCCN(CCC(=O)NCCN)CCC(=O)NCCN)c2cccc(C)n2)c(N2CCC(COc3cc([C@@H](CC(=O)O)C4CC4)ccn3)CC2)c1]

MS (ESI) m/z 1183.0[M+H]+. 1H NMR (400 MHz, DMSO-d6): δ 8.36 (t, NH, 2H), 8.12 (t, NH, 1H), 8.04-8.03 (d, 1H), 7.81 (br, NH3, 6H), 7.29-7.26 (m, 1H), 7.11-7.08 (d, 1H), 6.92-6.91 (d, 1H), 6.86-6.84 (d, 1H), 6.71 (s, 1H), 6.52-6.50 (d, 1H), 6.44 (br, 1H), 6.28 (br, 1H), 4.12 (m, 4H), 3.69 (s, 3H), 3.41-3.37 (m, 6H), 3.33-3.28 (m, 4H), 3.20-3.16 (m, 2H), 2.89-2.85 (m, 4H), 2.62-2.60 (d, 2H), 2.59-2.56 (m, 4H), 2.36 (s, 3H), 2.27-2.21 (m, 1H), 2.10-2.06 (t, 2H), 1.75 (br, 1H), 1.68-1.66 (m, 2H), 1.49-1.46 (m, 4H), 1.30-1.12 (m, 30H), 1.02-1.97 (m, 5H), 0.75 (s, 6H), 0.54-0.49 (m, 1H), 0.35-0.25 (m, 2H), 0.18-0.15 (m, 1H).

### Example 2

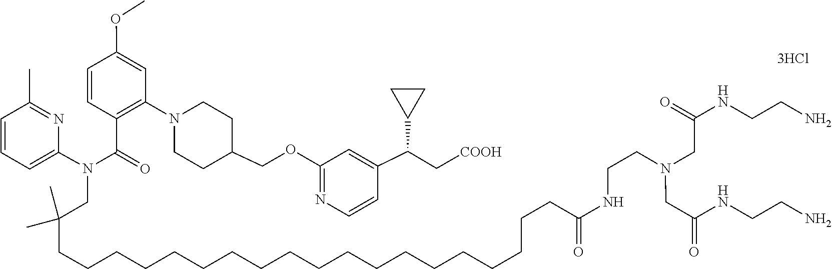 [SMILES: COc1ccc(C(=O)N(CC(C)(C)CCCCCCCCCCCCCCCCCCCC(=O)NCCN(CC(=O)NCCN)CC(=O)NCCN)c2cccc(C)n2)c(N2CCC(COc3cc([C@@H](CC(=O)O)C4CC4)ccn3)CC2)c1 ; "3HCl"]

MS (ESI) m/z 1155.0 [M+H]+. 1H NMR (400 MHz, DMSO-d6): δ 8.39 (t, NH, 2H), 8.04-8.03 (d, 1H), 7.93 (br, NH, 1H), 7.82 (br, NH3, 6H), 7.26-7.24 (br, 1H), 7.10-7.08 (d, 1H), 6.92-6.91 (d, 1H), 6.86-6.84 (d, 1H), 6.70 (s, 1H), 6.52-6.50 (d, 1H), 6.44-6.43 (br, 1H), 6.22 (s, 1H), 4.13-4.11 (m, 4H), 3.68 (s, 3H), 3.53-3.50 (s, 4H), 3.36-3.31 (m, 6H), 3.26-3.20 (m, 2H), 2.90-2.88 (m, 4H), 2.69-2.67 (d, 2H), 2.36 (s, 3H), 2.26-2.22 (m, 1H), 2.08-2.04 (t, 2H), 1.76-1.74 (m, 1H), 1.68-1.65 (m, 2H), 1.46-1.40 (m, 4H), 1.30-1.12 (m, 30H), 1.02-0.97 (m, 5H), 0.75 (s, 6H), 0.52-0.49 (m, 1H), 0.35-0.25 (m, 2H), 0.18-0.15 (m, 1H).

### Example 3

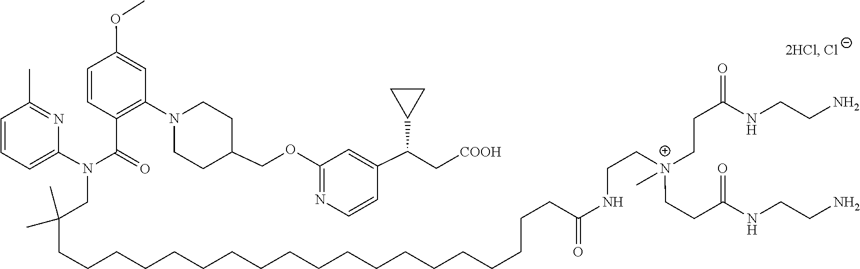 [SMILES: COc1ccc(C(=O)N(CC(C)(C)CCCCCCCCCCCCCCCCCCCC(=O)NCC[N+](C)(CCC(=O)NCCN)CCC(=O)NCCN)c2cccc(C)n2)c(N2CCC(COc3cc([C@@H](CC(=O)O)C4CC4)ccn3)CC2)c1 ; "2HCl, Cl"]

MS (ESI) m/z 1198.1 [M]+. 1H NMR (400 MHz, DMSO-d6): δ 8.38 (t, NH, 2H), 8.15 (t, NH, 1H), 8.04-8.03 (d, 1H), 7.89 (br, NH3, 6H), 7.27 (br, 1H), 7.10-7.08 (d, 1H), 6.92-6.91 (d, 1H), 6.86-6.84 (d, 1H), 6.71 (s, 1H), 6.52-6.51 (d, 1H), 6.50 (br, 1H), 6.22 (s, 1H), 4.14-4.12 (m, 4H), 3.68 (s, 3H), 3.59-3.56 (m, 4H), 3.46-3.44 (m, 2H), 3.33-3.24 (m, 6H), 3.03 (s, 3H), 2.90-2.85 (m, 4H), 2.69-2.67 (m, 6H), 2.36 (s, 3H), 2.27-2.20 (m, 1H), 2.10-2.06 (t, 2H), 1.75 (br, 1H), 1.68-1.65 (m, 2H), 1.49-1.46 (m, 4H), 1.35-1.14 (m, 30H), 1.02-0.97 (m, 5H), 0.75 (s, 6H), 0.53-0.49 (m, 1H), 0.35-0.25 (m, 2H), 0.18-0.15 (m, 1H).

### Example 4

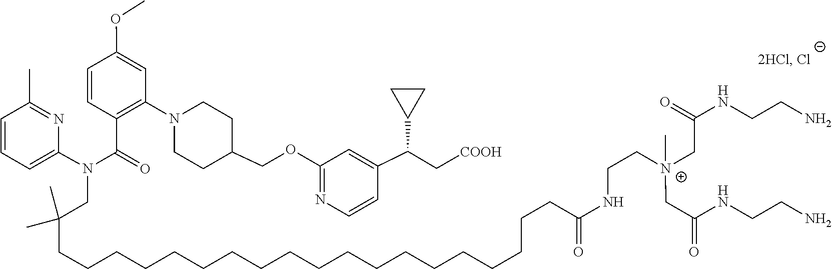 [SMILES: COc1ccc(C(=O)N(CC(C)(C)CCCCCCCCCCCCCCCCCCCC(=O)NCC[N+](C)(CC(=O)NCCN)CC(=O)NCCN)c2cccc(C)n2)c(N2CCC(COc3cc([C@@H](CC(=O)O)C4CC4)ccn3)CC2)c1]

MS (ESI) m/z 1026.8 [(M+H)/2]+. 1H NMR (400 MHz, DMSO-d6): δ 8.92 (t, NH, 2H), 8.21 (t, NH, 1H), 8.04-8.03 (d, 1H), 7.93 (br, NH3, 6H), 7.28-7.25 (br, 1H), 7.11-7.08 (d, 1H), 6.92-6.91 (d, 1H), 6.86-6.84 (d, 1H), 6.71 (s, 1H), 6.52-6.50 (d, 1H), 6.44-6.43 (br, 1H), 6.23 (s, 1H), 4.36 (s, 4H), 4.14-4.05 (m, 4H), 3.75-3.70 (m, 2H), 3.69 (s, 3H), 3.53-3.50 (m, 2H), 3.38 (s, 3H), 3.38-3.35 (m, 4H), 2.94-2.89 (m, 4H), 2.71-2.67 (m, 2H), 2.36 (s, 3H), 2.27-2.21 (m, 1H), 2.10-2.06 (t, 2H), 1.75 (br, 1H), 1.68-1.65 (m, 2H), 1.49-1.46 (m, 4H), 1.35-1.14 (m, 30H), 1.02-0.97 (m, 5H), 0.75 (s, 6H), 0.53-0.49 (m, 1H), 0.35-0.25 (m, 2H), 0.18-0.15 (m, 1H).

### Example 5

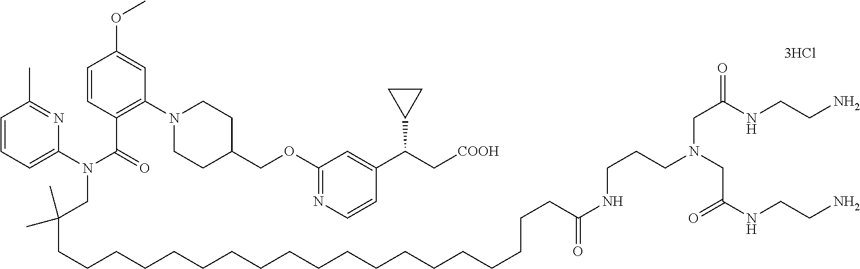 [SMILES: COc1ccc(C(=O)N(CC(C)(C)CCCCCCCCCCCCCCCCCCCC(=O)NCCCN(CC(=O)NCCN)CC(=O)NCCN)c2cccc(C)n2)c(N2CCC(COc3cc([C@@H](CC(=O)O)C4CC4)ccn3)CC2)c1]

MS (ESI) m/z 1167.5 [M+H]⁺. ¹H NMR (400 MHz, DMSO-d6): δ 8.82 (t, NH, 2H), 8.05-8.03 (d, 1H), 7.87 (br, NH3, 6H), 7.29-7.24 (br, 1H), 7.11-7.08 (d, 1H), 6.92-6.91 (d, 1H), 6.86-6.84 (d, 1H), 6.71 (s, 1H), 6.52-6.50 (d, 1H), 6.43 (br, 1H), 6.23 (s, 1H), 4.14-4.12 (d, 2H), 4.12 (br, 2H), 3.81 (br, 4H), 3.69 (s, 3H), 3.38-3.34 (m, 4H), 3.06-2.98 (m, 4H), 2.92-2.88 (m, 4H), 2.69-2.67 (d, 2H), 2.36 (s, 3H), 2.27-2.23 (m, 1H), 2.07-1.97 (m, 2H), 1.77-1.66 (m, 5H), 1.46-1.34 (m, 4H), 1.25-1.12 (m, 30H), 1.04-0.96 (m, 5H), 0.75 (s, 6H), 0.53-0.49 (m, 1H), 0.32-0.25 (m, 2H), 0.23-0.18 (m, 1H).

### Example 6

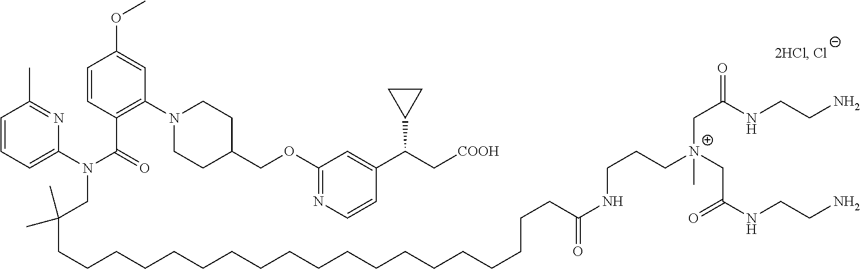 [SMILES: COc1ccc(C(=O)N(CC(C)(C)CCCCCCCCCCCCCCCCCCCC(=O)NCCC[N+](C)(CC(=O)NCCN)CC(=O)NCCN)c2cccc(C)n2)c(N2CCC(COc3cc([C@@H](CC(=O)O)C4CC4)ccn3)CC2)c1]

MS (ESI) m/z 1181.6 [M]⁺. ¹H NMR (400 MHz, DMSO-d₆): δ 8.85 (t, NH, 2H), 7.95 (t, NJ, 1H), 8.05-8.03 (d, 1H), 7.86 (br, NH3, 6H), 7.27-7.23 (br, 1H), 7.10-7.08 (d, 1H), 6.92-6.91 (d, 1H), 6.86-6.84 (d, 1H), 6.71 (s, 1H), 6.52-6.50 (d, 1H), 6.43 (br, 1H), 6.23 (s, 1H), 4.32-4.26 (m, 4H), 4.16-4.12 (m, 4H), 3.69 (s, 3H), 3.65-3.61 (m, 2H), 3.38-3.33 (m, 4H), 3.31 (s, 3H), 3.10-3.05 (m, 2H), 2.92-2.88 (m, 4H), 2.69-2.67 (d, 2H), 2.36 (s, 3H), 2.27-2.23 (m, 1H), 2.07-1.97 (m, 2H), 1.87-1.82 (m, 2H), 1.79-1.66 (m, 3H), 1.46-1.34 (m, 4H), 1.25-1.12 (m, 30H), 1.04-0.96 (m, 5H), 0.75 (s, 6H), 0.54-0.50 (m, 1H), 0.32-0.25 (m, 2H), 0.23-0.18 (m, 1H).

### Example 7

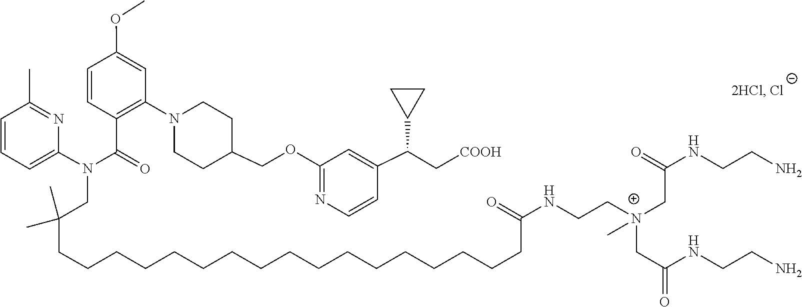 [SMILES: COc1ccc(C(=O)N(CC(C)(C)CCCCCCCCCCCCCCCCCC(=O)NCC[N+](C)(CC(=O)NCCN)CC(=O)NCCN)c2cccc(C)n2)c(N2CCC(COc3cc([C@@H](CC(=O)O)C4CC4)ccn3)CC2)c1]

MS (ESI) m/z 1139.5 [M]⁺. ¹H NMR (400 MHz, DMSO-do): 8.92 (t, NH, 2H), 8.20 (t, NH, 1H), 8.05-8.03 (d, 1H), 7.93 (br, NH3, 6H), 7.28 (br, 1H), 7.11-7.09 (d, 1H), 6.92-6.91 (d, 1H), 6.86-6.84 (d, 1H), 6.71 (s, 1H), 6.52-6.50 (d, 1H), 6.50 (br, 1H), 6.23 (br, 1H), 4.36 (s, 4H), 4.14-4.12 (d, 2H), 4.12 (br, 2H), 3.75-3.72 (m, 2H), 3.69 (s, 3H), 3.53-3.50 (m, 2H), 3.38 (s, 3H), 3.38-3.34 (m, 4H), 2.94-2.89 (m, 4H), 2.69-2.67 (d, 2H), 2.36 (s, 3H), 2.26-2.22 (m, 1H), 2.10-2.06 (t, 2H), 1.81-1.66 (m, 3H), 1.46-1.34 (m, 4H), 1.25-1.12 (m, 26H), 1.04-0.96 (m, 5H), 0.75 (s, 6H), 0.55-0.51 (m, 1H), 0.32-0.25 (m, 2H), 0.23-0.18 (m, 1H).

### Example 8

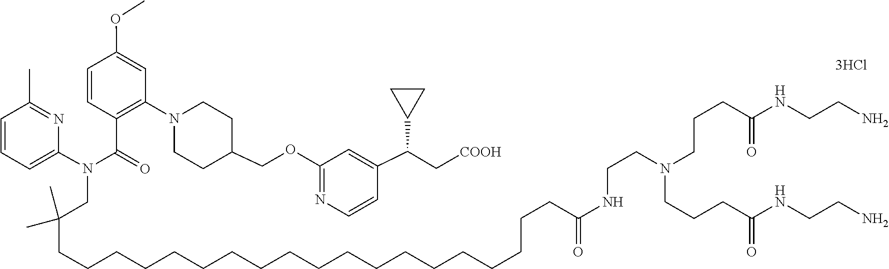 [SMILES: COc1ccc(C(=O)N(CC(C)(C)CCCCCCCCCCCCCCCCCCCC(=O)NCCN(CCCC(=O)NCCN)CCCC(=O)NCCN)c2cccc(C)n2)c(N2CCC(COc3cc([C@@H](CC(=O)O)C4CC4)ccn3)CC2)c1]

MS (ESI) m/z 1209.6 [M+H]⁺. ¹H NMR (400 MHz, DMSO-d6): δ 8.17 (t, NH, 2H), 8.12 (t, NH, 1H), 8.05-8.03 (d, 1H), 7.84 (br, NH3, 6H), 7.28 (br, 1H), 7.11-7.09 (d, 1H), 6.92-6.91 (d, 1H), 6.86-6.84 (d, 1H), 6.72 (s, 1H), 6.52-6.50 (d, 1H), 6.50 (br, 1H), 6.23 (br, 1H), 4.13-4.12 (d, 2H), 4.12 (br, 2H), 3.69 (s, 3H), 3.41-3.38 (m, 2H), 3.31-3.25 (m, 4H), 3.15-3.12 (s, 6H), 2.92-2.88 (m, 4H), 2.69-2.67 (d, 2H), 2.36 (s, 3H), 2.27-2.20 (m, 5H), 2.10-2.06 (t, 2H), 1.86-1.82 (m, 4H), 1.81-1.66 (m, 3H), 1.46-1.34 (m, 4H), 1.25-1.12 (m, 30H), 1.04-0.96 (m, 5H), 0.75 (s, 6H), 0.53-0.48 (m, 1H), 0.32-0.25 (m, 2H), 0.23-0.18 (m, 1H).

### Example 9

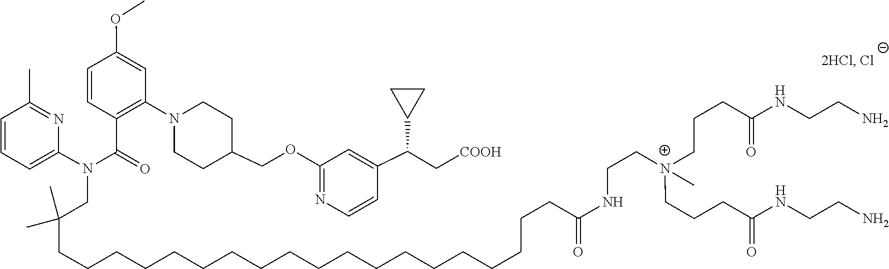 [SMILES: COc1ccc(C(=O)N(CC(C)(C)CCCCCCCCCCCCCCCCCCCC(=O)NCC[N+](C)(CCCC(=O)NCCN)CCCC(=O)NCCN)c2cccc(C)n2)c(N2CCC(COc3cc([C@@H](CC(=O)O)C4CC4)ccn3)CC2)c1]

MS (ESI) m/z 1223.6 [M]⁺. ¹H NMR (400 MHz, DMSO-d₆): δ 8.19 (t, NH, 2H), 8.15 (t, NH, 1H), 8.06-8.04 (d, 1H), 7.90 (br, NH3, 6H), 7.28 (br, 1H), 7.11-7.09 (d, 1H), 6.94-6.93 (d, 1H), 6.86-6.84 (d, 1H), 6.71 (s, 1H), 6.53-6.50 (dd, 1H), 6.4 (br, 1H), 6.24 (br, 1H), 0.15-4.13 (d, 2H), 4.10 (br, 2H), 3.69 (s, 3H), 3.48-3.45 (m, 2H), 3.32-3.27 (m, 10H), 3.02 (s, 3H), 2.89-2.85 (m, 4H), 2.70-2.67 (m, 2H), 2.36 (s, 3H), 2.28-2.18 (m, 5H), 2.10-2.06 (t, 2H), 1.92-1.88 (m, 2H), 1.81-1.66 (m, 3H), 1.50-1.34 (m, 4H), 1.25-1.12 (m, 30H), 1.04-0.96 (m, 5H), 0.75 (s, 6H), 0.53-0.48 (m, 1H), 0.32-0.25 (m, 2H), 0.23-0.18 (m, 1H).

### Example 10

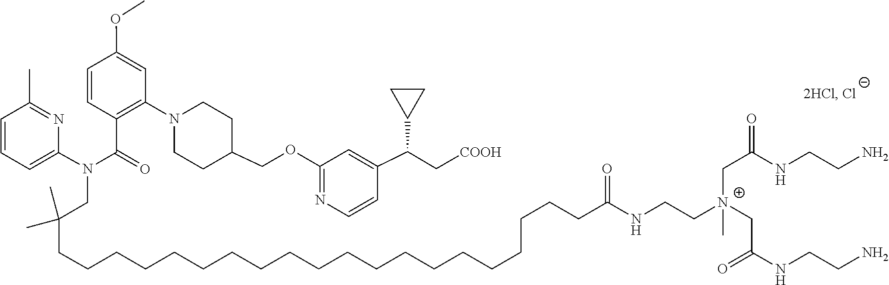 [SMILES: COc1ccc(C(=O)N(CC(C)(C)CCCCCCCCCCCCCCCCCCCCC(=O)NCC[N+](C)(CC(=O)NCCN)CC(=O)NCCN)c2cccc(C)n2)c(N2CCC(COc3cc([C@@H](CC(=O)O)C4CC4)ccn3)CC2)c1]

MS (ESI) m/z 1182.7 [M+H]⁺. ¹H NMR (400 MHz, DMSO-d₆): δ 8.89 (t, NH, 2H), 8.20 (t, NH, 1H), 8.04-8.03 (d, 1H), 7.91 (br, NH3, 6H), 7.26 (br, 1H), 7.10-7.08 (d, 1H), 6.92-6.91 (d, 1H), 6.86-6.84 (d, 1H), 6.71 (s, 1H), 6.52-6.49 (d, 1H), 6.44 (br, 1H), 6.23 (br, 1H), 4.35 (s, 4H), 4.14-4.12 (d, 2H), 4.09 (br, 2H), 3.75-3.71 (m, 2H), 3.68 (s, 3H), 3.53-3.50 (m, 2H), 3.38 (s, 3H), 3.38-3.34 (m, 4H), 2.93-2.86 (m, 4H), 2.69-2.67 (d, 2H), 2.36 (s, 3H), 2.26-2.22 (m, 1H), 2.10-2.06 (t, 2H), 1.81-1.66 (m, 3H), 1.46-1.34 (m, 4H), 1.25-1.12 (m, 32H), 1.04-0.96 (m, 5H), 0.75 (s, 6H), 0.53-0.49 (m, 1H), 0.32-0.25 (m, 2H), 0.23-0.18 (m, 1H).

### Example 11

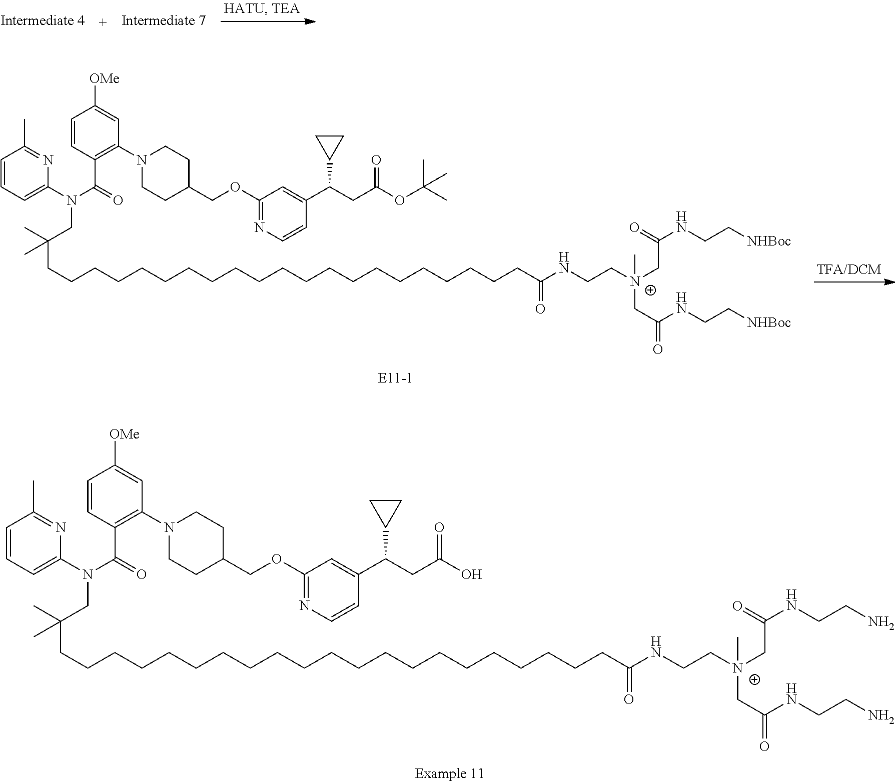 [SMILES: COc1ccc(C(=O)N(CC(C)(C)CCCCCCCCCCCCCCCCCCCCCC(=O)NCC[N+](C)(CC(=O)NCCNC(=O)OC(C)(C)C)CC(=O)NCCNC(=O)OC(C)(C)C)c2cccc(C)n2)c(N2CCC(COc3cc([C@@H](CC(=O)OC(C)(C)C)C4CC4)ccn3)CC2)c1 ; COc1ccc(C(=O)N(CC(C)(C)CCCCCCCCCCCCCCCCCCCCCC(=O)NCC[N+](C)(CC(=O)NCCN)CC(=O)NCCN)c2cccc(C)n2)c(N2CCC(COc3cc([C@@H](CC(=O)O)C4CC4)ccn3)CC2)c1]

Step 1. To a solution of Intermediate 4 (100 mg, 0.10 mmol) in THF (2 mL) was added HATU (76.4 mg, 0.20 mmol), TEA (30.5 mg, 0.30 mmol), and Intermediate 7 (95.5 mg, 0.20 mmol). The mixture was stirred at room temperature for 12 h, was then quenched with water and extracted with EA. The organic phase was washed with water and brine, dried over Na₂SO₄, filtered, and the filtrate was concentrated to give (S)—N-(2-(24-(2-(4-(((4-(3-(tert-butoxy)-1-cyclopropyl-3-oxopropyl)pyridin-2-yl)oxy)methyl)piperidin-1-yl)-4-methoxy-N-(6-methylpyridin-2-yl)benzamido)-23,23-dimethyltetracosanamido)ethyl)-2-((2-((tert-butoxycarbonyl)amino) ethyl)amino)-N-(2-((2-((tert-butoxycarbonyl)amino)ethyl) amino)-2-oxoethyl)-N-methyl-2-oxoethanaminium salt, E11-1, (98 mg, crude) as a yellow gum. MS (ESI) m/z 726.7 [M/2+H]+.

Step 2. To a solution of E11-1 (98 mg, 0.068 mmol) in DCM (2 mL) was added TFA/DCM (1 mL/1 mL) at 0° C. and the mixture was stirred at room temperature for 2 h. The reaction mixture was concentrated and purified by prep-HPLC to give (S)-2-((2-aminoethyl)amino)-N-(2-((2-aminoethyl)amino)-2-oxoethyl)-N-(2-(24-(2-(4-(((4-(2-carboxy-1-cyclopropylethyl) pyridin-2-yl)oxy)methyl)piperidin-1-yl)-4-methoxy-N-(6-methylpyridin-2-yl)benzamido)-23,23-dimethyltetracosanamido)ethyl)-N-methyl-2-oxoethanaminium trifluoroacetate, Example 11, as a white gum. MS (ESI) m/z 1195.6 [M]+. 1H NMR (400 MHz, DMSO-d6): δ 8.85 (t, NH, 2H), 8.17 (t, NH, 1H), 8.04-8.03 (d, 1H), 7.84 (br, NH3, 6H), 7.27 (br, 1H), 7.10-7.08 (d, 1H), 6.92-6.91 (d, 1H), 6.86-6.84 (d, 1H), 6.70 (s, 1H), 6.52-6.50 (d, 1H), 6.43 (br, 1H), 6.23 (br, 1H), 4.34 (s, 4H), 4.14-4.12 (d, 2H), 4.09 (br, 2H), 3.74-3.71 (m, 2H), 3.68 (s, 3H), 3.52-3.48 (m, 2H), 3.38 (s, 3H), 3.36-3.33 (m, 4H), 2.93-2.88 (m, 4H), 2.69-2.67 (d, 2H), 2.36 (s, 3H), 2.26-2.22 (m, 1H), 2.10-2.06 (t, 2H), 1.81-1.66 (m, 3H), 1.46-1.34 (m, 4H), 1.25-1.12 (m, 34H), 1.04-0.96 (m, 5H), 0.75 (s, 6H), 0.53-0.49 (m, 1H), 0.32-0.25 (m, 2H), 0.23-0.18 (m, 1H).

### Example 12

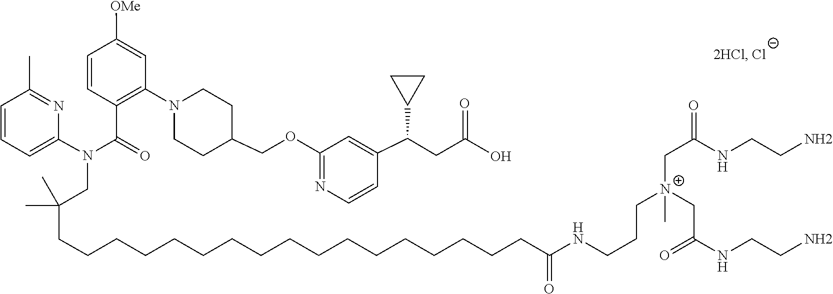 [SMILES: COc1ccc(C(=O)N(CC(C)(C)CCCCCCCCCCCCCCCCCC(=O)NCCC[N+](C)(CC(=O)NCCN)CC(=O)NCCN)c2cccc(C)n2)c(N2CCC(COc3cc([C@@H](CC(=O)O)C4CC4)ccn3)CC2)c1]

MS (ESI) m/z 577.3 [(M+H)/2]⁺. ¹H NMR (400 MHz, DMSO-d₆): δ 8.86 (t, NH, 2H), 7.96 (t, NH, 1H), 8.05-8.03 (d, 1H), 7.86 (br, NH3, 6H), 7.29-7.25 (br, 1H), 7.11-7.08 (d, 1H), 6.92-6.91 (d, 1H), 6.86-6.84 (d, 1H), 6.71 (s, 1H), 6.52-6.50 (d, 1H), 6.43 (br, 1H), 6.23 (s, 1H), 4.31-4.22 (q, 4H), 4.16-4.12 (m, 4H), 3.69 (s, 3H), 3.64-3.60 (m, 2H), 3.38-3.33 (m, 4H), 3.31 (s, 3H), 3.10-3.05 (m, 2H), 2.92-2.88 (m, 4H), 2.69-2.67 (d, 2H), 2.36 (s, 3H), 2.27-2.23 (m, 1H), 2.07-1.97 (m, 2H), 1.88-1.84 (m, 2H), 1.79-1.66 (m, 3H), 1.49-1.34 (m, 4H), 1.25-1.12 (m, 26H), 1.04-0.96 (m, 5H), 0.75 (s, 6H), 0.53-0.48 (m, 1H), 0.32-0.25 (m, 2H), 0.23-0.18 (m, 1H).

### Example 13

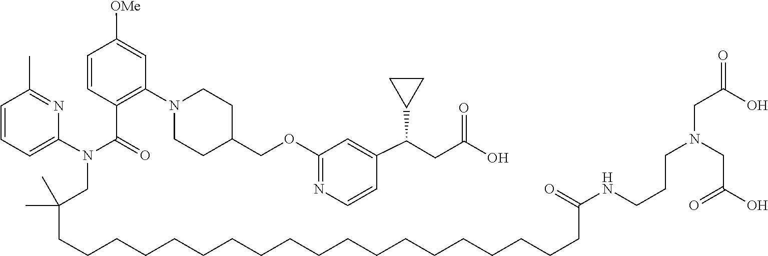 [SMILES: COc1ccc(C(=O)N(CC(C)(C)CCCCCCCCCCCCCCCCCCCC(=O)NCCCN(CC(=O)O)CC(=O)O)c2cccc(C)n2)c(N2CCC(COc3cc([C@@H](CC(=O)O)C4CC4)ccn3)CC2)c1]

MS (ESI) m/z 1084.4 [M+H]⁺. ¹H NMR (400 MHz, DMSO-d₆): δ 8.04-8.03 (d, 1H), 7.81 (t, NH, 1H), 7.27-7.25 (br, 1H), 7.11-7.08 (d, 1H), 6.92-6.91 (d, 1H), 6.86-6.84 (d, 1H), 6.71 (s, 1H), 6.52-6.50 (d, 1H), 6.43 (br, 1H), 6.23 (s, 1H), 4.14-4.12 (d, 2H), 4.12 (br, 2H), 3.95 (br, 4H), 3.69 (s, 3H), 3.08-3.03 (m, 4H), 2.69-2.67 (d, 2H), 2.36 (s, 3H), 2.27-2.23 (m, 1H), 2.07-1.97 (m, 2H), 1.77-1.66 (m, 5H), 1.48-1.34 (m, 4H), 1.25-1.12 (m, 30H), 1.04-0.96 (m, 5H), 0.75 (s, 6H), 0.53-0.48 (m, 1H), 0.32-0.25 (m, 2H), 0.23-0.18 (m, 1H).

### Example 14

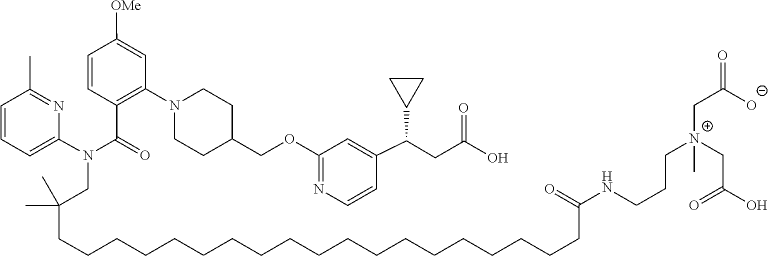 [SMILES: COc1ccc(C(=O)N(CC(C)(C)CCCCCCCCCCCCCCCCCCCC(=O)NCCC[N+](C)(CC(=O)[O-])CC(=O)O)c2cccc(C)n2)c(N2CCC(COc3cc([C@@H](CC(=O)O)C4CC4)ccn3)CC2)c1]

MS (ESI) m/z 1098.4 [M+H]⁺. ¹H NMR (400 MHz, DMSO-d6): δ 8.04-8.03 (d, 1H), 7.87 (t, NH, 1H), 7.27-7.25 (br, 1H), 7.11-7.08 (d, 1H), 6.92-6.90 (d, 1H), 6.86-6.84 (d, 1H), 6.70 (s, 1H), 6.52-6.50 (d, 1H), 6.43 (br, 1H), 6.23 (s, 1H), 4.46 (s, 4H), 4.14-4.12 (d, 2H), 4.12 (br, 2H), 3.69 (s, 3H), 3.69-3.62 (m, 2H), 3.30 (s, 3H), 3.10-3.06 (m, 2H), 2.69-2.67 (d, 2H), 2.38 (s, 3H), 2.27-2.22 (m, 1H), 2.05-2.00 (m, 2H), 1.86-1.78 (m, 3H), 1.68-1.64 (m, 2H), 1.49-1.46 (m, 4H), 1.25-1.12 (m, 30H), 1.04-0.96 (m, 5H), 0.75 (s, 6H), 0.78-0.74 (m, 1H), 0.32-0.25 (m, 2H), 0.23-0.18 (m, 1H).

### Example 15

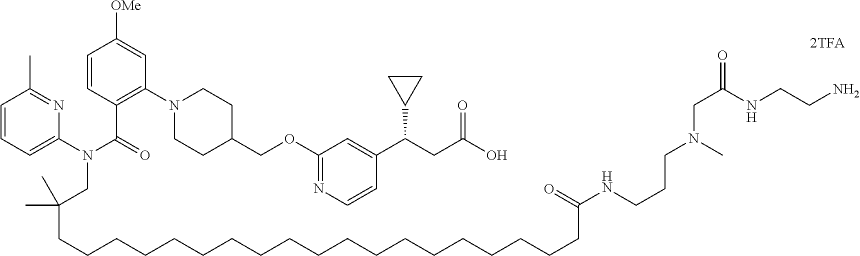 [SMILES: COc1ccc(C(=O)N(CC(C)(C)CCCCCCCCCCCCCCCCCCCC(=O)NCCCN(C)CC(=O)NCCN)c2cccc(C)n2)c(N2CCC(COc3cc([C@@H](CC(=O)O)C4CC4)ccn3)CC2)c1]

MS (ESI) m/z 1081.5 [M+H]⁺. ¹H NMR (400 MHz, DMSO-d6): δ 8.82 (t, NH, 1H), 8.04-8.03 (d, 1H), 8.00 (br, NH3, 3H), 7.97-7.94 (t, NH, 1H), 7.24 (t, 1H), 7.11-7.09 (d, 1H), 6.92-6.90 (d, 1H), 6.84-6.82 (d, 1H), 6.71 (s, 1H), 6.51-6.49 (d, 1H), 6.41 (br, 1H), 6.22 (br, 1H, 4.13-4.11 (d, 2H), 4.10 (br, 2H), 3.93-3.87 (br, 2H), 3.68 (s, 3H), 3.42-3.37 (m, 2H), 3.08-3.06 (m, 2H), 2.95-2.90 (m, 4H), 2.80 (s, 3H), 2.69-2.67 (d, 2H), 2.35 (s, 3H), 2.25-2.23 (m, 1H), 2.06-2.02 (t, 2H), 1.79-1.75 (m, 3H), 1.68-1.65 (m, 2H), 1.48-1.45 (m, 2H), 1.38-1.36 (m, 2H), 1.25-1.10 (m, 30H), 1.04-0.96 (m, 5H), 0.75 (s, 6H), 0.52-0.48 (m, 1H), 0.36-0.25 (m, 2H), 0.23-0.18 (m, 1H).

### Example 16

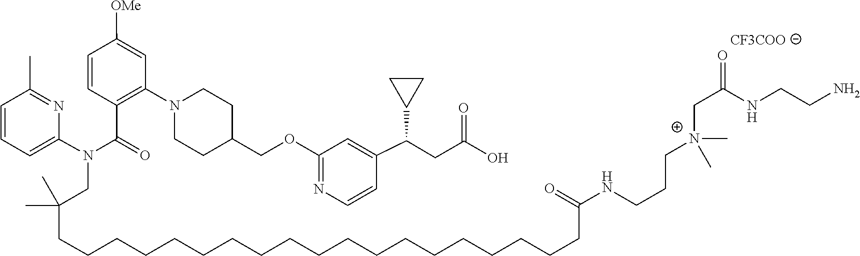 [SMILES: COc1ccc(C(=O)N(CC(C)(C)CCCCCCCCCCCCCCCCCCCC(=O)NCCC[N+](C)(C)CC(=O)NCCN)c2cccc(C)n2)c(N2CCC(COc3cc([C@@H](CC(=O)O)C4CC4)ccn3)CC2)c1 ; "CF3COO"]

MS (ESI) m/z 548.4 [(M+H)/2]⁺. ¹H NMR (400 MHz, DMSO-d6): δ 8.91 (t, NH, 1H), 8.05-8.04 (d, 1H), 7.97 (br, NH3, 3H), 7.96-7.94 (t, NH, 1H), 7.26 (t, 1H), 7.11-7.09 (d, 1H), 6.93-6.92 (d, 1H), 6.86-6.84 (d, 1H), 6.72 (s, 1H), 6.53-6.50 (d, 1H), 6.43 (br, 1H), 6.23 (br, 1H), 4.14-4.12 (d, 2H), 4.10 (br, 2H), 4.05 (br, 2H), 3.69 (s, 3H), 3.48-3.44 (m, 2H), 3.40-3.36 (m, 2H), 3.18 (s, 6H), 3.11-3.07 (m, 2H), 2.95-2.91 (m, 2H), 2.69-2.68 (d, 2H), 2.36 (s, 3H), 2.25-2.23 (m, 1H), 2.07-2.03 (t, 2H), 1.80-1.70 (m, 3H), 1.68-1.64 (m, 2H), 1.52-1.49 (m, 2H), 1.38 (m, 2H), 1.25-1.10 (m, 30H), 1.04-0.90 (m, 5H), 0.74 (s, 6H), 0.53-0.48 (m, 1H), 0.36-0.25 (m, 2H), 0.23-0.18 (m, 1H).

### Example 17

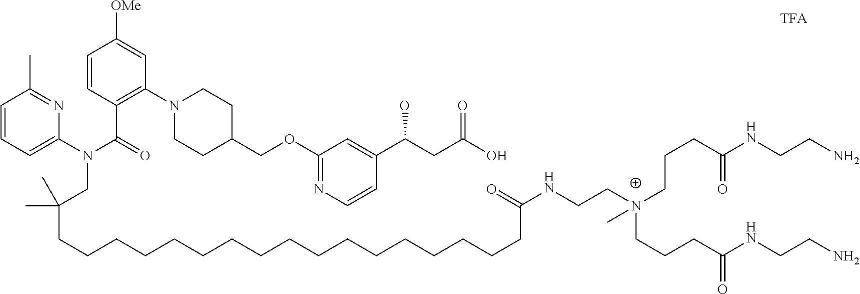

MS (ESI) m/z 1195.4 [M]⁺. ¹H NMR (400 MHz, DMSO-d6): δ 8.18 (t, NH, 2H), 8.15 (t, NH, 1H), 8.05-8.04 (d, 1H), 7.87 (br, NH3, 6H), 7.28 (br, 1H), 7.11-7.09 (d, 1H), 6.93-6.91 (dd, 1H), 6.86-6.84 (d, 1H), 6.72 (s, 1H), 6.53-6.50 (dd, 1H), 6.4 (br, 1H), 6.23 (br, 1H), 4.13-4.12 (d, 2H), 4.10 (br, 2H), 3.69 (s, 3H), 3.48-3.44 (m, 2H), 3.32-3.27 (m, 10H), 3.02 (s, 3H), 2.89-2.84 (m, 4H), 2.69-2.67 (m, 2H), 2.36 (s, 3H), 2.27-2.18 (m, 5H), 2.10-2.06 (t, 2H), 1.91-1.87 (m, 2H), 1.77-1.66 (m, 3H), 1.49-1.37 (m, 4H), 1.25-1.12 (m, 26H), 1.02-0.96 (m, 5H), 0.75 (s, 6H), 0.54-0.49 (m, 1H), 0.32-0.25 (m, 2H), 0.21-0.17 (m, 1H).

### Example 18

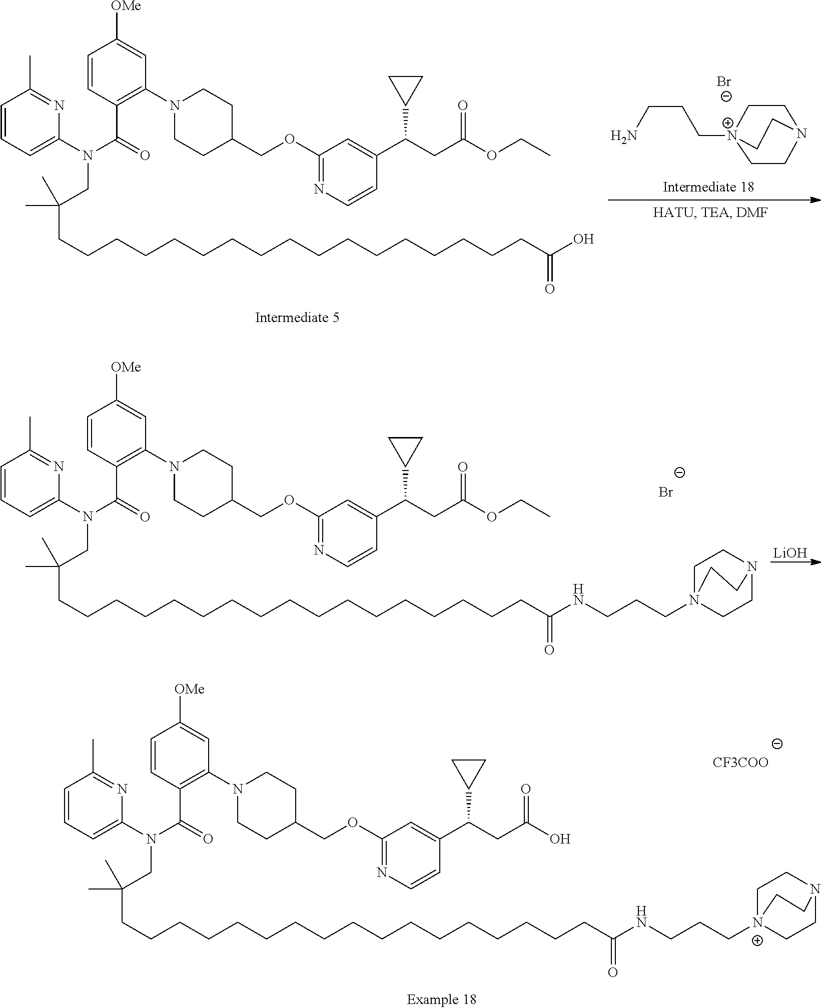

Step 1. To a mixture of Intermediate 5 (60 mg, 0.066 mmol) and Intermediate 18 (29 mg, 0.165 mmol) in THF (2 mL) was added HATU (30 mg, 0.079 mmol) and TEA (20 mg, 0.198 mmol). The resulting mixture was stirred for 12 hrs at r. t. Solvent was removed and the residue was treated with EA (20 mL), washed with water and brine, dried and concentrated to give crude (S)-1-(3-(20-(2-(4-(((4-(1-cyclopropyl-3-ethoxy-3-oxopropyl) pyridin-2-yl)oxy)methyl) piperidin-1-yl)-4-methoxy-N-(6-methylpyridin-2-yl)benzamido)-19,19-dimethylicosanamido) propyl)-1,4-diazabicyclo[2.2.2]octan-1-ium salt (90 mg, crude) as a yellow oil. MS (ESI) m/z 532.0 [(M+H)/2]+.

Step 2. A mixture of (S)-1-(3-(20-(2-(4-(((4-(1-cyclopropyl-3-ethoxy-3-oxopropyl)pyridin-2-yl)oxy)methyl)piperidin-1-yl)-4-methoxy-N-(6-methylpyridin-2-yl)benzamido)-19,19-dimethylicosanamido)propyl)-1,4-diazabicyclo[2.2.2]octan-1-ium salt (90 mg, 0.078 mmol) and LiOH·H₂O (14 mg, 0.333 mmol) in H₂O (1 mL), MeOH (1.5 mL) and THF (1.5 mL) was stirred for 14 hrs at r. t. Solvent was removed and the residue was purified by prep-HPLC to give the (S)-1-(3-(20-(2-(4-(((4-(2-carboxy-1-cyclopropylethyl) pyridin-2-yl)oxy)methyl)piperidin-1-yl)-4-methoxy-N-(6-methylpyridin-2-yl)benzamido)-19,19-dimethylicosanamido)propyl)-1,4-diazabicyclo[2.2.2]octan-1-ium TFA salt, Example 18, as a yellow gum. MS (ESI) m/z 1034.4 [M]⁺. ¹H NMR (400 MHz, DMSO-d₆): δ 8.05-8.03 (d, 1H), 7.88 (t, NH, 1H), 7.27-7.25 (br, 1H), 7.11-7.08 (d, 1H), 6.93-6.91 (d, 1H), 6.86-6.84 (d, 1H), 6.71 (s, 1H), 6.52-6.50 (d, 1H), 6.45 (br, 1H), 6.23 (s, 1H), 4.14-4.10 (m, 4H), 3.69 (s, 3H), 3.35-3.30 (m, 8H), 3.22-3.17 (m, 2H), 3.15-3.06 (m, 6H), 2.67-2.65 (d, 2H), 2.36 (s, 3H), 2.27-2.23 (m, 1H), 2.07-2.03 (m, 2H), 1.83-1.78 (m, 3H), 1.69-1.63 (m, 2H), 1.49-1.46 (m, 4H), 1.27-1.22 (m, 26H), 1.04-0.96 (m, 5H), 0.75 (s, 6H), 0.52-0.48 (m, 1H), 0.35-0.25 (m, 2H), 0.22-0.18 (m, 1H).

### Example 19

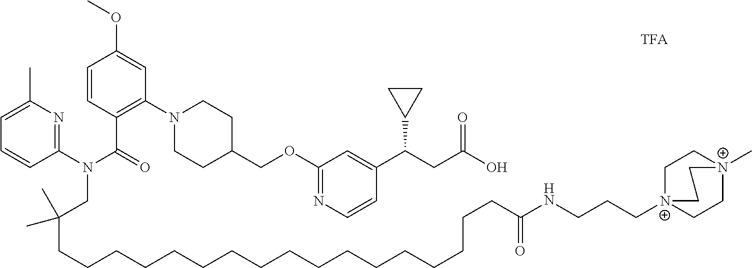 [SMILES: COc1ccc(C(=O)N(CC(C)(C)CCCCCCCCCCCCCCCCCC(=O)NCCC[N+]23CC[N+](C)(CC2)CC3)c2cccc(C)n2)c(N2CCC(COc3cc([C@@H](CC(=O)O)C4CC4)ccn3)CC2)c1]

MS (ESI) m/z 524.9 [M/2]⁺. ¹H NMR (400 MHz, DMSO-d6): δ 8.05-8.03 (d, 1H), 7.95 (t, NH, 1H), 7.29-7.25 (br, 1H), 7.11-7.09 (d, 1H), 6.92-6.91 (dd, 1H), 6.86-6.84 (d, 1H), 6.71 (s, 1H), 6.52-6.50 (dd, 1H), 6.43 (br, 1H), 6.23 (s, 1H), 4.14-4.10 (m, 4H), 3.85-3.77 (m, 12H), 3.69 (s, 3H), 3.48-3.44 (m, 2H), 3.26 (s, 3H), 3.13-3.08 (m, 2H), 2.69-2.67 (d, 2H), 2.36 (s, 3H), 2.26-2.20 (m, 1H), 2.08-2.04 (m, 2H), 1.85-1.77 (m, 3H), 1.68-1.66 (m, 2H), 1.49-1.46 (m, 4H), 1.25-1.12 (m, 26H), 1.02-0.96 (m, 5H), 0.75 (s, 6H), 0.52-0.48 (m, 1H), 0.35-0.25 (m, 2H), 0.22-0.18 (m, 1H).

### Example 20

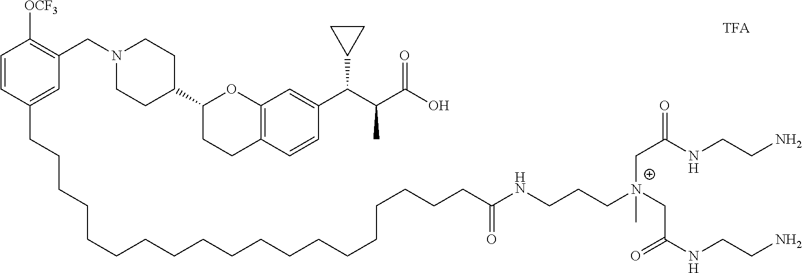 [SMILES: C[C@H](C(=O)O)[C@H](c1ccc2c(c1)O[C@@H](C1CCN(Cc3cc(CCCCCCCCCCCCCCCCCCCC(=O)NCCC[N+](C)(CC(=O)NCCN)CC(=O)NCCN)ccc3OC(F)(F)F)CC1)CC2)C1CC1]

MS (ESI) m/z 1099.5 [M+]⁺. ¹H NMR (400 MHz, DMSO-d6) δ 8.92 (m, 2H), 7.99-7.90 (m, 7H), 7.61 (s, 1H), 7.43-7.40 (m, 2H), 7.00-6.97 (m, 1H), 6.66 (d, 1H), 6.52 (s, 1H), 4.37-4.33 (m, 2H), 4.31 (s, 4H), 3.83-3.80 (m, 1H), 3.67-3.63 (m, 2H), 3.38-3.33 (m, 6H), 3.31 (s, 3H), 3.13-3.09 (m, 4H), 2.91-2.87 (m, 4H), 2.77-2.59 (m, 5H), 2.10-2.00 (t, 2H), 2.00-1.78 (m, 8H), 1.73-1.55 (m, 4H), 1.55-1.39 (m, 2H), 1.35-0.95 (m, 30H), 1.08-1.04 (m, 1H), 0.81 (d, 3H), 0.52-0.49 (m, 1H), 0.27-0.23 (m, 2H), −0.07~−0.12 (m, 1H).

### Example 21

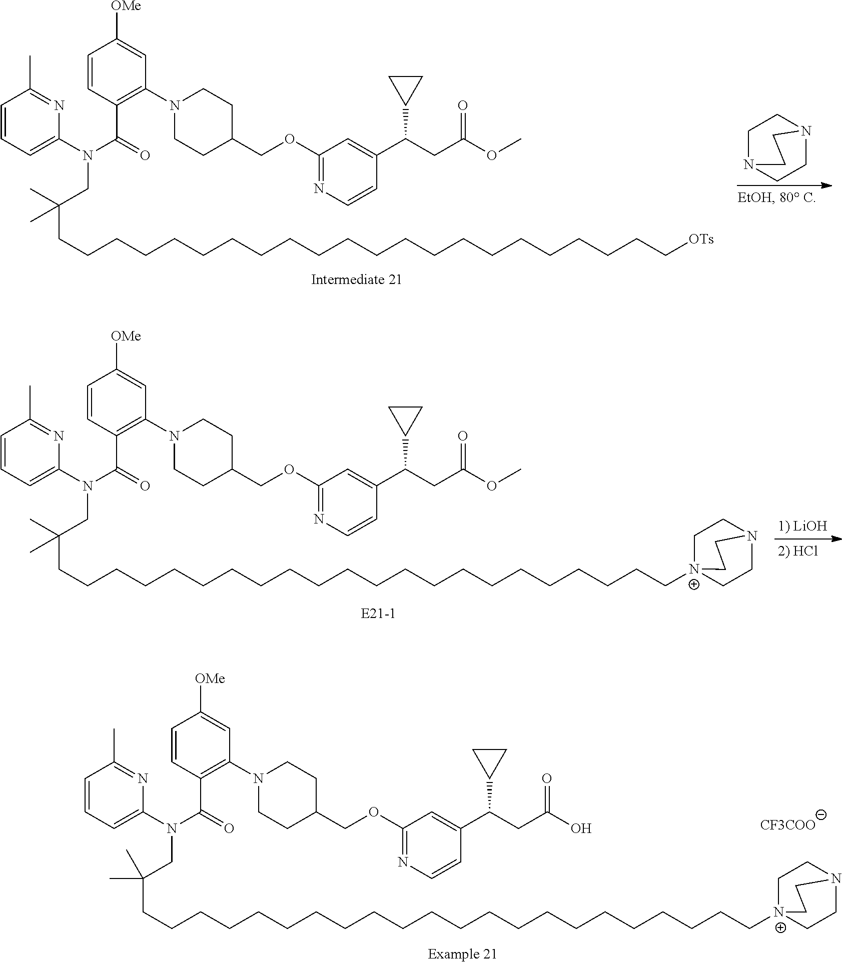 [SMILES: COC(=O)C[C@H](c1ccnc(OCC2CCN(c3cc(OC)ccc3C(=O)N(CC(C)(C)CCCCCCCCCCCCCCCCCCCCCCOS(=O)(=O)c3ccc(C)cc3)c3cccc(C)n3)CC2)c1)C1CC1 ; C1CN2CCN1CC2 ; COC(=O)C[C@H](c1ccnc(OCC2CCN(c3cc(OC)ccc3C(=O)N(CC(C)(C)CCCCCCCCCCCCCCCCCCCCCC[N+]34CCN(CC3)CC4)c3cccc(C)n3)CC2)c1)C1CC1 ; COc1ccc(C(=O)N(CC(C)(C)CCCCCCCCCCCCCCCCCCCCCC[N+]23CCN(CC2)CC3)c2cccc(C)n2)c(N2CCC(COc3cc([C@@H](CC(=O)O)C4CC4)ccn3)CC2)c1 ; "CF3COO"]

Step 1. To a solution of Intermediate 21 (80 mg, 0.077 mmol) in EtOH (2.5 mL) was added 1,4-diazabicyclo[2.2.2]octane (13 mg, 0.116 mmol). The reaction mixture was stirred at 80° C. for 16 hours. The reaction mixture was concentrated and the residue was purified by prep-TLC (DCM/MeOH=10/1) to give (S)-1-(24-(2-(4-(((4-(1-cyclopropyl-3-methoxy-3-oxopropyl)pyridin-2-yl)oxy)methyl)piperidin-1-yl)-4-methoxy-N-(6-methylpyridin-2-yl)benzamido)-23,23-dimethyltetracosyl)-1,4-diazabicyclo[2.2.2]octan-1-ium, E21-1, as a yellow gum. MS (ESI) m/z 517.5 [M/2+H]+.

Step 5. To a solution of E21-1 (70 mg, 0.067 mmol) in MeOH/H₂O (2 mL/1 mL) was added LiOH·H₂O (13.8 mg, 0.338 mmol). The reaction mixture was stirred at room temperature for 16 hours. LCMS indicated the completion of reaction. The reaction mixture was then acidified with 1 M HCl until pH reached 3-4 and concentrated. The residue was purined by prep-HPLC to give (S)-1-(24-(2-(4-(((4-(2-carboxy-1-cyclopropylethyl)pyridin-2-yl)oxy)methyl)piperidin-1-yl)-4-methoxy-N-(6-methylpyridin-2-yl)benzamido)-23,23-dimethyltetracosyl)-1,4-diazabicyclo[2.2.2]octan-1-ium, Example 21, as a yellow solid. ¹H NMR (400 MHz, DMSO-d6): δ=8.05-8.03 (d, 1H), 7.29-7.26 (br, 1H), 7.11-7.09 (d, 1H), 6.92-6.91 (d, 1H), 6.86-6.84 (d, 1H), 6.71 (s, 1H), 6.52-6.50 (d, 1H), 6.44 (br, 1H), 6.23 (s, 1H), 4.14-4.12 (d, 2H), 4.12 (br, 2H), 3.69 (s, 3H), 3.40-3.34 (m, 6H), 3.24-3.14 (m, 8H), 2.69-2.67 (d, 2H), 2.36 (s, 3H), 2.27-2.21 (m, 1H), 1.76 (br, 1H), 1.68-1.60 (m, 4H), 1.46-1.36 (m, 2H), 1.27-1.22 (m, 36H), 1.04-0.97 (m, 5H), 0.75 (s, 6H), 0.52-0.49 (m, 1H), 0.35-0.25 (m, 2H), 0.22-0.18 (m, 1H).

### Example 22

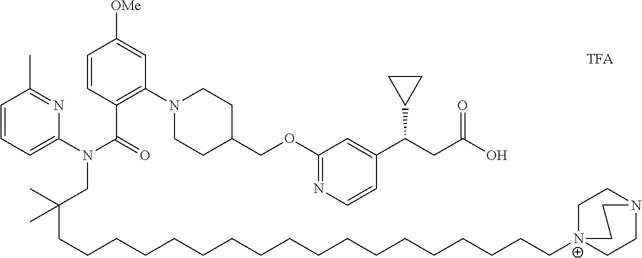 [SMILES: COc1ccc(C(=O)N(CC(C)(C)CCCCCCCCCCCCCCCCCC[N+]23CCN(CC2)CC3)c2cccc(C)n2)c(N2CCC(COc3cc([C@@H](CC(=O)O)C4CC4)ccn3)CC2)c1]

Example 22 was prepared in a similarly way as described for Example 21. MS (ESI) m/z 963.5 [M+]⁺. ¹H NMR (400 MHz, DMSO-d₆) δ 8.05-8.03 (d, 1H), 7.29-7.26 (br, 1H), 7.11-7.09 (d, 1H), 6.92-6.91 (d, 1H), 6.86-6.84 (d, 1H), 6.71 (s, 1H), 6.52-6.50 (d, 1H), 6.44 (br, 1H), 6.23 (s, 1H), 4.13-4.12 (d, 2H), 4.12 (br, 2H), 3.69 (s, 3H), 3.37-3.34 (m, 6H), 3.24-3.17 (m, 8H), 2.69-2.67 (d, 2H), 2.36 (s, 3H), 2.27-2.21 (m, 1H), 1.76 (br, 1H), 1.68-1.62 (m, 4H), 1.46-1.36 (m, 2H), 1.27-1.22 (m, 28H), 1.04-0.97 (m, 5H), 0.75 (s, 6H), 0.52-0.49 (m, 1H), 0.35-0.25 (m, 2H), 0.22-0.18 (m, 1H).

### Example 23

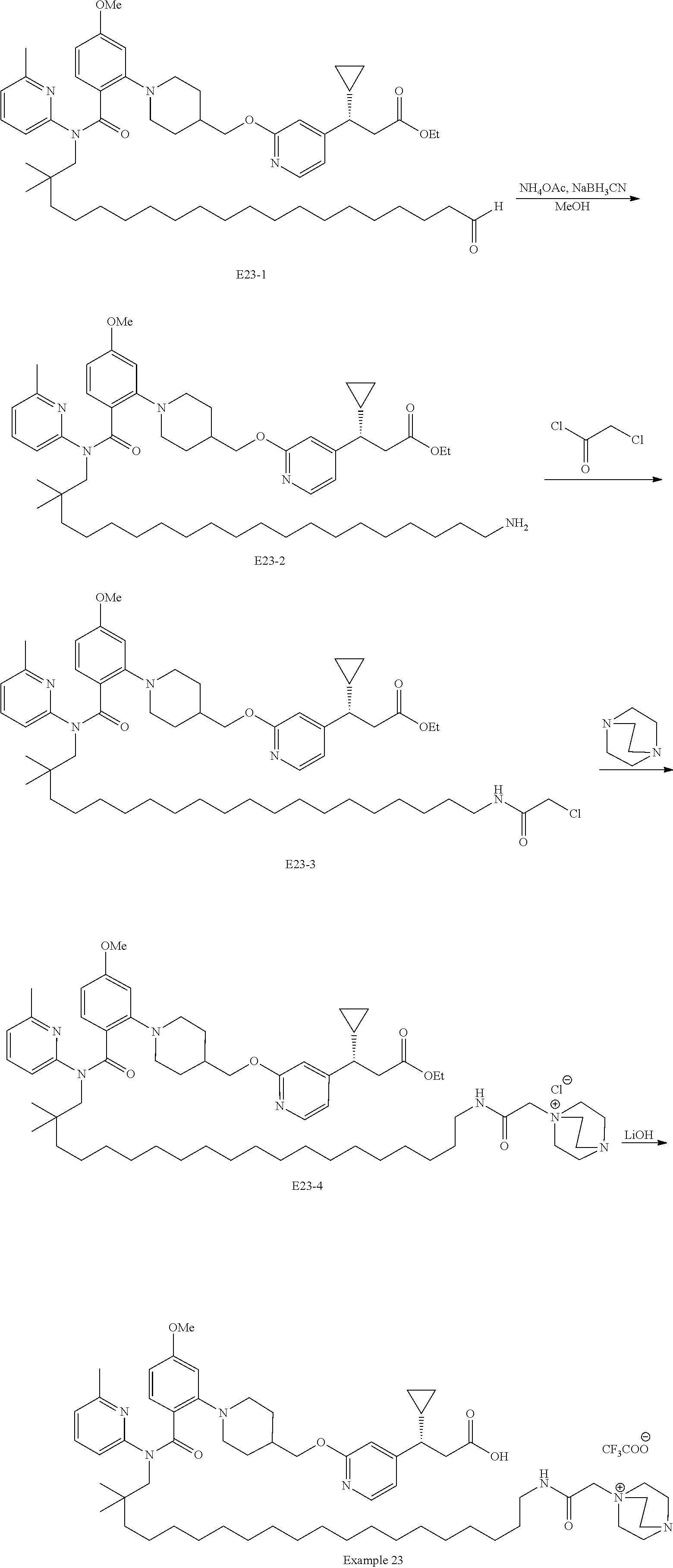

Step 1. A mixture of ethyl (S)-3-cyclopropyl-3-(2-((1-(2-((2,2-dimethyl-20-oxoicosyl)(6-methylpyridin-2-yl)carbamoyl)-5-methoxyphenyl)piperidin-4-yl)methoxy) pyridin-4-yl)propanoate, E23-1, (400 mg, 0.447 mmol) and ammonium acetate (103 mg, 1.342 mmol) in dry MeOH (20 ml) was stirred at 30° C. for 50 min. Then NaBH₃CN (86 mg, 1.342 mmol) was added in portions. After addition the resulting mixture was stirred for additional 16 h at 50° C. The mixture was filtered and the filtrate was concentrated. The residue was purified by pre-TLC (PE/EA=1:1) to give ethyl (S)-3-(2-((1-(2-((20-amino-2,2-dimethylicosyl)(6-methylpyridin-2-yl)carbamoyl)-5-methoxyphenyl)piperidin-4-yl)methoxy) pyridin-4-yl)-3-cyclopropylpropanoate, E23-2, as a yellow gum. MS (ESI) m/z 896.6 [M+H].

Step 2. To a mixture of E23-2 (270 mg, 0.301 mmol) and TEA (45 mg, 0.45 mmol) in dry DCM at 0° C. was added chloroacetyl chloride (41 mg, 0.36 mmol) dropwise. After addition the resulting mixture was stirred for 2 h at r. t. The reaction mixture was quenched with water and extracted with DCM twice. The organic phase was combined, dried and concentrated. The residue was purified by pre-TLC (DCM/MeOH=10:1) to give ethyl (S)-3-(2-((1-(2-((20-(2-chloroacetamido)-2,2-dimethylicosyl)(6-methylpyridin-2-yl)carbamoyl)-5-methoxyphenyl) piperidin-4-yl)methoxy)pyridin-4-yl)-3-cyclopropylpropanoate, E23-3, as a yellow gum. MS (ESI) m/z 972.6 [M+H]+.

Step 3. A mixture of E23-3 (165 mg, 0.172 mmol) and DABCO (98 mg, 0.874 mmol) in EtOH (6 mL) was heated at reflux (80° C.) for 16 h. Solvent was removed and the residue was purified by pre-TLC (DCM/MeOH=10:1) to give (S)-1-(2-((20-(2-(4-(((4-(1-cyclopropyl-3-ethoxy-3-oxopropyl)pyridin-2-yl)oxy)methyl)piperidin-1-yl)-4-methoxy-N-(6-methylpyridin-2-yl)benzamido)-19,19-dimethylicosyl)amino)-2-oxoethyl)-1,4-diazabicyclo [2.2.2]octan-1-ium, E23-4, as a yellow gum. MS (ESI) m/z 1048.8 [M+]+.

Step 4. To a solution of E23-4 (35 mg, 0.033 mmol) in MeOH/THF/H₂O (1.5 mL/1.5 mL/0.5 mL) was added LiOH·H₂O (7 mg, 0.167 mmol). The reaction mixture was stirred at room temperature for 14 hours. LCMS indicated the completion of reaction. The reaction mixture was then acidified with 1 M HCl until pH reached 2 and concentrated. The residue was purified by prep-HPLC to give (S)-1-(2-((20-(2-(4-(((4-(2-carboxy-1-cyclopropylethyl)pyridin-2-yl)oxy)methyl)piperidin-1-yl)-4-methoxy-N-(6-methylpyridin-2-yl)benzamido)-19,19-dimethylicosyl)amino)-2-oxoethyl)-1,4-diazabicyclo[2.2.2]octan-1-ium trifluoroacetate, Example 23, as a white solid. MS (ESI) m/z 1019.6 [M+]+. MS (ESI) m/z 1020.5 [M+]⁺. ¹H NMR (400 MHz, DMSO-d₆) δ 8.64-8.63 (d, 1H), 8.05-8.04 (d, 1H), 7.29-7.26 (br, 1H), 7.11-7.09 (d, 1H), 6.93-6.92 (d, 1H), 6.86-6.84 (d, 1H), 6.72 (s, 1H), 6.52-6.50 (d, 1H), 6.45 (br, 1H), 6.23 (s, 1H), 4.13-4.12 (d, 2H), 4.12 (br, 4H), 3.72-3.70 (m, 6H), 3.69 (s, 3H), 3.37-3.34 (m, 6H), 3.13-3.08 (m, 8H), 2.69-2.67 (d, 2H), 2.36 (s, 3H), 2.27-2.21 (m, 1H), 1.76 (br, 1H), 1.69-1.66 (m, 2H), 1.46-1.36 (m, 2H), 1.27-1.22 (m, 28H), 1.04-0.97 (m, 5H), 0.75 (s, 6H), 0.52-0.49 (m, 1H), 0.35-0.25 (m, 2H), 0.22-0.18 (m, 1H).

### Example 24

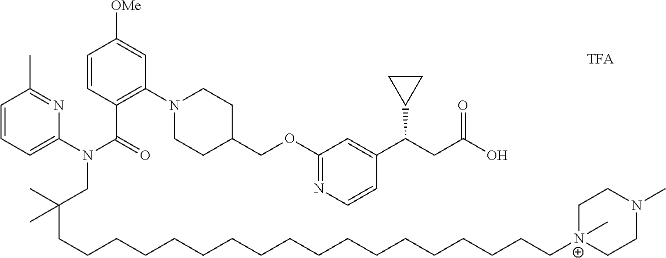

Example 24 was prepared in a similarly way as described for Example 21. MS (ESI) m/z 483.4 [(M+H]/2]⁺. ¹H NMR (400 MHz, DMSO-d₆) δ 8.06-8.04 (d, 1H), 7.29-7.26 (br, 1H), 7.11-7.09 (d, 1H), 6.94-6.92 (d, 1H), 6.86-6.84 (d, 1H), 6.73 (s, 1H), 6.53-6.50 (d, 1H), 6.44 (br, 1H), 6.24 (s, 1H), 4.15-4.13 (d, 2H), 4.10 (br, 2H), 3.69 (br, 7H), 3.63 (br, 4H), 3.47 (br, 2H), 3.18 (s, 3H), 2.89 (s, 3H), 2.70-2.68 (d, 2H), 2.37 (s, 3H), 2.28-2.22 (m, 1H), 1.77-1.75 (br, 1H), 1.74-1.67 (m, 4H), 1.46-1.36 (m, 2H), 1.27-1.22 (m, 28H), 1.04-0.97 (m, 5H), 0.75 (s, 6H), 0.52-0.49 (m, 1H), 0.35-0.25 (m, 2H), 0.22-0.18 (m, 1H).

### Example 25

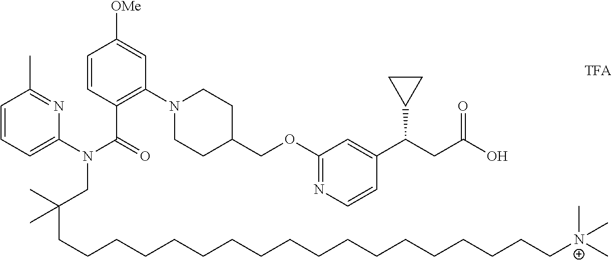 [SMILES: COc1ccc(C(=O)N(CC(C)(C)CCCCCCCCCCCCCCCCCC[N+](C)(C)C)c2cccc(C)n2)c(N2CCC(COc3cc([C@@H](CC(=O)O)C4CC4)ccn3)CC2)c1]

Example 25 was prepared in a similarly way as described for Example 21. MS (ESI) m/z 910.5 [M+]⁺. ¹H NMR (400 MHz, DMSO-d₆) δ 8.05-8.04 (d, 1H), 7.26 (br, 1H), 7.11-7.09 (d, 1H), 6.93-6.92 (d, 1H), 6.85-6.84 (d, 1H), 6.72 (s, 1H), 6.52-6.50 (d, 1H), 6.43 (br, 1H), 6.23 (s, 1H), 4.14-4.12 (d, 2H), 4.11 (br, 2H), 3.69 (s, 3H), 3.27-3.23 (m, 2H), 3.03 (s, 9H), 2.69-2.67 (d, 2H), 2.36 (s, 3H), 2.27-2.23 (m, 1H), 1.77 (br, 1H), 1.74-1.67 (m, 4H), 1.46-1.36 (m, 2H), 1.27-1.22 (m, 28H), 1.04-0.97 (m, 5H), 0.75 (s, 6H), 0.52-0.49 (m, 1H), 0.35-0.25 (m, 2H), 0.22-0.18 (m, 1H).

### Example 26

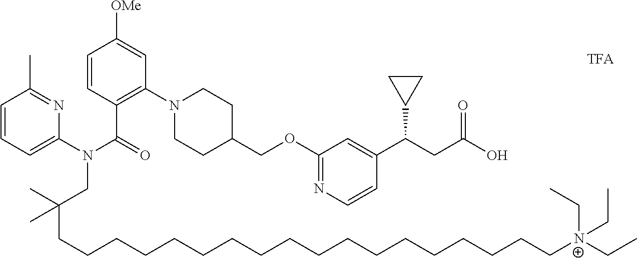 [SMILES: CC[N+](CC)(CC)CCCCCCCCCCCCCCCCCCC(C)(C)CN(C(=O)c1ccc(OC)cc1N1CCC(COc2cc([C@@H](CC(=O)O)C3CC3)ccn2)CC1)c1cccc(C)n1]

Example 26 was prepared in a similarly way as described for Example 21. MS (ESI) m/z 476.8 [(M+H)/2]⁺. ¹H NMR (400 MHz, DMSO-d₆) δ 8.05-8.04 (d, 1H), 7.26 (br, 1H), 7.11-7.08 (d, 1H), 6.93-6.91 (d, 1H), 6.86-6.84 (d, 1H), 6.71 (s, 1H), 6.52-6.50 (d, 1H), 6.45 (br, 1H), 6.23 (s, 1H), 4.14-4.12 (d, 2H), 4.11 (br, 2H), 3.69 (s, 3H), 3.24-3.19 (q, 6H), 3.11-3.07 (m, 2H), 2.69-2.67 (d, 2H), 2.36 (s, 3H), 2.27-2.23 (m, 1H), 1.79 (br, 1H), 1.74-1.67 (m, 4H), 1.46-1.36 (m, 2H), 1.27-1.10 (m, 37H), 1.04-0.97 (m, 5H), 0.75 (s, 6H), 0.52-0.49 (m, 1H), 0.35-0.25 (m, 2H), 0.22-0.18 (m, 1H).

### Example 27

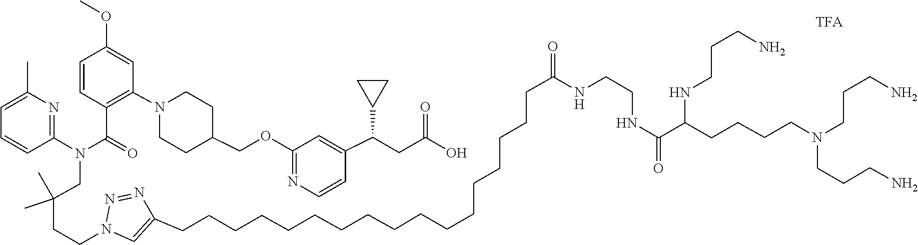 [SMILES: COc1ccc(C(=O)N(CC(C)(C)CCn2cc(CCCCCCCCCCCCCCCCCC(=O)NCCNC(=O)C(CCCCN(CCCN)CCCN)NCCCN)nn2)c2cccc(C)n2)c(N2CCC(COc3cc([C@@H](CC(=O)O)C4CC4)ccn3)CC2)c1]

MS (ESI) m/z 1320.0 [M+H]⁺. ¹H NMR (400 MHz, DMSO-d₆) δ 8.04-8.03 (d, 1H), 8.00 (br, 9H), 7.94 (br, 2H), 7.29-7.26 (br, 1H), 7.15-7.13 (d, 1H), 6.93-6.92 (d, 1H), 6.86-6.84 (d, 1H), 6.71 (s, 1H), 6.53-6.50 (dd, 1H), 6.46-6.45 (br, 1H), 6.213 (s, 1H), 4.34-4.30 (t, 2H), 4.18 (br, 4H), 4.13-4.11 (d, 2H), 3.75-3.72 (m, 1H), 3.69 (s, 3H), 3.12-3.05 (m, 10H), 3.02-3.00 (m, 2H), 2.90-2.86 (m, 6H), 2.69-2.67 (d, 2H), 2.35 (s, 3H), 2.26-2.21 (m, 1H), 2.06-2.03 (m, 2H), 1.98-1.87 (m, 6H), 1.80-1.76 (m, 3H), 1.65-1.62 (m, 6H), 1.46-1.36 (m, 2H), 1.27-1.22 (m, 28H), 1.04-0.97 (m, 1H), 0.75 (s, 6H), 0.52-0.49 (m, 1H), 0.35-0.25 (m, 2H), 0.22-0.18 (m, 1H).

### Example 28

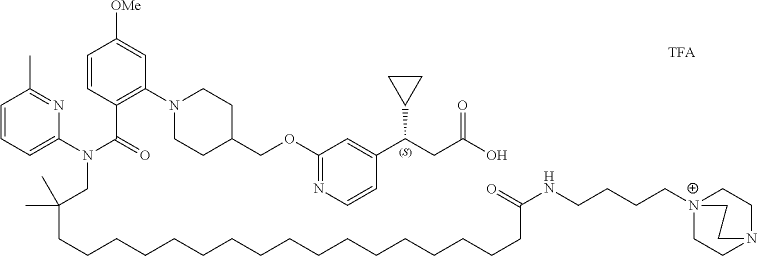

MS (ESI) m/z 525.0[(M+H)/2]⁺. ¹H NMR (400 MHz, DMSO-d₆) δ 8.05-8.03 (d, 1H), 7.80 (t, NH, 1H), 7.28-7.25 (br, 1H), 7.11-7.08 (d, 1H), 6.93-6.91 (d, 1H), 6.86-6.84 (d, 1H), 6.71 (s, 1H), 6.52-6.50 (d, 1H), 6.44 (br, 1H), 6.23 (s, 1H), 4.14-4.10 (m, 4H), 3.69 (s, 3H), 3.38-3.28 (m, 6H), 3.26-3.22 (m, 2H), 3.19-3.15 (m, 6H), 3.09-3.04 (m, 2H), 2.69-2.67 (d, 2H), 2.36 (s, 3H), 2.27-2.21 (m, 1H), 2.07-2.03 (m, 2H), 1.83-1.78 (m, 1H), 1.69-1.63 (m, 4H), 1.49-1.38 (m, 6H), 1.27-1.22 (m, 26H), 1.04-0.96 (m, 5H), 0.75 (s, 6H), 0.52-0.48 (m, 1H), 0.35-0.25 (m, 2H), 0.22-0.18 (m, 1H).

### Example 29

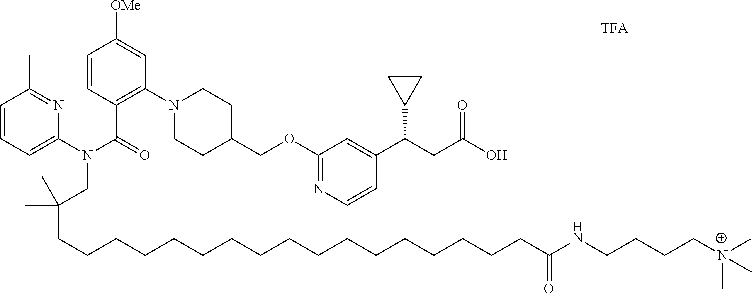 [SMILES: COc1ccc(C(=O)N(CC(C)(C)CCCCCCCCCCCCCCCCCC(=O)NCCCC[N+](C)(C)C)c2cccc(C)n2)c(N2CCC(COc3cc([C@@H](CC(=O)O)C4CC4)ccn3)CC2)c1]

MS (ESI) m/z 995.5 [M+]⁺. ¹H NMR (400 MHz, DMSO-d₆) δ 8.05-8.04 (d, 1H), 7.83 (t, NH, 1H), 7.29-7.25 (br, 1H), 7.11-7.09 (d, 1H), 6.94-6.93 (d, 1H), 6.86-6.84 (d, 1H), 6.74 (s, 1H), 6.53-6.50 (dd, 1H), 6.44 (br, 1H), 6.24 (s, 1H), 4.14-4.10 (m, 4H), 3.69 (s, 3H), 3.30-3.27 (m, 2H), 3.09-3.02 (m, 11H), 2.70-2.68 (d, 2H), 2.36 (s, 3H), 2.28-2.22 (m, 1H), 2.06-2.02 (m, 2H), 1.78 (br, 1H), 1.70-1.62 (m, 4H), 1.49-1.46 (m, 2H), 1.43-1.35 (m, 4H), 1.27-1.22 (m, 26H), 1.04-0.96 (m, 5H), 0.75 (s, 6H), 0.52-0.48 (m, 1H), 0.35-0.25 (m, 2H), 0.22-0.18 (m, 1H).

### Example 30

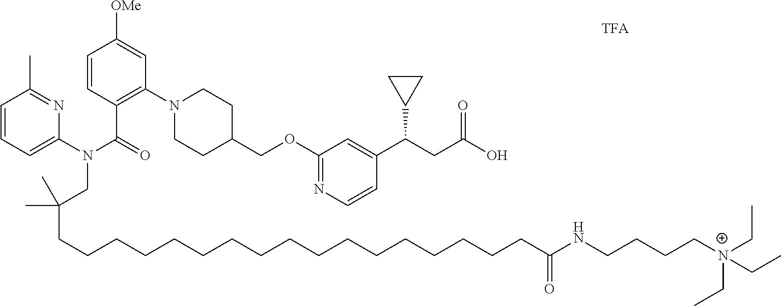 [SMILES: CC[N+](CC)(CC)CCCCNC(=O)CCCCCCCCCCCCCCCCCC(C)(C)CN(C(=O)c1ccc(OC)cc1N1CCC(COc2cc([C@@H](CC(=O)O)C3CC3)ccn2)CC1)c1cccc(C)n1]

MS (ESI) m/z 519.3 [(M+H)/2]⁺. ¹H NMR (400 MHz, DMSO-d₆) δ 8.05-8.03 (d, 1H), 7.81 (t, NH, 1H), 7.30-7.25 (br, 1H), 7.11-7.08 (d, 1H), 6.93-6.91 (d, 1H), 6.86-6.84 (d, 1H), 6.72 (s, 1H), 6.53-6.49 (dd, 1H), 6.44 (br, 1H), 6.23 (s, 1H), 4.14-4.10 (m, 4H), 3.69 (s, 3H), 3.24-3.17 (q, C), 3.14-3.04 (m, 4H), 2.69-2.67 (d, 2H), 2.36 (s, 3H), 2.27-2.22 (m, 1H), 2.06-2.02 (m, 2H), 1.80 (br, 1H), 1.76-1.66 (m, 2H), 1.56-1.53 (m, 2H), 1.46-1.38 (m, 4H), 1.27-1.22 (m, 35H), 1.04-0.96 (m, 5H), 0.75 (s, 6H), 0.52-0.48 (m, 1H), 0.35-0.25 (m, 2H), 0.22-0.18 (m, 1H).

### Example 31

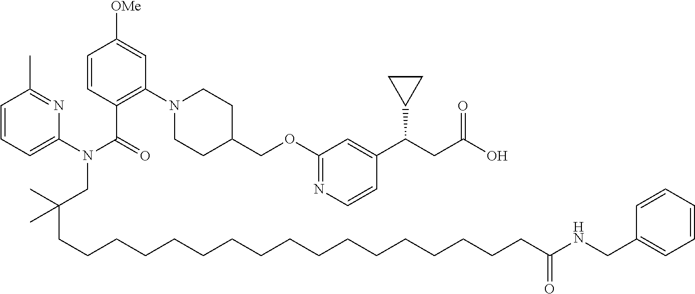 [SMILES: COc1ccc(C(=O)N(CC(C)(C)CCCCCCCCCCCCCCCCCC(=O)NCc2ccccc2)c2cccc(C)n2)c(N2CCC(COc3cc([C@@H](CC(=O)O)C4CC4)ccn3)CC2)c1]

MS (ESI) m/z 972.5 [M+H]⁺. ¹H NMR (400 MHz, DMSO-d6) δ 8.04-8.03 (d, 1H), 7.30-7.20 (m, 6H), 7.10-7.08 (d, 1H), 6.92-6.90 (dd, 1H), 6.86-6.84 (d, 1H), 6.70 (s, 1H), 6.52-6.50 (d, 1H), 6.43 (br, 1H), 6.23 (s, 1H), 4.25-4.24 (d, 2H), 4.22-4.00 (m, 4H), 3.69 (s, 3H), 2.67-2.65 (d, 2H), 2.36 (s, 3H), 2.26-2.21 (m, 1H), 2.13-2.10 (m, 2H), 1.73-1.62 (m, 3H), 1.52-1.37 (m, 4H), 1.27-1.22 (m, 26H), 1.04-0.96 (m, 5H), 0.75 (s, 6H), 0.52-0.48 (m, 1H), 0.35-0.25 (m, 2H), 0.22-0.18 (m, 1H).

### Example 32

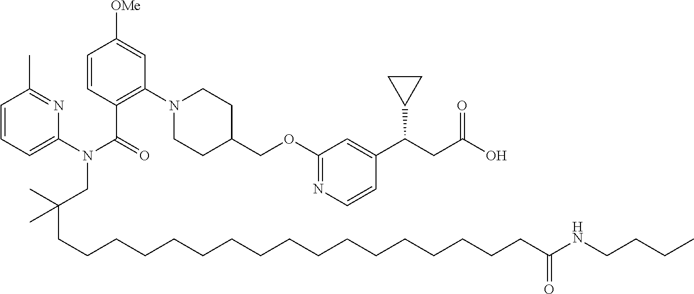 [SMILES: CCCCNC(=O)CCCCCCCCCCCCCCCCCC(C)(C)CN(C(=O)c1ccc(OC)cc1N1CCC(COc2cc([C@@H](CC(=O)O)C3CC3)ccn2)CC1)c1cccc(C)n1]

MS (ESI) m/z 938.5 [M+]⁺. ¹H NMR (400 MHz, DMSO-d₆) δ 8.04-8.03 (d, 1H), 7.29-7.25 (m, 1H), 7.11-7.08 (d, 1H), 6.92-6.91 (d, 1H), 6.86-6.84 (d, 1H), 6.70 (s, 1H), 6.52-6.50 (dd, 1H), 6.44 (br, 1H), 6.23 (s, 1H), 4.14-4.06 (m, 4H), 3.69 (s, 3H), 3.03-2.98 (q, 2H), 2.67-2.63 (d, 2H), 2.36 (s, 3H), 2.26-2.20 (m, 1H), 2.03-2.00 (m, 2H), 1.76 (br, 1H), 1.68-1.65 (m, 2H), 0.50-1.45 (m, 4H), 1.38-1.32 (m, 2H), 1.30-1.20 (m, 28H), 1.04-0.96 (m, 5H), 0.80 (t, 3H), 0.75 (s, 6H), 0.52-0.48 (m, 1H), 0.35-0.25 (m, 2H), 0.22-0.18 (m, 1H).

### Example 33

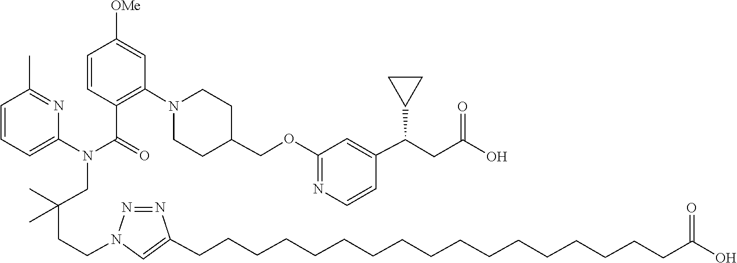 [SMILES: COc1ccc(C(=O)N(CC(C)(C)CCn2cc(CCCCCCCCCCCCCCCCCC(=O)O)nn2)c2cccc(C)n2)c(N2CCC(COc3cc([C@@H](CC(=O)O)C4CC4)ccn3)CC2)c1]

Example 33 is the Intermediate 59 of WO2023/134712.

### Example 34

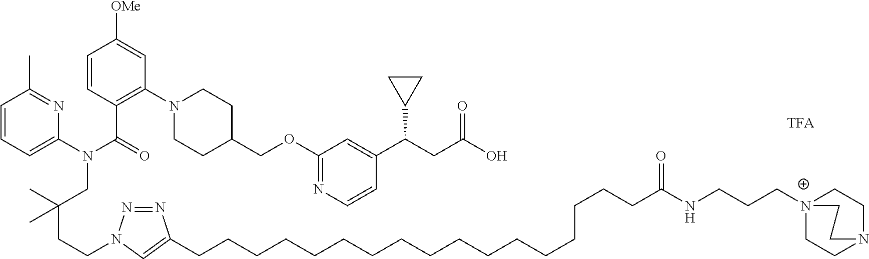 [SMILES: COc1ccc(C(=O)N(CC(C)(C)CCn2cc(CCCCCCCCCCCCCCCCCC(=O)NCCC[N+]34CCN(CC3)CC4)nn2)c2cccc(C)n2)c(N2CCC(COc3cc([C@@H](CC(=O)O)C4CC4)ccn3)CC2)c1]

Example 34 was synthesized in the same way as for Example 18, starting from Example 33. MS (ESI) m/z 1131.5 [M+]⁺. ¹H NMR (400 MHz, DMSO-d₆) δ 8.05-8.04 (d, 1H), 7.93-7.90 (t, 1H), 7.68 (s, 1H), 7.30-7.26 (br, 1H), 7.16-6.14 (d, 1H), 6.93-6.91 (d, 1H), 6.86-6.84 (d, 1H), 6.72 (s, 1H), 6.54-6.51 (dd, 1H), 6.45 (br, 1H), 6.21 (s, 1H), 4.35-4.30 (t, 2H), 4.20 (br, 2H), 4.13-4.12 (d, 2H), 3.69 (s, 3H), 3.45-3.41 (m, 6H), 3.27-3.24 (m, 8H), 3.12-3.08 (m, 2H), 2.69-2.67 (d, 2H), 2.58-2.55 (t, 2H), 2.36 (s, 3H), 2.27-2.21 (m, 1H), 2.07-2.04 (t, 2H), 1.84-1.70 (m, 3H), 1.69-1.63 (m, 4H), 1.60-1.45 (m, 4H), 1.38-1.25 (br, 2H), 1.30-1.20 (m, 26H), 1.02-0.99 (m, 1H), 0.78 (s, 6H), 0.52-0.49 (m, 1H), 0.36-0.25 (m, 2H), 0.17-0.13 (m, 1H).

### Example 35

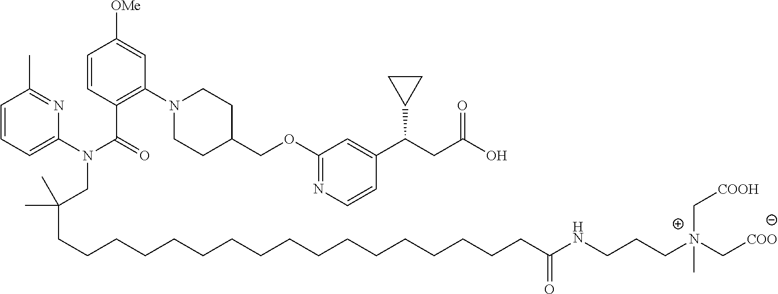

Example 35. MS (ESI) m/z 535.3 [(M+2H]/2]. ¹H NMR (400 MHz, DMSO-d₆) δ 8.05-8.03 (d, 1H), 7.88 (t, NH, 1H), 7.29-7.26 (m, 1H), 7.11-7.09 (d, 1H), 6.93-6.91 (d, 1H), 6.86-6.84 (d, 1H), 6.71 (s, 1H), 6.52-6.50 (dd, 1H), 6.44 (br, 1H), 6.23 (s, 1H), 4.46 (s, 4H), 4.14-4.07 (m, 4H), 3.69 (s, 3H), 3.66-3.62 (q, 2H), 3.31 (s, 3H), 3.10-3.05 (m, 2H), 2.69-2.67 (d, 2H), 2.36 (s, 3H), 2.26-2.20 (m, 1H), 2.05-2.00 (m, 2H), 1.88-1.75 (m, 3H), 1.69-1.65 (m, 2H), 1.50-1.40 (m, 4H), 1.25-1.15 (m, 26H), 1.04-0.96 (m, 5H), 0.75 (s, 6H), 0.52-0.48 (m, 1H), 0.35-0.25 (m, 2H), 0.22-0.18 (m, 1H).

### Example 36

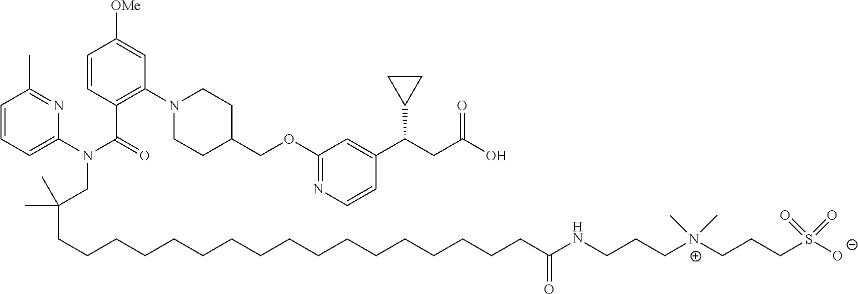 [SMILES: COc1ccc(C(=O)N(CC(C)(C)CCCCCCCCCCCCCCCCCC(=O)NCCC[N+](C)(C)CCCS(=O)(=O)[O-])c2cccc(C)n2)c(N2CCC(COc3cc([C@@H](CC(=O)O)C4CC4)ccn3)CC2)c1]

Example 35. MS (ESI) m/z 545.4 [(M+2H]/2]. ¹H NMR (400 MHz, DMSO-d₆) δ 8.05-8.04 (d, 1H), 7.87 (t, NH, 1H), 7.30-7.27 (m, 1H), 7.11-7.09 (d, 1H), 6.94-6.92 (dd, 1H), 6.86-6.85 (d, 1H), 6.73 (s, 1H), 6.53-6.50 (dd, 1H), 6.44 (br, 1H), 6.23 (s, 1H), 4.14-4.09 (m, 4H), 3.69 (s, 3H), 3.42-3.39 (m, 2H), 3.25-3.21 (m, 2H), 3.12-3.07 (m, 2H), 3.00 (s, 6H), 2.69-2.67 (d, 2H), 2.54-2.50 (m, 2H), 2.36 (s, 3H), 2.26-2.20 (m, 1H), 2.08-2.04 (m, 2H), 2.01-1.93 (m, 2H), 1.82-1.75 (m, 3H), 1.69-1.65 (m, 2H), 1.50-1.39 (m, 4H), 1.25-1.15 (m, 26H), 1.04-0.96 (m, 5H), 0.75 (s, 6H), 0.52-0.48 (m, 1H), 0.35-0.25 (m, 2H), 0.22-0.18 (m, 1H).

### Biological Example 1. Material and General Methods

IP1 accumulation assay was used to evaluate the potency of compounds. HEK293 cells stably expressing GPR40 were cultured in 5% CO₂ incubator (ThermoFisher) in maintenance media (Dulbecco's modified Eagle's medium with 4.5 g/L of glucose, 10% fetal bovine serum, 100 μg/mL Hygromycin, and Penicillin (100 U/mL)/Streptomycin (100 μg/mL)) till 100% confluency. Cells were harvested freshly, spun down at 300×g for 5 min, and resuspended in pre-warmed 1×stimulant buffer from Cisbio IP-One HTRF Detection kit (Cisbio). Cell density was adjusted to 2.0×10⁶ cells/mL. DMSO was used as blank control and AMG-1638 (CAS #: 1142214-62-7) as positive control. Compounds were prepared at 10 mM in DMSO and 5 nL of 3×serially diluted compounds (10 concentrations) were added to each well of the 384-LDV assay plate (Corning) by using ECHO 550 (Labcyte). Five μL of cells in suspension were transferred into each well by using Multidrop Combi Reagent Dispenser (ThermoFisher). Assay plate was then sealed and incubated at 37° C. for 2 hours. IP-d2 reagent and anti-IP1 reagent were prepared following the manual (Cisbio). Five μL of IP1-d2 and then 5 μL of anti-IP1 antibody was added to each well sequentially. Assay plate was incubated at room temperature for 60 min and then read at 665 nm/615 nm on an Envision plate reader (PerkinElmer). The ratio of values obtained at 665 nm and 615 nm was used for calculation of IP1 accumulation: % Effect=(Ratioₛₐₘₚₗₑ−Ratio_{blank})/(Ratio_{AMG-1638}−Ratio_{blank})×100. Dose curve was fitted and EC50 of each compound was calculated by using XLFit.

All exemplified compounds of the present disclosure, Examples 1-36, were tested according to Biological Example 1 and the EC50 values are <50 nM. Additional exemplary data tested according to Biological Example 1 are shown in the table below:

Biological Example 1: EC50 values of
exemplary compounds

| | EC50 |
|---|---|
| Example # | (nM) |
| 1 | 1.23 |
| 2 | 2.00 |
| 3 | 1.79 |
| 4 | 2.14 |
| 5 | 1.73 |
| 6 | 0.84 |
| 7 | 0.53 |
| 8 | 0.28 |
| 9 | 0.35 |
| 10 | 3.65 |
| 11 | 1.34 |
| 12 | 0.17 |
| 13 | 0.76 |
| 14 | 0.63 |
| 15 | 0.67 |
| 16 | 0.48 |
| 17 | 0.11 |
| 18 | 0.24 |
| 19 | 0.07 |
| 20 | 3.57 |
| 21 | 2.04 |
| 22 | 0.36 |
| 23 | 2.80 |
| 24 | 1.15 |
| 25 | 0.45 |
| 26 | 0.36 |
| 27 | 0.71 |
| 28 | 0.49 |
| 29 | 0.32 |
| 30 | 0.43 |
| 31 | 163 |
| 32 | 385 |
| 33 | >152 |
| 34 | 0.32 |
| 35 | 0.63 |
| 36 | 0.78 |

### Biological Example 2. Pharmacokinetics

PK Methods of in-Life

Male ICR (CD-1) mice at around 4-6 weeks with body weight of around 25 grams (n=3) were purchased from VT-River (Zhejiang, China). After 1-week acclimation, animals were dosed with exemplary compounds intravenously at 1 mpk or orally at 10 mpk (0.5% methylcellulose in PBS as vehicle for both routes of administration). Blood samples (30 μL) were collected via saphenous vein puncture at 5 (IV only), 15, 30, 60, 120, 240, 480, and 1440 min post dose. The blood samples were saved in 0.5 M K₂EDTA coated tubes and centrifuged at 4,600 rpm at 4° C. for 5 min. Plasma samples were collected and stored in −80° C. till PK analysis.

### PK Analysis by LC-MS/MS:

Take 10 μL of K₂EDTA-treated plasma samples of CD-1 mice, add 200 μL of precipitation solution with internal standard (5 ng/mL Terfenadine in MeOH/ACN), mix well by vortexing for 1 min at room temperature followed by centrifuge at 4° C. and 4000 rpm for 15 min. Save the supernatant as injection for LC-MS/MS quantification of exemplary compounds in plasma samples of mice.

Establish a standard curve before and after each analytical batch, accompanied by quality controls of low, medium, and high concentrations. The number of quality control samples is not less than 6 and should be equal to or more than the number of samples of each batch, and interspersed in sample measurement. Standard curve and quality control samples of each batch must meet the acceptance criteria.

Exemplary data obtained according to Biological Example 2 are shown in the table below:

Biological Example 2: Pharmacokinetic Data in Mice

| | IV (1 mpk) | | | | PO (10 mpk) | | |
|---|---|---|---|---|---|---|---|
| | AUC | Cl | Vss | T1/2 | | Tmax | Cmax |
| Example # | uM * hr | ml/mL/kg | L/kg | hr | F % | hr | nM |
| 4 | 54.1 | 0.22 | 0.14 | 8.2 | 0.16% | 5.3 | 66.1 |
| 12 | 33.9 | 0.15 | 0.10 | 8.9 | 0.17% | 5.3 | 80.7 |
| 13 | 88.9 | 0.14 | 0.07 | 6.5 | 0.52% | 8.0 | 268 |
| 14 | 68.0 | 0.20 | 0.09 | 6.7 | 0.37% | 8.0 | 164 |
| 17 | 103.5 | 0.10 | 0.08 | 10.5 | 0.41% | 6.7 | 238 |
| 18 | 33.7 | 0.32 | 0.11 | 5.1 | 0.15% | 4.0 | 42 |
| 19 | 28.5 | 0.43 | 0.11 | 4.7 | 0.31% | 4.0 | 77 |
| 21 | 18.5 | 0.67 | 0.36 | 7.2 | 0.34% | 6.7 | 37 |

### Biological Example 3. oGTT Study

Male C57BL/6 mice were purchased from Zhejiang Vital River Laboratories Co., Ltd (a branch of Charles River Laboratories) at around 7-8 weeks of age (n=8). Animal were kept under regular light/dark cycle (12 h light:12 h dark, LD) at room temperature and fed regular chow diet and tap water ad libitum in facility for more than one week. On study day, animals were fasted by removing food at 7:00 AM. Blood glucose was measured at 9:00 AM by a glucometer (Roche) via tail bleeding and mice were sorted to groups of treatment based on average blood glucose (−240 min). The vehicle (0.5% methylcellulose) or exemplary compounds solutions (30 mpk dose) were mixed well and dosed by oral gavage (dosing volume=10 ml/kg body weight). At 1:00 PM, blood glucose was measured by a glucometer (0 min). Animals were then PO dosed with 5 g/kg glucose for oral glucose challenge. Blood glucose was then measured at 20, 40, 60, and 120 min by glucometer. At the end of study, 50 μl tail blood from treatment groups were collected and mixed with 4% sodium citrate (vol 9:1). Tubes were centrifuged in a benchtop centrifuge for 10 min at 8,000 RPM at 4° C. Plasma samples were separated and stored in −80° C. freezer for measurements of PK by LC-MS/MS.

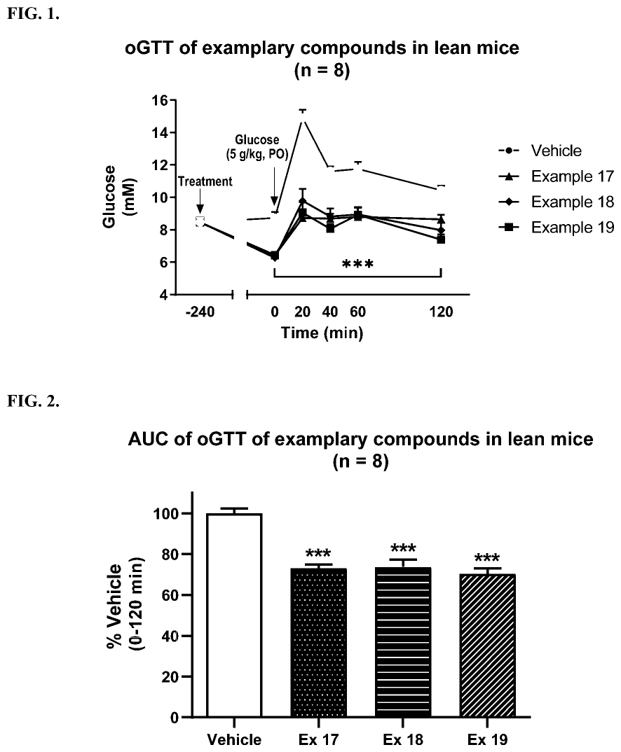

The test results are shown in FIGS. 1 and 2. The table below also shows the Percent change over vehicle of AUC and drug concentration at 6 h post dose:

| | | % change in AUC | Drug conc |
|---|---|---|---|
| Compounds | Dose | over vehicle | (ng/ml) |
| Example 17 | 30 mpk | −26.9% | 237 ± 2 |
| Example 18 | 30 mpk | −26.4% | 274 ± 99 |
| Example 19 | 30 mpk | −29.7% | 347 ± 125 |

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt or ester thereof: 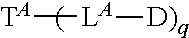
wherein:
D is a residue of a ligand of a membrane bound protein, such as a GPCR, preferably, D is a residue of a GPR40 agonist;
q is an integer of 1-10, preferably, 1 or 2;
L^{A} is a hydrophobic linker; and
T^{A} is a group characterized as having one or more (e.g., 1, 2, or 3) hydrophilic polar groups, preferably, T^{A} is a group characterized as having one or more (e.g., 1, 2, or 3) charged groups, such as having one or more quaternary amine, one or more carboxylic acid, one or more phosphoric acid, and/or one or more sulfonic acid,
wherein the compound is charge balanced as necessary.

2. The compound of claim 1, or a pharmaceutically acceptable salt or ester thereof, wherein q is 1.

3. The compound of claim 1 or 2, or a pharmaceutically acceptable salt or ester thereof, wherein T^{A} is a hydrophilic group having a terminal atom(s) selected from N, O, S, P, or C, which is covalently bonded with a first end atom of L^{A}, wherein (1) when the terminal atom(s) is N of a basic primary or secondary amine group, then the corresponding compound T^{A}-(C(O)—CH₃)_{q} has a c Log P of less than 0, preferably, less than −1, wherein the —C(O)—CH₃ is bonded with the terminal N atom(s); (2) when the terminal atom(s) is N of a basic tertiary amine group, then the corresponding compound [T^{A}-CH₃]⁺ has a c Log P of less than 0, preferably, less than −1, wherein the —CH₃ is bonded with the terminal N atom(s); (3) when the terminal atom(s) is C of a C(O) group, then the corresponding compound T^{A}-(OH)_{q} has a c Log P of less than 1, wherein the —OH is bonded with the terminal C atom(s); (4) when the terminal atom(s) is S of a SO₂ group, then the corresponding compound T^{A}-(OH)_{q} has a c Log P of less than 1, wherein the —OH is bonded with the terminal S atom(s); or (5) when (1)-(4) do not apply, then the corresponding compound T^{A}-H_{q} has a c Log P of less than 1.

4. The compound of claim 3, or a pharmaceutically acceptable salt or ester thereof, wherein T^{A} is a hydrophilic group having a terminal N atom, which is covalently bonded with the first end atom of L^{A}, wherein the terminal N atom is that of a basic primary or secondary amine group, and the corresponding compound T^{A}-(C(O)—CH₃)_{q} has a c Log P of less than 0, preferably, less than −1, wherein the —C(O)—CH₃ is bonded with the terminal N atom.

5. The compound of claim 3, or a pharmaceutically acceptable salt or ester thereof, wherein T^{A} is a hydrophilic group having a terminal N atom, which is covalently bonded with the first end atom of L^{A}, wherein the terminal N atom is that of a basic tertiary amine group, and the corresponding compound [T^{A}-CH₃]⁺ has a c Log P of less than 0, preferably, less than −1, wherein the —CH₃ is bonded with the terminal N atom.

6. The compound of any of claims 1-5, or a pharmaceutically acceptable salt or ester thereof, wherein L^{A} is a linker characterized in that the maximum length between the two end atoms of L^{A} is at least the maximum length between the two end carbon atoms of —(CH₂)₁₀—, wherein (1) when both end atoms of L^{A} are C of a C(O) or S of a SO2 group, then the corresponding compound HO-L^{A}-OH has a c Log P of at least 3, wherein each —OH is bonded with the end C(O) or SO2 group; (2) when only one end atom of L^{A} is C of a C(O) or S of a SO2 group, then the corresponding compound H-L^{A}-OH has a c Log P of at least 3, wherein the —OH is bonded with the C(O) or SO2 group; or (3) when neither (1) and (2) applies, then the corresponding compound H-L^{A}-H has a c Log P of at least 3.

7. The compound of any of claims 1-5, or a pharmaceutically acceptable salt or ester thereof, wherein only one end atom of L^{A} is C of a C(O) or S of a SO2 group, and the corresponding compound H-L^{A}-OH has a c Log P of at least 3, preferably, at least 4, wherein the —OH is bonded with the C(O) or SO2 group.

8. The compound of any of claims 1-7, or a pharmaceutically acceptable salt or ester thereof, wherein the covalent bond(s) between the terminal atom(s) of T^{A} and the first end atom of L^{A} is an amide bond.

9. The compound of any of claims 1-7, or a pharmaceutically acceptable salt or ester thereof, wherein the covalent bond(s) between the terminal atom(s) of T^{A} and the first end atom of L^{A} is a non-amide carbon-nitrogen bond, an ester bond, a non-ester carbon-oxygen bond, a carbon-carbon bond, or a carbon-sulfur bond.

10. The compound of any of claims 1-9, or a pharmaceutically acceptable salt or ester thereof, wherein L^{A} is characterized in that the maximum length between the two end atoms of L^{A} is at least that between the two end carbon atoms of —(CH₂)₁₂—, preferably, at least that of —(CH₂)₁₄—, more preferably, at least that of —(CH₂)₁₆—.

11. The compound of any of claims 1-9, or a pharmaceutically acceptable salt or ester thereof, wherein L^{A} is characterized in that the maximum length between the two end atoms of L^{A} is between (i) the maximum length between the two end carbon atoms of —(CH₂)₁₂— and (ii) the maximum length between the two end carbon atoms of —(CH₂)₅₀—.

12. The compound of any of claims 1-11, or a pharmaceutically acceptable salt or ester thereof, wherein T^{A} has a formula according to M-1 or M-2: 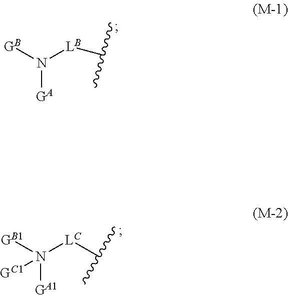
wherein:
each of L^{B} and L^{C} at each occurrence is independently null or represents a divalent group;
wherein, in M-1:
(i) one of G^{A} and G^{B} is hydrogen or is selected from C₁₋₄ alkyl, C₂₋₄ alkenyl, C₂₋₄ alkynyl, or a 3-14 membered ring, each of which is optionally substituted, and the other of G^{A} and G^{B} is a moiety having the structure of M-2, M-3, or M-4 as defined in this claim; or
(ii) G^{A} and G^{B}, together with the nitrogen atom they are both attached to, are joined to form an optionally substituted 4-14 membered ring; or
(iii) each of G^{A} and G^{B} independently represents a moiety having the structure of M-2, M-3, or M-4 as defined in this claim;
wherein, in M-2
(i) G^{A1}, G^{B1}, and G^{C1} each independently represents C₁₋₄ alkyl, C₂₋₄ alkenyl, C₂₋₄ alkynyl, a 3-14 membered ring, or a structure according to M-3 or M-4; wherein each of the C₁₋₄ alkyl, C₂₋₄ alkenyl, C₂₋₄alkynyl, and 3-14 membered ring is optionally substituted;
(ii) G^{A1} and G^{B1}, together with the nitrogen atom they are both attached to, are joined to form an optionally substituted 4-14 membered ring; and G^{C1} is as defined in (i); or
(iii) G^{A1}, G^{B1}, and G^{C1} together with the nitrogen atom they are all attached to, are joined to form an optionally substituted 5-14 membered ring;
wherein M-3 has a structure of 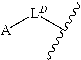
and M-4 has a structure of 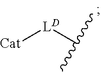
wherein:
L^{D} is null or represents a divalent group;
A represents a moiety having an anionic group or a conjugated acid thereof, preferably, the anionic group is selected from COO⁻, SO₃⁻, HPO₃⁻ or PO₃²⁻; and
Cat represents a moiety having a cationic group that is positively charged regardless of pH or positively chargeable at pH of 7, preferably, the cationic group is a quaternary amine.

13. The compound of claim 12, or a pharmaceutically acceptable salt or ester thereof, wherein T^{A} has a formula according to M-1, wherein one of G^{A} and G^{B} is hydrogen or C₁₋₄ alkyl, and the other of G^{A} and G^{B} has a structure according to M-2, M-3, or M-4 as defined above.

14. The compound of claim 12 or 13, or a pharmaceutically acceptable salt or ester thereof, wherein L^{B} is null.

15. The compound of claim 12 or 13, or a pharmaceutically acceptable salt or ester thereof, wherein L^{B} is a C₁₋₆ alkylene or a C₁₋₆ heteroalkylene having one or two heteroatoms independently selected from N, O, P, and S, wherein the P or S is optionally oxidized.

16. The compound of claim 12, or a pharmaceutically acceptable salt or ester thereof, wherein T^{A} has a formula according to M-2.

17. The compound of any of claims 12-16, or a pharmaceutically acceptable salt or ester thereof, wherein G^{A1} and G^{B1}, together with the nitrogen atom they are both attached to, are joined to form an optionally substituted 3-14 membered ring; and G^{C1} is C₁₋₄ alkyl, e.g.,

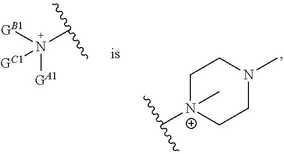

18. The compound of any of claims 12-16, or a pharmaceutically acceptable salt or ester thereof, wherein G^{A1}, G^{B1}, and G^{C1} together with the nitrogen atom they are all attached to, are joined to form an optionally substituted 5-14 membered ring, preferably, a fused or bridged bicyclic ring, e.g.,

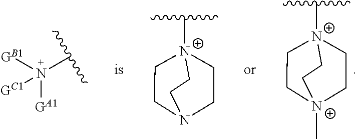

19. The compound of any of claims 12-18, or a pharmaceutically acceptable salt or ester thereof, wherein L^{C} is null.

20. The compound of any of claims 12-18, or a pharmaceutically acceptable salt or ester thereof, wherein L^{C} is a C₁₋₆ alkylene or a C₁₋₆ heteroalkylene having one or two heteroatoms independently selected from N, O, P, and S, wherein the P or S is optionally oxidized.

21. The compound of any of claims 12-16, or a pharmaceutically acceptable salt or ester thereof, wherein M-2 represents 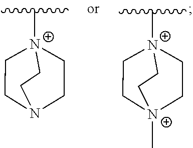
or M-2 represents 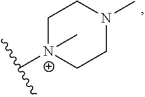
[N(CH₃)₃]⁺, or [N(CH₂CH₃)₃]⁺.

22. The compound of any of claims 1-11, or a pharmaceutically acceptable salt or ester thereof, wherein T^{A} represents 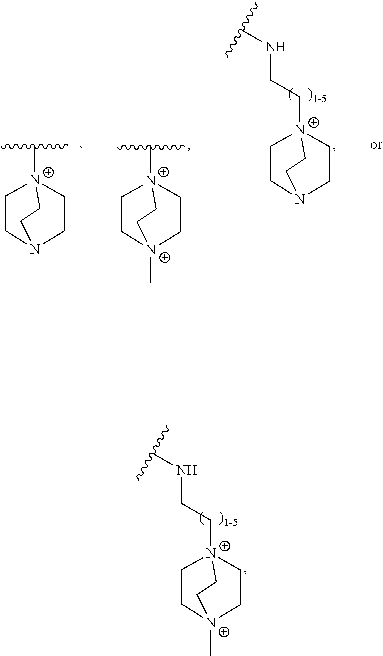
or T^{A} represents 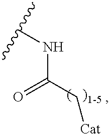
wherein Cat is 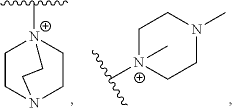
[N(CH₃)₃]⁺, or [N(CH₂CH₃)₃]⁺.

23. The compound of any of claims 1-11, or a pharmaceutically acceptable salt or ester thereof, wherein T^{A} represents one of the following structures: 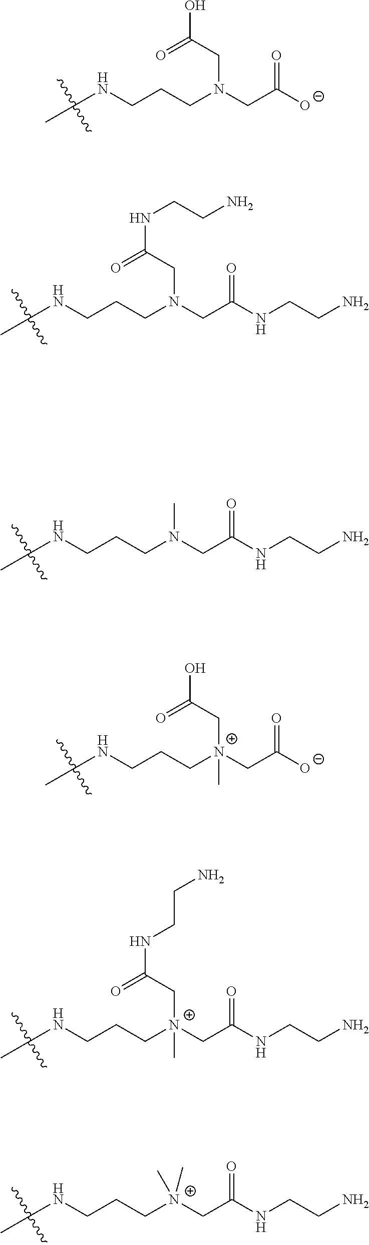 [SMILES: *NCCCN(CC(=O)[O-])CC(=O)O ; *NCCCN(CC(=O)NCCN)CC(=O)NCCN ; *NCCCN(C)CC(=O)NCCN ; *NCCC[N+](C)(CC(=O)[O-])CC(=O)O ; *NCCC[N+](C)(CC(=O)NCCN)CC(=O)NCCN ; *NCCC[N+](C)(C)CC(=O)NCCN] 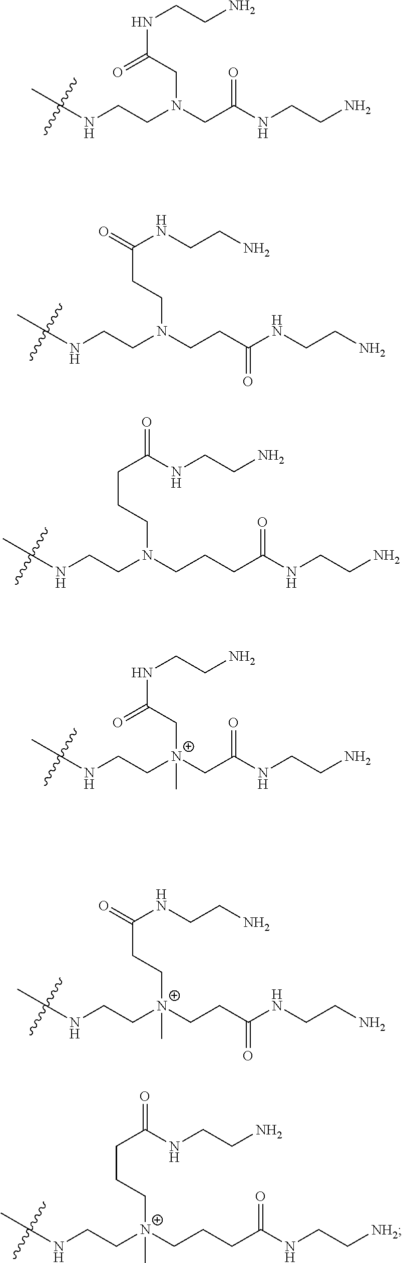 [SMILES: *NCCN(CC(=O)NCCN)CC(=O)NCCN ; *NCCN(CCC(=O)NCCN)CCC(=O)NCCN ; *NCCN(CCCC(=O)NCCN)CCCC(=O)NCCN ; *NCC[N+](C)(CC(=O)NCCN)CC(=O)NCCN ; *NCC[N+](C)(CCC(=O)NCCN)CCC(=O)NCCN ; *NCC[N+](C)(CCCC(=O)NCCN)CCCC(=O)NCCN]
T^{A} represents 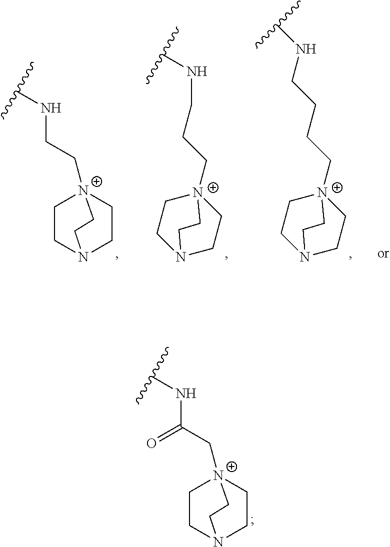 [SMILES: *NCC[N+]12CCN(CC1)CC2 ; *NCCC[N+]12CCN(CC1)CC2 ; *NCCCC[N+]12CCN(CC1)CC2 ; *NC(=O)C[N+]12CCN(CC1)CC2]
T^{A} represents 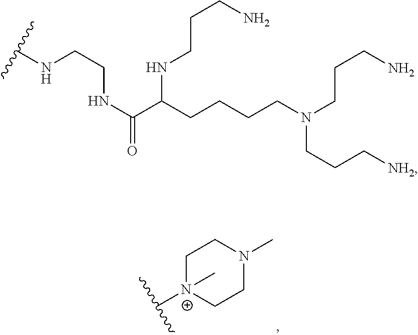
[N(CH₃)₃]⁺, [N(CH₂CH₃)₃]⁺,

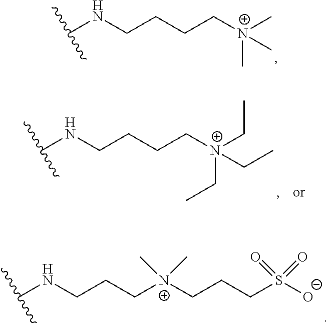

24. The compound of any of claims 1-23, or a pharmaceutically acceptable salt or ester thereof, wherein L^{A} is (X)ₘ, wherein X at each occurrence is independently CR₂, C(═O), —C(R)═C(R)—, 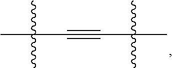 [SMILES: *C#C*]
SiR₂, O, S, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy, typically R is hydrogen or C₁₋₄ alkyl, and the integer m is at least 10, such as at least 12, at least 14, at least 16, at least 18, at least 20, at least 50, such as 12-50, 16-50, etc.

25. The compound of any of claims 1-23, or a pharmaceutically acceptable salt or ester thereof, wherein L^{A} is —(X)ₘ₋₁—C(O)—, wherein the C(O) end is bonded with T^{A}, wherein X at each occurrence is independently CR₂, C(═O), —C(R)═C(R)—, 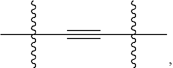 [SMILES: *C#C*]
SiR₂, O, S, SO₂, NR, [NR₂]⁺ or a ring structure, preferably 3-10 membered ring structure, provided that the end X group is not C(O) or SO₂, wherein R at each occurrence is independently hydrogen, halogen, optionally substituted C₁₋₄ alkyl, or optionally substituted C₁₋₄ alkoxy, typically R is hydrogen or C₁₋₄ alkyl, and the integer m is at least 10, such as at least 12, at least 14, at least 16, at least 18, at least 20, at least 50, such as 12-50, 16-50, etc., and the hydrophobicity of —(X)ₘ₋₁—C(O)— is characterized in that the corresponding compound H—(X)ₘ₋₁—COOH has a c Log P of at least 3, such as between 3-15, preferably, at least 4.

26. The compound of claim 24 or 25, or a pharmaceutically acceptable salt or ester thereof, wherein X at each occurrence is independently CR₂, —C(R)═C(R)—, 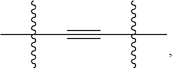 [SMILES: *C#C*]
or a 3-10 membered ring, wherein R at each occurrence is independently hydrogen or C₁₋₄ alkyl; more preferably, X at each occurrence is independently CR₂, wherein R at each occurrence is independently hydrogen or C₁₋₄ alkyl.

27. The compound of any of claims 1-23, or a pharmaceutically acceptable salt or ester thereof, wherein L^{A} is —C₁₂₋₃₀ alkylene- or —C₁₂₋₃₀ alkylene-C(O)—, wherein the —C₁₂₋₃₀ alkylene- is optionally substituted, wherein the optional substituents can optionally be joined together to form a double bond, triple bond, or a ring structure.

28. The compound of any of claims 1-23, or a pharmaceutically acceptable salt or ester thereof, wherein L^{A} is a 12-30 membered heteroalkylene or -(12-30 membered heteroalkylene)-C(O)—, wherein the 12-30 membered heteroalkylene is optionally substituted and contains 1-6 heteroatoms independently selected from O, N, and S, wherein the sulfur atom(s), if present, is optionally oxidized, wherein the optional substituents can optionally be joined together to form a double bond, triple bond, or a ring structure.

29. The compound of any of claims 1-28, or a pharmaceutically acceptable salt or ester thereof, wherein D is a residue having the formula of D-1: 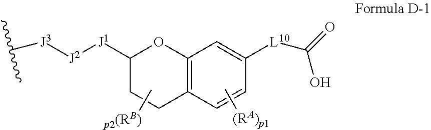
wherein:
L¹⁰ is an alkylene (e.g., a C₁₋₆ alkylene), optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{B} at each occurrence is independently halogen, hydroxyl, amino, substituted amino, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{B} are joined to form an optionally substituted ring structure; p2 is 0, 1, 2, 3, or 4;
J¹ is a bond, an optionally substituted aryl or heteroaryl ring, C₁₋₆alkylene-N(R¹⁰⁰)—, 3-14 membered optionally substituted heterocyclylene containing at least one ring nitrogen atom, or —C₁₋₆alkylene-(3-14 membered optionally substituted heterocyclylene containing at least one ring nitrogen atom)-, wherein R¹⁰⁰ is hydrogen, optionally substituted alkyl, or optionally substituted cycloalkyl;
J² is a bond or an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, or two substituents are joined to form an optionally substituted ring structure; and
J³ is an optionally substituted cycloalkyl, heterocyclyl, aryl or heteroaryl ring.

30. The compound of claim 29, or a pharmaceutically acceptable salt or ester thereof, wherein p1 is 0.

31. The compound of claim 29 or 30, or a pharmaceutically acceptable salt or ester thereof, wherein p1 is 1, and R^{A} is F, Cl, CN, C₁₋₄ alkyl optionally substituted with 1-3 fluorine, or C₁₋₄ alkoxy optionally substituted with 1-3 fluorine.

32. The compound of any of claims 29-31, or a pharmaceutically acceptable salt or ester thereof, wherein p2 is 0.

33. The compound of any of claims 29-31, or a pharmaceutically acceptable salt or ester thereof, wherein p2 is 1 or 2, and R^{B} at each occurrence is independently F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

34. The compound of 29, or a pharmaceutically acceptable salt or ester thereof, wherein D has a formula according to D-1-A: 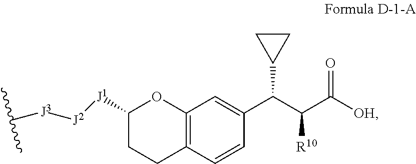
wherein R¹⁰ is hydrogen or C₁₋₄ alkyl (preferably methyl).

35. The compound of any of claims 29-34, or a pharmaceutically acceptable salt or ester thereof, wherein J¹ is a 4-12 membered optionally substituted heterocyclic ring having one or two ring nitrogen atoms.

36. The compound of claim 35, or a pharmaceutically acceptable salt or ester thereof, wherein JP is a 4-8 membered optionally substituted monocyclic saturated heterocyclic ring having one or two ring heteroatoms independently selected from S, O, and N, provided at least one of the ring heteroatom is nitrogen.

37. The compound of claim 36, or a pharmaceutically acceptable salt or ester thereof, wherein JP is selected from: 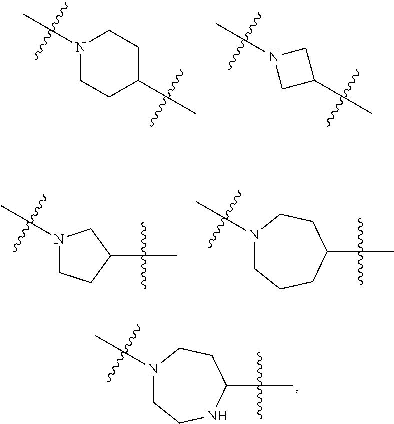 [SMILES: *C1CCN(*)CC1 ; *C1CN(*)C1 ; *C1CCN(*)C1 ; *C1CCCN(*)CC1 ; *C1CCN(*)CCN1]
each of which is optionally substituted with 1-2 substituents independently selected from F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

38. The compound of claim 36, or a pharmaceutically acceptable salt or ester thereof, wherein JP is bicyclic or polycyclic 6-12 membered optionally substituted saturated heterocyclic ring having one or two ring heteroatoms independently selected from S, O, and N, provided at least one of the ring heteroatom is nitrogen.

39. The compound of any of claims 29-38, or a pharmaceutically acceptable salt or ester thereof, wherein J² is a straight chain or branched C₁₋₄ alkylene, optionally substituted with 1-3 fluorine.

40. The compound of any of claims 29-39, or a pharmaceutically acceptable salt or ester thereof, wherein J² is CH₂ or —CH(CH₃)—.

41. The compound of any of claims 29-40, or a pharmaceutically acceptable salt or ester thereof, wherein J³ is an aryl or heteroaryl ring, each of which is unsubstituted or substituted with one or more (e.g., 1, 2, or 3) substituents independently selected from 1) halogen, CN, —CF₃, OH, amino, substituted amino, ester, amide, carbonate, or carbamate; and 2) C₁₋₆ alkyl, C₂₋₆ alkenyl, C₂₋₆ alkynyl, C₁₋₆ heteroalkyl, C₃₋₆ cycloalkyl, C₁₋₆ alkoxy, C₃₋₆ cycloalkoxy, aryl, heteroaryl, 3-8 membered heterocycloalkyl having one or two ring heteroatoms independently selected from N, O, and S, wherein each of which is optionally substituted with one or more (e.g., 1, 2, or 3) substituents independently selected from F, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy.

42. The compound of any of claims 29-41, or a pharmaceutically acceptable salt or ester thereof, wherein J³ is a phenyl ring, which is substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

43. The compound of any of claims 29-41, or a pharmaceutically acceptable salt or ester thereof, wherein J³ is a 5-10 membered monocyclic or bicyclic heteroaryl ring, which is substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

44. The compound of any of claims 29-41, or a pharmaceutically acceptable salt or ester thereof, wherein J³ is selected from: 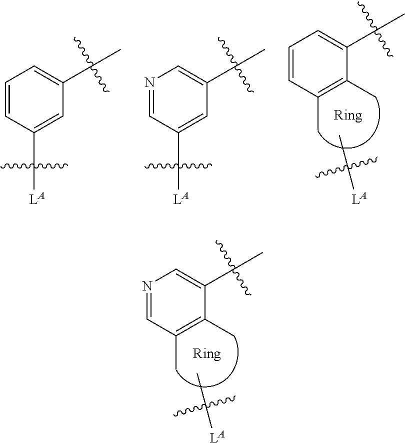
wherein: Ring represents an aromatic or non-aromatic ring structure,
wherein each of the phenyl, pyridyl, or fused ring structure is optionally substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

45. The compound of any of claims 29-41, or a pharmaceutically acceptable salt or ester thereof, wherein J³ is selected from: 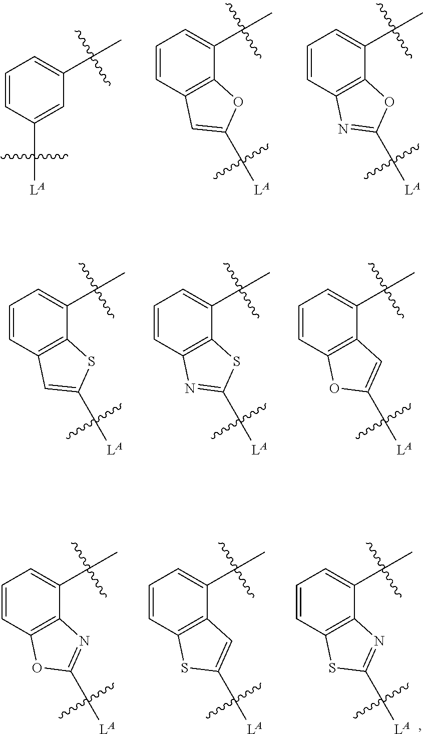
wherein each of which is optionally substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

46. The compound of claim 45, or a pharmaceutically acceptable salt or ester thereof, wherein J³ is selected from: 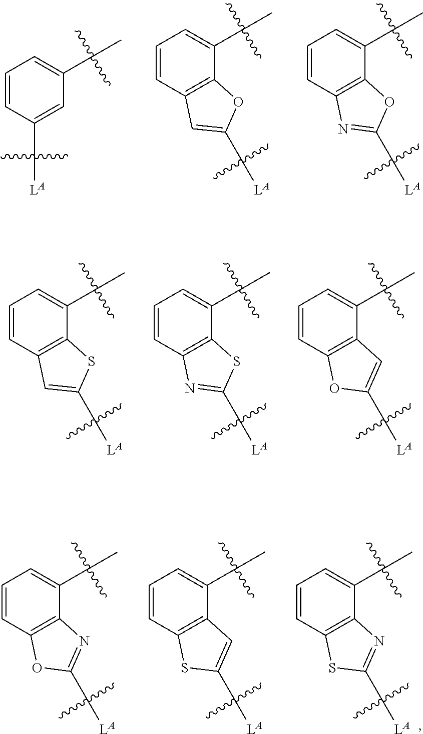
wherein each of which is optionally substituted with 1-3 substituents independently selected from F, Cl, CN, OH, C₁₋₆ alkyl optionally substituted with F (e.g., CF₃), cyclopropyl, cyclobutyl, C₁₋₆ alkoxy optionally substituted with F (e.g., —O—CF₃), or C₃₋₆ cycloalkoxy.

47. The compound of claim 29, or a pharmaceutically acceptable salt or ester thereof, wherein D is characterized as having a structure according to Formula D-1-A-1, D-1-A-2, D-1-A-3, D-1-A-4, or D-1-A-5: 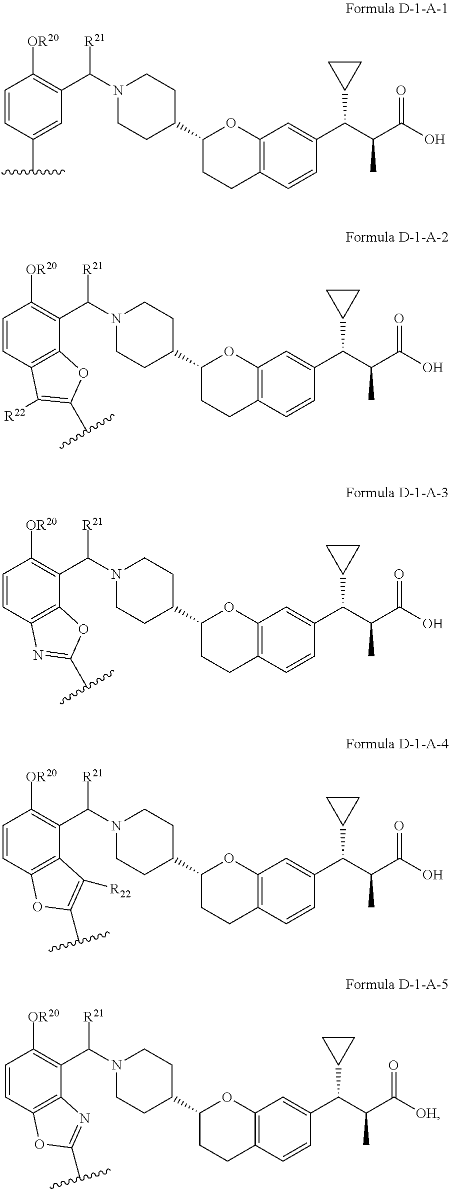
wherein:
R²⁰ is C₁₋₆ alkyl or fluorine substituted C₁₋₆ alkyl, R²¹ is hydrogen or C₁₋₆ alkyl, and R²² is hydrogen, halogen, CN, C₁₋₆ alkyl or fluorine substituted C₁₋₆ alkyl or a C₃₋₆ cycloalkyl.

48. The compound of claim 29, or a pharmaceutically acceptable salt or ester thereof, wherein D is characterized as having a structure according to Formula D-1-A-6, D-1-A-7, D-1-A-8, D-1-A-9, or D-1-A-10: 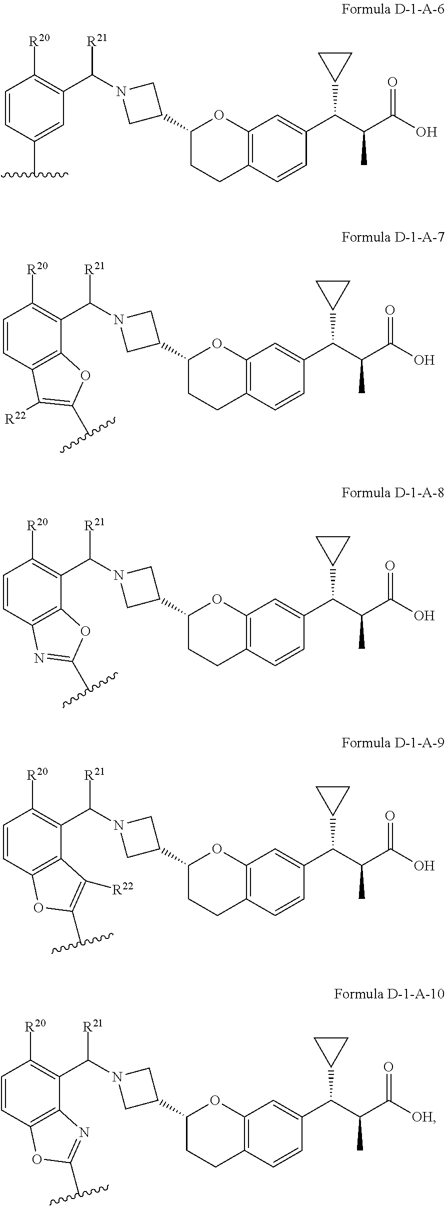
wherein:
R²⁰ is C₁₋₆ alkyl or fluorine substituted C₁₋₆ alkyl, R²¹ is hydrogen or C₁₋₆ alkyl, and R²² is hydrogen, halogen, CN, C₁₋₆ alkyl or fluorine substituted C₁₋₆ alkyl or a C₃₋₆ cycloalkyl.

49. The compound of any of claims 1-28, or a pharmaceutically acceptable salt or ester thereof, wherein D is a residue having the formula of D-2-A or D-2-B: 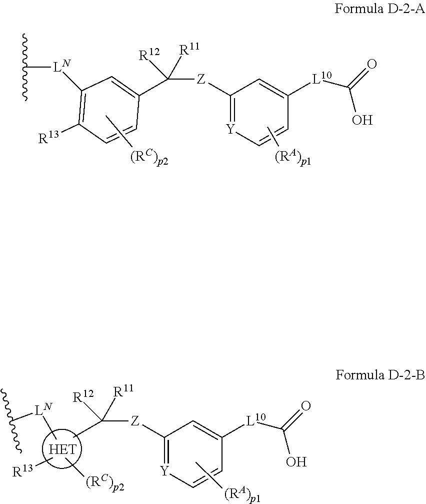
wherein:
Y is CH, CR^{A}, or N;
Z is O, S, NH, or N(C₁₋₄ alkyl);
HET ring stands for an optionally substituted heteroaryl ring (e.g., a 5 or 6-membered heteroaryl, such as a triazole ring);
R¹¹ and R¹² are each independently hydrogen or C₁₋₄ alkyl;
L^{N} is null, an optionally substituted C₁₋₆ alkylene, or an optionally substituted C₁₋₆ heteroalkylene having 1-3 heteroatoms;
L¹⁰ is an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{C} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two RC are joined to form an optionally substituted ring structure; p2 is 0, 1, 2, or 3; and
R¹³ is hydrogen, an optionally substituted phenyl or an optionally substituted heteroaryl.

50. The compound of claim 49, or a pharmaceutically acceptable salt or ester thereof, wherein D is a residue having the formula of D-2-A-1 or D-2-B-1:

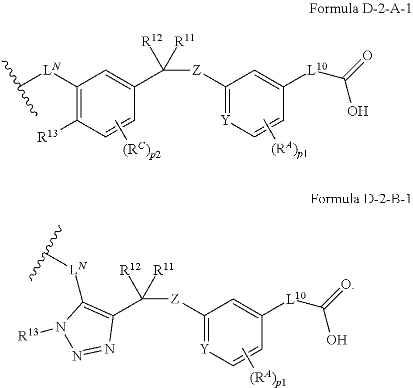

51. The compound of claim 49 or 50, or a pharmaceutically acceptable salt or ester thereof, wherein L^{N} is (i) null; (ii) a branched or straight chained C₁₋₆ alkylene, such as 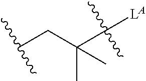
(L^{A} is shown to show direction of connection); or (iii) a branched or straight chained C₁₋₆ heteroalkylene having one or two oxygen atoms, such as 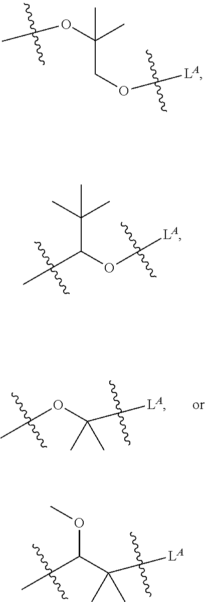
(L^{A} is shown to show direction of connection);

52. The compound of claim 49 or 50, or a pharmaceutically acceptable salt or ester thereof, wherein L^{N} has a structure of 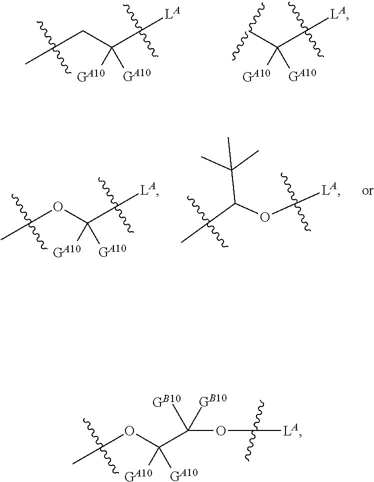
wherein G^{A10} at each occurrence is independently hydrogen or an optionally substituted C₁₋₄ alkyl, or two G^{A10} are joined to form a 3-6 membered ring, such as a cyclopropyl or cyclobutyl ring, preferably, G^{A10} at each occurrence is methyl; wherein G^{B10} at each occurrence is independently hydrogen or an optionally substituted C₁₋₄ alkyl, or two G^{B10} or one G^{A10} and one G^{B10} are joined to form a 3-6 membered ring, such as a cyclopropyl or cyclobutyl ring.

53. The compound of any of claims 49-52, or a pharmaceutically acceptable salt or ester thereof, wherein p1 is 0, or p1 is 1, and R^{A} at each occurrence is independently F, Cl, CN, C₁₋₄ alkyl optionally substituted with 1-3 fluorine, or C₁₋₄ alkoxy optionally substituted with 1-3 fluorine.

54. The compound of any of claims 49-53, or a pharmaceutically acceptable salt or ester thereof, wherein p2 is 0.

55. The compound of any of claims 49-53, or a pharmaceutically acceptable salt or ester thereof, wherein p2 is 1, and R^{C} is F, Cl, CN, C₁₋₄ alkyl optionally substituted with 1-3 fluorine, or C₁₋₄ alkoxy optionally substituted with 1-3 fluorine.

56. The compound of any of claims 49-55, or a pharmaceutically acceptable salt or ester thereof, wherein Y is CH.

57. The compound of any of claims 49-56, or a pharmaceutically acceptable salt or ester thereof, wherein Z is O.

58. The compound of any of claims 49-57, or a pharmaceutically acceptable salt or ester thereof, wherein R¹¹ and R¹² are both hydrogen.

59. The compound of any of claims 49-58, or a pharmaceutically acceptable salt or ester thereof, wherein L¹⁰ is 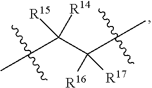
wherein CR¹⁰R¹⁷ is bonded to the COOH group, and wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S.

60. The compound of any of claims 49-59, or a pharmaceutically acceptable salt or ester thereof, wherein R¹³ is a phenyl ring, which is unsubstituted or substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

61. The compound of any of claims 49-59, or a pharmaceutically acceptable salt or ester thereof, wherein R¹³ is a 6-membered heteroaryl ring, such as a pyridyl ring, which is unsubstituted or substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

62. The compound of any of claims 49-52, or a pharmaceutically acceptable salt or ester thereof, wherein D has a structure according to Formula D-2-A-2 or Formula D-2-B-2: 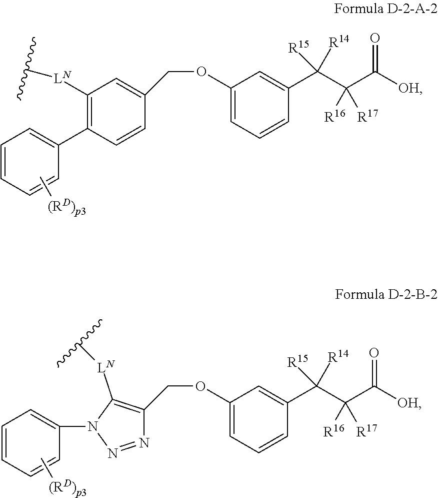
wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S;
R^{D} at each occurrence is independently F, Cl, C₁₋₄ alkyl optionally substituted with 1-3 F, or C₁₋₄ alkoxy optionally substituted with 1-3 F, and
wherein p3 is 0, 1, 2, or 3.

63. The compound of claim 62, or a pharmaceutically acceptable salt or ester thereof, wherein R¹⁶ and R¹⁷ are both hydrogen, or one of R¹⁶ and R¹⁷ is hydrogen and the other of R¹⁶ and R¹⁷ is methyl.

64. The compound of claim 62 or 63, or a pharmaceutically acceptable salt or ester thereof, wherein one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl.

65. The compound of claim 62 or 63, or a pharmaceutically acceptable salt or ester thereof, wherein R¹⁴ and R¹⁵ are joined to form a C₃₋₆ cycloalkyl.

66. The compound of any of claims 49-52, or a pharmaceutically acceptable salt or ester thereof, wherein D has a structure according to Formula D-2-A-3 or D-2-B-3:

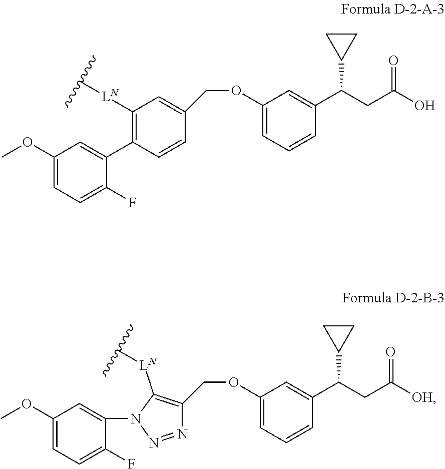

67. The compound of any of claims 1-28, or a pharmaceutically acceptable salt or ester thereof, wherein D has a structure according to Formula D-3-A or D-3-B: 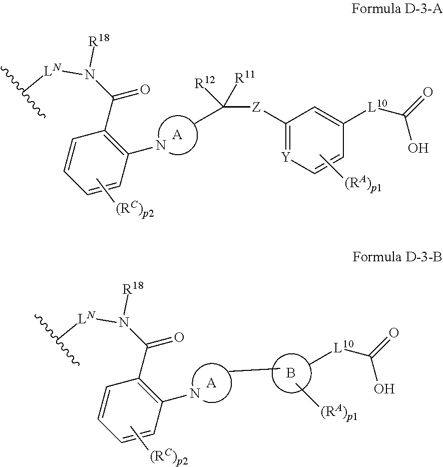
wherein:
Y is CH, CR^{A}, or N;
Z is O, S, NH, or N(C₁₋₄ alkyl);
R¹¹ and R¹² are each independently hydrogen or C₁₋₄ alkyl;
Ring A is an optionally substituted 4-12 membered nitrogen-containing ring;
Ring B is an optionally substituted monocyclic heteroaryl or a bicyclic aryl or heteroaryl ring, such as a benzofuran ring;
L^{N} is null, an optionally substituted C₁₋₆ alkylene, or an optionally substituted C₁₋₆ heteroalkylene having 1-3 heteroatoms;
L¹⁰ is an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{C} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two RC are joined to form an optionally substituted ring structure; p2 is 0, 1, 2, or 3; and
R¹⁸ is an optionally substituted phenyl or an optionally substituted heteroaryl.

68. The compound of claim 67, or a pharmaceutically acceptable salt or ester thereof, wherein D has a structure according to Formula D-3-A-1 or D-3-B-1:

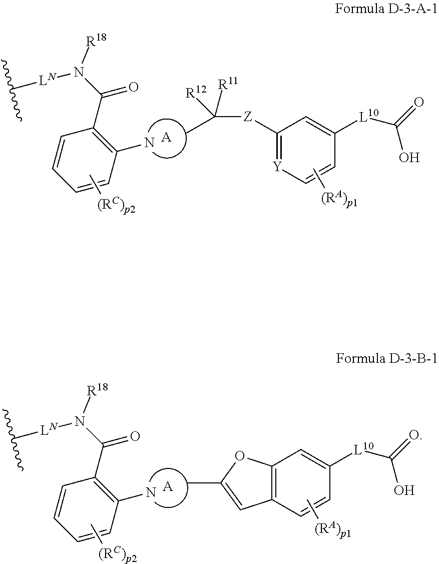

69. The compound of claim 67 or 68, or a pharmaceutically acceptable salt or ester thereof, wherein L^{N} is (i) null; or (ii) a branched or straight chained C₁₋₆ alkylene, such as 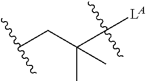
(L^{A} is shown to show direction of connection).

70. The compound of claim 67 or 68, or a pharmaceutically acceptable salt or ester thereof, wherein L^{N} has a structure of 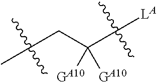
wherein G^{A10} at each occurrence is independently hydrogen or an optionally substituted C₁₋₄ alkyl, or two G^{A10} are joined to form a 3-6 membered ring, such as a cyclopropyl or cyclobutyl ring, preferably, G^{A10} at each occurrence is methyl.

71. The compound of any of claims 67-70, or a pharmaceutically acceptable salt or ester thereof, wherein p1 is 0 or p1 is 1, and R^{A} at each occurrence is independently F, Cl, CN, C₁₋₄ alkyl optionally substituted with 1-3 fluorine, or C₁₋₄ alkoxy optionally substituted with 1-3 fluorine.

72. The compound of any of claims 67-71, or a pharmaceutically acceptable salt or ester thereof, wherein p2 is 0.

73. The compound of any of claims 67-72, or a pharmaceutically acceptable salt or ester thereof, wherein p2 is 1, and R^{C} is F, Cl, CN, C₁₋₄ alkyl optionally substituted with 1-3 fluorine, or C₁₋₄ alkoxy optionally substituted with 1-3 fluorine.

74. The compound of any of claims 67-73, or a pharmaceutically acceptable salt or ester thereof, wherein Y is N.

75. The compound of any of claims 67-74, or a pharmaceutically acceptable salt or ester thereof, wherein Z is O.

76. The compound of any of claims 67-75, or a pharmaceutically acceptable salt or ester thereof, wherein R″ and R¹² are both hydrogen.

77. The compound of any of claims 67-76, or a pharmaceutically acceptable salt or ester thereof, wherein L¹⁰ is 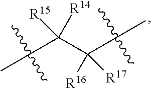
wherein CR¹⁶R¹⁷ is bonded to the COOH group, and wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S.

78. The compound of any of claims 67-77, or a pharmaceutically acceptable salt or ester thereof, wherein R¹⁸ is a 6-membered heteroaryl ring, e.g., 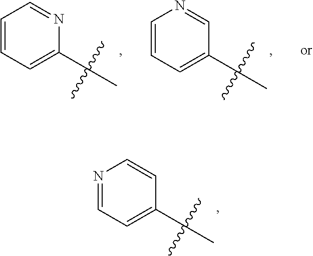 [SMILES: *c1ccccn1 ; *c1cccnc1 ; *c1ccncc1]
which is optionally substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

79. The compound of any of claims 67-78, or a pharmaceutically acceptable salt or ester thereof, wherein Ring A is a 4-8 membered optionally substituted monocyclic saturated heterocyclic ring having one or two ring heteroatoms independently selected from S, O, and N, provided at least one of the ring heteroatom is nitrogen.

80. The compound of claim 79, or a pharmaceutically acceptable salt or ester thereof, wherein Ring A is selected from: 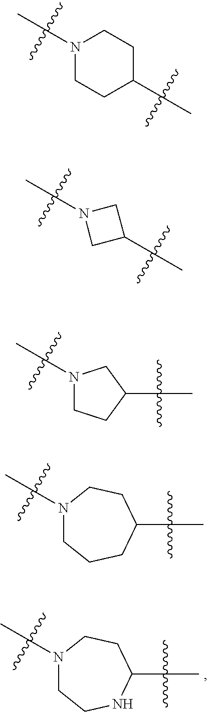 [SMILES: *C1CCN(*)CC1 ; *C1CN(*)C1 ; *C1CCN(*)C1 ; *C1CCCN(*)CC1 ; *C1CCN(*)CCN1]
each of which is optionally substituted with 1-2 substituents independently selected from F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

81. The compound of any of claims 67-78, or a pharmaceutically acceptable salt or ester thereof, wherein Ring A is bicyclic or polycyclic 6-12 membered optionally substituted saturated heterocyclic ring having one or two ring heteroatoms independently selected from S, O, and N, provided at least one of the ring heteroatom is nitrogen.

82. The compound of any of claims 67-79, or a pharmaceutically acceptable salt or ester thereof, which D has a structure according to Formula D-3-A-2 or D-3-B-2: 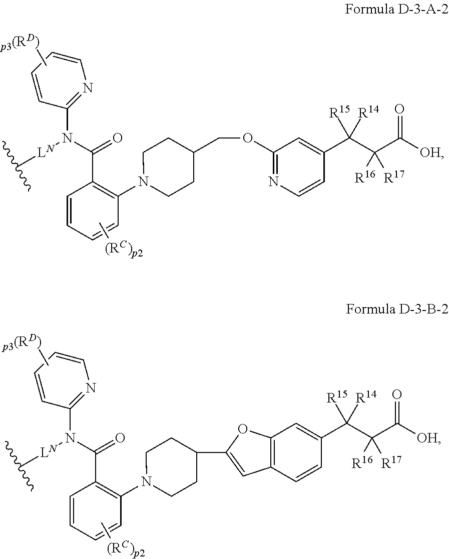
wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S;
R^{D} at each occurrence is independently F, Cl, C₁₋₄ alkyl optionally substituted with 1-3 F, or C₁₋₄ alkoxy optionally substituted with 1-3 F, and
wherein p3 is 0, 1, 2, or 3.

83. The compound of claim 82, or a pharmaceutically acceptable salt or ester thereof, wherein R¹⁶ and R¹⁷ are both hydrogen, or one of R¹⁶ and R¹⁷ is hydrogen and the other of R¹⁶ and R¹⁷ is methyl.

84. The compound of claim 82 or 83, or a pharmaceutically acceptable salt or ester thereof, wherein one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl.

85. The compound of claim 82 or 83, or a pharmaceutically acceptable salt or ester thereof, wherein R¹⁴ and R¹⁵ are joined to form a C₃₋₆ cycloalkyl.

86. The compound of any of claims 67-70, or a pharmaceutically acceptable salt or ester thereof, wherein D has a structure according to Formula D-3-A-3 or D-3-B-3:

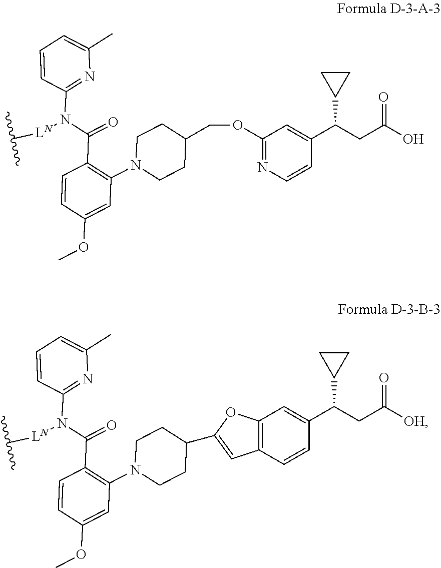

87. A compound selected from any of the compounds in Table 1 herein, or a compound according to Examples 1-36 herein, or a pharmaceutically acceptable salt or ester thereof.

88. A pharmaceutical composition comprising the compound of any of claims 1-87 or a pharmaceutically acceptable salt or ester thereof and optionally a pharmaceutically acceptable carrier.

89. A method of treating or preventing a disorder, condition or disease that may be responsive to the agonism of the G-protein-coupled receptor 40 in a subject in need thereof comprising administration of a therapeutically effective amount of the compound of any of claims 1-87 or a pharmaceutically acceptable salt or ester thereof, or the pharmaceutical composition of claim 88.

90. A method of treating type 2 diabetes mellitus in a subject in need of treatment comprising administering to the subject a therapeutically effective amount of the compound of any of claims 1-87 or a pharmaceutically acceptable salt or ester thereof, or the pharmaceutical composition of claim 88.

91. The method of claim 89 or 90, further comprising administering to the subject one or more additional therapeutic agents.

92. The method of claim 91, wherein the one or more additional therapeutic agents are selected from PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; α-glucosidase inhibitors; agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARα agonists, (v) cholesterol absorption inhibitors, (vi) acyl CoA:cholesterol acyltransferase (ACAT) inhibitors, (vii) CETP inhibitors, (viii) PCSK9 inhibitor or antibodies; (ix) apolipoproteins inhibitors; (x) phenolic anti-oxidants; PPARα/γ dual agonists; PPARδ agonists; PPAR α/δ partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists (peptide and small-molecule); GLP-1/GIP receptor dual agonists; GLP-1/glucagon receptor dual agonists; GLP-1/GIP/insulin receptor triple agonists; GLP-1/GIP/glucagon receptor triple agonists; GIP receptor antibody; GLP-1 analog/GIP receptor antibody; PYY analog; amylin analogs; GPR119 agonist; TGR5 agonist; SSTR2 and/or SSTR5 antagonist or inverse agonist; THRβ agonists; HSD-1 inhibitors; HSD-17 inhibitors and degraders; PNPLA3 inhibitors and degraders; SGLT-2 inhibitors; SGLT-1/SGLT-2 inhibitors; enteric alpha-glucosidase inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin; (xi) anti-amyloid beta antibody; (xii) anti-inflammatory agents including but not limited to PDE4 inhibitors, JAK inhibitors, TYK2 inhibitors, SIP receptor modulators, NLRP3 inhibitors, BTK inhibitors, IRAK1 inhibitors, IRAK4 inhibitors, glucocorticoids, anti-TNFα antibodies, anti-IL-12/IL-23 antibodies, (xiii) anti-integrin antibodies or small-molecule inhibitors of integrins including α4β7, α4, β7, MAdCAM-1, αvβ6 and αvβ1.
